(19) United States
(12) Patent Application Publication
(10) Pub. No.: US 2025/0122482 A1
(43) Pub. Date: Apr. 17, 2025

(54) PRODUCTION AND PURIFICATION OF COVALENTLY SURFACE MODIFIED ADENO-ASSOCIATED VIRUS

(71) Applicant: Regeneron Pharmaceuticals, Inc., Tarrytown, NY (US)

(72) Inventors: Garima Thakur, White Plains, NY (US); Zhe Zhang, Tarrytown, NY (US); Sheldon Robert Mink, Hawthorne, NY (US); Andrew David Tustian, Millwood, NY (US)

(21) Appl. No.: 18/827,121

(22) Filed: Sep. 6, 2024

Related U.S. Application Data
(60) Provisional application No. 63/657,304, filed on Jun. 7, 2024.
(60) Provisional application No. 63/654,328, filed on May 31, 2024.
(60) Provisional application No. 63/620,585, filed on Jan. 12, 2024.
(60) Provisional application No. 63/620,002, filed on Jan. 11, 2024.
(60) Provisional application No. 63/537,170, filed on Sep. 7, 2023.

(51) Int. Cl. C12N 7/02; C07K 16/46; G01N 33/50

(52) U.S. Cl. CPC C12N 7/02; C07K 16/46; G01N 33/5008; C07K 2319/30; C12N 2710/10022; C12N 2750/14122; C12N 2750/14143; C12N 2750/14151

(57) ABSTRACT

The present inventions provide covalently surface modified adeno-associated viruses can comprise gene of interest (GOIs) and advantageously can be targeted to certain cell and tissue types for preventative and therapeutic purposes. The present inventions further provide systems and methods for engineering adeno-associated virus (AAV) to create covalently surface modified adeno-associated viruses, and methods of purifying such covalently surface modified adeno-associated viruses. The inventions further include covalently surface modified adeno-associated viruses and preparations and products comprising such covalently surface modified adeno-associated viruses.

This application claims priority to U.S. Application Ser. No. 63/657,304, filed Jun. 7, 2024; U.S. Application Ser. No. 63/654,328, filed May 31, 2024; U.S. Application Ser. No. 63/620,585, filed Jan. 12, 2024; U.S. Application Ser. No. 63/620,002, filed Jan. 11, 2024; and U.S. Application Ser. No. 63/537,170, filed Sep. 7, 2023. These applications are incorporated by reference in their entirety.

### FIELD OF THE INVENTIONS

Recombinant AAV of any serotype (rAAV) containing gene(s) of interest (GOI) are increasingly being used in preventative and therapeutic capacities, such as in vaccines and in gene therapy. The present inventions provide systems and methods for engineering adeno-associated virus (AAV) to produce covalently surface modified adeno-associated viruses, and methods of purifying such covalently surface modified adeno-associated viruses. The inventions further include covalently surface modified adeno-associated viruses and preparations and products comprising such covalently surface modified adeno-associated viruses, and improved approaches for purification. Covalently surface modified adeno-associated viruses can comprise a gene of interest (GOI) and advantageously can be targeted to certain cell and tissue types for preventative and therapeutic purposes, according to the inventions described herein.

### REFERENCE TO ELECTRONIC SEQUENCE LISTING

The application contains a Sequence Listing which has been submitted electronically in .XML format and is hereby incorporated by reference in its entirety. This .XML copy, created on Aug. 27, 2024, is named “11546.xml” and is 400,620 bytes in size. The sequence listing contained in this .XML file is part of the specification and is incorporated by reference in its entirety.

### BACKGROUND OF THE INVENTIONS

Adeno-associated virus (AAV) is a non-enveloped, single-stranded DNA virus and is used as a gene delivery vector for both research and therapeutics. Weitzman and Linden, Adeno-Associated Virus Biology (chapter 1), Meth. Molec. Biol. 807: 1-23 (2011). There are numerous AAV serotypes and variants thereof. AAV serotypes include, for example, AAV1, 2, 3, 4, 5, 6, 7, 8, 9, 10, 11, 12, 13, rh74 as well as variants thereof. AAV serotypes share common properties, structure, and genomic sequence and organization. See, for example, Issa et al, Cells 12: 285 (2023); Goedeker et al., Ther. Adv. Neurol. Disord. 16: 1-7 (2023).

Gene transfer vectors based on AAV have demonstrated promise for human gene therapy based on their safety profile and potential to achieve long-term efficacy in animal models. Wang et al., Nature, 18: 358-78 (2019). Recombinant AAVs (rAAVs) containing genes of interest (GOIs) are increasingly being used in preventative and therapeutic capacities, such as in vaccines and in gene therapy. Additionally, recombinant AAVs containing genes of interest (GOIs), but lacking surface modifications, are currently being used in preventative and therapeutic capacities, such as in vaccines and in gene therapy. However, such AAVs have limited tissue specificity and a propensity to accumulate in the liver.

The wild type AAV genome includes a capsid gene referred to as “Cap” or “cap”. Cap in nature is translated to produce, via alternative start codons and transcript splicing, three size-variant structural proteins referred to as VP1 (about 90 kDa), VP2 (about 72 kDa) and VP3 (about 60 kDa). An AAV capsid contains 60 subunits total of the VP proteins. A ratio of 1:1:10 is considered the most typical ratio for VP1:VP2:VP3, with a stoichiometry of 5 VP1 subunits:5 VP2 subunits:50 VP3 subunits. However, there can be variation. Wörner et al., Nature Communications 12:1642 (2021).

Recombinant AAV (rAAV) has been produced in HEK 293, BHK, human amniotic (for example, epithelial cells such as HAEpiC), CHO, HeLa and Sf9 lines. First generation rAAV was comprised of a GOI replacing the AAV Cap and Rep genes. The GOI would be flanked by AAV inverted terminal repeats (ITRs) so that the GOI could be packaged within an AAV capsid.

Different AAV serotypes are known to preferentially transduce different tissues. Tissue specificity is limited, and AAV is known to preferentially transduce the liver, which can be a safety and efficacy concern. In view of these natural limitations, there exist needs to vary the targeting of rAAVs to control target specificity, including at least lessening the targeting of the liver. These goals were not met until the present inventions.

The production of covalently surface modified AAV requires the production of both the AAV vector and the retargeting surface modification. Producing the two entities separately requires separate production vessels and purification trains, which can be resource demanding and labor intensive. The present invention provides methods to simultaneously produce all necessary covalently surface modified AAV components within the same producing cell line, for example, in a suspension cultured fed-batch production bioreactor. A serum free suspension platform has been widely adopted in traditional biologics, and is increasing in popularity for cell and gene therapy industry. It allows increased safety and consistency from batch to batch, and process scale up to 1 kL scale or greater.

Depth filtration is used for the purification of AAVs. In current manufacturing processes for rAAV, expensive endonuclease treatment is used prior to depth filtration to break down large amounts of chromatin post-cellular lysis. Without this treatment, traditional depth filters tend to foul rapidly, making clarification impractical without the use of excessive filter area. The present inventions provide for improvements in depth filtration to enhance the efficiency and lower costs of rAAV purification.

Purification of surface-modified AAVs also requires effective methods to capture surface-modified AAVs from the bioreactor harvest mixture. Challenges in affinity capture include difficulty in binding to the AAV surface epitopes in case of heavy conjugation, and difficulty in eluting the surface-modified AAV without destabilizing the molecule leading to aggregation, precipitation, and/or fragmentation. These difficulties often lead to an imbalance in the level of conjugation in the affinity capture load material relative to the eluate stream; that is, a loss of the some or all of the conjugated species during affinity capture due to preferential purification of the less conjugated/unconjugated AAVs. The goal of high-yield affinity capture of conjugated AAV species was not met until the present inventions.

### SUMMARY OF THE INVENTIONS

Many potential gene therapy applications would benefit from cell/tissue-specific retargeting and liver detargeting that is beyond current AAV vector control capabilities. The current inventions advantageously employ covalently surface modified AAV for retargeting. Additionally, according to the inventions, mutations can be introduced into the AAV Cap proteins to detarget the liver.

Genetic components encoding for the covalently surface modified AAV can be introduced via transient transfection of six plasmids into cells, referred to as a “hexad transfection.” The AAV vector and surface modification components interact and form covalent bonds at the end of production after cell lysis. The plasmid concentration and ratios used during the hexad transfection are optimized using multivariate design of experiment (DOE) approaches to achieve high AAV titer and fast conjugation reaction rates. The hexad transfection provides all the necessary components, namely AAV rep and cap functional and structural proteins, helper polynucleotide sequences, retargeting molecule components (such as antibody heavy chain and light chains), sequences encoding specific binding pairs, genes of interest, and detargeting mutations (if desired). One or more retargeting molecules can bind to one or more targets.

The inventions further provide methods of purifying the covalently surface modified AAV of the inventions described herein using approaches including improved depth filtration, improved tangential flow filtration, improved affinity capture and improved ionic exchange chromatography. Depth filtration can be employed with lowered or without endonuclease treatment. Affinity capture with optimized loading and elution conditions to achieve high yield without loss of heavily conjugated species.

Modulation of the level of surface modification is demonstrated through the introduction of different plasmid ratios during the transient hexad transfection. Optimal level of AAV surface modification is identified to improve tissue specificity and transduction efficiency.

The inventions provide methods of producing a covalently surface modified adeno-associated viruses (AAV), wherein the methods comprise the steps of:
(A) transfecting a cell with: (i) a plasmid comprising a gene of interest flanked by AAV inverted terminal repeats; (ii) a plasmid comprising an AAV rep gene and an AAV cap gene; (iii) a plasmid AAV rep and cap genes and a polynucleotide sequence encoding a first member of a specific binding pair (“first member”); (iv) a plasmid comprising one or more helper polynucleotide sequences; (v) a plasmid comprising a polynucleotide sequence encoding a first portion of a retargeting molecule and a polynucleotide encoding a second cognate member of the specific binding pair (“second member”); and (vi) a plasmid comprising a polynucleotide encoding a second portion of a retargeting molecule; (B) culturing the transfected cell to allow expression of plasmids (i) to (vi) and assembly of components to form covalently surface modified adeno-associated virus; and (C) harvesting the covalently surface modified adeno-associated virus. The cells can be mammalian cells, such as human and rodent cells. Insect cells also can be used. The cells can be a mammalian cell, such as a human cell, for example HEK293 (both adherent and suspension), HeLa and amniotic cells.

The retargeting molecules can be Fc-containing proteins having, such as monoclonal antibodies, which have portions. For example, the first portion of the retargeting molecule can be an antibody heavy chain, and the second portion of the retargeting molecule is an antibody light chain. Optionally, the first portion of the retargeting molecule can be an antibody light chain, and the second portion of the retargeting molecule can be an antibody heavy chain.

The specific binding pair can be SpyTag-SpyCatcher, for example. In such a case, the first member of a specific binding pair can be a SpyTag peptide For example, the polynucleotide encoding the SpyTag peptide can be inserted into the AAV cap gene to encode recombinant capsid proteins. The second cognate member of the specific binding pair can be a SpyCatcher protein, for example.

The methods also can include having the cells express at least one recombinant capsid protein selected from the group consisting of a recombinant VP1 protein comprising a SpyTag amino acid sequence, a recombinant VP2 protein comprising a SpyTag amino acid sequence, and a recombinant VP3 protein comprising a SpyTag amino acid sequence. Additionally, the VP1 protein can be mutated in a galactose binding domain to detarget liver cells, such as a detargeting mutation is at least one selected from the group consisting of N272A and W503A, for example. Other detargeting mutations can be employed as well, including but not limited to those disclosed herein.

The methods also provide for a covalently surface modified adeno-associated virus comprising a plurality of first members bound to second cognate members, wherein the second cognate members also are bound to retargeting molecules; and a gene of interest. The first members can be SpyTag peptides and second cognate members at SpyCatcher proteins. The retargeting molecules can be antibodies, antibody fragments or antibody derivatives.

Covalently surface modified adeno-associated viruses can be provided according to the inventions by having the cells express at least one recombinant capsid protein selected from the group consisting of a recombinant VP1 protein comprising a SpyTag amino acid sequence, a recombinant VP2 protein comprising a SpyTag amino acid sequence, and a recombinant VP3 protein comprising a SpyTag amino acid sequence. Additionally, the VP1 protein can be mutated in a galactose binding domain to detarget liver cells, such as a detargeting mutation is at least one selected from the group consisting of N272A and W503A, including but not limited to those disclosed herein.

The inventions further provide covalently surface modified adeno-associated virus comprising a plurality of first members bound to second cognate members, wherein the second cognate members also are bound to retargeting molecules, and a gene of interest, wherein the covalently surface modified adeno-associated virus is made by a method comprising the steps of: A) transfecting a cell with: (i) a plasmid comprising a gene of interest flanked by AAV inverted terminal repeats; (ii) a plasmid comprising an AAV rep gene and an AAV cap gene; (iii) a plasmid comprising AAV rep and cap genes and a polynucleotide sequence encoding a first member of a specific binding pair; (iv) a plasmid comprising one or more helper polynucleotide sequences; (v) a plasmid comprising a polynucleotide sequence encoding a first portion of a retargeting molecule and a polynucleotide encoding a second cognate member of the specific binding pair; and (vi) a plasmid comprising a polynucleotide encoding a second portion of a retargeting molecule; (B) culturing the transfected cell to allow expression of plasmids (i) to (vi) and assembly of proteins to form covalently surface modified adeno-associated virus; and (C) harvesting the covalently surface modified adeno-associated virus.

The retargeting molecules can be Fc-containing proteins having, such as monoclonal antibodies, which have portions. For example, the first portion of the retargeting molecule can be an antibody heavy chain, and the second portion of the retargeting molecule is an antibody light chain. Optionally, the first portion of the retargeting molecule can be an antibody light chain, and the second portion of the retargeting molecule can be an antibody heavy chain.

The specific binding pair can be SpyTag-SpyCatcher, for example. In such a case, the first member of a specific binding pair can be a SpyTag peptide For example, the polynucleotide encoding the SpyTag peptide can be inserted into the AAV cap gene to encode recombinant capsid proteins. The second cognate member of the specific binding pair can be a SpyCatcher protein, for example.

The cells can be a mammalian cell, such as a human cell, for example HEK293 (both adherent and suspension), HeLa and amniotic cells.

The covalently surface modified adeno-associated viruses can comprise a plurality of first members bound to second cognate members, wherein the second cognate members also are bound to retargeting molecules; and a gene of interest. The first members can be SpyTag peptides and second cognate members at SpyCatcher proteins. The retargeting molecules can be antibodies, antibody fragments or antibody derivatives. During production, covalently surface modified adeno-associated viruses can be subjected to the step of (D) purifying the covalently surface modified adeno-associated virus using depth filtration. The depth filtration can be performed without an endonuclease. During production, covalently surface modified adeno-associated viruses can be subjected to the step of comprise the step of (E) purifying the covalently surface modified adeno-associated viruses using affinity chromatography.

The covalently surface modified adeno-associated virus can comprise a plurality of first members bound to second cognate members, wherein the second cognate members also are bound to retargeting molecules; and a gene of interest. The first members can be SpyTag peptides and second cognate members can be SpyCatcher proteins. The retargeting molecules can be antibodies, antibody fragments or antibody derivatives. The helper polynucleotide sequences can encode adenovirus E4, adenovirus E2, and VA RNA, for example, or be from another virus, such as herpes simplex virus (UL5, UL8, UL9, UL29, UL30, UL42 and UL52 polynucleotides), human papilloma virus (E1 or E1 carboxyl domain polynucleotides), bocavirus or baculovoirus.

The inventions also provide methods of screening retargeting molecules for production of a covalently surface modified adeno-associated virus species, wherein the method comprises the steps of: (I) providing (A) a first plurality of nucleic acids encoding retargeting molecules that are different from one another and (B) a second plurality of DNA barcodes that are different from one another, wherein each individual DNA barcode of the second plurality is assigned to an individual covalently surface modified adeno-associated virus comprising a retargeting molecule of the first plurality for creating a covalently surface modified adeno-associated virus species; (II) producing covalently surface modified adeno-associated virus species by (A) transfecting a cell with: (i) a plasmid comprising a polynucleotide that comprises a DNA barcode, wherein the polynucleotide that comprises the DNA barcode is flanked by AAV inverted terminal repeats; (ii) a plasmid comprising an AAV rep gene and an AAV cap gene; (iii) a plasmid comprising AAV rep and cap genes and a polynucleotide sequence encoding a first member of a specific binding pair; (iv) a plasmid comprising one or more helper polynucleotide sequences; (v) a plasmid comprising a polynucleotide sequence encoding a first portion of a retargeting molecule of the first plurality and a polynucleotide encoding a second cognate member of the specific binding pair; and (vi) a plasmid comprising a polynucleotide encoding a second portion of a retargeting molecule of the first plurality; (B) culturing the transfected cell to allow expression of plasmids (i) to (vi) and assembly of components to form a covalently surface modified adeno-associated virus species; (C) harvesting the covalently surface modified adeno-associated virus species, wherein the covalently surface modified adeno-associated virus species will become part of a covalently surface modified adeno-associated virus library; (D) repeating steps (A) to (C) to produce each covalently surface modified adeno-associated virus species to form the covalently surface modified adeno-associated viruses library; and (E) detecting each DNA barcode of the second plurality in order to screen each species to determine a property.

The AAV library produced can be used to dose model animals (for example, rodents or non-human primates), the target tissue is then extracted and sequenced to quantify enrichment of the DNA barcodes. The distribution of barcodes in different body parts/tissues can be assessed to see which covalently surface modified adeno-associated virus species are best at delivering GOI payloads (containing barcodes) to a specific body part, preventing the delivery of excess GOI payloads to unwanted body parts such as the liver and/or characterizing genomic titer.

The retargeting molecule can be an Fc-containing protein, such as a monoclonal antibody, including multispecific antibodies, such as bispecific and trispecific antibodies. The Fc-containing protein can be an Fc-fusion protein, such as a receptor-Fc-fusion protein, which includes trap proteins. The retargeting molecule also can be selected from the group consisting of an fab, f(ab′), f(Ab′)2, a single chain antibody and a mini-trap protein, for example. The helper polynucleotide sequences can encode adenovirus E4, adenovirus E2, and VA RNA, for example. Other viruses can be the source of helper polynucleotide sequences encoding helper products, such as herpes simplex virus (UL5, UL8, UL9, UL29, UL30, UL42 and UL52 polynucleotides), human papilloma virus (E1 or E1 carboxyl domain polynucleotides), bocavirus or baculovoirus.

The inventions further provide methods of screening covalently surface modified adeno-associated virus species, wherein the method comprises the steps of: (I) providing (A) a first plurality of nucleic acids encoding a component of a covalently surface modified adeno-associated virus species (component), wherein each component of the first plurality are different from one another and (B) a second plurality of DNA barcodes that are different from one another, wherein each individual DNA barcode of the second plurality is assigned to an individual component of the first plurality for creating a covalently surface modified adeno-associated virus species; (II) producing covalently surface modified adeno-associated virus species by (A) transfecting a cell with: (i) a plasmid comprising a polynucleotide that comprises a DNA barcode, wherein the polynucleotide that comprises the DNA barcode is flanked by AAV inverted terminal repeats; (ii) a plasmid comprising an AAV rep gene and an AAV cap gene; (iii) a plasmid comprising AAV rep and cap genes and a polynucleotide sequence encoding a first member of a specific binding pair; (iv) a plasmid comprising one or more helper polynucleotide sequences; (v) a plasmid comprising a polynucleotide sequence encoding a first portion of a retargeting molecule and a polynucleotide encoding a second cognate member of the specific binding pair; and (vi) a plasmid comprising a polynucleotide encoding a second portion of a retargeting molecule; (B) culturing the transfected cell to allow expression of plasmids (i) to (vi) and assembly of components to form a covalently surface modified adeno-associated virus species; (C) harvesting the covalently surface modified adeno-associated virus species, wherein the covalently surface modified adeno-associated virus species will become part of a covalently surface modified adeno-associated virus library; (D) repeating steps (A) to (C) to produce each covalently surface modified adeno-associated virus species to form the covalently surface modified adeno-associated viruses library; and (E) detecting each DNA barcode of the second plurality in order to screen each species to determine a property. The component can be selected from the group consisting of a retargeting molecule, an AAV cap gene, an AAV rep gene, an ITR, a helper polynucleotide sequence(s), a first member of a specific binding pair, and a second cognate member of a specific binding pair. The helper sequences can encode adenovirus E4, adenovirus E2, and VA RNA, for example. Other viruses can be the source of helper polynucleotide sequences encoding helper products, such as herpes simplex virus (UL5, UL8, UL9, UL29, UL30, UL42 and UL52 polynucleotides), human papilloma virus (E1 or E1 carboxyl domain polynucleotides), bocavirus or baculovoirus.

The inventions further comprise covalently surface modified adeno-associated viruses (AAV) produced and/or purified by the methods.

The inventions also provide AAV preparations produced by any of the above the methods, and drug products made from the AAV preparations.

The inventions are amendable to use with all AAV serotypes and variants, including but not limited to AAV1, AAV2, AAV2quad(Y-F), AAV3, AAV4, AAV5, AAV6, AAV7, AAV8, AAV9, AAV10, AAV11, AAV12, AAV13, rh10, rh39, rh43, rh74,

AAV preparations comprising the covalently surface modified adeno-associated viruses and biologic drug products comprising the covalently surface modified adeno-associated virus also are provided.

### BRIEF DESCRIPTION OF THE FIGURES

FIG. 1 schematically depicts a covalently surface modified AAV bound to a target cells. In FIG. 1, the covalently surface modified AAV comprises a plurality of SpyTag (SpyT) peptide sequences on the capsid surface. The SpyTag amino acid sequences can bind to the SpyCatcher (SpyC) protein. The SpyCatcher protein and retargeting molecule are expressed as a fusion protein, and are later allowed to conjugate with the SpyTag peptide in the AAV capsid. For example, where the retargeting molecule is a monoclonal antibody (mAb), the SpyCatcher peptide sequences a preferably fused at the Fc portion of the mAb. The mAb at the Fv portion recognizes a target on the surface of a target cell, such as a tissue specific surface antigen, to be transduced by the AAV. The AAV can further comprise a GOI (not shown).

FIGS. 2A and 2B schematically depict and describe hexad transfection systems and methods for producing covalently surface modified AAV in a eukaryotic cell, such as HEK 293F. As described in FIG. 2A, the hexad transfection system utilizes six plasmids (schematically depicted) as follows:
(i) pGOI—a plasmid comprising a GOI (for example, a transgene) flanked by two AAV inverted terminal repeats (ITRs), and optionally a selection marker gene, such as an antibiotic resistance gene (for example, ampicillin resistance);
(ii) pRC—a plasmid comprising AAV (here AAV9 is used as an example) rep and cap genes, and optionally a selection marker gene, such as an antibiotic resistance gene (for example, ampicillin resistance);
(iii) pRC-SpyT—a plasmid as above in (ii) and further comprising a polynucleotide sequence encoding a 13 amino acid Spy Tag peptide (a first member if a specific binding pair), and optionally a selection marker gene, such as an antibiotic resistance gene (for example, ampicillin resistance). This is an exemplary plasmid pRC-first member (pRC-FM);
(iv) pHELP—a plasmid comprising adenovirus helper genes E4 and E2, and VA RNA (exemplary helper genes), and a optionally selection marker gene, such as an antibiotic resistance gene (for example, ampicillin resistance);
(v) pHC-SpyC—a plasmid comprising polynucleotides encoding a mAb heavy chain sequence and a SpyCatcher protein sequence (a second cognate member if a specific binding pair), and optionally a first selection marker gene, such as an antibiotic resistance gene (for example, ampicillin resistance). This is an exemplary plasmid pRC-second cognate member (pRC-SCM); and
(vi) pLC—a plasmid comprising a polynucleotide encoding a mAB light chain sequence, and optionally a first selection marker gene, such as an antibiotic resistance gene (for example, ampicillin resistance). Promoters that are functional in eukaryotic cells, such as AAV P5, CMV, lac, CAG, CAGG, and SV40 promoters can be used to initiate transcription, but are not shown. Additionally, internal ribosome entry site (IRES), recombinase recognition sites (RRS), ehancers and operators optionally can be included, but are not shown.

FIG. 2B schematically depicts at the top a pRC-SpyTag plasmid comprising the rep and cap genes and the p40 promoter, and at the bottom schematically shows the insertion of a first member of a specific binding pair, here the SpyTag 13 amino acid peptide sequence as an example, to form a fusion protein comprising SpyTag and Cap peptide sequences, thereby resulting in mutant VP1, VP2 and VP3 proteins fused to the SpyTag peptide sequence. The AAV still possesses the 5:5:50 stoichiometry of the VP1, 2 and 3 proteins.

FIG. 3 schematically depicts a first Design of Experiment for initial optimization of three plasmid transfection prior to the introduction of pRC-SpyT and mAb HC and LC plasmids

FIG. 4 schematically depicts a second Design of Experiment to investigate the impact of bioreactor pH and agitation speed at three different phases of production and the transition timings between phases on vector titer and quality

FIGS. 5A-5C schematically depict a comparison of covalently surface modified AAVs with different detargeting mutations and varying levels of antibody conjugation. FIG. 5A schematically describes and depicts AAVs as follows:
Lane 1—AAV without a detargeting mutation, a SpyTag insertion nor mAb;
Lane 2—AAV without a SpyTag insertion nor mAb conjugation, but has a detargeting mutation (N272A);
Lane 3—AAV without an mAb, but has a SpyTag insertion at some VP proteins and detargeting mutations (N272A or W503A). The fraction of the pRC-SpyT plasmid used during transfection is ⅛ of the total RC plasmids.
Lanes 5 to 10—AAV with a SpyTag insertion at some VP proteins, a detargeting mutation (N272A) and an mAb. The fraction of the pRC-SpyT plasmid used during transfection is ¼, ⅕, 1/6.7, ⅛, 1/10 and 1/20 of the total RC plasmid in the direction of Lane 5 to Lane 10; and
Lanes 11 and 12—AAV with a SpyTag insertion at some VP proteins, a detargeting mutation (W503A) and an mAb. The fraction of the pRC-SpyT plasmid used during transfection is ¼ of the total RC plasmid in Lane 11 and ⅛ in Lane 12.

FIG. 5B shows antibody conjugated capsids only in lanes 5 to 12, and unconjugated capsids in lanes 1 to 12.

FIG. 5C uses green fluorescent protein labeled calcium voltage-gated channel auxiliary subunit gamma 1 (CACNG1) in a transduction assay. CACNG1 was bound by the mAb of the covalently surface modified AAV. FIG. 5C shows the infectivity results of the covalently surface modified (in this case anti-CACNG1 mAb conjugated) AAV with varying levels of antibody conjugation. CACNG1 positive HEK293 cells was dosed with the respective AAV virus preps in each lane, and the intensity of the fluorescent signal indicates infectivity of the AAV virus prep. Lower SpyTag plasmid fractions corresponded to higher infectivity.

FIGS. 6A-6C schematically depict the effect of the SpyCatcher-mAb titer on the conjugation efficiency of the AAV. Fractions at ⅕ and ⅛ of SpyT-RepCap plasmid (pRC-SpyT) were tested. FIG. 6A is a bar graph depicting mAb titers affected by the concentration mAb plasmid DNA used during hexad transfection. FIG. 6B is a bar graph of genomic titers showing that SpyTag AAV titer was not affected by SpyCatcher mAb titer within the range studied. FIG. 6C shows gel electrophoresis of capsids. VP1, VP2 and VP3 of unmodified capsids ran further on the gel than antibody-modified capsids. The data show that increasing the SpyCatcher mAb plasmid DNA led to a more efficient conjugation of AAV.

FIG. 7 is a bar graph schematically depicting data relating to the selection of SpyTag fraction and SpyCatcher mAb DNA concentration. Spy Tag fractions ranged from ⅕ to 1/30. The percent change in mAb DNA concentration ranged 0% to 200% Lane 5, which had a 1/10 SpyTag fraction and a 100% increase in mAb DNA (corresponding to mAb plasmid DNA concentration of 0.56 μg/mL of cell culture during hexad transfection) showed the highest transduction efficiency. The signal was generated using a green fluorescent reporter gene.

FIG. 8 schematically depicts data from a low pH hold used to increase the efficiency of the conjugation reaction.

FIGS. 9A and 9B schematically depict data on filtrate quality analytics for Harvest RC (HRC) and C0SP filtration of rAAV8 (3×10⁶ cells/ml) and rAAV9 (1.5×10⁶ cells/ml). FIG. 9A is a bar graph for AAV8 depicting in order bars for bioreactor lysate (white bars), filtrate pool where transmembrane pressure (TMP) is 5 psi (light gray bars), and filtrate pool where TMP is 15 psi (black bars). HRC (solid bars) and C0SP (a standard filter used as a control) (hatched and labeled bars) are filtration trains with different endonuclease and salt conditions in the load. FIG. 9B is a bar graph for AAV9 depicting in order bars for bioreactor lysate (white bars), filtrate pool where TMP is 5 psi (light gray bars), and filtrate pool where TMP is 15 psi (black bars). HRC (solid bars) are filtration trains with different endonuclease and salt conditions in the load. In FIG. 9A, significant breakthrough of HCDNA into the filtrate was observed for C0SP at the “C0SP Negative Control”, and for HRC at 250 mM NaCl and 0 U/ml endonuclease (dark gray bars). In FIG. 9B, significant breakthrough of HCDNA into the filtrate was observed for HRC at 250 mM NaCl and 0 U/ml endonuclease (dark gray bars) and 250 mM NaCl and 10 U/ml endonuclease (dark gray bars).

FIGS. 10A-10F schematically depict data for depth filtration treatment at various salt and endonuclease conditions. FIGS. 10A-10C provide data from testing of AAV8 and FIGS. 10D-10F provide data from testing of AAV9.

FIG. 11 schematically depicts data set forth in FIGS. 10A-10F. Throughputs for endonuclease at the 10 U/ml condition were midway between the 0 U/ml and 100 U/ml endonuclease conditions.

FIG. 12 schematically depicts data set forth in FIGS. 10A-10F regarding the effects of salt.

FIG. 13 schematically depicts data showing that salt addition reduces load turbidity and increases throughput at higher pressures.

FIG. 14 schematically depicts data obtained from FIGS. 10A-10F showing that a combined cake-fiber fouling model is a good representation for the mechanism of Harvest RC filtration (depth filtration).

FIGS. 15A-15C schematically depict data obtained from FIGS. 10A-10F. Cake formation parameters (K_{C}) vs NaCl (mM) added with 0 U/ml endonuclease (FIG. 15A), 10 U/ml endonuclease (FIG. 15B), and 100 U/ml endonuclease (FIG. 15C).

FIG. 16 schematically depicts three productions performed using the suspension miniature bioreactor system, and the expected transduction assay luciferase signal for the three productions alone and the pooled material with or without soluble SpyTag quenching. Triangles attached to an AAV represent SpyTag peptide inserted into the VP proteins. Triangles not attached to an AAV represent soluble SpyTag peptide.

FIG. 17 schematically depicts the luciferase signal from the transduction assay when cells are dosed with the respective AAV products at a series of multiplicity of infection (MOI) levels. The 8D3 is a particular mouse-TfR Fab construct.

FIG. 18 schematically depicts data using a POROS CaptureSelect AAV9 resin to capture AAV9-SpyT-SpyC-mAb. Monoclonal antibodies against CACNG1, ASGR1 and Fel d1 were used as retargeting molecules. Less conjugated AAV9 ( 1/20 and 1/30) achieved higher yields.

FIG. 19 schematically depicts and compares various buffers used for affinity capture of an AAV-SpyT-SpyC-Tfr Fab. A Poros CaptureSelect AAV9 column loaded with 10¹³ to 10¹⁵ capsids per milliliter was employed. The yield of the affinity capture step was heavily dependent on the choice of elution buffer. Low yields were achieved in many elution buffers along with loss of the heaviest conjugated species, whereas certain buffers were able to achieve yield >90% along with successful capture of the heavily-conjugated AAV species.

FIG. 20 schematically illustrates a two-step affinity capture protocol utilizing (i) affinity to AAV surface epitopes and (ii) affinity to the antibody surface epitopes. The two affinity capture steps can be employed in any order. Resins used for affinity capture via AAV surface epitopes can comprise POROS CaptureSelect AAVX, POROS CaptureSelect AAV8, POROS CaptureSelect AAV9, Capto AVB, AVB Sepharose, Avipure AAV2, Avipure AAV8, Avipure AAV9, among others. Resins used for affinity capture via antibody surface epitopes can comprise mAbSelectSuRe, mAbSelect PrimaA, Capto L, mAbSelect VL, KappaSelect, among others. The two-step capture protocol allows effective removal of unconjugated antibody as well as unconjugated AAV, resulting in a pool with only conjugated AAV. Pools include eluents.

FIG. 21 schematically depicts a purification train.

FIG. 22 schematically depicts data showing that using the hexad transfection production process to create a library comprising unique covalently surface modified AA species. The average vector genome titer obtained from the bioreactor lysate was 1.8×10¹¹ vg/ml. Unique DNA barcodes were employed as a biomarker encoded inside the AAV vector, and was matched to a Fab candidate (a type of retargeting molecule) conjugated to the outside of the AAV vector. The library contained species that exhibited various genomic titers, which is a measurement of production.

FIG. 23 schematically depicts production purification trains. The top train uses a batch tangential flow filtration unit where repeated passes are required to exchange buffer and concentrate the retentate. The bottom section replaces the batch tangential flow filtration unit with a single pass tangential flow filtration unit. Ionic exchange chromatography of different modalities can be used following TFF, and anion exchange is depicted as an exemplar.

FIG. 24A schematically shows a batch tangential flow filtration (Batch TFF) (top), where the retentate is repeatedly cycled through a feed tank and pump to repeatedly passed through a membrane, with the concentrated permeate being removed after repeated cycles. A single pass tangential flow unit (Single-Pass TFF or SPTFF) removes material from the feed tank through a pump to a multi-stage membrane module that separate the retentate from the permeate, while concentrating the permeate. FIG. 24B is a graph schematically comparing Batch TFF and Single-Pass TFF. Single-Pass TFF achieves higher concentration and is faster as compared to Batch TFF. Single-Pass TFF continuously delivers biological material (such as AAV) to the next operation in the purification train, whereas Batch TFF does not deliver biological material (such as AAV) until the end of the batch cycle.

FIG. 25 schematically compares the batch operation to a continuous operation in terms of Cell lysis, Clarification, TFF (Batch or Single-Pass) and Affinity Capture. The continuous process can be completed in less than a day, whereas the batch process can be multi-day.

FIG. 26 schematically depicts exemplary arrangements for multi-stage membrane module cassettes to be used with Single-Pass TFF. The configurations depict four to seven tiers of membrane module cassettes where the initial tiers (left side) contain more or same number of membrane module cassettes as the succeeding tiers (moving towards the right side). Total area and path length of the membrane module cassettes also are set forth.

FIG. 27 is a graph schematically depicting volumetric concentration factor (VCF) versus transmembrane pressure (TMP) using the 4-in-series, 5-in-series, 6-in-series and 7-in-series exemplary configurations depicted in FIG. 26 with a feed comprising an exemplary AAV, here AAV9 comprising a SpyTag insert.

FIG. 28 schematically depicts data from a 5-in-series configuration according to FIG. 26 at flow rates of 90 ml/minute, 120 ml/minute and 150 ml/minute. The log best-fit equation of VCF=A In (TMP-B) using the values at each flow rate set forth near the plot (and rounded off in the included table) can be used to parameterize the data. At the right side of the figure, there is a graph of parameter value (A, B) and feed flow rate in liters per square meter of membrane per hour (LMH) for 4-in-series and 5-in-series exemplary configurations of FIG. 26 and allows optimized conditions to be selected in silico using an exemplary AAV, here AAV9, comprising a SpyTag insert. This model can be used to predict the VCF for any flow rate and TMP for an in-series configuration of interest.

FIG. 29A schematically depicts a design space model based on FIGS. 27 and 28 using the 5-in-series configuration of FIG. 26. Here, the process target was 35 LMH, and the intersecting lines indicate a VCF of 8 and a TMP of 10 psi. An exemplary acceptable zone would be a VCF of 6-10 and a TMP of 7.5 to 12.5 psi. FIG. 29B is an exemplary comparison of process parameters between SPTFF and Batch TFF. With Batch TFF, typically there would be one batch before the next operation. However, depending on the scheduling of upstream production bioreactors and bioreactor titers, there could be pooling of multiple batches before the next operation

FIG. 30 schematically depicts data from a bench-scale trial to determine the number of buffer washes need to attain about a 90% recovery of AAV, here AAV9 with integrated SpyTag, in a low-TMP process. On average, the exemplary AAV9 contained an average of 6 SpyTag peptids per capsid. Capsid titer in retenate (cp/ml) versus SPTFF operating time (minutes) was measured using four buffer flushes. As the right side of the figure shows, it was determined that only two buffer flushes were required to achieve about a 90% recovery with a VCF of 8×.

FIG. 31 is a graph schematically depicting Permeate Flux (LMH), Throughput (L/m2), Feed Flow Rate (L/hr) and TMP (psi) in a pilot-scale trial. The data showed flux decline and TMP build up. To mitigate TMP increase beyond 12.5 psi, feed flow rate was slowed. This resulted in a longer process time of 180 minutes rather than the expected 90 minutes and an overall VCF of 5× was achieved rather than the target of 8×.

FIG. 32 schematically depicts a tween micelle build-up on the TFF membrane, which is believed to be the cause of an unexpected flux decline of about 50%. This figure also set forth the approximate size of AAV, Host Cell protein aggregates (HCP) and Tween-20 micelles. Detergents, such as Tweens, are a common component of cell lysis buffers used in the purification of AAV.

FIG. 33 is a graph schematically depicting fold presence of Tween-20 on the retentate side of membrane and the Permeate side of the membrane for both Batch TFF and SPTFF.

FIG. 34 is a graph schematically depicting the flux decline after two hours with varying percentages of Tween-20 in the lysis buffer. In addition to Tween-20, the buffer contained 20 mM Tris, 2 mM MgCl₂ at a pH of 7.4. The feed flow rate was 35 LMH and the TMP was about 5 to 10 psi.

FIG. 35 schematically depicts and compares control with the retentate valve to control with a permeate pump. Option 1 with the retentate valve found that TMP reached 22 psi, and after which the flow had to be reduced from 40 LMH to 30 LMH. VCF dropped from about 10× to about 6×. Option 2 with the permeate pump was superior. TMP was controlled to well under 10 psi and a VCF of 8× was maintained. At the right side to the figure Option 1 (SPTFF with retentate valve) and Option 2 (SPTFF with permeate pump) were compared to a Batch TFF. Option 1 did not perform as well as Option 2 and Batch TFF. Option 2 was superior to Batch TFF and Option 1 in terms of capsid yield and percent aggregation.

FIG. 36 schematically depicts the overall pilot scale process, and is similar to parts of the production process of FIG. 23.

FIG. 37 schematically depicts and compares VCFs (1-14), SPTFF retentate flow rates and residence time in affinity capture. VCFs of 7 to 13 and SPTFF retentate flow rates of 75-40 provided an exemplary range of residence time suitable for affinity loading (2.7 to 5.0 minutes).

FIG. 38 schematically depicts how UV280 profile of affinity capture flow can be used for process monitoring of VCF and process stability using SPTFF for continuous processing. Three different runs were performed for comparison purposes. Run 1 was performed without a permeate pump and achieved a VCF of only 5×. Run 2 was performed with a permeate pump with a feed to retentate flush (with recirculation) and achieved a VCF of 8×. Run 3 was performed with a permeate pump with a feed to retentate flush (with recirculation) and a permeate to retentate flush and achieved a VCF of 10×.

### DETAILED DESCRIPTION OF THE INVENTIONS

### Definitions

Unless defined otherwise, all technical and scientific terms used herein have the same meaning as commonly understood by one of ordinary skill in the art to which this invention belongs.

The term “about” in the context of numerical values and ranges refers to values or ranges that approximate or are close to the recited values or ranges such that the invention can perform as intended, such as having a desired rate, amount, density, degree, increase, decrease, percentage, ratio, value, purity, pH, concentration, presence of a form or variant, temperature or amount of time, as is apparent from the teachings contained herein. For example, “about” can signify values either above or below the stated value in a range of approx. +/−10% or more or less depending on the ability to perform. Thus, this term encompasses values beyond those simply resulting from systematic error.

“Antibodies” (also referred to as “immunoglobulins”) are examples of proteins having multiple polypeptide chains and extensive post-translational modifications. The canonical immunoglobulin protein (for example, IgG) comprises four polypeptide chains—two light chains and two heavy chains. Each light chain is linked to one heavy chain via a cysteine disulfide bond, and the two heavy chains are bound to each other via two cysteine disulfide bonds. Immunoglobulins produced in mammalian systems are also glycosylated at various residues (for example, at asparagine residues) with various polysaccharides, and can differ from species to species, which may affect antigenicity for therapeutic antibodies. Butler and Spearman, “The choice of mammalian cell host and possibilities for glycosylation engineering”, Curr. Opin. Biotech. 30:107-112 (2014).

An antibody includes immunoglobulin molecules comprised of four polypeptide chains, two heavy (H) chains and two light (L) chains inter-connected by disulfide bonds. Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3. The term “high affinity” antibody refers to those antibodies having a binding affinity to their target of at least 10⁻⁹ M, at least 10⁻¹⁰ M; at least 10⁻¹¹ M; or at least 10⁻¹² M, as measured by surface plasmon resonance, for example, BIACORE™ or solution-affinity ELISA.

The phrase “bispecific antibody” includes an antibody capable of selectively binding two or more epitopes. Bispecific antibodies generally comprise two different heavy chains, with each heavy chain specifically binding a different epitope—either on two different molecules (for example, antigens) or on the same molecule (for example, on the same antigen). If a bispecific antibody is capable of selectively binding two different epitopes (a first epitope and a second epitope), the affinity of the first heavy chain for the first epitope will generally be at least one to two, three or four orders of magnitude lower than the affinity of the first heavy chain for the second epitope, or vice versa. The epitopes recognized by the bispecific antibody can be on the same or a different target (for example, on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain. A typical bispecific antibody has two heavy chains each having three heavy chain CDRs, followed by (N-terminal to C-terminal) a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding or one or both of the heavy chains to one or both epitopes.

The term “components” refers to any constituent molecules needed to produce a covalently surface modified adeno-associated viruses and include, but are not limited to promoters, polyadenylation signals, transgenes, genes encoding retargeting molecules, AAV cap genes, AAV rep genes, ITRs, helper polynucleotide sequence(s), genes encoding a first member of a specific binding pair and a second cognate member of a specific binding pair, as well as peptides encoded by the genes and sequences. Optional sequences include detargeting mutation sequences, IRESs, RRSs, introns, operators and enhancers.

The phrase “assembly of components” refers to peptide components that assemble together by way of bonds, forces, interactions and/or attractions. Examples include the assembly of heavy and light chains to form antibodies, capsid proteins and isopeptide bonds formed during conjugation of specific binding pairs.

The phrase “DNA barcode” refers to types of unique nucleotide sequences that can be used for identification purposes. DNA barcodes typically contain the same amount of base pairs (for example, 32 base pair pairs), but each type will have an unique sequence, and are commercially available. The DNA barcodes can include terminal single stranded hairpins. Exemplary DNA barcode sequences are disclosed in Example 34, which sets forth one strand of the unique basepairs.

The phrase “heavy chain,” or “immunoglobulin heavy chain” includes an immunoglobulin heavy chain constant region sequence from any organism, and unless otherwise specified includes a heavy chain variable domain. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a CH1 domain, a hinge, a CH2 domain, and a CH3 domain. A functional fragment of a heavy chain includes a fragment that is capable of specifically recognizing an antigen (for example, recognizing the antigen with a KD in the micromolar, nanomolar, or picomolar range), that is capable of expressing and secreting from a cell, and that comprises at least one CDR.

The phrase “light chain” includes an immunoglobulin light chain constant region sequence from any organism, and unless otherwise specified includes human kappa and lambda light chains. Light chain variable (VL) domains typically include three light chain CDRs and four framework (FR) regions, unless otherwise specified. Generally, a full-length light chain includes, from amino terminus to carboxyl terminus, a VL domain that includes FR1-CDR1-FR2-CDR2-FR3-CDR3-FR4, and a light chain constant domain. Light chains that can be used with these inventions include those, for example, that do not selectively bind either the first or second antigen selectively bound by the antigen-binding protein. Suitable light chains include those that can be identified by screening for the most commonly employed light chains in existing antibody libraries (wet libraries or in silico), where the light chains do not substantially interfere with the affinity and/or selectivity of the antigen-binding domains of the antigen-binding proteins. Suitable light chains include those that can bind one or both epitopes that are bound by the antigen-binding regions of the antigen-binding protein.

“Recombinase recognition sites” (RRS), also known as “heterospecific recombination sites,” are used in recombinase mediated cassette exchange (RMCE). Cre/Lox Dre/Rox, VCre/Vlox, SCre/Slox and Flp/Frt are suitable systems, for example. Suitable RRSs for use according to the inventions include Lox P, Lox 66, Lox 71, Lox 511, Lox 2272, Lox 2372, Lox 5171, Lox M2, Lox M3, lox M7 and Lox M11. These sites can be referred to generically as first (1), second (2), third (3), fourth (4), fifth (5), sixth (6), seventh (7), eighth (8), ninth (9), tenth (10), etc., as is apparent from the context of usage.

An “intron” is a section of DNA that is not protein encoding, and typically is located between “exons”, which encode protein regions. An intron is removed to form a mature messenger RNA, which is translated to form protein. Some introns are those that can affect the starting point of translation, and exemplars are the hCMV-IE intron (Human cytomegalovirus immediate early protein) and FMDV intron (Foot and Mouth Disease Virus).

“Intronic selection” refers to the optional use of recombinase recognition sites located in intronic regions to allow for integration of multiple cassettes to form a construct. See Published applications US 2019/0263937 A1 and US 2019/0233544 A1. For example, selection markers and reporter genes can be engineered to include introns with RRSs contained therein. Intronic selection can be used to create constructs sectionally. For instance, a large construct containing multiple cassettes can be created by using smaller, constituent constructs.

“Antibody derivatives and fragments” include, but are not limited to: antibody fragments (for example, Fab, ScFv-Fc, dAB-Fc, half antibodies and other combinations of heavy and/or light chains), multispecifics (for example, bispecifics, IgG-ScFv, IgG-dab, ScFV-Fc-ScFV, trispecifics).

The phrase “Fc-containing protein” includes antibodies, bispecific antibodies, antibody derivatives containing an Fc, antibody fragments containing an Fc, Fc-fusion proteins, immunoadhesins, and other binding proteins that comprise at least a functional portion of an immunoglobulin CH2 and CH3 region. A “functional portion” refers to a CH2 and CH3 region that can bind a Fc receptor (for example, an FcyR; or an FcRn, (neonatal Fc receptor), and/or that can participate in the activation of complement. If the CH2 and CH3 region contains deletions, substitutions, and/or insertions or other modifications that render it unable to bind any Fc receptor and also unable to activate complement, the CH2 and CH3 region is not functional. Fc-fusion proteins include, for example, Fc-fusion (N-terminal), Fc-fusion (C-terminal), mono-Fc-fusion and bispecific Fc-fusion proteins.

“Fc” stands for fragment crystallizable, and is often referred to as a fragment constant. Antibodies contain an Fc region that is made up of two identical protein sequences. IgG has heavy chains known as γ-chains. IgA has heavy chains known as α-chains, IgM has heavy chains known as μ-chains. IgD has heavy chains known as σ-chains. IgE has heavy chains known as ε-chains. In nature, Fc regions are the same in all antibodies of a given class and subclass in the same species. Human IgGs have four subclasses and share about 95% homology amongst the subclasses. In each subclass, the Fc sequences are the same. For example, human IgG1 antibodies will have the same Fc sequences. Likewise, IgG2 antibodies will have the same Fc sequences; IgG3 antibodies will have the same Fc sequences; and IgG4 antibodies will have the same Fc sequences. Alterations in the Fc region create charge variation.

“Fc-fusion proteins” comprise part or all of two or more proteins, one of which is an Fc portion of an immunoglobulin molecule, that are not fused in their natural state. Fc-fusion proteins include Fc-Fusion (N-terminal), Fc-Fusion (C-terminal), Mono Fc-Fusion and Bi-specific Fc-Fusion. Preparation of fusion proteins comprising certain heterologous polypeptides fused to various portions of antibody-derived polypeptides (including the Fc domain) has been described, for example, by Ashkenazi et al., Proc. Natl. Acad. Sci. USA 88: 10535-39 (1991); Byrn et al., Nature 344:677-70, 1990; and Hollenbaugh et al., “Construction of Immunoglobulin Fusion Proteins”, in Current Protocols in Immunology, Suppl. 4, pages 10.19.1-10.19.11 (1992). “Receptor Fc-fusion proteins” comprise one or more of one or more extracellular domain(s) of a receptor coupled to an Fc moiety, which in some embodiments comprises a hinge region followed by a CH2 and CH3 domain of an immunoglobulin. In some embodiments, the Fc-fusion protein contains two or more distinct receptor chains that bind to a single or more than one ligand(s). Some receptor Fc-fusion proteins may contain ligand binding domains of multiple different receptors. Receptor Fc-fusion proteins are also referred to as “traps,” “trap molecules” or “trap proteins.” For example, such trap proteins include an IL-1 trap (for example, Rilonacept, which contains the IL-IRAcP ligand binding region fused to the IL-1R1 extracellular region fused to Fc of hIgGI; see U.S. Pat. No. 6,927,044, or a VEGF Trap (for example, Aflibercept, which contains the Ig domain 2 of the VEGF receptor FltI fused to the Ig domain 3 of the VEGF receptor FlkI fused to Fc of hIgG1 See U.S. Pat. Nos. 7,087,411 and 7,279,159.

“Polynucleotide” includes a sequence of nucleotides covalently joined, and includes RNA and DNA. Oligonucleotides are considered shorter polynucleotides. Genes are DNA polynucleotides (polydeoxyribonucleic acid) that ultimately encode polypeptides, which are translated from RNA (polyribonucleic acid) that was typically transcribed from DNA. DNA polynucleotides also can encode RNA polynucleotides that is not translated, but rather function as RNA “products”. The type of polynucleotide (that is, DNA or RNA) is apparent from the context of the usage of the term. A polynucleotide referred to or identified by the polypeptide it encodes sets forth and covers all suitable sequences in accordance with codon degeneracy. Polynucleotides, including those disclosed herein, include percent identity sequences and homologous sequences when indicated.

“Polypeptide” or “peptide” refers to sequence(s) of amino acids covalently joined. Polypeptides include natural, semi-synthetic and synthetic proteins and protein fragments. “Polypeptide” and “protein” can be used interchangeably. Oligopeptides are considered shorter polypeptides.

A “gene of interest” (GOI) encodes a “protein of interest” or “polypeptide of interest” and optionally can include other associated sequences. The sequences can be natural, semi-synthetic or synthetic. Native sequences, mutant sequences and degenerate sequences can be GOIs. A gene of interest also can be referred to as a “transgene.”

A “nucleotide of interest” includes GOIs and sequences encoding non-translated RNAs/non-coding RNAs (such as, but not limited to, antisense RNA, small interfering RNA, micro RNA, catalytic RNA and ribozymes). NOIs and GOIs also can be referred to as “payloads.”

“Protein of interest” or “polypeptide of interest” (POI) can have any amino acid sequence, and includes any protein, polypeptide, or peptide that is desired to be expressed, typically for gene therapy purposes. Protein types can include, but are not limited to, receptors, fusion proteins, agonists, antagonists, activators, inhibitors, enzymes (such as those used in enzyme replacement therapy), factors and co-factors, repressors, activators, ligands, protein hormones, therapeutic proteins, suicide proteins, structural proteins, storage proteins, transport proteins, signal proteins, neurotransmitters and contractile proteins. Derivatives, components, domains, chains and fragments of the above also are included. The sequences can be natural, semi-synthetic or synthetic.

“Purification” in its various grammatical forms includes, but is not limited to, the use of one or more procedures such as depth filtration, tangential flow filtration, affinity capture, ionic exchange and the like.

The term “recombinant capsid protein” includes a capsid protein that has at least one mutation in comparison to the corresponding capsid protein of the wild-type virus, which wild-type may be a reference and/or control virus for comparative study. A recombinant capsid protein includes a capsid protein that comprises a heterologous amino acid sequence, which may be inserted into and/or displayed by the capsid protein. “Heterologous” in a general context means heterologous as compared to the virus, from which the capsid protein is derived. The inserted amino acids can simply be inserted between two given amino acids of the capsid protein. An insertion of amino acids can also go along with a deletion of given amino acids of the capsid protein at the site of insertion, for example, 1 or more capsid protein amino acids are substituted by 5 or more heterologous amino acids). An example of a heterologous amino acid sequence that can be inserted is a member of a specific binding pair, such SpyTag.

“Detargeting” refers to reducing or abolishing AAV natural preferential transduction by mutating Cap proteins. For example, mutations in the galactose binding domain of VP1 assist in detargeting the liver. These mutations are optional and can be referred to as “detargeting mutations,” and are discussed herein in greater detail.

By way of example, different AAV serotypes are known to preferentially transduce the cells of different tissues. Tissue specificity is limited, and AAV is known to preferentially transduce the liver, which can be a safety and efficacy concern in some contexts. The inventions further provide mutations in the VP1 Protein of AAV9, for example, to lower the AAV preferential transduction of the liver. The AAV9 mutations include N272A and W503A substitutions, where alanine replaces both asparagine at position 272 of VP1 and tryptophan at position 503 of VP1. One or both of the mutations can be undertaken in the VP1 protein. Optionally, other amino acids, such as glutamic acid, serine or others, can be used instead of alanine for substitution. Additional detargeting mutation sites include, but are not limited to, N470, D271, and Y446. The inventions provide exemplary mutations for other AAVs are as follows:
AAV1—N500E;
AAV2—R585A and R588A;
AAV5—T571 S;
AAV6—N500E, K531A and K531E.

These and others are set forth in the chart below:

| AAV | Insertion | Exemplary |
|---|---|---|
| serotype | Sites | Mutations |
| AAV2 | 1, 34, 138, 139, 161, 261, | R484, R487, R585A, |
| | 266, 381, 447, 448, 453, | R588A and K532. |
| | 459, 471, 520, 534, 570, | R484A, R487A, R487G, |
| | 573, 584, 587, 588, 591, | K532A, K532D, R585A, |
| | 657, 664, 713, 716 | R585S, R585Q, R588A, |
| | | R588T |
| AAV9 | 272, 453, 503, 587, 589 | N272A, W503A |
| AAV1 | 587, 589 | N500E |
| AAV3 | 585 | |
| AAV4 | 584, 585 | |
| AAV5 | 531, 571, 575, 585 | K531A, K531E, T571S |
| AAV6 | 500, 531 | N500E, K531A, K531E |
| Avian AAV | 444, 580 | |
| Sea lion | 429, 430, 431, 432, 433, | |
| AAV | 434, 436, 437, 565 | |
| Bearded | 573, 436 | |
| Dragon AAV | | |

Other mutations are available in publications and otherwise available, and can be used according to the inventions.

“Retargeting” or “redirecting” may include a situations in which the wildtype vector targets several cells within a tissue and/or several organs within an organism, which general targeting of the tissue or organs is reduced or abolished by provision of a retargeting molecule, which retargets the covalently surface modified AAV to a different, and optionally more specific, cell in the tissue or a specific organ in the organism.

The term “retargeting molecule” (Rm) is a molecule useful for targeting an antigen, receptor, protein, including glycoproteins, and/or ligand (collectively “targets’) found on the surface of a cell, referred to as a “target cell.” The retargeting molecule is bound to a polypeptide that is part of a specific binding pair. For example, a retargeting molecule could be bound to SpyCatcher in order to utilize the SpyTag-SpyCatcher system. The retargeting molecule can target the cell that has the antigen, receptor and/or ligand that the retargeting molecule can bind to, and thereby direct a recombinant AAV to that cell. Fc-containing proteins, such as antibodies, monoclonal antibodies (including derivatives, fragments, half antibodies and other heavy chain and/or light chain combinations), multispecific antibodies (for example, bispecifics, IgG-ScFv, IgG-dab, ScFV-Fc-ScFV, trispecifics), Fc-fusion proteins, receptor-Fc fusion proteins, trap proteins can be useful as retargeting molecules. Mini-trap proteins also can be useful as retargeting molecules.

All human and non-human antibody classes can be used as retargeting molecules. IgA, IgD IgE, IgG and IgM can be used as retargeting molecules. IgG is a preferred class, and includes subclasses IgG1 (including IgG1λ and IgG1κ), IgG2, IgG3, and IgG4. Further antibody types include a human antibody, a humanized antibody, a chimeric antibody, a monoclonal antibody, a multispecific antibody, a bispecific antibody, a trispecific antibody, an antigen binding antibody fragment, a single chain antibody, a diabody, triabody, tetrabody, Fab, F(ab′), F(ab′)2 and a half antibody.

“Specific binding pair,” “protein:protein binding pair” and the like includes two proteins (that is, a first member, such as a first polypeptide, and a second cognate member, such as a second polypeptide) that interact to form a covalent isopeptide bond under conditions that enable or facilitate isopeptide bond formation, wherein the term “cognate” refers to components that function together by to reacting together to form an isopeptide bond. Thus, two proteins that react together efficiently to form an isopeptide bond under conditions that enable or facilitate isopeptide bond formation can also be referred to as being a “complementary” pair of peptide linkers. Specific binding pairs capable of interacting to form a covalent isopeptide bond are reviewed in Veggiani et al. (2014) Trends Biotechnol. 32:506, and include, for example, peptide:peptide binding pairs such as SpyTag:SpyCatcher, SpyTag002:SpyCatcher002, SpyTag:KTag, isopeptag:pilin C, SnoopTag:SnoopCatcher and others. Spy Tag002:SpyCatcher002 and SpyTag003:SpyCatcher003 are different iterations of Spy Tag:Spy Catcher.

The term “isopeptide bond” refers to an amide bond between a carboxyl or carboxamide group and an amino group at least one of which is not derived from a protein main chain or alternatively viewed is not part of the protein backbone. An isopeptide bond may form within a single protein or may occur between two peptides or a peptide and a protein. Thus, an isopeptide bond may form intramolecularly within a single protein or intermolecularly, that is between two peptide/protein molecules, such as between two peptide linkers. Typically, an isopeptide bond may occur between a lysine residue and an asparagine, aspartic acid, glutamine, or glutamic acid residue or the terminal carboxyl group of the protein or peptide chain or may occur between the alpha-amino terminus of the protein or peptide chain and an asparagine, aspartic acid, glutamine or glutamic acid. Each residue of the pair involved in the isopeptide bond is referred to herein as a reactive residue. An isopeptide bond may form between a lysine residue and an asparagine residue or between a lysine residue and an aspartic acid residue. Particularly, isopeptide bonds can occur between the side chain amine of lysine and carboxamide group of asparagine or carboxyl group of an aspartate.

“Reporter proteins” as used herein, refers to any protein capable of generating directly or indirectly a detectable signal. Reporter proteins typically fluoresce, or catalyze a colorimetric, bioluminescence, or fluorescent reaction, and often are referred to as “color proteins,” “bioluminescent proteins” or “fluorescent proteins.” However, a reporter protein also can be non-enzymatic and non-fluorescent as long as it can be detected by another protein or moiety, such as a cell surface protein detected with a fluorescent ligand. A reporter protein also can be an inactive protein that is made functional through interaction with another protein that is fluorescent or catalyzes a reaction. Accordingly, any suitable reporter protein, as understood by one of skill in the art, could be used. The reporter protein can be selected from fluorescent protein, luciferase, alkaline phosphatase, β-galactosidase, β-lactamase, dihydrofolate reductase, ubiquitin, and variants thereof. Fluorescent proteins are useful for the recognition of gene cassettes that have or have not been successfully inserted and/or replaced, as the case may be. Fluid cytometry and fluorescence-activated cell sorting are suitable for detection. Examples of fluorescent proteins are well-known in the art, including, but not limited to Discosoma coral (DsRed), green fluorescent protein (GFP), enhanced green fluorescent protein (eGFP), cyano fluorescent protein (CFP), enhanced cyano fluorescent protein (eCFP), yellow fluorescent protein (YFP), enhanced yellow fluorescent protein (eYFP) and far-red fluorescent protein (e.g. mKate, mKate2, mPlum, mRaspberry or E2-crimson. See, for example, U.S. Pat. No. 9,816,110. Reporter proteins are encoded by polynucleotides, and are referred to herein as “reporter genes” or “reporter protein genes.” Reporter genes and proteins can be referred to generically as first (1), second (2), third (3), fourth (4), fifth (5), sixth (6), seventh (7), eighth (8), ninth (9), tenth (10), etc., as is apparent from the context of usage. Reporters can be considered a type of marker. “Color” or “fluorescent,” in their various grammatical forms, also can be used the more specifically refer to a reporter protein or gene. Where multiple plasmids are used in a transfection, the plasmids can collectively comprise the same reporter genes or different reporter genes.

“Selectable” or “selection” marker proteins include proteins conferring certain traits, including but not limited to drug resistance or other selective advantages. Selection markers can give the cell receiving the selectable marker gene resistance towards a certain toxin, drug, antibiotic or other compound and permit the cell to produce protein and propagate in the presence of the toxin, drug, antibiotic or other compound, and are often referred to as “positive selectable markers.” Suitable examples of antibiotic resistance markers include, but are not limited to, proteins that impart resistance to various antibiotics, such as kanamycin, spectinomycin, neomycin, gentamycin (G418), ampicillin, tetracycline, chloramphenicol, puromycin, hygromycin, zeocin, and/or blasticidin. There are other selectable markers, often referred to as “negative selectable markers,” which cause a cell to stop propagating, stop protein production and/or are lethal to the cell in the presence of the negative selectable marker proteins. Thymidine kinase and certain fusion proteins can serve as negative selectable markers, including but not limited to GyrB-PKR. See White et al., Biotechniques, 50: 303-309 (May 2011). Selectable marker proteins and corresponding genes can be referred to generically as first (1), second (2), third (3), fourth (4), fifth (5), sixth (6), seventh (7), eighth (8), ninth (9), tenth (10), etc., as is apparent from the context of usage. Where multiple plasmids are used in a transfection, the plasmids can collectively comprise the same selection marker genes or different selection marker genes.

The term “target cells” includes any cells in which expression of a nucleotide of interest is desired or tolerated. Preferably, target cells exhibit a “target,” such as a receptor, ligand, protein, including glycoproteins, and/or antigen, including complexes thereof, on their surface that allows the cell to be targeted. Exemplary targets are calcium voltage-gated channel auxiliary subunit gamma 1 (CACNG1), asialoglycoprotein receptor 1 (ASGR1), Fel d 1, ENTPD3, PTPRA, CD20, CD63 and Her2. Additional targets include GAB A, transferrin receptor, CD3, CD34, integrin, adipophilin, AIM-2, ALDHIAI, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNKIAI, CTAGI, CTAG2, cyclin DI, Cyclin-AI, dek-can fusion protein, DKK1, EFTUD2, Elongation factor 2, ENAH (hMena), Ep-CAM, EpCAM, EphA3, epithelial tumor antigen (“ETA”), ETV6-AML1 fusion protein, EZH2, E6, E7, FGF5, FLT3-ITD, FN1, G250/MN/CAIX, GAGE-1,2,8, GAGE-3,4,5,6,7, GAS7, glypican-3, GnTV, gplOO/Pmel 17, GPNMB, HAUS3, Hepsin, HER-2/neu, HERV-K-MEL, HLA-A11, HLA-A2, HLA-DOB, hsp70-2, IDOI, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferase AS fusion protein, Lengsin, M-CSF, MAGE-A1, MAGE-A 10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, ME1, Mel an-A/MART-1, Meloe, Midkine, MMP-2, MMP-7, MUC1, MUC5AC, mucin, MUM-1, MUM-2, MUM-3, Myosin, Myosin class I, N-raw, NA88-A, neo-PAP, NFYC, NY-BR-1, NY-ESO-I/LAGE-2, OA1, OGT, OS-9, P polypeptide, p53, PAP, PAX5, PBF, pml-RARalpha fusion protein, polymorphic epithelial mucin (“PEM”), PPP1R3B, PRAME, PRDX5, PSA, PSMA, PTPRK, RAB 38/N Y-MEL-1, RAGE-1, RBAF600, RGS5, RhoC, R F43, RU2AS, SAGE, secernin 1, SIRT2, SNRPD1, SOX10, Sp17, SPA17, SSX-2, SSX-4, STEAPI, survivin, SYT-SSX1 or -SSX2 fusion protein, TAG-1, TAG-2, Telomerase, TGF-betaRII, TPBG, TRAG-3, Triosephosphate isomerase, TRP-1/gp75, TRP-2, TRP2-INT2, tyrosinase, tyrosinase (“TYR”), VEGF, WT1, XAGE-Ib/GAGED2a, Kras, NY-ESOI, MAGE-A3, HPV E2, HPV E6, HPV E7, WT-1 antigen (in lymphoma and other solid tumors), ErbB receptors, Melan A [MARTI], gp 100, tyrosinase, TRP-I/gp 75, and TRP-2 (in melanoma); MAGE-1 and MAGE-3 (in bladder, head and neck, and non-small cell carcinoma); HPV EG and E7 proteins (in cervical cancer); Mucin [MUC-1] (in breast, pancreas, colon, and prostate cancers); prostate-specific antigen [PSA] (in prostate cancer); carcinoembryonic antigen [CEA] (in colon, breast, and gastrointestinal cancers), and such shared tumor-specific antigens as MAGE-2, MAGE-4, MAGE-6, MAGE-10, MAGE-12, BAGE-1, CAGE-1,2,8, CAGE-3 TO 7, LAGE-1, NY-ESO-I/LAGE-2, NA-88, GnTV, TRP2-INT2, E6, E7, human glucagon receptor (hGCGR) and. I human ectonucleoside triphosphate diphosphohydrolase 3 (hENTPD3). Other targets can be selected by the person skilled in the art. See WO 2019/006046.

All numerical limits and ranges set forth herein include all numbers or values thereabout or there between of the numbers of the range or limit. The ranges and limits described herein expressly denominate and set forth all integers, decimals and fractional values defined and encompassed by the range or limit. Thus, a recitation of ranges of values herein are intended to serve as a way of referring individually to each separate value falling within the range, unless otherwise indicated herein, and each separate value is incorporated into the specification as if it were individually recited herein.

### DESCRIPTION

The inventions advantageously employ one or more “specific binding pairs,” also referred to as “protein:protein binding pairs.” An exemplary system is the SpyTag-SpyCatcher system. The SpyTag-SpyCatcher system was developed using the Streptococcus pyogenes second immunoglobulin-like collagen adhesion domain (CnaB2) from the fibronectin binding protein FbaB. An isopeptide bond can be formed spontaneously between the SpyTag protein and the SpyCatcher protein. The SpyCatcher peptide is about 15 kD in size. However, the SpyTag protein is only 13 amino acids long. The small size of the SpyTag protein makes it amenable for insertion into the AAV genome, which has a total packing capacity of only about 4.7 kilobases. These systems, such as SpyTag-SpyCatcher, allow a retargeting molecule to be bound to an AAV.

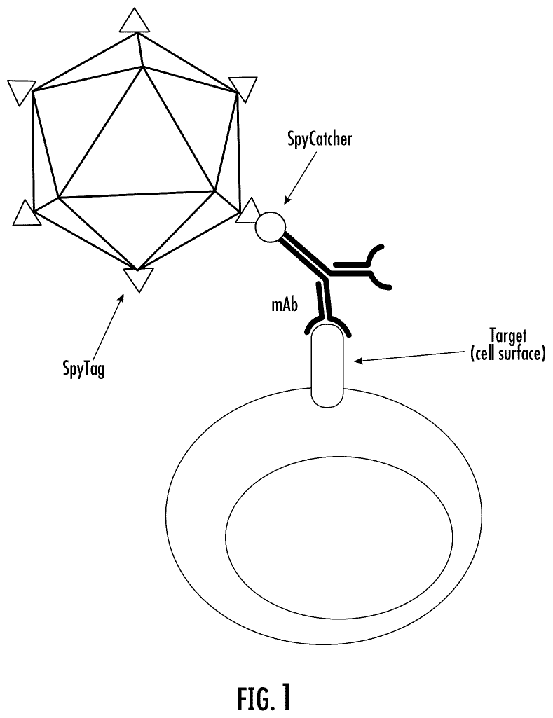

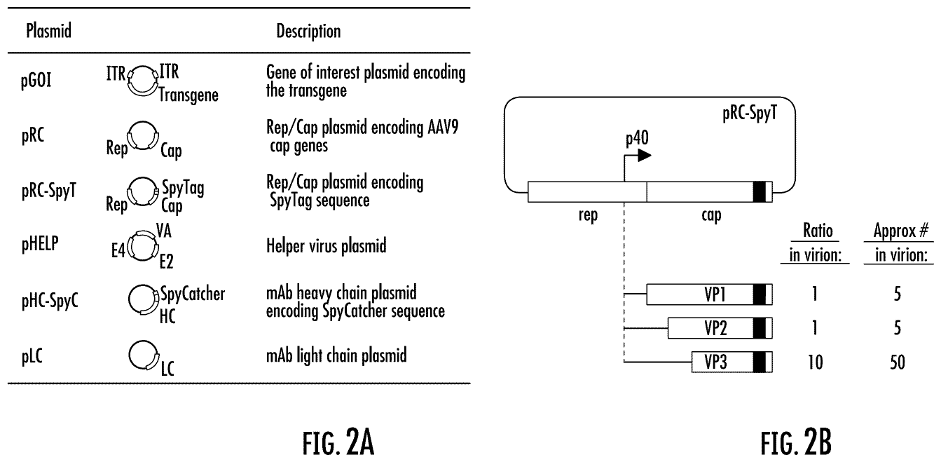

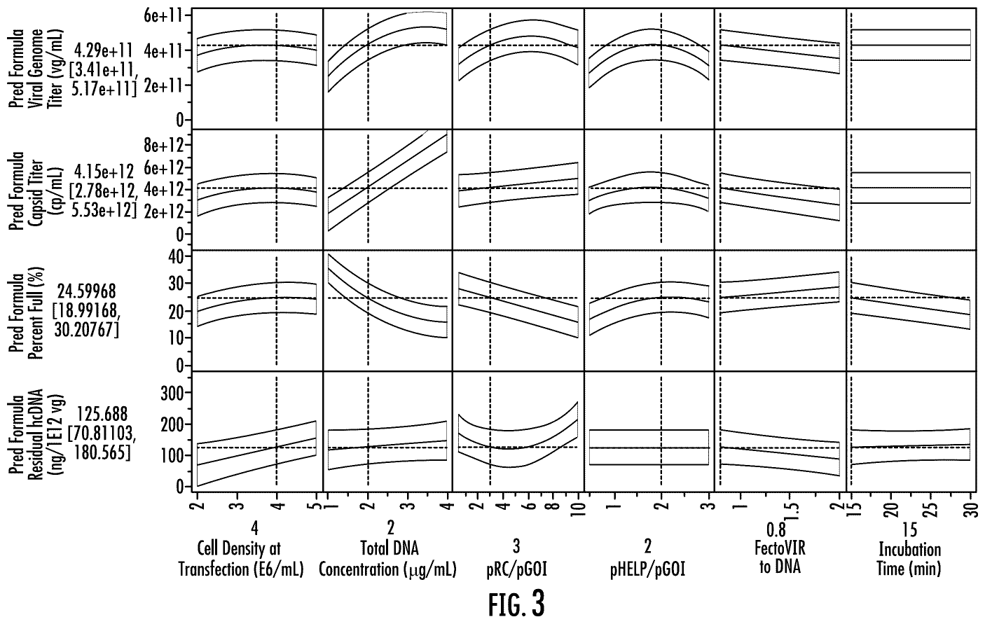

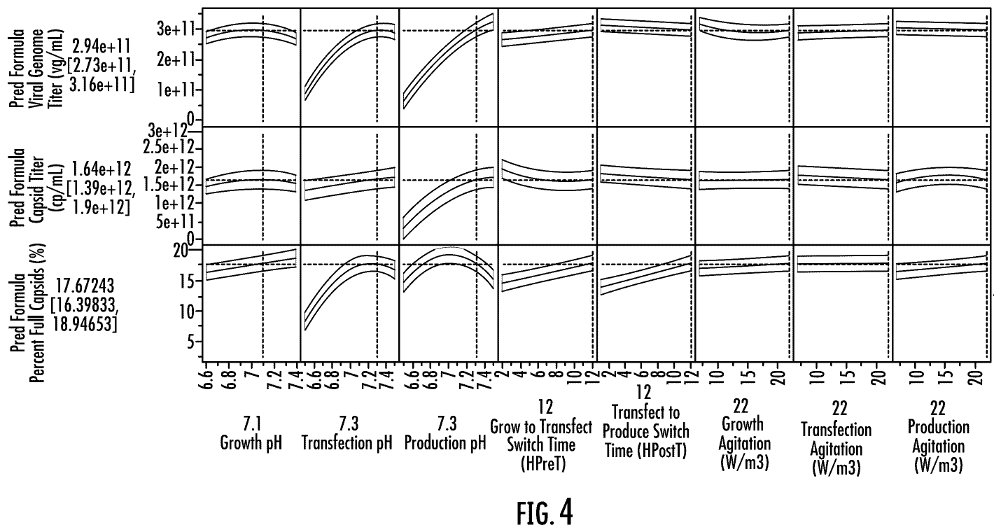

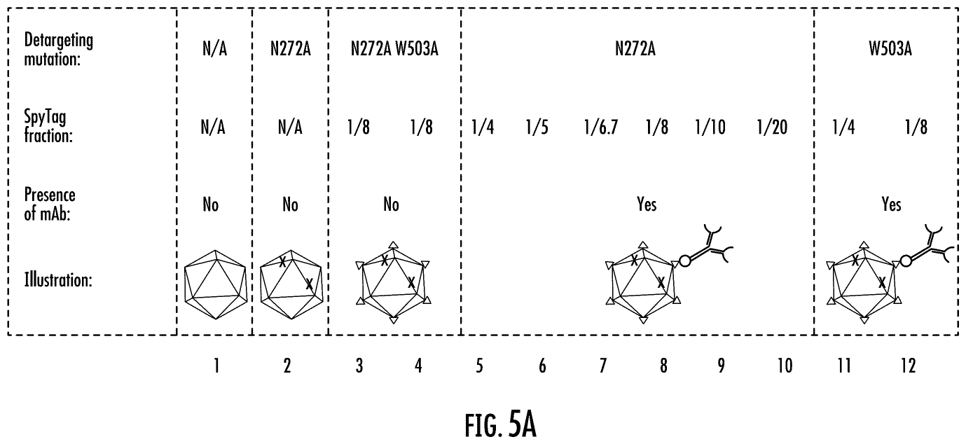

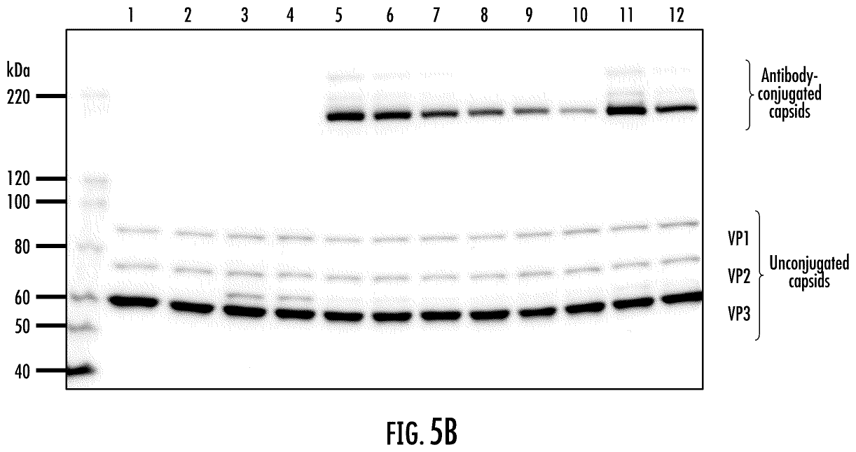

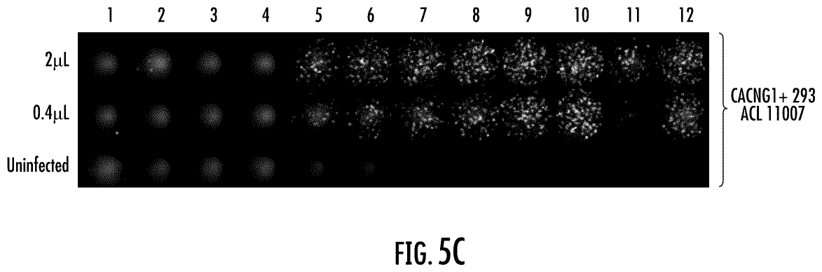

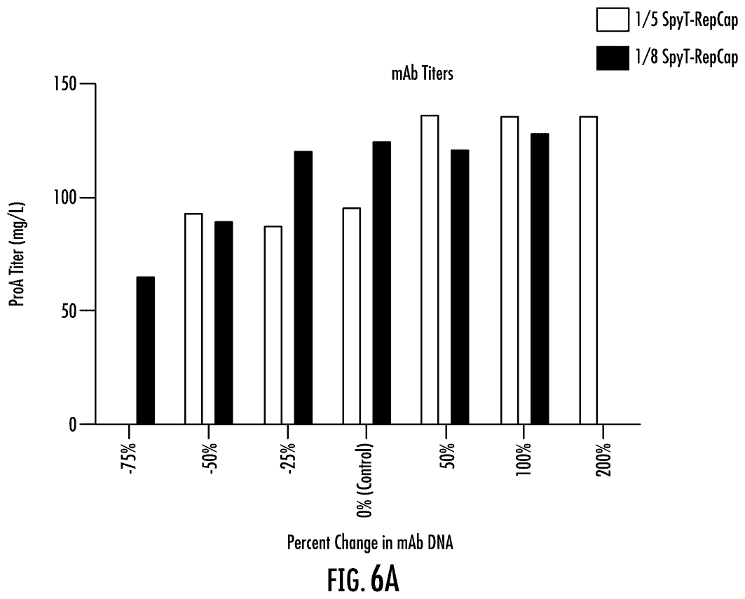

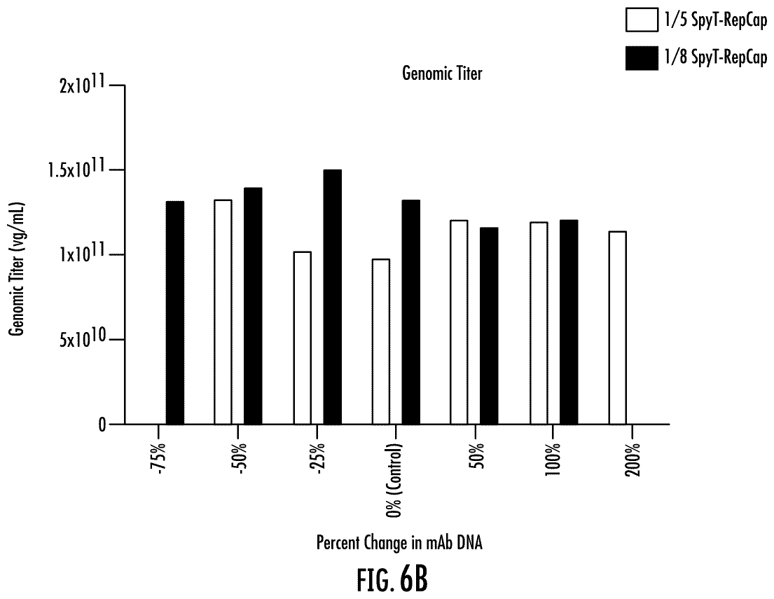

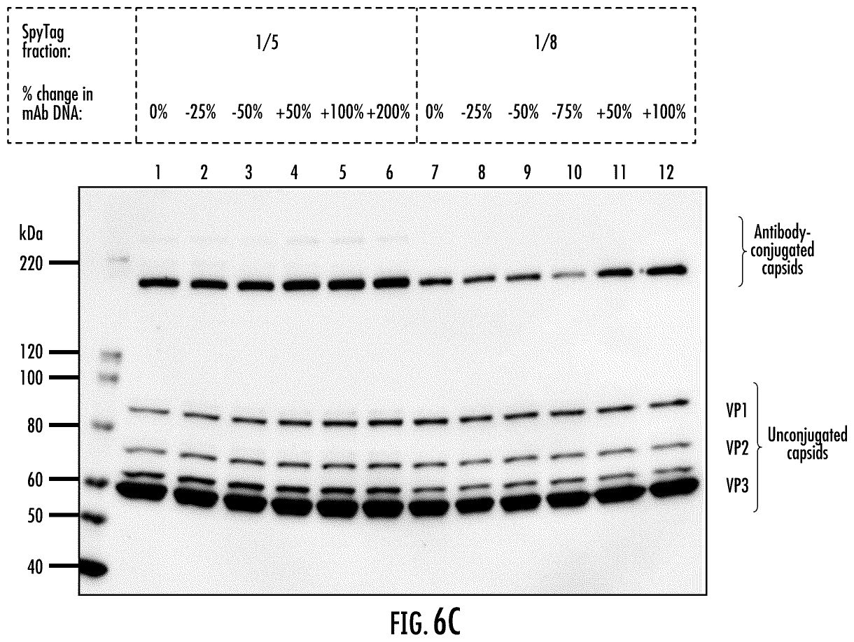

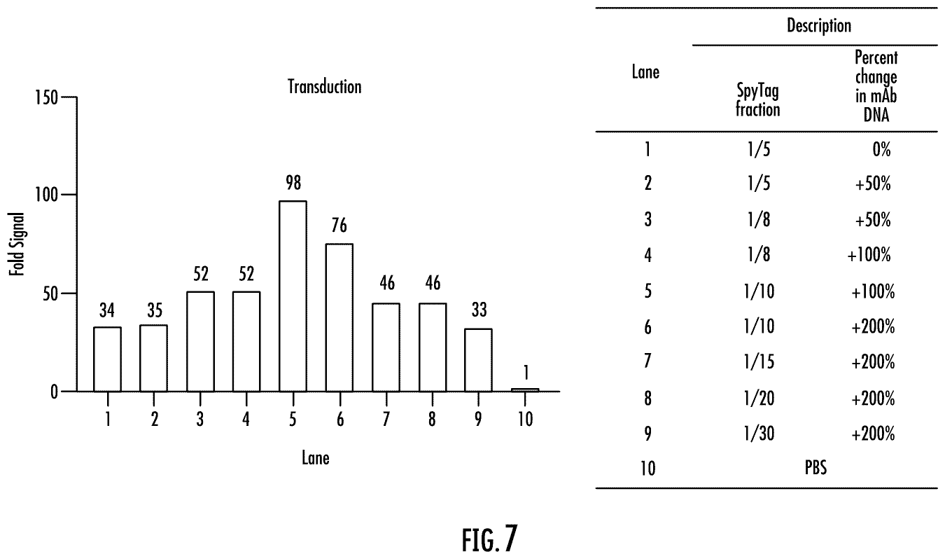

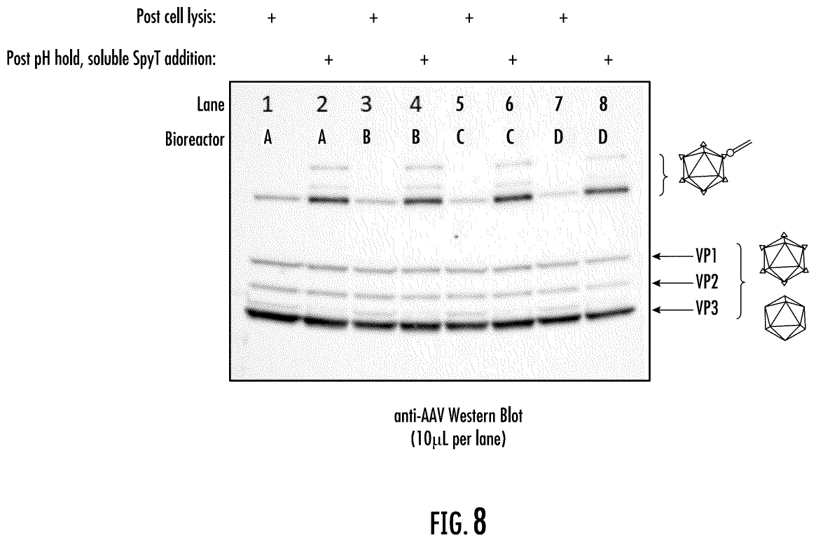

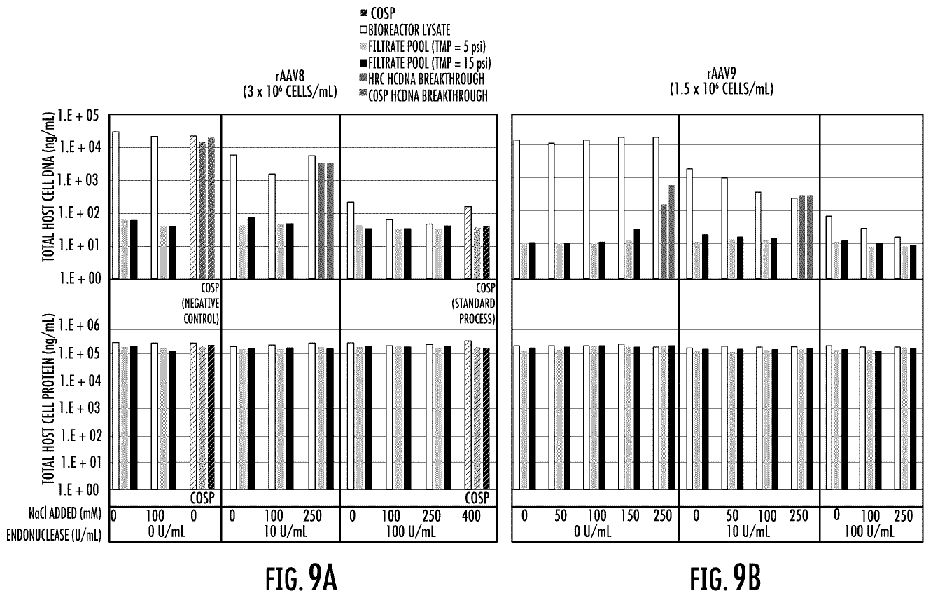

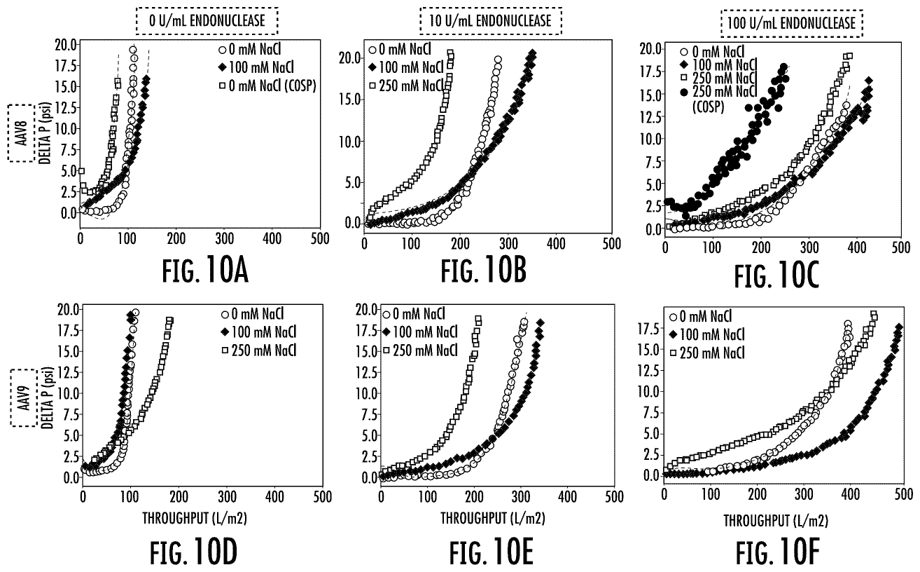

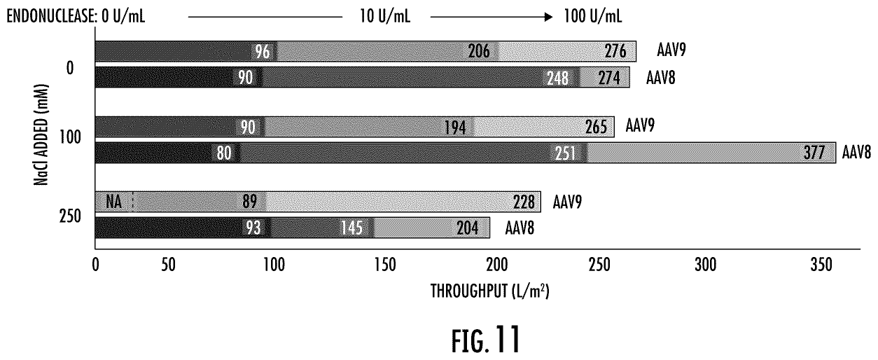

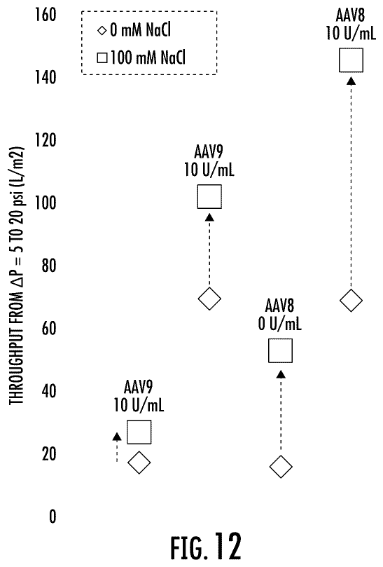

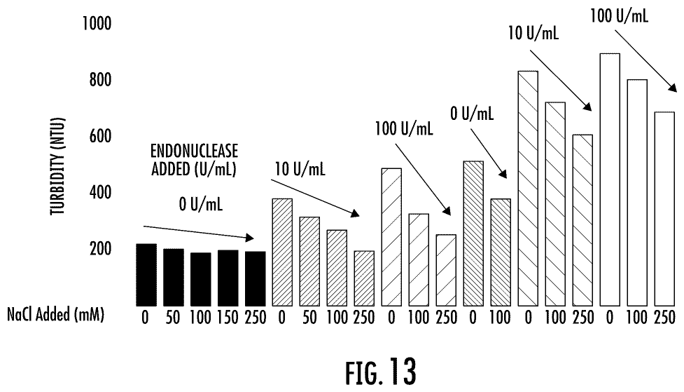

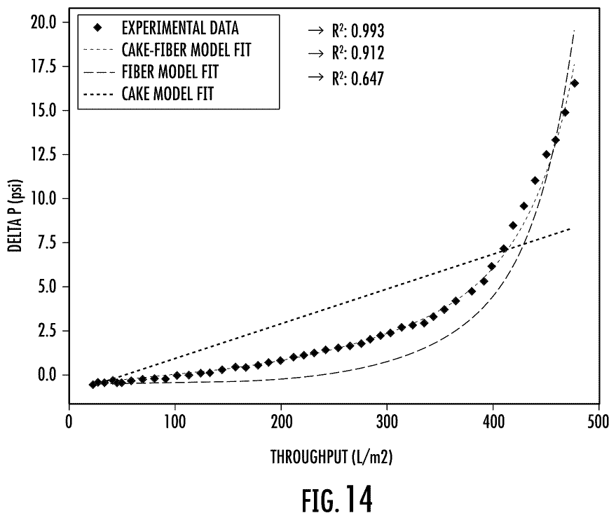

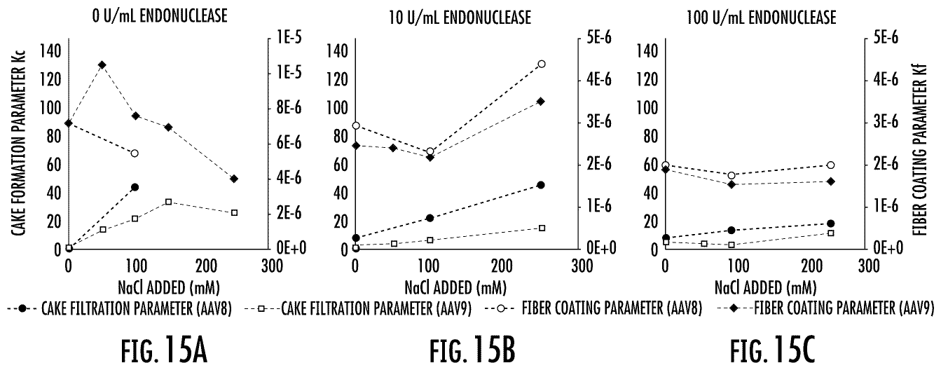

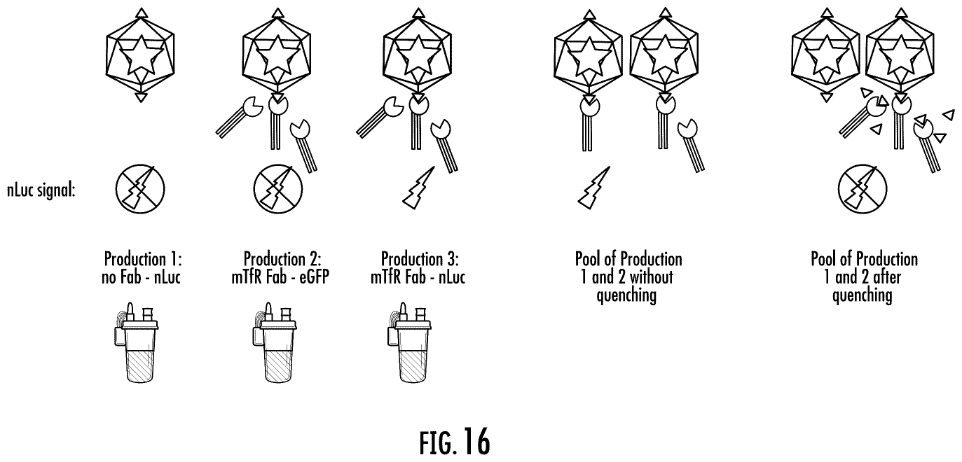

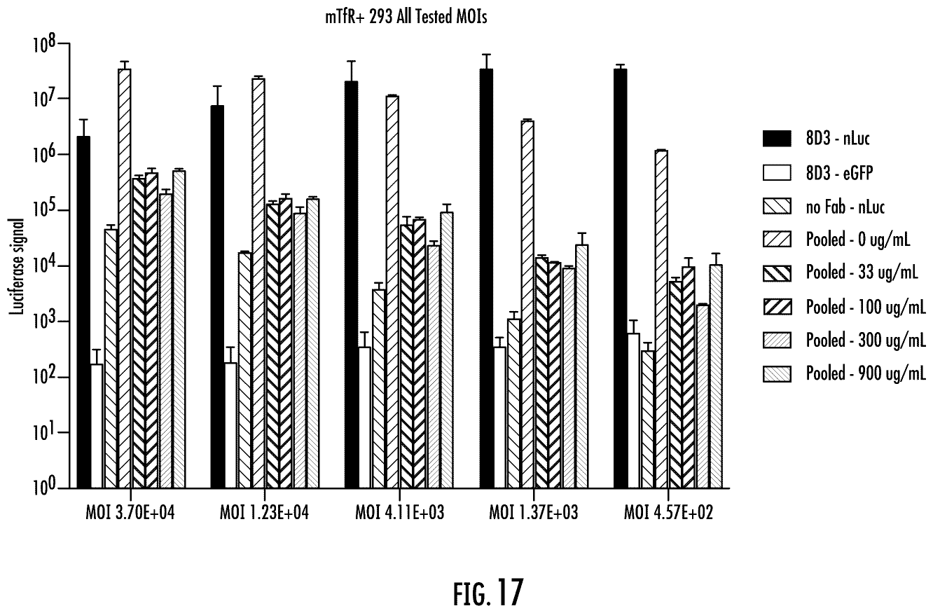

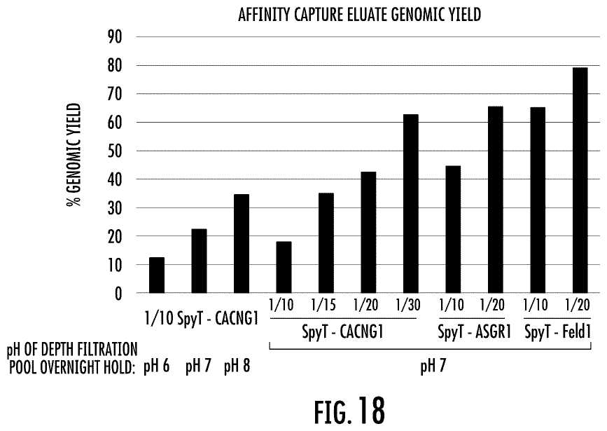

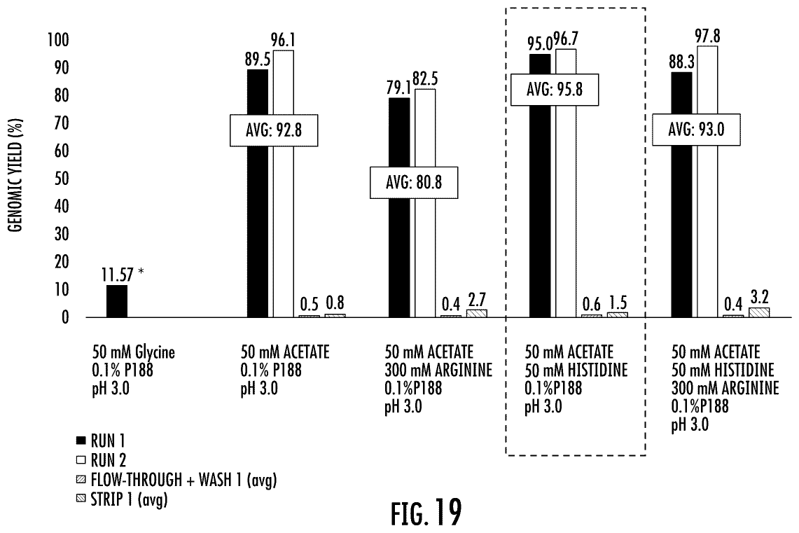

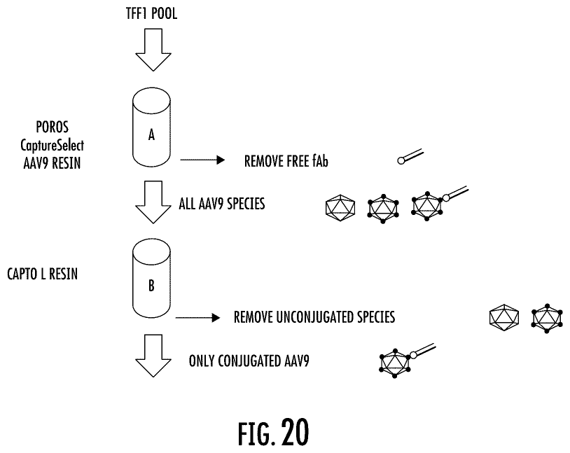

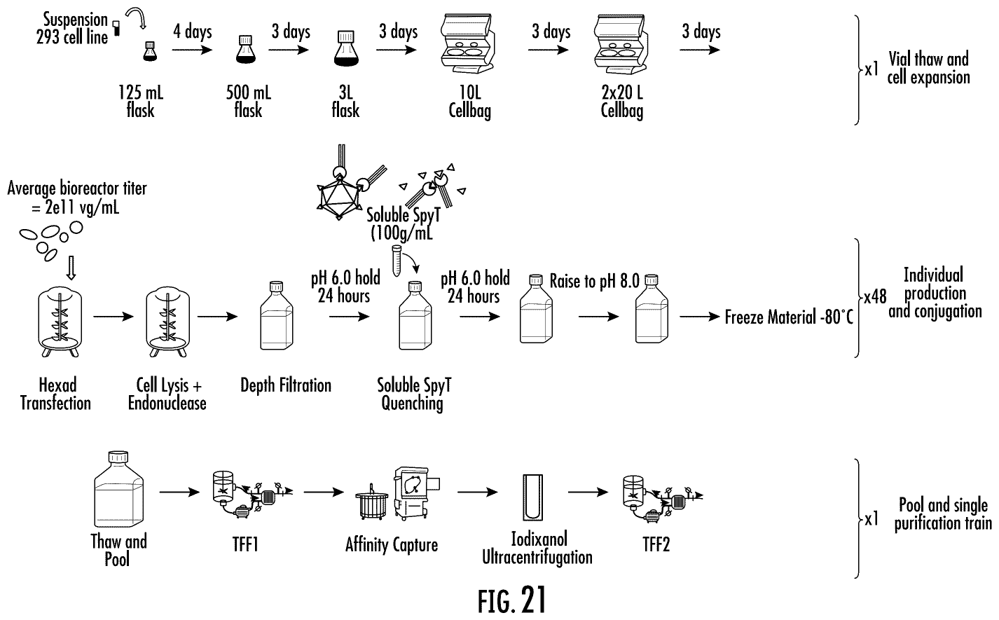

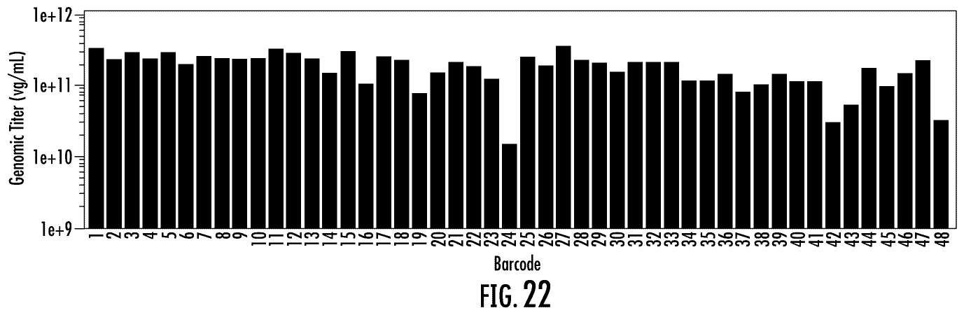

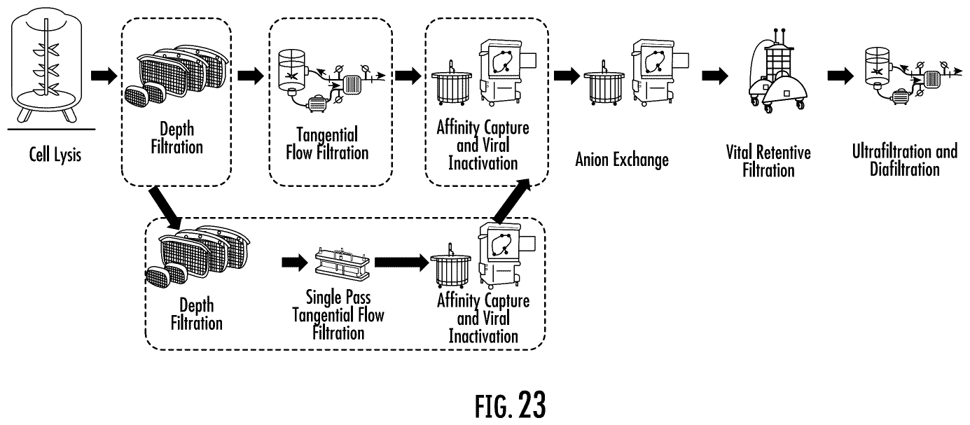

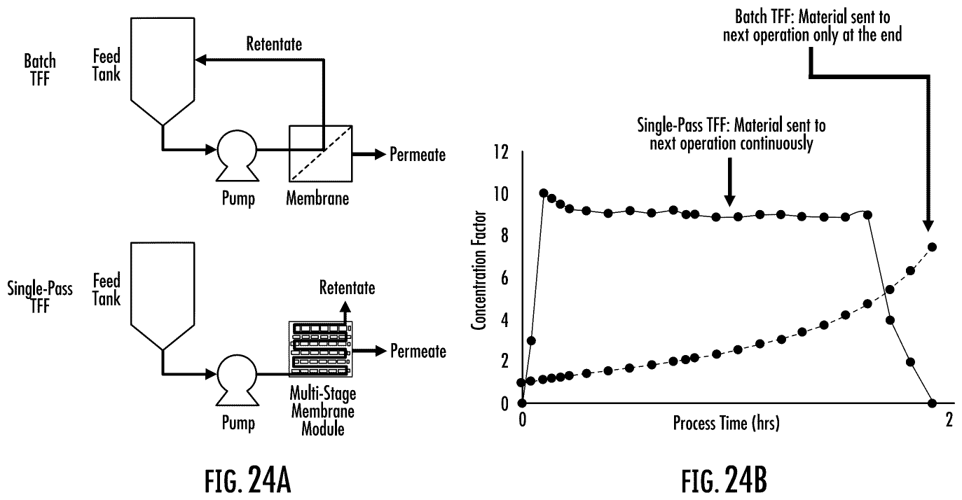

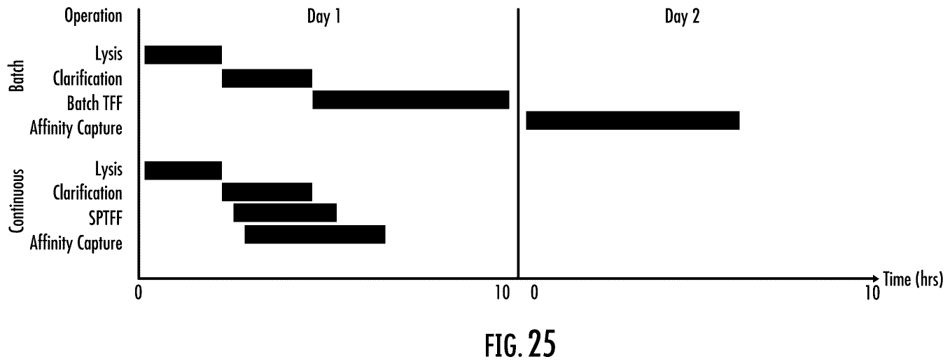

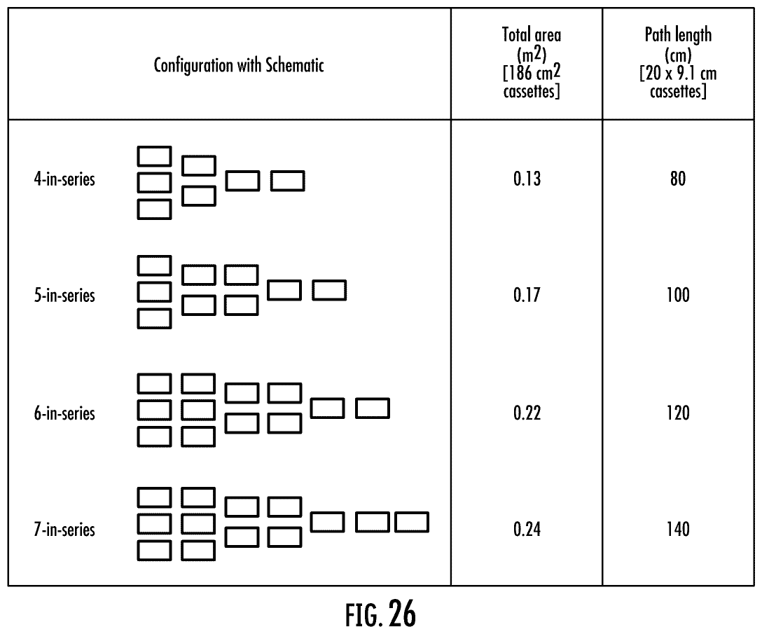

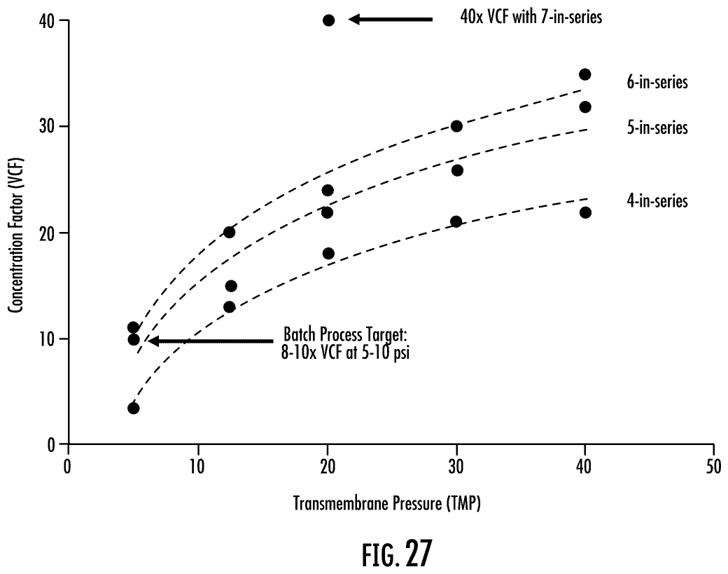

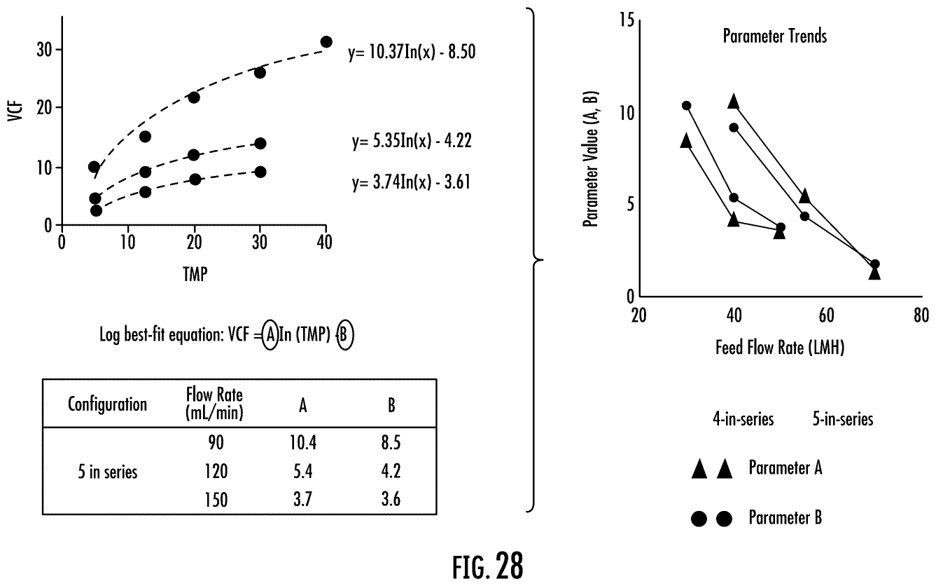

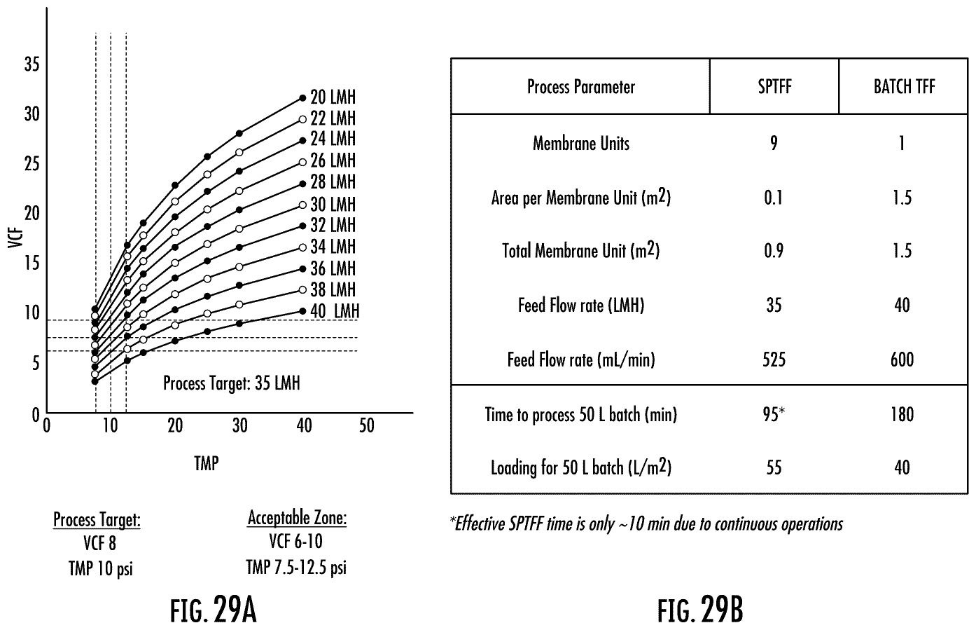

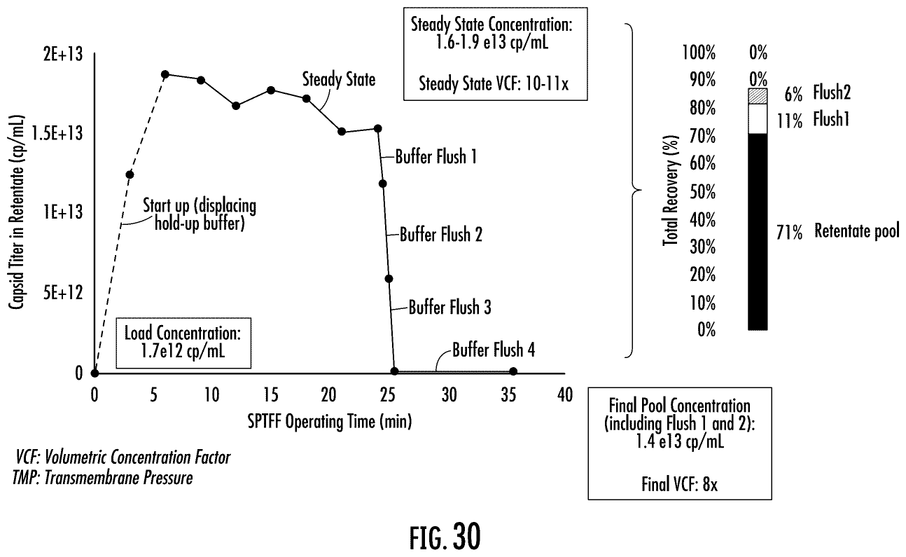

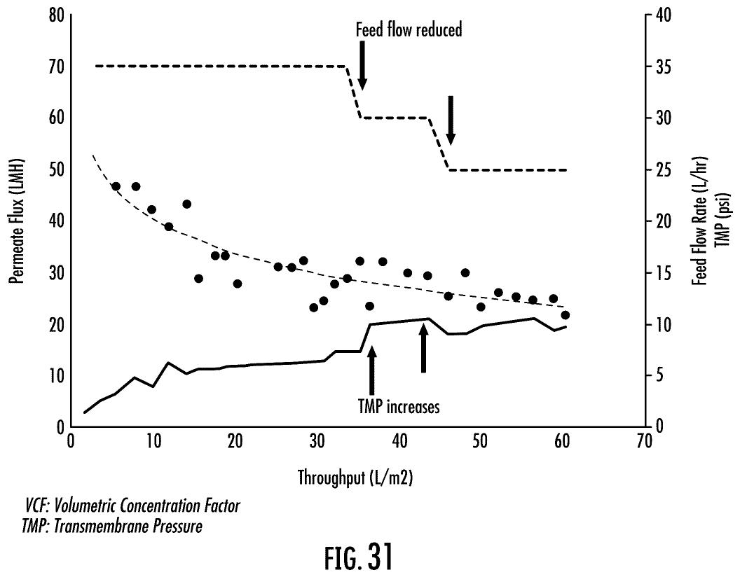

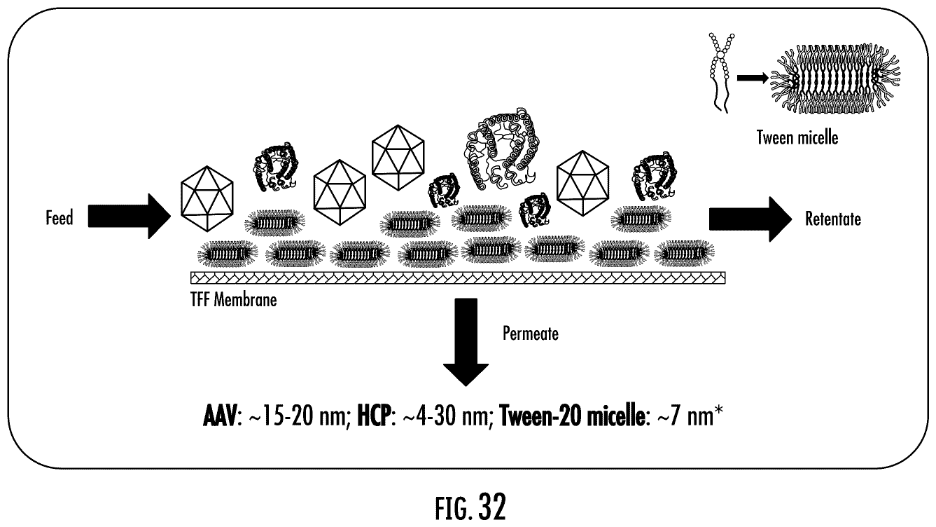

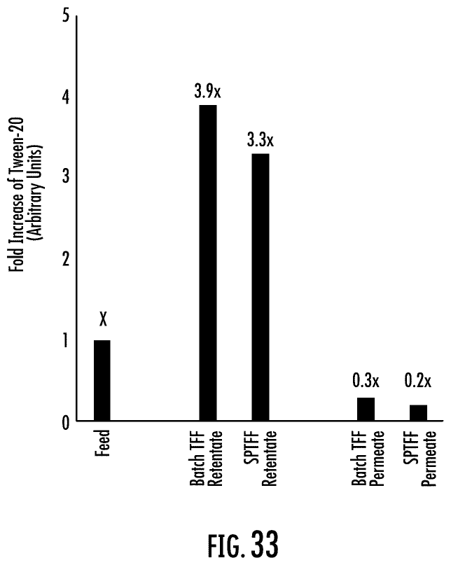

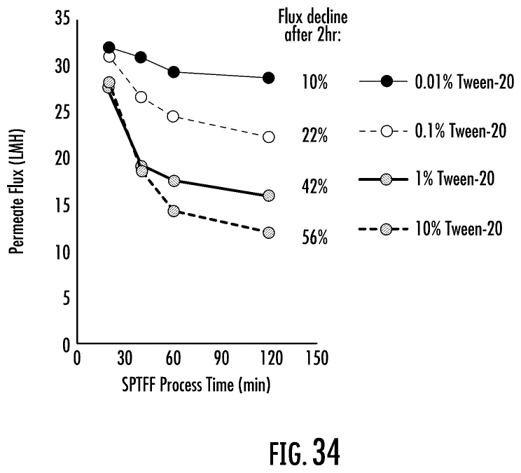

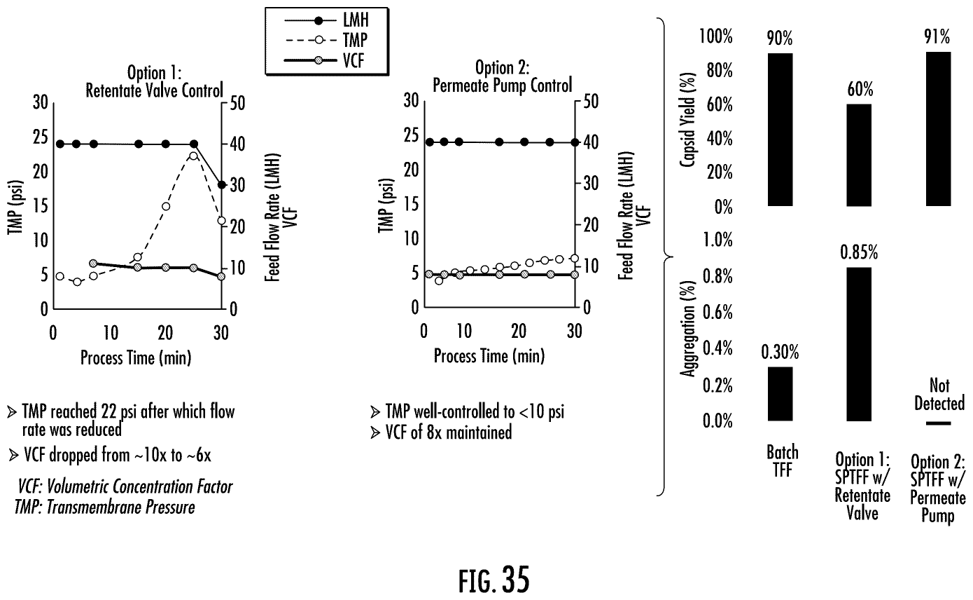

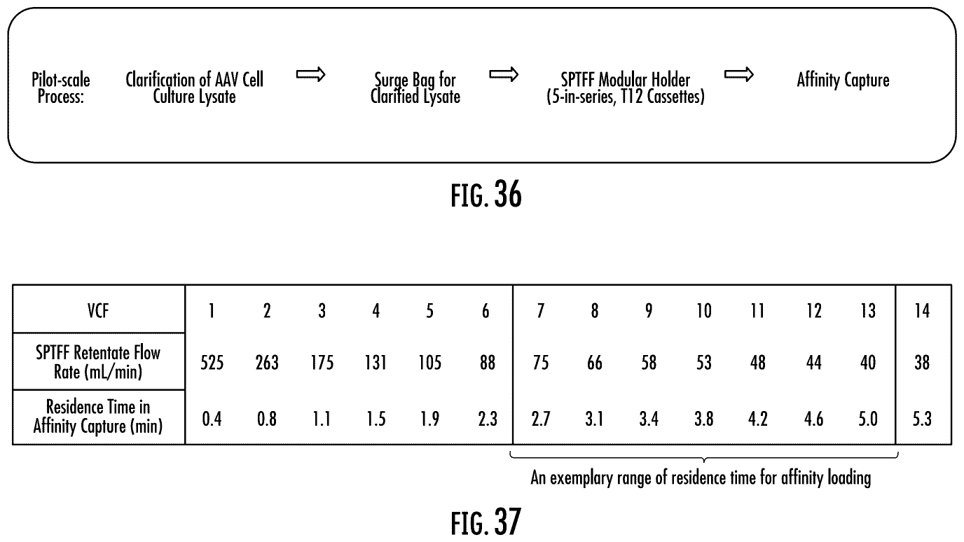

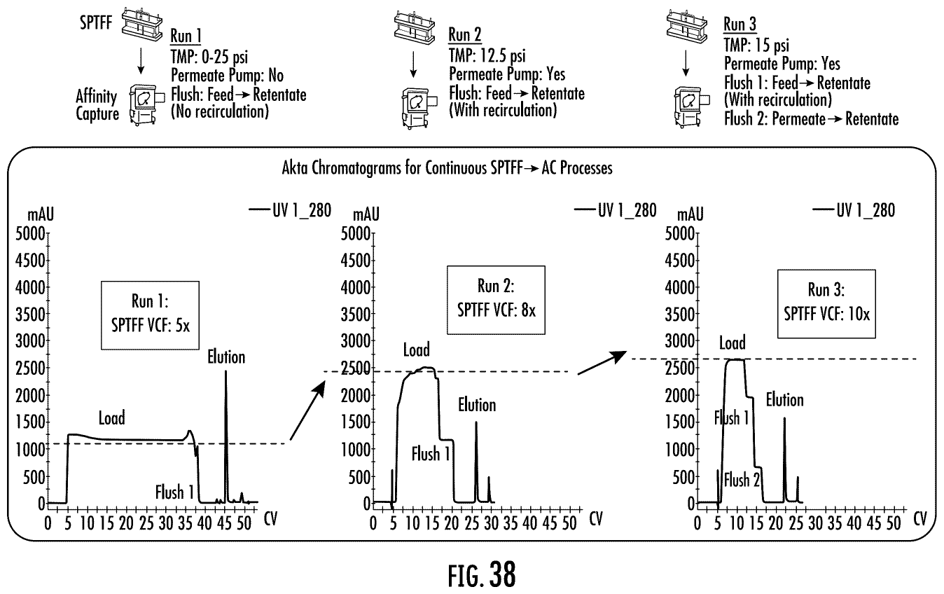

FIG. 1 contains a schematic depiction of a covalently surface modified AAV bound to a target cells. In FIG. 1, the AAV comprises a plurality of SpyTag amino acid sequences on the capsid surface. The SpyTag sequences can bind to the SpyCatcher protein. The SpyCatcher protein is expressed as a fusion protein with a retargeting molecule, here a monoclonal antibody (mAb) in FIG. 1. Preferably the SpyCatcher protein is fused to the heavy chain at the Fc portion of the mAb. The mAb at the Fv portion recognizes a target on the surface of a target cell to be transduced by the AAV. The AAV can further comprise a GOI (not shown) to be expressed once the covalently surface modified AAV.

FIGS. 2A and 2B depict and describe hexad transfection systems and methods for producing covalently surface modified AAV in a eukaryotic cell, such as HEK 293. As described in FIG. 2A, the hexad transfection system utilizes six plasmids (schematically depicted) as follows:
(i) pGOI—a plasmid comprising a GOI (for example, a transgene) flanked by two AAV inverted terminal repeats (ITRs), and optionally a first selection marker gene, such as an antibiotic resistance gene (for example, ampicillin resistance);
(ii) pRC—a plasmid comprising AAV (here AAV9 for example) rep and cap genes, and optionally a first selection marker gene, such as an antibiotic resistance gene (for example, ampicillin resistance);
(iii) pRC-SpyT—a plasmid as above in (ii) and further comprising a polynucleotide sequence encoding a 13 amino acid Spy Tag peptide (a first member of a specific binding pair), and optionally a first selection marker gene, such as an antibiotic resistance gene (for example, ampicillin resistance). This is an exemplary plasmid pRC-first member (pRC-FM);
(iv) pHELP—a plasmid comprising adenovirus helper genes E4 and E2, and VA RNA, (exemplary helper genes), and optionally a first selection marker gene, such as an antibiotic resistance gene (for example, ampicillin resistance);
(v) pHC-SpyC—a plasmid comprising polynucleotides encoding a mAb heavy chain sequence and a SpyCatcher protein sequence (a second cognate member of a specific binding pair), and optionally a first selection marker gene, such as an antibiotic resistance gene (for example, ampicillin resistance). This is an exemplary plasmid pRC-second cognate member (pRC-SCM); and
(vi) pLC—a plasmid comprising a polynucleotide encoding a mAB light chain sequence, and optionally a first selection marker gene, such as an antibiotic resistance gene (for example, ampicillin resistance). The following chart provides a helpful summation of this example:

| Plasmid type | Specifics |
|---|---|
| pGOI | Any gene(s) of interest flanked by |
| | AAV ITRs. |
| pRC | For example, comprises AAV 9 rep |
| | and cap genes. |
| pRC-FM | pRC-Spy Tag. Provides VP1, VP2 |
| | and VP3 proteins fused to Spy Tag |
| | protein |
| pHELP | Comprises one or more adenovirus |
| | helper genes |
| pHC-SCM | pHC-Spy Catcher. Provides an |
| | antibody heavy chain fused |
| | to Spy Catcher. |
| pLC | Comprises an antibody light chain. |

Transfection is preferably undertaken in mammalian cells, preferably human cell lines. Human cell lines include amniotic cells (such as Human Amniotic Epithelial cells), Hela cells, Per.C6 cells and HEK 293 cells. Examples of HEK 293 cells include, but are not limited, to HEK 293, HEK 293A, HEK 293E, HEK 293F, HEK 293FT, HEK 293FTM, HEK 293H, HEK 293MSR, HEK 293S, HEK 293SG, HEK 293SGGD, HEK 293T and mutants and variants thereof. Rodent cells and insect cells also can be used.

Promoters that are functional in eukaryotic cells, such as AAV P5, CMV, lac, CAG, CAGG, and SV40 promoters, are provided where needed to initiate transcription, but are not shown. Additionally, internal ribosome entry sites (IRESs), recombinase recognition sites (RRSs), enhancers and operators optionally can be included, but are not shown. For constructing plasmids having large polynucleotides, such as those encoding antibody chains, intronic selection can be employed, preferably with a second selection marker gene that is different from the first selection marker gene.

FIG. 2B schematically depicts at the top a pRC-SpyT plasmid comprising the rep and cap genes, and at the bottom schematically shows the insertion of the SpyTag 13 amino acid peptide sequence to form a fusion protein comprising SpyTag and Cap peptide sequences, thereby resulting in mutant VP1, VP2 and VP3 proteins fused to the SpyTag peptide sequence. The AAV still possesses the 5:5:50 stoichiometry of the VP1, 2 and 3 proteins.

Covalently surface modified recombinant AAV can be produced using any AAV serotype, for example, AAV1, AAV2,), AAV3, AAV4, AAV5, AAV6, AAV7, AAV8, AAV9, AAV10, AAV11, AAV12, AAV13, AAV rh10, AAV rh39, AAV rh43, and AAV rh74, and any other variants AAVs (for example, AAV2 7m8 or AAV2quad(Y-F), can be modified with genes of interest. Recombinant AAV capsids with modified viral capsid proteins to permit retargeting of AAV are disclosed in WO 2019/006046.

Modified capsid protein approaches according to the inventions utilize a first member and a second cognate member of a specific binding pair, which first member and second cognate member specifically interact to form a chemical, preferably covalent, bond. The first member, when displayed on a capsid protein, acts as a scaffold for any retargeting molecule (often referred to as a targeting ligand, see Yan et al., Pharmaceutics 2024 16, 248) fused to the second cognate member, but upon binding of the first member and second cognate member, an isopeptide bond forms, and the recombinant viral particle acts as a targeting vector. The covalently surface modified AAVs also can comprise GOIs and ITRs.

The second cognate member can be operably linked to a retargeting molecule. The first member can be flanked by a first and/or second linker that link(s) the first member to the capsid protein, and wherein the first and/or second linker is each independently at least one amino acid in length. The first and second linker can be identical or non-identical.

Retargeting molecules bind to targets, which are antigens, receptors and/or ligands found on the surface of a target cell. Exemplary targets are calcium voltage-gated channel auxiliary subunit gamma 1 (CACNG1), asialoglycoprotein receptor 1 (ASGR1), Fel d 1, ENTPD3, PTPRA, CD20, CD63 and Her2. Additional targets include GAB A, transferrin receptor, CD3, CD34, integrin, adipophilin, AIM-2, ALDHIAI, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNKIAI, CTAGI, CTAG2, cyclin DI, Cyclin-AI, dek-can fusion protein, DKK1, EFTUD2, Elongation factor 2, ENAH (hMena), Ep-CAM, EpCAM, EphA3, epithelial tumor antigen (“ETA”), ETV6-AML1 fusion protein, EZH2, E6, E7, FGF5, FLT3-ITD, FN1, G250/MN/CAIX, GAGE-1,2,8, GAGE-3,4,5,6,7, GAS7, glypican-3, GnTV, gplOO/Pmel 17, GPNMB, HAUS3, Hepsin, HER-2/neu, HERV-K-MEL, HLA-A11, HLA-A2, HLA-DOB, hsp70-2, IDOI, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferase AS fusion protein, Lengsin, M-CSF, MAGE-A1, MAGE-A 10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, ME1, Mel an-A/MART-1, Meloe, Midkine, MMP-2, MMP-7, MUC1, MUC5AC, mucin, MUM-1, MUM-2, MUM-3, Myosin, Myosin class I, N-raw, NA88-A, neo-PAP, NFYC, NY-BR-1, NY-ESO-I/LAGE-2, OA1, OGT, OS-9, P polypeptide, p53, PAP, PAX5, PBF, pml-RARalpha fusion protein, polymorphic epithelial mucin (“PEM”), PPP1 R3B, PRAME, PRDX5, PSA, PSMA, PTPRK, RAB 38/N Y-MEL-1, RAGE-1, RBAF600, RGS5, RhoC, R F43, RU2AS, SAGE, secernin 1, SIRT2, SNRPD1, SOX10, Sp17, SPA17, SSX-2, SSX-4, STEAPI, survivin, SYT-SSX1 or -SSX2 fusion protein, TAG-1, TAG-2, Telomerase, TGF-betaRII, TPBG, TRAG-3, Triosephosphate isomerase, TRP-1/gp75, TRP-2, TRP2-INT2, tyrosinase, tyrosinase (“TYR”), VEGF, WT1, XAGE-Ib/GAGED2a, Kras, NY-ESOI, MAGE-A3, HPV E2, HPV E6, HPV E7, WT-1 antigen (in lymphoma and other solid tumors), ErbB receptors, Melan A [MARTI], gp 100, tyrosinase, TRP-1/gp 75, and TRP-2 (in melanoma); MAGE-1 and MAGE-3 (in bladder, head and neck, and non-small cell carcinoma); HPV EG and E7 proteins (in cervical cancer); Mucin [MUC-1] (in breast, pancreas, colon, and prostate cancers); prostate-specific antigen [PSA] (in prostate cancer); carcinoembryonic antigen [CEA] (in colon, breast, and gastrointestinal cancers), and such shared tumor-specific antigens as MAGE-2, MAGE-4, MAGE-6, MAGE-10, MAGE-12, BAGE-1, CAGE-1,2,8, CAGE-3 TO 7, LAGE-1, NY-ESO-I/LAGE-2, NA-88, GnTV, TRP2-INT2, E6, E7, human glucagon receptor (hGCGR) and. I human ectonucleoside triphosphate diphosphohydrolase 3 (hENTPD3). Other targets can be selected by the person skilled in the art. See WO 2019/006046.

Systems to facilitate retargeting include the SpyTag:SpyCatcher system is described in U.S. Pat. No. 9,547,003 and Zakeri et al. (2012) PNAS 109:E690-E697, is derived from the CnaB2 domain of the Streptococcus pyogenes fibronecting-binding protein FbaB. See WO 2019/006046.

SpyTag002:SpyCatcher002 system is described in Keeble et al (2017) Angew Chem Int Ed Engl 56:16521-25. See WO 2019/006046.

SpyTag003:Spay Catcher003 also has been created. Spy Tag002:SpyCatcher002 and SpyTag003:SpyCatcher003 are different iterations of Spy Tag:Spy Catcher.

The SnoopTag:SnoopCatcher system is described in Veggiani (2016) PNAS 113:1202-07. The D4 Ig-like domain of RrgA, an adhesion from Streptococcus pneumoniae, was split to form SnoopTag. Incubation of SnoopTag and SnoopCatcher results in a spontaneous isopeptide bond that is specific between the complementary proteins. Veggiani (2016)), supra. See WO 2019/006046.

The Isopeptag:Pilin-C specific binding pair was derived from the major pilin protein Spy0128 from Streptococcus pyogenes. (Zakeir and Howarth (2010) J. Am. Chem. Soc. 132:4526-27). See WO 2019/006046.

Other systems to facilitate retargeting can be based upon the splitting and engineering of RegA domain 4. These have led to SnoopTagJr:SnoopCatcher, DogTag:DogCatcher and Snoop Ligase. Other systems include Isopeptag:Pilin-N, SdyTg:SdyCatcher, Jo:In, 3kptTag: 3kptCatcher, 4oq1Taq/4oq1 Catcher, NGTag/Catcher, Rumtrunk/Mooncake, GalacTag, Cpe, Ececo, Corio and all others based upon isopeptide binding pairs.

The present inventions are amenable for production in mammalian cell culture. Exemplary rodent cell lines are CHO, Per.C6 cells, Sp2/0 cells, and HEK293 cells. CHO cells include, but are not limited to, CHO-ori, CHO-K1, CHO-s, CHO-DHB11, CHO-DXB11, CHO-K1 SV, and mutants and variants thereof. HEK293 cells, a preferred human cell line, include, but are not limited, to HEK293, HEK293A, HEK293E, HEK293F, HEK293FT, HEK293FTM, HEK293H, HEK293MSR, HEK293S, HEK293SG, HEK293SGGD, HEK293T and mutants and variants thereof. Other suitable cells include, but are not limited to BHK (baby hamster kidney) cells, HeLa cells and Human Amniotic cells, such as Human Amniotic Epithelial cells. Other cell types for production include insect cells, such as Sf9.

Covalently surface modified AAV vectors can be produced using a suspension cultured HEK293 derived cell line, such as HEK293F, using a commercial media CTS LV-MAX (Thermo Fisher Scientific). These viral vectors have been successfully produced in fed-batch bioreactors at various scales from 200 mL to 50 L with temperature controlled at about 37° C., pH controlled between about 6.7-7.5, agitation power input of about 22 W/m3, dissolved oxygen level of about 30%. Cells are typically transfected at about 24 hours post inoculation of the production bioreactor, and harvested at 3-5 days post transfection.

Adherent HEK 293 cells also can be used for production of covalently surface modified AAV according to the inventions. HEK 293 suspension cultured cells were derived from HEK 293 adherent cells. See Maim et al., Scientific Reports 10: 18996 (2020).

All major antibody classes, namely IgG, IgA, IgM, IgD and IgE, can be used as targeting molecules. IgG is a preferred class, and includes subclasses IgG1 (including IgG1A and IgG1K), IgG2, IgG3, and IgG4. Further antibody types include a human antibody, a humanized antibody, a chimeric antibody, a monoclonal antibody, a multispecific antibody, a bispecific antibody, a trispecific antibody, an antigen binding antibody fragment, a single chain antibody, a diabody, triabody or tetrabody, a Fab, F(ab′) or a F(ab′)2, an IgD antibody, an IgE antibody, an IgM antibody, an IgG antibody, an IgG1 antibody, an IgG2 antibody, an IgG3 antibody, or an IgG4 antibody. Derivatives, components, domains, chains and fragments of the above also are included as types of targeting molecules.

As stated above, different AAV serotypes are known to preferentially transduce the cells of different tissues. Tissue specificity is limited, and AAV is known to preferentially transduce the liver, which can be a safety and efficacy concern in some contexts. The inventions further provide mutations in the VP1 Protein of AAV9 to lower the AAV preferential transduction of the liver. The mutations include N272A and W503A substitutions, where alanine replaces both asparagine at position 272 of VP1 and tryptophan at position 503 of VP1. One or both of the mutations can be undertaken in the VP1 protein. Optionally, other amino acids, such as glutamic acid, serine or others, can be used instead of alanine for substitution. Other detargeting mutation sites include, but are not limited to, N470, D271, and Y446. The inventions further provide mutations for other AAVs are as follows:
AAV1—N500E
AAV2—R585A and R588A
AAV5—T571 S
AAV6—N500E, K531A and K531E.

These and others are set forth in the chart below:

| AAV | Insertion | Exemplary |
|---|---|---|
| serotype | Sites | Mutations |
| AAV2 | 1, 34, 138, 139, 161, 261, | R484, R487, R585A, |
| | 266, 381, 447, 448, 453, | R588A and K532. |
| | 459, 471, 520, 534, 570, | R484A, R487A, R487G, |
| | 573, 584, 587, 588, 591, | K532A, K532D, R585A, |
| | 657, 664, 713, 716 | R585S, R585Q, R588A, |
| | | R588T |
| AAV9 | 272, 453, 503, 587, 589 | N272A, W503A |
| AAV1 | 587, 589 | N500E |
| AAV3 | 585 | |
| AAV4 | 584, 585 | |
| AAV5 | 531, 571, 575, 585 | K531A, K531E, T571S |
| AAV6 | 500, 531 | N500E, K531A, K531E |
| Avian AAV | 444, 580 | |
| Sea lion | 429, 430, 431, 432, 433, | |
| AAV | 434, 436, 437, 565 | |
| Bearded | 573, 436 | |
| Dragon AAV | | |

Still other mutations for all AAV serotypes are available to the person skilled in the art.

The AAVs were evaluated using affinity chromatography. Available affinity capture resins include, but are not limited to: POROS CaptureSelect AAVX, POROS CaptureSelect AAV8, POROS CaptureSelect AAV9, Capto AVB, AVB Sepharose, Avipure AAV2, Avipure AAV8, Avipure AAV9, among others. Resins used for affinity capture via antibody surface epitopes can comprise mAbSelectSuRe, mAbSelect PrimaA, Capto L, mAbSelect VL, and KappaSelect.

The inventions provide the skilled person with the ability to modulate the percent of SpyTag inserted capsid and subsequently the level of antibody conjugation through changing the fraction of the pRC-SpyT plasmid during hexad transfection. Level of antibody conjugation correlates with in-vitro infectivity.

A low pH hold in a bioreactor harvest has been found to be beneficial in order to drive the conjugation reaction to completion; that is, to ensure that any available SpyT peptides on AAV capsids are conjugated to SpyC-Antibodies, which are expressed in excess, as described herein.

The inventions are further described by the following Examples, which do not limit the inventions in any manner and are applicable to all sections of the descriptions of the inventions and the aspects of the inventions. The order of performance of the below Examples can be altered or combined as determined by the person of skill in the art in view of the teachings and data contained herein.

The AAV serotype sequences and component sequence types that are used or can be used according to the inventions, and are discussed in the Description, Summary, Examples and Figures are representative and not limiting. Any AAV serotype sequence and component sequence can be used according to the inventions. These include but are not limited to sequences for promoters, markers, specific binding pairs, helper proteins, AAV sequences (Cap, Rep and ITRs), retargeting molecules, detargeting mutations, IRESs, RRSs, introns, operators, enhancers and polydenylation signals.

### Example 1—Transfection Parameters—Design of Experiment 1

At 24 hours post inoculation of the production bioreactor, cells can be transiently transfected with 6 plasmids to produce the rAAV and additional components for covalent surface modification. The FectoVIR-AAV (Polyplus) is selected as the transfection reagent to deliver the plasmids into the cells. To achieve high yield production of rAAV, parameters defining the transfection procedure should to be optimized, including the cell density at the time of transfection, plasmid and transfection reagent concentration and ratios, and transfection complex incubation time.

A triple transfection using three plasmids (pGOI, pRC, pHELP) was used as a simplified platform to optimize the relative amounts of AAV transgene, functional and structural gene Rep and Cap, and helper polynucleotide sequences as a first step, prior to the introduction of plasmids encoding for the various components for covalent surface modification. A total of 6 factors were evaluated in a 24-run D-optimal custom Design of Experiment (DOE) study, referred to as DOE 1. Additional 10 bioreactor runs were added to augment the study design to further characterize the effects on the edge of the studied factor ranges. Table 1 summarizes the range studied and the optimal value identified for each factor to maximize the rAAV vector genome titer and percent of full rAAV capsids. FIG. 3 depicts the prediction profiler from Design of Experiment study 1 on the effect of transfection parameters on rAAV titer, percent full capsids, and residual host cell DNA (hcDNA). Optimal amount of RC plasmid identified from this study was used for subsequent hexad transfection but split at a fixed ratio between pRC and pRC-SpyT. The parameters are set forth below in Table 1:

TABLE 1

| | Preferred | Preferred | Additional |
|---|---|---|---|
| Factor | Ranges | values | Ranges |
| Cell Density at | 2 × 10⁶ to | 4 × 10⁶ | 1 × 10⁶ to |
| Transfection | 6 × 10⁶ | cells/ml | 8 × 10⁶ |
| (cells/mL) | cells/ml | | cells/ml |
| Total DNA (μg/ml) | 1 to 4.5 | 2 | 0.5 to 8 |
| pRC/pGOI Ratio* | 0.5 to 10 | 3 | 0.2-10 |
| (by mass) | | | |
| pHELP/pGOI Ratio* | 0.5 to 3 | 2 | 0.2-5 |
| (by mass) | | | |
| FectoVIR/DNA | 0.8 to 2 μl/μg | 0.8 μl/μg | 0.5-3 μl/μg |
| (vol/mass) | | | |
| Complexation Time | 15 to 30 | 15 | 5-30 |
| (minutes) | | | |

*The basis of the ratios are the GOI plasmid (pGOI).

Significant models were obtained for viral genomic titer (R_{adj}²=0.791, RMSE=7.1×10¹⁰ vector genomes (vg)/mL, p<0.0001), capsid titer (R_{adj}²=0.815, RMSE=1.3×10¹² cp/mL, p<0.0001) and percentage of full capsids (R_{adj}²=0.842, RMSE=4.4%, p<0.0001) using a standard least squares fit algorithm.

The data indicate that increasing the total DNA concentration and pRC/pGOI ratio and lowering the transfection reagent to DNA ratio increased the vector genome titer, but lowered the percentage of filled capsids. Reducing the transfection complex incubation time had no impact on vector genome titer, but improved the percent of full capsids. Increasing the cell density at transfection and the pHELP/pGOI ratio to a medium range improved both titer and percent of full capsids. Total RC plasmid to pGOI ratio should be about between 3.5-6.5, and pHELP to pGOI ratio should be about 0.4-2.0. Optimal parameter values are identified to achieve a balance in the optimization objectives. The predicted titer is 4.3×10¹¹ vg/mL and percent full capsids of 25%

### Example 2—Bioreactor Operating Parameters-Design of Experiment 2

Controlling the cell culture environment at optimal state in respect to temperature, aeration, and pH can significantly enhance viral production. Specifically, pH of the bioreactor can be optimized to meet the demands of various phases of the viral production process, with the pH suited for cell growth potentially being different from the optimal pH for transfection complex internalization and viral vector production. Similarly, the agitation speed of the bioreactor can be studied to identify the optimal agitation speed for cell growth with minimal cell aggregation, and the agitation speed suitable for transfection without significant shear force to the transfection complex. To evaluate the impact of these two bioreactor parameters in detail, a total of 8 factors were studied in a 46-run D-optimal custom Design of Experiment study (DOE 2) to investigate the impact of the bioreactor pH and agitation speed at three different phases of production and the transition timings between phases on vector titer and quality. Table 2 summarizes the range studied and the optimal value identified for each factor to maximize the rAAV vector genome titer and percent of full rAAV capsids.

FIG. 4 depicts the prediction profiler from DOE 2 on the effect of the bioreactor parameters on rAAV titer and percent full capsids. The SpyTag AAV (here, AAV9) was produced in this experiment without the introduction of any covalent surface modification. Optimal bioreactor setpoints identified from this study is validated on the production of conjugated AAV9. RC plasmid ratio used in this study is identified from the previous transfection multivariate study, but split between the pRC and pRC-SpyT at a fixed ratio of 9:1 ( 1/10 SpyTag fraction).

TABLE 2

| | Preferred Ranges | | Additional Ranges |
|---|---|---|---|
| | (which sets forth | | (which sets forth |
| | and contains all | | and contains all |
| | values within | Preferred | values within |
| Factor | the range) | values | the range) |
| Growth pH | 6.6-7.4 | 7.1 | 6.6-7.6 |
| Transfection pH | 6.6-7.4 | 7.3 | 6.6-7.6 |
| Production pH | 6.6-7.4 | 7.3 | 6.6-7.6 |
| Grow to Transfect | 2-12 | 12 | 1-24 |
| Switch Time | | | |
| (HPreT) | | | |
| Transfect to | 1-12 | 1-12 | 1-24 |
| Produce Switch | | | |
| Time (HPostT) | | | |
| Growth Agitation | 7-22 | 22 | 7-25 |
| (W/m³) | | | |
| Transfect Agitation | 7-22 | 22 | 7-25 |
| (W/m³) | | | |
| Produce Agitation | 7-22 | 22 | 7-25 |
| (W/m³) | | | |

HPreT, hours pre-transfection; HPostT, hours post-transfection. Agitation speed is expressed in power input per unit volume in W/m³ unit.

The data indicate that controlling the bioreactor pH at a high level of 7.3 during the transfection complex update phase and production of AAV improves the vector titer, while a modest pH of 7.1 is ideal for the cell growth phase prior to transfection. Shifting the pH from the growth pH up to transfection/production pH at an earlier time point is beneficial to improve percent of full capsids. Agitation speed, on the other hand, has minimal impact on viral production within the range studied. Optimal bioreactor pH and agitation set points are identified to achieve a balance between improving vector genome titer and percent of full capsids. Using these optimal parameter values, the model predicted a vector genome titer of 2.9×10¹¹ vg/mL and a percent of full capsids of 17.7%.

### Example 3—The Effect of Varying pRC-SpyT to pRC Plasmid Ratios

The average number of covalent surface modifications on a single AAV is dominated by the number of SpyTag insertions on the VP proteins that make up the AAV capsid. These SpyTag insertions provide available docking sites for the free SpyCatcher antibodies to bind and form covalent bonds. The percentage of VP proteins that contain the SpyTag insertion is dictated at the genetic level by the relative amount of the RC plasmids with or without the SpyTag sequence that are introduced to the cells.

This study demonstrated for the first time the production of antibody conjugated AAV9 using a suspension cultured HEK293 cell line in a controlled fed-batch bioreactor system. The SpyTag AAV (here, AAV9) and SpyCatcher-Rm (anti-CACNG1) are produced simultaneously following the hexad transfection of plasmids encoding for all necessary components. A range of SpyTag fractions were used to demonstrate the ability to tune the percentage of SpyTag insertion to the AAV capsid and subsequently the level of antibody conjugation through the introduction of the pRC-SpyT and pRC plasmids at different ratios during hexad transfection. The AAV9 capsids with two point mutations (N272A and W503A) were produced in separate productions to support the investigation of AAV9 detargeting from the liver tissue. Various control conditions were included in the production study design to progressively introduce the detargeting and retargeting components and facilitate understanding of the impact of these components on viral production titer.

As shown in FIG. 2A, there are two plasmids encoding AAV Rep and Cap. Plasmid RC (pRC) encodes Rep and Cap, but not SpyT, whereas pRC-SpyT encodes Rep and Cap (with Spy-T inserted into Cap) (see FIG. 2B).

FIG. 5A shows AAV with no SpyTag insertion (lanes 1-2); AAV with ⅛ SpyT insertion and a N272A detargeting mutation (lane 3) or a W503A detargeting mutation (lane 4); AAV with ¼ to 1/20 SpyT insertion, antibody sequences and a N272A detargeting mutation (lanes 5-10); AAV with ¼ to ⅛ SpyT insertion, antibody sequences and a W503A detargeting mutation (lanes 11 and 12).

The SpyTag fraction refers to the part of pRC-SpyT plasmid of the total RC plasmids at plasmid level. SpyTag Insertion refers to the addition of SpyTag to the VP proteins. The SpyTag insertion is on a percentage of VP proteins and is largely dependent on the fraction of the pRC-SpyT plasmid used during transfection. “Conjugation” refers to the linking of SpyCatcher-Rm (for example, a mAb or Fab) to the SpyTag AAV.

The illustrations at the bottom of FIG. 5A depict the concept of capsid modification. Samples were stored at 4° C. for 10 days to permit conjugation to complete.

FIG. 5B shows that unmodified capsids are produced with each construct (bottom set of bands). SpyTag AAV capsids are produced in lanes 3-4 reflected by the additional VP3 band with slightly increased molecular weight. Antibody modified capsids are produced in lanes 5-12 when antibody plasmids are introduced during the hexad transfection. The antibody conjugation to the AAV capsid proteins results in a shift in molecular weight (top set of bands). The concurrent lack of the bands corresponding to the SpyTag AAV capsids indicate that antibody conjugation is close to completion and few un-conjugated SpyTag AAV is remaining following the extended hold for 10 days at 4° C.

FIG. 5C shows the infectivity results of the covalently surface modified (in this case anti-CACNG1 mAb conjugated) AAV with varying levels of antibody conjugation. In this cell-based assay, HEK293 cells that express CACNG1 surface antigen are dosed with the respective AAV vector preps in each lane. Two different spiking volumes of the vector preps are tested to screen for appropriate multiplicity of infection for this assay. The AAV vectors produced in this study carry a recombinant viral payload containing a CAGG promoter and a transgene encoding for green fluorescent protein. Cells that are transduced with the AAV vectors will receive the recombinant viral payload and express GFP, and the intensity of the fluorescent signal indicates infectivity of the AAV virus prep.

Antibody conjugation led to greater infectivity than control AAV (lanes 1 and 2) and AAV with only detargeting mutations (lanes 3 and 4). Infectivity of the vector preps negatively correlates with level of antibody conjugation. Lower SpyTag plasmid fractions, resulting in less antibody conjugation, corresponded to higher infectivity.

### Example 4—The Effect of Increasing Rm and SpyCatcher-Rm Plasmids

The conjugation of the SpyCatcher-Rm (for example, a mAb or Fab) to SpyTag AAV occurs predominantly at the end of the fed-batch production when cells are lysed apart using chemical detergent. SpyTag AAV that primarily retained inside the cells comes together with the SpyCatcher Rm that is secreted outside of the cells, and the spontaneous conjugation reaction occurs. The rate of the conjugation reaction is dependent on the temperature, pH and detergent concentration of the reaction mixture (Zakeri B, Howarth M, et al., 2012, PNAS), as well as the concentrations of the SpyTag AAV and SpyCatcher-Rm as reaction substrates. A faster conjugation kinetic is beneficial to allow completion of reaction well within the time frame of the processing step and reduce production batch to batch variability in case the process intermediate hold time is varied.

Using a total DNA concentration of 2 μg/mL for the four plasmids encoding for SpyTag AAV, and a total DNA concentration of 0.28 μg/mL for the pHC-SpyC (heavy chain and SpyCatcher-Rm) and pLC (light chain) plasmids encoding for the SpyCatcher-Rm during transfection, the cells produce on average 30-fold SpyCatcher-Rm in excess in molar concentration relative to SpyTag AAV. At this ratio of SpyCatcher-Rm over SpyTag AAV in the clarified cell lysate, the conjugation reaction progresses at a suboptimal rate, showing close to complete conjugation only after extended incubation for 10 days at 4° C.

To study the effect of SpyCatcher-Rm level on rate of the conjugation reaction in the clarified lysis, the total DNA concentration of the pHC-pSpyC and pLC antibody plasmids used during transfection were changed. The mass ratio of pHC:pLC was kept the same at 1:2. A total of six different concentration conditions are studied in addition to the control condition, by lowering the total DNA concentration (represented by negative sign) by 75%, 50%, 25% or by increasing the concentration by 50%, 100% or 200%.

FIG. 6A is a bar graph depicting the impact of mAb plasmid DNA concentration used in transfection on SpyCatcher-Rm (here, SpyCatcher-mAb) production titer. Increasing the mAb plasmid DNA concentration across the entire studied range improved the mAb titer by 45-100%. FIG. 6B is a bar graph depicting SpyTag AAV titer was not affected by SpyCatcher mAb titer within the range studied. FIG. 6C shows that Increasing the SpyCatcher mAb plasmid DNA (pHC-SpyC) led to more efficient conjugation of AAV in the ⅛ SpyTag fraction condition. In FIG. 6C, lanes 1 to 6 are for the ⅕ fraction of SpyT (pRC-SpyT plasmids to pRC plasmids is 1:4), and lanes 7 to 12 are for the ⅛ ratio of SpyT (pRC-SpyT plasmids to pRC plasmids is 1:7). To allow faster conjugation kinetics while maintaining the AAV production titer, the total concentration of the antibody plasmids is doubled (+100% condition) to 0.56 ug/mL during transfection in the final production process.

### Example 5—Transduction Efficiency

The plasmid concentration and ratios used during transient transfection were further evaluated for their indirect impact on transduction efficiency of the vector product. See FIG. 7. The range of the SpyTag fraction was extended to 1/30, and the range of mAb DNA concentration was kept above control level (positive sign) to enhance rate of conjugation. Spy Tag fractions ranged from ⅕ (pRC-SpyT to pRC is 1:4) to 1/30 (pRC-SpyT to pRC is 1:29). The percent change in mAB DNA concentration ranged 0% to 200%, meaning 0%=0.28 μg/mL, 100%=0.56 μg/mL, 200%=1.12 μg/mL. Lane 5, which had a 1/10 SpyTag fraction (pRC-Spy T to pRC is 1:9) and a 100% increase in mAb DNA showed the highest transduction efficiency. The signal was generated using a green fluorescent reporter gene.

### Example 6—Low pH Hold

The hexad transfection process involves supplying AAV, AAV-SpyT, and SpyC-Antibody plasmids simultaneously to mammalian cells, such as HEK293 cells, and therefore results in expression of AAV capsids, AAV-SpyT capsids, and SpyC-Antibodies in the bioreactor. While some of the AAV-SpyT capsids are conjugated to SpyC-Antibodies naturally in the bioreactor, a proportion remain unconjugated. This is disadvantageous as the objective is to achieve maximum production of conjugated AAV-SpyT-SpyC-Antibody species.

A low pH hold in the clarified bioreactor harvest has been found to be beneficial in order to drive the conjugation reaction to completion; that is, to ensure that any available SpyT peptides on AAV capsids are conjugated to SpyC-Antibodies, which are expressed in excess. FIG. 8 sets forth data from an experiment showing the usefulness of low pH hold to drive the conjugation reaction to completion for material from four different 2 liter bioreactors (Bioreactor A, B, C, D) cultivated using the hexad transfection approach with different SpyC-Antibody plasmids in each bioreactor. Experiments were conducted at pH values of 6, 7 and 8, and pH 6 performed the best.

Lanes 1, 3, 5 and 7 show the bioreactor lysate material directly after clarification for Bioreactors A, B, C and D, respectively. It can be seen that there is a faint band above the “VP3” band, indicating the presence of VP3-SpyT; that is, a denatured VP3 capsid protein with the SpyT peptide included in its sequence. There is also a faint band in the upper half of the gel indicating the presence of VP3-SpyT-SpyC-Ab; that is, the VP3 capsid protein conjugated to a SpyC-antibody.

Lanes 2, 4, 6, and 8 show the material from Bioreactors A, B, C, and D, respectively, after clarification followed by an overnight hold at pH 6. In these lanes, the VP3-SpyT band no longer appears, and the bands in the upper half of the gel are darker. This indicates that the unconjugated VP1/2/3-SpyT proteins have been completely conjugated to SpyC-Ab, causing the higher molecular weight bands to become darker in the upper half of the gel. Thus, the low pH hold can be seen to be a part of the production process for conjugated AAV-SpyT-SpyC-Ab species using the hexad transfection approach, and is useful for driving the conjugation reaction to completion and resulting in maximum yield of conjugated species.

### Example 7—Depth Filtration

Cell lysis is an early part of AAV purification. Detergents can be used to lyse cells to release proteins and viruses contained with the cell. Detergents to lyse cells are usually considered mild detergents and include: sodium dodecyl sulphate (SDS), NP-40, Tweens (for example 20 and 80), Tritons (for example X-100 and X-114), CHAPS, CHAPSO, Brij (for example, 35 and 58), Octyl thioglucoside, Octyl Glucoside, deoxycholate, and alkyl sulfates, for example.

Depth filtration typically follows lysis. Single-use chromatographic clarification uses a next-generation synthetic fibrous anion-exchange chromatographic clarification media to simultaneously conduct depth filtration, removal of cellular debris, soluble negatively charged impurities, and sterilize filter cell harvest material. Although traditionally used for mAb processing, these filters offer a unique benefit for rAAV production as they can process chromatin-containing material without excessive reduction in capacity, presenting an opportunity to eliminate expensive endonuclease (for example, Benzoase) treatment.

Harvest RC (3M) is a single-use chromatographic clarification unit consisting of synthetic fibrous anion exchange (AEX) media and a 0.2 μm polyethersulfone (PES) membrane. Cells are bound inside the fibrous AEX media by, allowing efficient retention of large and small particles without caking that rapidly fouls the filter. This structure also captures large strands of DNA without rapid fouling, unlike conventional depth filters which require the DNA to be digested by endonuclease prior to clarification.

Tests with the Harvest RC (HRC) filters were conducted under different conditions of endonuclease addition and salt treatment. Comparison runs were also conducted with traditional C0SP depth filtration media made of polypropylene fibers. HRC data and C0SP data are set forth in FIG. 9A (rAAV8 at 3×10⁶ cells/ml) and FIG. 9B (rAAV9 at 1.5×10⁶ cells/ml). The top figures are for Total Host Cell DNA (ng/ml) and the bottom figures are for Total Host Cell Protein (ng/ml).

FIG. 9A is a bar graph for AAV8 depicting in order bars for bioreactor lysate (white bars), filtrate pool where transmembrane pressure (TMP) is 5 psi (light gray bars), and filtrate pool where TMP is 15 psi (black bars). HRC (solid bars) and C0SP (a standard filter used as a control) (hatched and labeled bars) are filtration trains with different endonuclease and salt conditions in the load. FIG. 9B is a bar graph for AAV9 depicting in order bars for bioreactor lysate (white bars), filtrate pool where TMP is 5 psi (light gray bars), and filtrate pool where TMP is 15 psi (black bars). HRC (solid bars) are filtration trains with different endonuclease and salt conditions in the load. In FIG. 9A, significant breakthrough of HCDNA into the filtrate was observed for C0SP at the “C0SP Negative Control”, and for HRC at 250 mM NaCl and 0 U/ml endonuclease (dark gray bars). In FIG. 9B, significant breakthrough of HCDNA into the filtrate was observed for HRC at 250 mM NaCl and 0 U/ml endonuclease (dark gray bars) and 250 mM NaCl and 10 U/ml endonuclease (dark gray bars).

Notably, the C0SP filter was unable to achieve significant removal of Host Cell DNA in the endonuclease-free train, with greater than 10⁴ ng/mL present in both the 5 and 15 psi fractions. In contrast, the HRC filters successfully removed Host Cell DNA to less than 10² ng/mL in all filtration trains in low-salt conditions for rAAV8 (FIG. 9A). In particular, for the endonuclease-free-trains a 100-fold reduction in host cell DNA (“HCDNA”) was achieved, from greater than 10⁴ ng/mL in the load material to less than 102 ng/mL in the filtrate fractions.

Additionally, capsid binding was not observed in any of the experiments on bioreactor harvest material, likely due the presence of sufficient HCDNA and other negatively charged impurities to compete with the capsids for the charged binding sites on the Harvest RC filter.

However, in the high salt condition when 250 mM NaCl was added to the load material, the HRC filter was unable to achieve effective Host Cell DNA (HCDNA) removal for the medium-endonuclease case, though the high salt level did not affect the filtrate quality in the high-endonuclease case. This is likely due to the salt competing with the Host Cell DNA for binding onto the anion exchange sites on the HRC filter, lowering the binding capacity. Finally, no significant reduction or impact on Host Cell Protein was observed between the load material and the filtrate fractions for any of the tested conditions.

For rAAV9, the low-salt and endonuclease-free conditions once again performed remarkably well, with 1000-fold reduction of Host Cell DNA from greater than 10⁴ ng/mL in the load material to 10¹ ng/mL in both the 5 and 15 psi filtrate fractions for 0-100 mM NaCl. See FIG. 9B. In all runs, it was observed that high salt conditions of 250 mM NaCl led to significant Host Cell DNA breakthrough into the filtrate for both the 0 U/mL and 10 U/mL endonuclease filtration trains, showing that limiting salt addition is critical for endonuclease-free clarification to be successful. The data with AAV8 and AAV9 show that an endonuclease-free clarification process can provide reduction of Host Cell DNA to comparable levels as conventional processes that use 100 U/mL of endonuclease in the bioreactor during lysis.

FIGS. 10A-10F provide data for depth filtration treatment at various salt and endonuclease conditions. FIGS. 10A-10C provide data from HRC depth filtration of AAV8 and FIGS. 10D-10F provide data from HRC depth filtration of AAV9. FIGS. 10A and 10D have no endonuclease added. FIGS. 10B and 10E have 10 units/ml of an endonuclease added. FIGS. 10C and 10F have 100 units/ml of an endonuclease added. Each of FIGS. 10B-10F contain data with concentrations of 0 mM NaCl, 100 mM NaCl and 250 mM NaCl. FIG. 10A contains data with concentrations of 0 mM NaCl and 100 mM NaCl with HRC and 0 mM NaCl with C0SP. FIG. 10C data with concentrations of 0 mM NaCl, 100 mM NaCl and 250 mM NaCl for HRC and 250 mM NaCl for C0SP.

Data from HRC and C0SP with AAV8 and AAV9 indicate Host Cell DNA breakthrough in FIG. 10A (0 mM NaCl with 0 U/m endonuclease) (C0SP) (AAV8), FIG. 10B (250 mM NaCl with 10 U/m endonuclease) (HRC) (AAV8), FIG. 10D (250 mM NaCl with 0 U/m endonuclease) (HRC) (AAV9), and FIG. 10E (250 mM NaCl with 10 U/m endonuclease) (HRC) (AAV9). Experimental characterization of the effect of salt shows that it is important to limit salt addition to prevent Host Cell DNA breakthrough into the filtrate. However, an amount of salt (for example, less than 100 mM) can be beneficial to increase overall process throughput and prevent the differential pressure across the filter from increasing too steeply.

Overall, this approach enables endonuclease-free clarification of rAAV. Thus, using salt conditions of 0-100 mM, for example, clarification can be undertaken with low endonuclease or without an endonuclease altogether, and thereby significantly reduce purification costs.

FIG. 11 depicts data set forth in FIGS. 10A-10F. A three-fold in throughput (liters filtered per meters² of filter area) at 5 pounds per square inch (psi) differential pressure at different endonuclease conditions, specifically at 0 U/ml, 10 U/ml and 100 U/ml of endonuclease, using AAV8 and AAV9. The 10 U/ml condition was midway between the 0 U/ml and 100 U/ml endonuclease conditions. Capsid and genomic yields in the filtrate pool of all runs were greater than 90% and comparable throughputs were observed at scales of 3.2 cm² and 25 cm².

FIG. 12 depicts data set forth in FIGS. 10A-10F regarding the effects of salt (NaCl). The pressure range from 5-20 psi is a safe zone for flush and filter blowdown operations with depth filtration. If the pressure increases too sharply from 5-20 psi, there can be a loss of hold-up material because the modules should not be operated above a limit of 20 psi. Salt conditions, such as about 1 mM to 110 mM or more, but less than 250 mM. Preferably 1 mM to 100 mM, more preferably 1 mM to 75 mM, still more preferably 1 mM to 50 mM, and yet more preferably 1 mM to 25 mM. For example, preferred salt conditions such as 5 mM, 10 mM, 15 mM, 20 mM, 25 mM, 30 mM, 35 mM, 40 mM, 45 mM, 50 mM, 60 mM, 65 mM, 70 mM, 75 mM, 80 mM, 85 mM, 90 mM, 95 mM, 100 mM, 105 mM, and 110 mM, and ranges formed between any of these values (for example, 25 mM to 100 mM, 50 mM to 100 mM, 75 mM to 100 mM, 90 mM to 110 mM) can be selected.

FIG. 13 depicts data showing that salt (NaCl) addition (See FIG. 12) reduces load turbidity and increases throughput at higher pressures. For example, salt conditions, such as about 1 mM to 110 mM or more, but less than 250 mM. Preferably 1 mM to 100 mM, more preferably 1 mM to 75 mM, still more preferably 1 mM to 50 mM, and yet more preferably 1 mM to 25 mM. For example, preferred salt conditions such as about 5 mM, 10 mM, 15 mM, 20 mM, 25 mM, 30 mM, 35 mM, 40 mM, 45 mM, 50 mM, 60 mM, 65 mM, 70 mM, 75 mM, 80 mM, 85 mM, 90 mM, 95 mM, 100 mM, 105 mM, and 110 mM, and ranges formed between any of these values (for example, 25 mM to 100 mM, 50 mM to 100 mM, 75 mM to 100 mM, 90 mM to 110 mM) can be selected to reduce turbidity and increase throughput at higher pressures.

Treatment with 250 mM NaCl resulted in host cell DNA breakthrough into the filtrate for low and zero-endonuclease conditions.

FIG. 14 depicts data set forth in FIGS. 10A-10F showing that a combined cake-fiber fouling model is a good representation for the mechanism of Harvest RC filtration (depth filtration). In FIG. 14, a filtration train with 0 U/ml endonuclease and 100 mM NaCl with AAV9 was analyzed.

Harvest RC filters contain fibrous anion exchange media layered above a flat sheet sterile filter. Low to zero endonuclease results in long strands of host cell DNA that are expected to bind to the AEX fibers, thereby causing fiber coating. Cake formation is expected at the flat-sheet filter and between tightly packed fibers. A cake-fiber fouling model resulted in excellent fit with R²>0.95 and was better than cake formation or fiber coating models individually.

The following model can be used:

$$\frac{Δ P}{Δ {P}_{0}} = \frac{[ 1 + \frac{( 1 - {∅}_{0} )}{{∅}_{0}} {K}_{f} V ]}{{( 1 - {K}_{f} V )}^{3}} + {K}_{c} {J}_{0} V$$

K_{c}=cake filtration constant. s/m²
K_{f}=fiber coating constant, 1/m
V=volume filtered, m³/m²
P=pressure, kg/ms²
J=solvent flux, m/s
Ø=filter solid or fiber volume fraction

See G. R. Bolton, D. LaCasse, M. J. Lazzara, and R. Kuriyel, “The fiber-coating model of biopharmaceutical depth filtration,” AIChE Journal, vol. 51, no. 11. Wiley, pp. 2978-2987 (2005).

FIGS. 15A-15C depict data set forth in FIGS. 10A-10F. Cake formation parameters (K_{C}) vs NaCl (mM) added with 0 U/ml endonuclease (FIG. 15A), 10 U/ml endonuclease (FIG. 15B), and 100 U/ml endonuclease (FIG. 15C).

Empirical parameters extracted from the best-fit model curves were plotted for the different load conditions. The cake-fiber fouling model uses two parameters, namely cake formation parameter K_{c} and fiber coating parameter K_{f}. K_{c} increased with increasing salt for all endonuclease conditions. This is expected as Cl⁻ competes with HCDNA for AEX binding sites and causes more of a cake filtration than fiber coating mechanism. K_{f} decreased two-fold between the 0 U/mL and the 10 U/mL endonuclease conditions, from an average of 6×10⁻⁶ to 3×10⁻⁶. This suggests that the fiber coating effect is stronger in the endonuclease-free condition. That is, the long strands of undigested HCDNA are binding strongly onto the AEX fibers and creating a thickening effect.

The methods of the inventions provided a reduction of HCDNA from 5×10⁴ ng/mL to 50 ng/mL for rAAV8 and 2×10⁴ ng/mL to 10 ng/mL for rAAV9 in endonuclease-free conditions. This is comparable to traditional depth filtration media with 100 U/mL endonuclease treatment in the load.

Overall, this approach enables endonuclease-free clarification of rAAV. Thus, using salt conditions of 0-100 mM, for example, clarification can be undertaken with low endonuclease or without an endonuclease altogether, and thereby significantly reduce purification costs.

Following depth filtration, the pool can be subjected to tangential flow filtration (TFF) for concentration and buffer exchange before further chromatography, such as affinity chromatography. Conventional TFF can be employed, which requires a batch approach and repeated cycling through the membrane to create a permeate and a retentate. Alternatively, single-pass TFF can be employed to allow for a continuous process. According to the inventions, TFF can advantageously employ permeate pumps to reduce TMP buildup and flux decline. Although not to be bound by any hypothesis or theory, it is believed that detergents, such as Tweens, build up on the retentate side of a TFF membrane and cause TMP buildup and flux decline. Single-Pass TFF units are available from Pall/Cytiva, Repligen and Millipore

### Example 8—Removal of Excess SpyCatcher-Rm

The hexad transfection production process represents an efficient method for the production of covalently modified AAV for retargeting. A population of retargeted AAVs with different covalent modifications (that is, covalently surface modified AAV species) can be produced using this method, and combined together (pooling) to form a mixture for in vivo screening of best retargeting candidate. The pooling step ideally would occur prior to purification unit operations so the subsequent purification process can be completed in a single train. To enable pooling of different production material prior to purification, excess SpyCatcher-Rm (non-conjugated) within each production should be inactivated/quenched to prevent cross conjugation to unintended, unconjugated SpyTag-AAV after pooling.

The present inventions can utilize a 13 amino acid SpyTag peptide in soluble form (Example 33) to bind to the excess SpyCatcher-Rm (here SpyCatcher Fabs) and achieve a two log reduction in unintended cross conjugation between the SpyTag AAV and SpyCatcher Fab. The concentration of the soluble SpyTag was screened and the effect was demonstrated as discussed below. See FIGS. 16-17.

In this study, three suspension cultured fed-batch productions were completed in miniature 250 ml bioreactors, producing 1) an unconjugated SpyTag AAV species carrying a nano luciferase (nLuc) reporter gene, 2) a mouse-TfR Fab (8D3) conjugated SpyTag AAV carrying an alternative transgene (in this case gene encoding for green fluorescent protein), 3) a mouse-TfR Fab (8D3) conjugated SpyTag AAV carrying a nano luciferase reporter gene (nLuc). The production material was assessed in a transduction assay using HEK293 cells expressing the mouse-TfR cell surface receptor (mTfR+HEK293) and luciferase as the reporter signal. Production 1 and 2 material alone showed low luciferase signal due to the absence of mTfR Fab conjugation or absence of relevant reporter gene, while production 3 material showed intense luciferase signal as a positive control. The soluble SpyTag at a concentration of 0, 33, 100, 300, and 900 ug/ml was co-incubated with the production 1 and 2 material separately at room temperature, pH 6.0 for 24 hours prior to pooling of the two production material together. The combined material was then assayed in the transduction assay. Comparing with no soluble SpyTag quenching condition (yellow), the soluble SpyTag at concentrations of 33-900 ug/mL (green) resulted in two log reduction in cross conjugation signal.

FIGS. 16 and 17 schematically depict a proof of concept study demonstrating the use of soluble SpyTag peptide to quench excess SpyCatcher Fab. FIG. 16 illustrates the three productions performed using the suspension miniature bioreactor system, and the anticipated transduction assay luciferase signal (nLuc) for the three productions alone and the pooled material with or without soluble SpyTag quenching. Triangles attached to an AAV represent SpyTag peptide inserted into the VP proteins. Triangles not attached to an AAV represent soluble SpyTag peptide. FIG. 17 depicts the luciferase signal from the transduction assay when cells are dosed with the respective AAV products at a series of multiplicity of infection (MOI) levels. The 8D3 is a particular mouse-TfR Fab candidate.

### Parameters and Ranges:

Concentration ranges for the soluble SpyTag include 2-1000 ug/mL
pH ranges include 5.0-8.0
Duration of quenching 1-48 hours
Temperature for quenching 2-37° C.
Higher soluble SpyTag concentration (100-900 ug/mL) and longer duration of quenching (12-48 hours) are preferred under unfavorable conjugation conditions (pH>=7.0). Lower soluble SpyTag concentrations (2-100 ug/mL) and shorter duration of quenching (1-24 hours) may be sufficient under favorable conjugation conditions (pH 5-7). This approach is useful with the Hexad transfection and other transfection regimens.

### Example 9—Genomic Yield with Affinity Capture

Purification of covalently surface modified AAVs (also referred to as “retargeted AAVs”) requires effective methods to capture surface-modified AAVs from the bioreactor harvest mixture. Challenges in affinity capture include difficulty in binding to the AAV surface epitopes in case of heavy conjugation, or difficulty in eluting the surface-modified AAV without destabilizing the molecule, thereby leading to aggregation, precipitation, and/or fragmentation. These difficulties often lead to an imbalance in the level of conjugation in the affinity capture load material relative to the eluate stream; that is, a loss of the some to all of the conjugated species during affinity capture due to preferential purification of the less conjugated and unconjugated AAVs. The goal of high-yield affinity capture of conjugated AAV species was not met until the present inventions.

Several screening runs were carried out on different resins and with different elution buffers. Poros CaptureSelect AAVX did not demonstrate good binding affinity for conjugated rAAV species, for example, AAV9-SpyT-SpyC-mAb. In contrast, Poros CaptureSelect AAV9 resin did exhibit good binding with conjugated rAAV species, for example, AAV9-SpyT-SpyC-mAb, but the yield was dependent on the choice of elution buffers.

Low yields were achieved in many elution buffers, such as yields of approximately 10% for glycine buffers along with loss of the heaviest conjugated species. A buffer comprising about: 50 mM acetate, 50 mM histidine, 0.001% P188, pH 3.0 was able to achieve yield of greater than 90% along with successful capture of the heavily conjugated AAV species. Notably, this buffer also resulted in highest yield for unconjugated AAV9-SpyTag species as produced in the post-processing conjugation system instead of the hexad transfection system.

FIG. 18 depicts data using a POROS CaptureSelect AAV9 resin to capture AAV9-SpyT-SpyC-mAb. Monoclonal antibodies against CACNG1, ASGR1 and Fel d 1 were used as retargeting molecules. Average column loading was 2.5×10¹² viral genomes/ml resin and 9.2×10¹³ capsids/ml resin.

Less conjugated AAV9 ( 1/20 and 1/30) achieved higher yields. It is believed that affinity chromatography favored less conjugated AAV9 because such AAV9 would have more available epitopes.

FIG. 19 compares various buffers used for affinity capture of an AAV-SpyT-SpyC-Tfr Fab. The buffers left to right comprised (i) glycine, (ii) acetate, (iii) acetate and arginine, (iv) acetate and histidine, and (v) acetate, histidine and arginine. Buffer (iv) acetate and histidine provided the highest yield. A POROS CaptureSelect AAV9 column loaded with 10¹³ to 10¹⁵ capsids per milliliter was employed.

FIG. 20 compares the level of conjugation in the eluate of affinity capture using (i) a single affinity capture step specific to AAV surface epitopes, and (ii) after a second affinity capture step specific to antibody surface epitopes. POROS CaptureSelect AAV9 was useful for removing free antibody (fAb). The two-step affinity capture (POROS CaptureSelect AAV9 and Capto L) strategy resulted in removal of unconjugated AAV species. See FIG. 20 and Table 3.

TABLE 3

| | Fab-AAV9 | AAV9 | Overall Yield |
|---|---|---|---|
| | Peak Area | Peak Area | (droplet digital |
| Sample | (SEC-MALS | (SEC-MALS | PCR) |
| POROS | 3950 | 3548 | 95% |
| CaptureSelect | | | |
| AAV9 | | | |
| POROS | 4025 | Not | 55% |
| CaptureSelect | | detected | |
| AAV9 and Capto L | | | |

Following affinity capture, the pool optionally can be subjected to tangential flow filtration for concentration. Thereafter, the pool optionally can be subjected to centrifugation, such as Iodixanol Gradient Centrifugation to enrich for full capsids and then subjected again to Tangential Flow Filtration (TFF) to concentrate and a buffer change to a storage buffer, or other chromatographic methods, such as AEX. TFF is a generic term and includes, but is not limited to, ultrafiltration/diafiltration (UF/DF) and newer approaches such as single-pass tangential flow filtration (SPTFF).

### Example 10—Production of Fab Conjugated Barcoded AAV Library

The hexad transfection methodology allows simultaneous production of SpyTag AAV and SpyCatcher-Rm within the same production bioreactor, and is an efficient way to produce a library of covalently surface modified AAV species for screening studies when the AAV and/or conjugated retargeting molecules (Rm) are uniquely different.

Using the hexad transfection method, a library of 48 Fab conjugated AAV vectors (which are covalently surface modified AAV species) were produced for a non-human primate study to screen for top Fab candidates to improve tissue specificity and transduction efficiency. Each of these conjugated AAV vectors carries a polynucleotide, such as transgene comprising a unique 32 base pair (bp) sequence as a “DNA barcode,” which are commercially available from companies such as PacBio.

Each unique DNA barcode serves as a biomarker encoded inside the AAV vector, and is matched to a Fab candidate conjugated on the outside of the AAV vector. The library of Fab conjugated AAV vectors were pooled and injected into mice and non-human primates. At the end of the study, the tissue of interest was harvested, and the enrichment of DNA barcodes in the target tissue, representing the preferential transduction of the AAV vector retargeted by the corresponding Fab candidate, was evaluated using next generation sequencing technologies.

Production of the library of Fab conjugated AAV vectors starts with thawing of a vial of the suspension cultured HEK293 cell line followed by a series of cell expansion steps to gradually increase the cell culture volume. Cells were then inoculated into the production bioreactors, with each conjugated species produced separately in a fed-batch production bioreactor using the hexad transfection method. At the end of the productions, cells were lysed and clarified via depth filtration. The material was held at pH 6.0 at room temperature for 24 hours to allow conjugation reaction to reach completion.

Prior to the pooling of the conjugated AAV vectors from different productions, the excess free SpyCatcher Fabs were quenched by the addition of soluble SpyTag followed by another 24-hour room temperature hold at pH 6.0. The small soluble peptide bound to the excess SpyCatcher Fab and prevented it from cross conjugating to unintended AAV vectors. The soluble SpyTag quenched material was then pooled and further purified through a purification train comprising tangential flow filtration (TFF), affinity capture, iodixanol ultracentrifugation and a final TFF. The process flow diagram is shown in FIG. 21. Using the hexad transfection production process, the average vector genome titer obtained from the bioreactor lysate was 1.8×10¹¹ vg/mL (FIG. 22). The 48 different DNA Bar codes used to identify each Fab conjugated AAV vector are set forth in Example 34.

### Example 11—Production of Covalently Surface Modified AAV Using Adherent HEK 293T Cells

Adherent HEK 293T cells were used to produce covalently surface modified AAVs:

### Splitting & Platin of Adherent 293T Cells (at Day 3 or Day 4 of Growth):

### Lifting & Passaging

1. Prepare & warm fresh formulated media
   a. 500 ml DMEM, 50 ml FBS, 5 ml NEAA, 5 ml PenStrep
      i. Final concentrations of: 10%-FBS, 1% NEAA, 1% PenStrep, good for one month;
2. Remove confluent HEK293T T225s from incubator, check cells under microscope, and aspirate spent media w/ vacuum line;
3. Add 5 ml of TrypLE Select/T225, evenly disperse over cells, and wait 2-5 minutes for cells to dissociate from flask surface (cells do not need to be rinsed with phosphate buffered saline (PBS) prior to addition of TrypLE Select);
4. Lightly tap flasks to loosen cells and add 5 ml of formulated DMEM per T225;
5. Repeatedly rinse/wash cells off w/ pipette controller and transfer cells to appropriately sized vessel (50 ml-200 ml conical tube);
6. Count cells w/ Countess and seed 4-6 new T225 flasks for next passaging cycle
   a. 5×10⁶ cells for 4 days, 1×10⁷ cells for 3 days (in 30 ml formulated DMEM/T225);
7. Return cells to 37° C. incubator for 3-4 days (based on seeding density).

### Seeding (10-Layer Cell-Stack)

Seed HEK293T cells for AAV production according to below table and incubate at 37° C.:
a. For cell-stacks, mark off cap used for adding media & cells. Make sure to use same side for transfection;
b. For 10-layer cell-stacks, evenly divide cells between the 2 media bottles.

| | Seeding | | |
|---|---|---|---|
| Cell-Stack/ | ml formulated | Cells required | Cells required |
| Plate Size | media/vessel | (3-day seed) | (4-day seed) |
| 10-Layer | 1120 (2x bottles) | 8.48 × 10⁷ | 4.24 × 10⁷ |

Note:
each layer in a cell-stack is ~4 15 cm plates

### Transfection of 293T Cells (Day 0):

### 10-Layer Cell-Stack

1. For each cell-stack, add about 15 ml of unformulated DMEM to 2×50 ml conical tubes
   a. 1×15 ml (PEI tube), 1×15 ml (DNA tube;)
2. To DNA tube & PEI tube, add appropriate amounts of DNA and PEI (see table below) & vortex each for 10 seconds
   a. 1 ug DNA: 4 μg polyethylenimine (PEI) ratio;
3. pRC-SpyT to pRC Ratio 1/8

| | Number of plates | | | |
|---|---|---|---|---|
| | 1 × 15 cm plate | 1 × 10-L CS | | |
| | 1 | 42 | | |
| | DNA Amount | DNA Amount | Plasmid Conc. | Volume for |
| Plasmids | (μg) | (μg) | (mg/ml) | transcription (μl) |
| Heavy Chain | 1.5 | 63 | 1 | 63.0 |
| Light Chain | 3 | 126 | 1 | 126.0 |
| pAd Helper | 16 | 672 | 1 | 672.0 |
| Capsid (Mixer) Rep and | 8 | 294 | 1 | 294.0 |
| Cap from AAV9 with W503A | | | | |
| Capsid (Spytag) Rep and | | 42 | 1 | 42.0 |
| Cap from AAV9 with W503A | | | | |
| SpyTag and linker 10 | | | | |
| CAG promoter and eGFP | 8 | 336 | 1 | 336.0 |
| as GOI | | | | |

| | Amount (μg) | Amount (ml) |
|---|---|---|
| PEI Max | 36.5 | 6.13 |

4. Combine DNA and PEI solutions and vortex for 10 seconds. Incubate mixture for 10 minutes at room temperature;
5. During the 10-minute incubation: Label cell-stacks appropriately and bring multi-Liter bucket into hood for waste;
6. 1 minute left in incubation: Unscrew marked cap. Pour spent media into the plastic bucket;
7. When 10 minutes has elapsed, gently swirl DNA-PEI complexes, and add to formulated media. Swirl formulated media+complexes gently to mix
   a. For 10-layer cell-stacks, evenly divide complexes between the 2 media bottles;
8. Slowly add formulated media+complexes into the cell stack through labeled cap side;
9. Tilt the cell stack on the long side (unmarked cap side). Wait for media levels to equalize. Slowly turn to the short side (side without caps). Finally, slowly lay cell stack flat and gently rock to cover cells with media and complexes;
10. Check that the layers appear similar in volume and return to 37° C. incubator for 3-4 days.

### Collections (Day 3 or 4):

### 10-Layer Cell-Stack (Lysate Collection & Processing)

1. Sterile-filter portion of antibody-containing supernatant (˜100 ml) and pour remainder into appropriately sized waste bucket for disposal;
2. Add appropriate amount (see below) of PBS+4 mm EDTA to cell-stack, evenly distribute over cells, and incubate at 37° C. for ˜5 minutes
   a. 10-Layer (˜800 ml);
3. Shake cell-stack side-to-side and up/down to dislodge cells. Ensure that cells are lifted and pour PBS/EDTA+cells into appropriate number of 200-225 ml conical tubes;
4. Pellet cells and debris at 4000 rpm for 25 minutes;
5. Carefully pour off supernate and re-suspend in appropriate volume of lysis buffer (see below)
   a. 10-Layer (35 ml);
6. Transfer lysate to appropriate #of 50 ml conical tubes
   a. Use 2×50 ml tube (17.5 ml of resuspended lysate in each);
7. Vortex and freeze in dry ice+100% EtOH bath;
8. Thaw cells in 37° C. water bath;
9. Repeat for a total of 3 freeze/thaw cycles
   a. Cell lysates can be kept frozen at −80° C. at any point for processing later;
10. Add 35 ml total of supernatant back to lysate (17.5 ml per 50 ml conical)
   a. total volume now about 70 ml, split into 2×50 ml conicals containing ˜35 ml each);
11. Add total 25 μL of Denarase total to complexing AAVs (about 12.5 μL per 50 ml conical);
12. Vortex to mix and incubate at 37° C. for 1 hour with occasional vortexing;
13. Pellet at 10,000×g for 10 minutes;
14. Filter through 0.2 μm PES filter or 50 ml MDI filter and discard pellet.

### Iodixanol Gradient Ultracentrifugation (for Isolation of Full AAV Capsids, 80-90% Full)

1. Using 10 ml syringe+16 or 19-gauge stainless steel canula needle, prepare iodixanol (IDX) gradients by underlaying iodixanol solutions in ascending order of density:
   a. 7 mL 15% Iodixanol
   b. 6 mL 25% Iodixanol
   c. 7 mL 40% Iodixanol
   d. 5 mL 60% Iodixanol
      Note: dispense IDX solutions slowly to avoid mixing/disturbing the different layers, and mark interface between layers at conclusion of gradient laying;
2. Gently load concentrated medium or cell lysate onto gradient with pipette controller and fill remaining volume as necessary with 1×PBS+0.001% Pluronic (for media) or lysis buffer (for lysate)
   a. Capacity for each IDX column is ˜12-13 ml (˜10 plates worth of volume)
   b. Weigh and balance tubes in buckets with caps to minimize centrifugation error;
3. Insert into Beckman Coulter Type SW 32 Ti Swinging-Bucket Rotor;
4. Ultracentrifuge virus for 16-18 hours overnight at 29,600 rpm at 10° C. Use max acceleration and deceleration of “5”;
5. After centrifugation, carefully remove tubes from the rotor with disposable forceps
   a. Spray buckets with Envirocide, and then 70% ethyl alcohol to clean;
6. Gently insert sterile beveled 1.5″ 19G needle with 3 mL syringe roughly 0.5 mL below interface of 40% and 60% iodixanol solutions. Draw 3 mL of volume
   a. Use 3D-printed AAV IDX extractor for support;
7. Remove needle, block puncture, and discard tube in a waste bottle.

### Buffer Exchange and Concentration (for Lower Titer Serotypes)

(Reagent Consideration: 1×PBS+0.1% Pluronic, 1×PBS+0.01% Pluronic, 1×PBS+0.001% Pluronic, 100 kDa Amicon concentrator (15 ml))
1. Add 1×PBS+0.1% Pluronic to 100 kDa Amicon concentrator, allow concentration filter to incubate for 5 minutes, and centrifuge at 4000×g for 2 minutes. Discard flow-through;
2. Add 1×PBS+0.01% Pluronic to 100 kDa Amicon and centrifuge at 4000×g for 2 minutes. Discard flow-through;
3. Add 1×PBS+0.001% Pluronic to 100 kDa Amicon and centrifuge at 4000×g for 2 minutes. Discard flow-through;
4. Dilute 3 ml iodixanol fraction to 15 mL with 1×PBS+0.001% Pluronic;
5. Centrifuge sample at 4000×g until volume is reduced (<1.5 mL) and discard flow-through
   a. 2-3 fractions of the same AAV can be combined for use in a single Amicon (dilute appropriately). Additional fractions will increase spin times dramatically
   b. Likely no more than 5-10 minutes needed for initial spin with a single gradient;
6. Resuspend to top of Amicon with additional quantity of sample or 1×PBS+0.001% Pluronic and repeat centrifugation;
7. Continue to refill and centrifuge an additional 3×;
8. Reduce to desired volume (refer to final concentration table) and filter using 0.2 μm Spin-X tube (10,000 RPM, 1 minute);
9. Store purified AAV at 4° C. for short term use (<2 weeks) or aliquot and store at −80° C.
   The yield of this protocol for AAV9 was 1 to 2.5×10¹³ vg after Iodixanol (IDX) purification, with greater than 50% full capsids.

### Example 12—Process Analytical Technology and Improved Purification

FIG. 23 depicts exemplary production purification trains for AAV, such as recombinant AAV. The top train uses a batch process where repeated passes are required to exchange buffer and concentrate the retentate, which contains the desired biological material, such as AAV. See Adams et al., Biotech. Bioeng. 117: 3199-3211 (2020).

The bottom section of FIG. 23 replaces the batch tangential flow filtration unit with a single-pass tangential flow filtration unit (SPTFF unit), which permits a continuous process. It was surprising how well SPTFF performed with AAV, as taught herein,

The Batch TFF approach can take multiple days (for example, 2 days) due to the repeated cycling through the conventional TFF unit to achieve concentration prior to further purification. The SPTFF approach is a continuous approach, and is significantly faster than the Batch TFF approach, and can be performed in several hours, such as 3 to 5 hours. The SPTFF approach provides faster concentration, while minimizing sheer stress and damage to AAVs. The SPTFF approach also is amenable to the use of Process Analytical Technology (PAT) and automation.

For further comparison, FIG. 24A schematically shows a Batch TFF (top), where the retentate is repeatedly cycled through a feed tank and pump to repeatedly passed through a membrane, with the concentrated retentate being removed after repeated cycles. A Single-Pass TFF removes biological material from the feed tank through a pump to a multi-stage membrane module that separate the retentate from the permeate, while concentrating the retentate.

FIG. 24B is a graph comparing Batch TFF and Single-Pass TFF. Single-Pass TFF achieves higher concentration and is faster as compared to Batch TFF. Single-Pass TFF continuously sends biological material to the next operation in the purification train, whereas Batch TFF does not send biological material until the end of the batch cycle.

FIG. 25 schematically and qualitatively compares the batch operation to a continuous operation for AAV purification in terms of Cell lysis, Clarification (depth filtration), TFF (Batch or Single-Pass) and Affinity Capture. The continuous process (SPTFF) can be completed in less than a day, whereas the batch process can be multi-day.

The cell lysis step, typically using a detergent such as Tween-20, typically takes up to about two hours, and is depicted as the same for both the Batch and the SPTFF (continuous) process. Following lysis, clarification takes about 1 hour. The processes then diverge at the TFF step.

For the Batch process, TFF takes about 3 hours per batch due to the repeated cycling. Not until a batch is complete can the concentrated biological material in a buffer be passed on the affinity capture, which takes about 2 to 3 hours per batch. Because multiple batches are required, the affinity chromatography is typically not completed until the next day.

For the Continuous process, clarification, SPTFF and affinity capture can take place substantially simultaneously. Biological material continuously flows to clarification (about 1 hour), SPTFF (about 1½ hours) and affinity capture (about 2 hours to 3 hour). Accordingly, when an early portion of biological material is in affinity capture, later portions of biological material are in SPTFF or clarification.

FIG. 26 schematically depicts exemplary arrangements for multi-stage membrane module cassettes to be used with Single-Pass TFF. The configurations depict four to seven tiers of membrane module cassettes where the initial tiers (left side) contain more or same number of membrane module cassettes as the succeeding tiers (moving towards the right side), in the manner suggested by the manufacturer, here Pall/Cytiva. Total area and path length of the membrane module cassettes also are set forth. Other arrangement of membranes, flow rates and transmembrane pressure (TMP) can be selected by the person skilled in the art.

FIG. 27 is a graph depicting volumetric concentration factor (VCF) versus transmembrane pressure (TMP) using the 4-in-series, 5-in-series, 6-in-series and 7-in-series exemplary configurations depicted in FIG. 26 with a feed comprising an exemplary AAV, here AAV9 comprising a SpyTag insert. A Batch process target would be 8-10×VCF at a TMP of 5 to 10 psi.

FIG. 28 depicts data from a 5-in-series configuration according to FIG. 26 at flow rates of 90 ml/minute, 120 ml/minute and 150 ml/minute. The log best-fit equation of VCF=A In (TMP-B) using the values at each flow rate set forth near the plot (and rounded off in the included table) can be used to parameterize the data. At the right side of the figure, there is a graph of parameter value (A, B) and feed flow rate in liters per square meter of membrane per hour (LMH) for 4-in-series and 5-in-series exemplary configurations of FIG. 26 and allows optimized conditions to be selected in silico using an exemplary AAV, here AAV9 comprising a SpyTag insert. This model can be used to predict the VCF for any flow rate and TMP for an in-series configuration of interest.

FIG. 29A is a design space model based on FIGS. 27 and 28 using the 5-in-series configuration of FIG. 26. Here, the process target was 35 LMH, and the intersecting lines indicate a VCF of 8 and a TMP of 10 psi. An exemplary acceptable zone would be a VCF of 6-10 and a TMP of 7.5 to 12.5 psi. FIG. 29B is an exemplary comparison of process parameters between SPTFF and Batch TFF. With Batch TFF, typically there would be one batch before the next operation. However, depending on the scheduling of upstream production bioreactors and bioreactor titers, there could be pooling of multiple batches before the next operation. Effective residence time of a given portion of biological material in the SPTFF is only about 10 minutes, and the overall time is for all biological material to pass thought the SPTFF.

FIG. 30 depicts data from a bench-scale trial to determine the number of buffer washes need to attain about a 90% recovery of AAV, here AAV9 with integrated SpyTag, in a low-TMP process. On average, the AAV9 here contained 6 SpyTag peptides per viral capsid. The load concentration was 1.7×10¹² capsids/ml (cp/ml). The steady state concentration using SPTFF was 1.6-1.9×10¹³ cp/ml, yielding a steady state VCF of 10 to 11×. Capsid titer in retentate (cp/ml) versus SPTFF operating time (minutes) was measured using four buffer flushes. The final pool flush (1 and 2) achieved 1.4×10¹³ cp/ml. As the right side of the figure shows, 71% of capsids were recovered in the retentate pool, 11% of capsids were recovered by flush 1 and 6% of capsids were recovered with flush 2. It was determined that only two buffer flushes were required to achieved about a 90% recovery with a VCF of 8×.

FIG. 31 is a graph depicting Permeate Flux (LMH), Throughput (L/m²), Feed Flow Rate (L/hr) and TMP (psi) in a pilot-scale trial. Using continuous SPTFF, the data demonstrated flux decline and TMP build up. To mitigate TMP increase beyond 12.5 psi, feed flow rate was slowed. This resulted in a longer process time of 180 minutes rather than the expected 90 minutes and an overall VCF of 5× was achieved rather than the target VCF of 8×.

FIG. 32 depicts a tween micelle build-up on the TFF membrane. Without being bound by any theory or hypothesis, it is believed that detergent micelle buildup (here, Tween-20) is the cause of an unexpected flux decline of about 50% using SPTFF to concentrate AAV. Typically, a 20% flux decline is expected when concentrating antibodies. This figure also set forth the approximate size of AAV, Host Cell Protein aggregates (HCP) and Tween-20 micelles. The micelle concentration of Tween-20 was determined to be about 0.7%. See Basheva et al, J. Physical Chemistry Chemical Physics, Issue 38 (2007) (discusses properties of films formed by Brij 35 and Tween 20). Detergents, such as Tweens, are a common component of cell lysis buffers used in the production of AAV.

FIG. 33 is a graph depicting fold presence of Tween-20 on the retentate side of membrane and the permeate side of the membrane for both Batch TFF and SPTFF. Most Tween-20 is on the retentate side.

FIG. 34 is a graph depicting the flux decline after two hours with varying percentages of Tween-20 in the lysis buffer. The lower the percentage of Tween-20, the lower the percentage of flux decline encountered. In addition to Tween-20, the buffer contained 20 mM Tris, 2 mM MgCl₂ at a pH of 7.4. The feed flow rate was 35 LMH and the TMP was about 5 to 10 psi.

With Batch TFF, flux decline can be address by increasing processing time. However, with SPTFF immediate control is desired.

FIG. 35 compares control with the retentate valve to control with a Permeate pump. Option 1 with the retentate valve found that TMP reached 22 psi, and after which the flow had to be reduced from 40 LMH to 30 LMH. VCF dropped from about 10× to about 6×. Option 2 with the permeate pump was superior, which acts as a suction pump. TMP was controlled to well under 10 psi and a VCF of 8× was maintained. At the right side of the figure, Option 1 (SPTFF with retentate valve) and Option 2 (SPTFF with permeate pump) were compared to a Batch TFF. Option 1 did not perform as well as Option 2 and Batch TFF. Option 2 was superior to Batch TFF and Option 1 in terms of capsid yield and percent aggregation. The permeate pump flow should be set within the VCF design space to avoid negative permeate pressure buildup. Thus, the flow rate of operation of the permeate pump should be within the range established in FIG. 29A

FIG. 36 depicts the overall pilot scale process, and is similar to parts of the production process of FIG. 23.

FIG. 37 compares VCFs (1-14), SPTFF retentate flow rates and residence time in affinity capture. VCFs of 7 to 13 and SPTFF retentate flow rates of 75-40 provided an exemplary range of residence time suitable for affinity loading (2.7 to 5.0 minutes). The flow rate should be selected to avoid depleting or overwhelming the affinity column. This calculation was based on a pilot-scale trial with a 525 ml/minute feed flow in a 5-in-one series SPTFF module and then loaded on to a 200 ml POROS CaptureSelect AAV9 column.

FIG. 38 depicts how UV280 profile of affinity capture flow can be used for process monitoring of VCF and process stability using SPTFF for continuous processing. Three different runs were performed for comparison purposes. Run 1 was performed without a permeate pump and achieved a VCF of only 5×. Run 2 was performed with a permeate pump with a feed to retentate flush (with recirculation) and achieved a VCF of 8×. Run 3 was performed with a permeate pump with a feed to retentate flush (with recirculation) and a permeate to retentate flush, which achieved a VCF of 10×. Most chromatography systems have built-in UV280 sensors that can detect load concentration, and provide an indication of VCF and process stability. Any needed correction, such as pump and/or valve control, can be based upon the data received through process analytical technology. See Thakur et al., J. Membrane. Sci. 613: 118492 (2020) discuss the use of process analytical technology with SPTFF.

Advantages and Aspects of SPTFF include:
Greater efficiency in AAV manufacturing from harvest to final capture and purification;
Use of a permeate pump provides real-time control over TMP and maximizes AAV yield and minimizes AAV aggregation;
Detergents, such as Tween-20, can decrease permeate flux, which can be best managed through use of a permeate pump; and
Volumetric concentration factor (VCF) depends on flow rate, TMP and SPTFF membrane module configuration, which can be addressed by the empirical modeling of VCF vs. TMP curves for optimization based upon the teachings contained herein.

### Examples of Covalently Surface Modified AAV

The following examples provide teachings on how to produce covalently surface modified AAV of all serotypes using exemplary sequences according to the inventions. Pertinent polynucleotide and amino acid sequences are widely available in the published literature, and therefore the inventions are not limited to the polynucleotide and amino acid sequences set forth in the specification and the sequence listing. Rep and cap genes from the same serotype are exemplified below, but the inventions also provide for use or rep and cap genes from different serotypes.

### Example 13—AAV1

Covalently surface modified AAV1 can be made by transfecting a cell with plasmids as set forth below, followed by culturing the transfected cells and then harvesting the covalently surface modified AAV1. The first chart below uses SpyTag-SpyCatcher as specific binding pairs, and Adenovirus Helper Genes.

Modifications are possible and the considerations include:

| Plasmid type | Specifics |
|---|---|
| pGOI | Any gene(s) of interest flanked by |
| | AAV ITRs. |
| pRC | AAV 1 rep and cap genes. See |
| | Example 30. |
| pRC-FM | pRC-Spy Tag. Provides VP1, VP2 |
| | and VP3 proteins fused to Spy Tag |
| | protein. See Examples 33 and 35. |
| pHELP | Comprises one or more adenovirus |
| | helper genes. See Example 31. |
| pHC-SCM | pHC-Spy Catcher. Provides an |
| | antibody heavy chain fused to Spy |
| | Catcher. |
| pLC | Comprises an antibody light chain. |

(1) Plasmid pGOI can comprise one or more genes that are desired to be expressed in covalently surface modified AAV1;
(2) Plasmids pRC-FM and pHC-SCM can be modified to use other specific binding pairs. The first member (FM) and the second cognate member (SCM) should be complementary in order to bond.
(3) Plasmids pHC-SCM and pLC can be modified to use other Retargeting Molecules. For example, the heavy chain (HC) and the light chain (LC) should be complementary in order form a functional Retargeting Molecule.
(4) Detargeting mutations can be undertaken as disclosed herein and in publications.

The second chart below cites to exemplary modifications to the above chart based upon the descriptions and sequence examples contained herein:

| | |
|---|---|
| pRC | AAV 1 rep and cap genes. See |
| | Example 30. |
| pRC-FM | See Examples 33 and 35 to obtain |
| | VP1, VP2 and VP3 proteins fused to |
| | a an FM (e.g., an FM (e.g., tag-type |
| | protein). protein). protein. |
| pHELP | See Example 31 concerning helper |
| | genes from herpes simplex virus |
| | (HSV), human papilloma virus |
| | (HPV), bocavirus, and baculovirus. |
| pHC-SCM | See Examples 32, 33 and 35 to |
| | obtain catcher-type sequences. |
| pLC | Comprises an antibody light chain. |

### Example 14—AAV2

Covalently surface modified AAV2 can be made by transfecting a cell with plasmids as set forth below, followed by culturing the transfected cells and then harvesting the covalently surface modified AAV2. The first chart below uses SpyTag-SpyCatcher as specific binding pairs, and Adenovirus Helper Genes.

Modifications are possible and the considerations include:

| Plasmid type | Specifics |
|---|---|
| pGOI | Any gene(s) of interest flanked by |
| | AAV ITRs. |
| pRC | AAV 2 rep and cap genes. See |
| | Example 30. |
| pRC-FM | pRC-Spy Tag. Provides VP1, VP2 |
| | and VP3 proteins fused to Spy Tag |
| | protein. See Examples 33 and 35. |
| pHELP | Comprises one or more adenovirus |
| | helper genes. See Example 31. |
| pHC-SCM | pHC-Spy Catcher. Provides an |
| | antibody heavy chain fused to Spy |
| | Catcher. |
| pLC | Comprises an antibody light chain. |

(1) Plasmid pGOI can comprise one or more genes that are desired to be expressed in covalently surface modified AAV2;
(2) Plasmids pRC-FM and pHC-SCM can be modified to use other specific binding pairs. The first member (FM) and the second cognate member (SCM) should be complementary in order to bond.
(3) Plasmids pHC-SCM and pLC can be modified to use other Retargeting Molecules. For example, the heavy chain (HC) and the light chain (LC) should be complementary in order form a functional Retargeting Molecule.
(4) Detargeting mutations can be undertaken as disclosed herein and in publications.

The second chart below cites to exemplary modifications to the above chart based upon the descriptions and sequence examples contained herein:

| | |
|---|---|
| pRC | AAV2 rep and cap genes. See |
| | Example 30. |
| pRC-FM | See Examples 33 and 35 to obtain |
| | VP1, VP2 and VP3 proteins fused to |
| | a an FM (e.g., an FM (e.g., tag-type |
| | protein). protein). protein. |
| pHELP | See Example 31 concerning helper |
| | genes from herpes simplex virus |
| | (HSV), human papilloma virus |
| | (HPV), bocavirus, and baculovirus. |
| pHC-SCM | See Examples 32, 33 and 35 to |
| | obtain catcher-type sequences. |
| pLC | Comprises an antibody light chain. |

Serotype AAV2 7m8: AAV2 7m8 (also referred to as AAV2.7m8) can be used and is characterized by a 10-amino acid peptide ‘LALGETTRPA’, referred to as ‘7m8’, inserted at position 588 of the AAV2 capsid protein sequence. (Dalkara, D., et al. “In vivo-directed evolution of a new adeno-associated virus for therapeutic outer retinal gene delivery from the vitreous. Sci Transl Med. 2013; 5: 189ra76.” Gene therapy restores vision in a canine model of childhood blindness. Nat Genet 28.1 (2001): 92-5).

Serotype AAV2-4Y-F: AAV2 Quad Y-F can be used and is referred to a modified AAV2 comprising a mutated AAV2 VP3 capsid protein comprising phenylalanines (F) at each of the positions corresponding to Y272, Y444, Y500, and Y730 in a wild type AAV2 VP3 capsid protein (Petrs-Silva, Hilda, et al. “High-efficiency transduction of the mouse retina by tyrosine-mutant AAV serotype vectors.” Molecular therapy 17.3 (2009): 463-471).

### Example 15—AAV3

Covalently surface modified AAV3 can be made by transfecting a cell with plasmids as set forth below, followed by culturing the transfected cells and then harvesting the covalently surface modified AAV3. The first chart below uses SpyTag-SpyCatcher as specific binding pairs, and Adenovirus Helper Genes.

Modifications are possible and the considerations include:

| Plasmid type | Specifics |
|---|---|
| pGOI | Any gene(s) of interest flanked by AAV ITRs. |
| pRC | AAV 3 rep and cap genes. See Example 30. |
| pRC-FM | pRC-Spy Tag. Provides VP1, VP2 and VP3 proteins |
| | fused to Spy Tag protein. See Examples 33 and 35. |
| pHELP | Comprises one or more adenovirus helper genes. |
| | See Example 31. |
| pHC-SCM | pHC-Spy Catcher. Provides an antibody heavy |
| | chain fused to Spy Catcher. |
| pLC | Comprises an antibody light chain. |

(1) Plasmid pGOI can comprise one or more genes that are desired to be expressed in covalently surface modified AAV1;
(2) Plasmids pRC-FM and pHC-SM can be modified to use other specific binding pairs. The first member (FM) and the second cognate member (SCM) should be complementary in order to bond.
(3) Plasmids pHC-SCM and pLC can be modified to use other Retargeting Molecules. For example, the heavy chain (HO) and the light chain (LO) should be complementary in order form a functional Retargeting Molecule.
(4) Detargeting mutations can be undertaken as disclosed herein and in publications.

The second chart below cites to exemplary modifications to the above chart based upon the descriptions and sequence examples contained herein:

| | |
|---|---|
| pRC | AAV 3 rep and cap genes. See Example 30. |
| pRC-FM | See Examples 33 and 35 to obtain VP1, VP2 and VP3 |
| | proteins fused to a an FM (e.g., tag-type protein). |
| | protein. |
| pHELP | See Example 31 concerning helper genes from herpes |
| | simplex virus (HSV), human papilloma virus (HPV), |
| | bocavirus, and baculovirus. |
| pHC-SCM | See Examples 32, 33 and 35 to obtain catcher-type |
| | sequences. |
| pLC | Comprises an antibody light chain. |

### Example 16—AAV4

Covalently surface modified AAV4 can be made by transfecting a cell with plasmids as set forth below, followed by culturing the transfected cells and then harvesting the covalently surface modified AAV4. The first chart below uses SpyTag-SpyCatcher as specific binding pairs, and Adenovirus Helper Genes.

Modifications are possible and the considerations include:

| Plasmid type | Specifics |
|---|---|
| pGOI | Any gene(s) of interest flanked by AAV ITRs. |
| pRC | AAV 4 rep and cap genes. See Example 30. |
| pRC-FM | pRC-Spy Tag. Provides VP1, VP2 and VP3 proteins |
| | fused to Spy Tag protein. See Examples 33 and 35. |
| pHELP | Comprises one or more adenovirus helper genes. |
| | See Example 31. |
| pHC-SCM | pHC-Spy Catcher. Provides an antibody heavy chain |
| | fused to Spy Catcher. |
| pLC | Comprises an antibody light chain. |

(1) Plasmid pGOI can comprise one or more genes that are desired to be expressed in covalently surface modified AAV4;
(2) Plasmids pRC-FM and pHC-SCM can be modified to use other specific binding pairs. The first member (FM) and the second cognate member (SCM) should be complementary in order to bond.
(3) Plasmids pHC-SCM and pLC can be modified to use other Retargeting Molecules. For example, the heavy chain (HC) and the light chain (LC) should be complementary in order form a functional Retargeting Molecule.
(4) Detargeting mutations can be undertaken as disclosed herein and in publications.

The second chart below cites to exemplary modifications to the above chart based upon the descriptions and sequence examples contained herein:

| | |
|---|---|
| pRC | AAV4 rep and cap genes. See Example 30. |
| pRC-FM | See Examples 33 and 35 to obtain VP1, VP2 and VP3 |
| | proteins fused to a an FM (e.g., tag-type protein) |
| | protein. |
| pHELP | See Example 31 concerning helper genes from herpes |
| | simplex virus (HSV), human papilloma virus (HPV), |
| | bocavirus, and baculovirus. |
| pHC-SCM | See Examples 32, 33 and 35 to obtain catcher-type |
| | sequences. |
| pLC | Comprises an antibody light chain. |

### Example 17—AAV5

Covalently surface modified AAV5 can be made by transfecting a cell with plasmids as set forth below, followed by culturing the transfected cells and then harvesting the covalently surface modified AAV5. The first chart below uses SpyTag-SpyCatcher as specific binding pairs, and Adenovirus Helper Genes.

Modifications are possible and the considerations include:

| Plasmid type | Specifics |
|---|---|
| pGOI | Any gene(s) of interest flanked by AAV ITRs. |
| pRC | AAV 5 rep and cap genes. See Example 30. |
| pRC-FM | pRC-Spy Tag. Provides VP1, VP2 and VP3 proteins |
| | fused to Spy Tag protein. See Examples 33 and 35. |
| pHELP | Comprises one or more adenovirus helper genes. |
| | See Example 31. |
| pHC-SCM | pHC-Spy Catcher. Provides an antibody heavy chain |
| | fused to Spy Catcher. |
| pLC | Comprises an antibody light chain. |

(1) Plasmid pGOI can comprise one or more genes that are desired to be expressed in covalently surface modified AAV1;
(2) Plasmids pRC-FM and pHC-SCM can be modified to use other specific binding pairs. The first member (FM) and the second cognate member (SCM) should be complementary in order to bond.
(3) Plasmids pHC-SCM and pLC can be modified to use other Retargeting Molecules. For example, the heavy chain (HC) and the light chain (LC) should be complementary in order form a functional Retargeting Molecule.
(4) Detargeting mutations can be undertaken as disclosed herein and in publications.

The second chart below cites to exemplary modifications to the above chart based upon the descriptions and sequence examples contained herein:

| | |
|---|---|
| pRC | AAV 5 rep and cap genes. See Example 30. |
| pRC-FM | See Examples 33 and 35 to obtain VP1, VP2 and VP3 |
| | proteins fused to a an FM (e.g., tag-type protein). |
| | protein. |
| pHELP | See Example 31 concerning helper genes from herpes |
| | simplex virus (HSV), human papilloma virus (HPV), |
| | bocavirus, and baculovirus. |
| pHC-SCM | See Examples 32, 33 and 35 to obtain catcher-type |
| | sequences. |
| pLC | Comprises an antibody light chain. |

### Example 18—AAV6

Covalently surface modified AAV6 can be made by transfecting a cell with plasmids as set forth below, followed by culturing the transfected cells and then harvesting the covalently surface modified AAV6. The first chart below uses SpyTag-SpyCatcher as specific binding pairs, and Adenovirus Helper Genes.

Modifications are possible and the considerations include:

| Plasmid type | Specifics |
|---|---|
| pGOI | Any gene(s) of interest flanked by AAV ITRs. |
| pRC | AAV 6 rep and cap genes. See Example 30. |
| pRC-FM | pRC-Spy Tag. Provides VP1, VP2 and VP3 proteins fused |
| | to Spy Tag protein. See Examples 33 and 35. |
| pHELP | Comprises one or more adenovirus helper genes. |
| | See Example 31. |
| pHC-SCM | pHC-Spy Catcher. Provides an antibody heavy chain |
| | fused to Spy Catcher. |
| pLC | Comprises an antibody light chain. |

(1) Plasmid pGOI can comprise one or more genes that are desired to be expressed in covalently surface modified AAV1;
(2) Plasmids pRC-FM and pHC-SCM can be modified to use other specific binding pairs. The first member (FM) and the second cognate member (SCM) should be complementary in order to bond.
(3) Plasmids pHC-SCM and pLC can be modified to use other Retargeting Molecules. For example, the heavy chain (HC) and the light chain (LC) should be complementary in order form a functional Retargeting Molecule.
(4) Detargeting mutations can be undertaken as disclosed herein and in publications.

The second chart below cites to exemplary modifications to the above chart based upon the descriptions and sequence examples contained herein:

| | |
|---|---|
| pRC | AAV 6 rep and cap genes. See Example 30. |
| pRC-FM | See Examples 33 and 35 to obtain VP1, VP2 and VP3 |
| | proteins fused to a an FM (e.g., tag-type protein). |
| | protein. |
| pHELP | See Example 31 concerning helper genes from herpes |
| | simplex virus (HSV), human papilloma virus (HPV), |
| | bocavirus, and baculovirus. |
| pHC-SCM | See Examples 32, 33 and 35 to obtain catcher-type |
| | sequences. |
| pLC | Comprises an antibody light chain. |

### Example 19—AAV7

Covalently surface modified AAV7 can be made by transfecting a cell with plasmids as set forth below, followed by culturing the transfected cells and then harvesting the covalently surface modified AAV7. The first chart below uses SpyTag-SpyCatcher as specific binding pairs, and Adenovirus Helper Genes.

Modifications are possible and the considerations include:

| Plasmid type | Specifics |
|---|---|
| pGOI | Any gene(s) of interest flanked by AAV ITRs. |
| pRC | AAV 7 rep and cap genes. See Example 30. |
| pRC-FM | pRC-Spy Tag. Provides VP1, VP2 and VP3 proteins |
| | fused to Spy Tag protein. See Examples 33 and 35. |
| pHELP | Comprises one or more adenovirus helper genes. |
| | See Example 31. |
| pHC-SCM | pHC-Spy Catcher. Provides an antibody heavy chain |
| | fused to Spy Catcher. |
| pLC | Comprises an antibody light chain. |

(1) Plasmid pGOI can comprise one or more genes that are desired to be expressed in covalently surface modified AAV7;
(2) Plasmids pRC-FM and pHC-SCM can be modified to use other specific binding pairs. The first member (FM) and the second cognate member (SCM) should be complementary in order to bond.
(3) Plasmids pHC-SCM and pLC can be modified to use other Retargeting Molecules. For example, the heavy chain (HC) and the light chain (LC) should be complementary in order form a functional Retargeting Molecule.
(4) Detargeting mutations can be undertaken as disclosed herein and in publications.

The second chart below cites to exemplary modifications to the above chart based upon the descriptions and sequence examples contained herein:

| | |
|---|---|
| pRC | AAV 7 rep and cap genes. See Example 30. |
| pRC-FM | See Examples 33 and 35 to obtain VP1, VP2 and VP3 |
| | proteins fused to a an FM (e.g., tag-type protein). |
| | protein. |
| pHELP | See Example 31 concerning helper genes from herpes |
| | simplex virus (HSV), human papilloma virus (HPV), |
| | bocavirus, and baculovirus. |
| pHC-SCM | See Examples 32, 33 and 35 to obtain catcher-type |
| | sequences. |
| pLC | Comprises an antibody light chain. |

### Example 20—AAV8

Covalently surface modified AAV8 can be made by transfecting a cell with plasmids as set forth below, followed by culturing the transfected cells and then harvesting the covalently surface modified AAV8. The first chart below uses SpyTag-SpyCatcher as specific binding pairs, and Adenovirus Helper Genes.

Modifications are possible and the considerations include:

| Plasmid type | Specifics |
|---|---|
| pGOI | Any gene(s) of interest flanked by AAV ITRs. |
| pRC | AAV8 rep and cap genes. See Example 30. |
| pRC-FM | pRC-Spy Tag. Provides VP1, VP2 and VP3 proteins fused |
| | to Spy Tag protein. See Examples 33 and 35. |
| pHELP | Comprises one or more adenovirus helper genes. |
| | See Example 31. |
| pHC-SCM | pHC-Spy Catcher. Provides an antibody heavy chain |
| | fused to Spy Catcher. |
| pLC | Comprises an antibody light chain. |

(1) Plasmid pGOI can comprise one or more genes that are desired to be expressed in covalently surface modified AAV8;
(2) Plasmids pRC-FM and pHC-SCM can be modified to use other specific binding pairs. The first member (FM) and the second cognate member (SCM) should be complementary in order to bond.
(3) Plasmids pHC-SCM and pLC can be modified to use other Retargeting Molecules. For example, the heavy chain (HE) and the light chain (LC) should be complementary in order form a functional Retargeting Molecule.
(4) Detargeting mutations can be undertaken as disclosed herein and in publications.

The second chart below cites to exemplary modifications to the above chart based upon the descriptions and sequence examples contained herein:

| | |
|---|---|
| pRC | AAV 8 rep and cap genes. See Example 30. |
| pRC-FM | See Examples 33 and 35 to obtain VP1, VP2 and VP3 |
| | proteins fused to a an FM (e.g., tag-type protein). |
| | protein. |
| pHELP | See Example 31 concerning helper genes from herpes |
| | simplex virus (HSV), human papilloma virus (HPV), |
| | bocavirus, and baculovirus. |
| pHC-SCM | See Examples 32, 33 and 35 to obtain catcher-type |
| | sequences. |
| pLC | Comprises an antibody light chain. |

### Example 21—AAV9

Covalently surface modified AAV9 can be made by transfecting a cell with plasmids as set forth below, followed by culturing the transfected cells and then harvesting the covalently surface modified AAV9. The first chart below uses SpyTag-SpyCatcher as specific binding pairs, and Adenovirus Helper Genes.

Modifications are possible and the considerations include:

| Plasmid type | Specifics |
|---|---|
| pGOI | Any gene(s) of interest flanked by AAV ITRs. |
| pRC | AAV9 rep and cap genes. See Example 30. |
| pRC-FM | pRC-Spy Tag. Provides VP1, VP2 and VP3 proteins fused |
| | to Spy Tag protein. See Examples 33 and 35. |
| pHELP | Comprises one or more adenovirus helper genes. |
| | See Example 31. |
| pHC-SCM | pHC-Spy Catcher. Provides an antibody heavy chain |
| | fused to Spy Catcher. |
| pLC | Comprises an antibody light chain. |

(1) Plasmid pGOI can comprise one or more genes that are desired to be expressed in covalently surface modified AAV9;
(2) Plasmids pRC-FM and pHC-SM can be modified to use other specific binding pairs. The first member (FM) and the second cognate member (SCM) should be complementary in order to bond.
(3) Plasmids pHC-SCM and pLC can be modified to use other Retargeting Molecules. For example, the heavy chain (HO) and the light chain (LO) should be complementary in order form a functional Retargeting Molecule.
(4) Detargeting mutations can be undertaken as disclosed herein and in publications.

The second chart below cites to exemplary modifications to the above chart based upon the descriptions and sequence examples contained herein:

| | |
|---|---|
| pRC | AAV9 rep and cap genes. See Example 30. |
| pRC-FM | See Examples 33 and 35 to obtain VP1, VP2 and VP3 |
| | proteins fused to a an FM (e.g., tag-type protein). |
| | protein. |
| pHELP | See Example 31 concerning helper genes from herpes |
| | simplex virus (HSV), human papilloma virus (HPV), |
| | bocavirus, and baculovirus. |
| pHC-SCM | See Examples 32, 33 and 35 to obtain catcher-type |
| | sequences. |
| pLC | Comprises an antibody light chain. |

### Example 22—AAV10

Covalently surface modified AAV10 can be made by transfecting a cell with plasmids as set forth below, followed by culturing the transfected cells and then harvesting the covalently surface modified AAV10. The first chart below uses SpyTag-SpyCatcher as specific binding pairs, and Adenovirus Helper Genes.

Modifications are possible and the considerations include:

| Plasmid type | Specifics |
|---|---|
| pGOI | Any gene(s) of interest flanked by AAV ITRs. |
| pRC | AAV10 rep and cap genes. See Example 30. |
| pRC-FM | pRC-Spy Tag. Provides VP1, VP2 and VP3 proteins fused |
| | to Spy Tag protein. See Examples 33 and 35. |
| pHELP | Comprises one or more adenovirus helper genes. |
| | See Example 31. |
| pHC-SCM | pHC-Spy Catcher. Provides an antibody heavy chain |
| | fused to Spy Catcher. |
| pLC | Comprises an antibody light chain. |

(1) Plasmid pGOI can comprise one or more genes that are desired to be expressed in covalently surface modified AAV10;
(2) Plasmids pRC-FM and pHC-SCM can be modified to use other specific binding pairs. The first member (FM) and the second cognate member (SCM) should be complementary in order to bond.
(3) Plasmids pHC-SCM and pLC can be modified to use other Retargeting Molecules. For example, the heavy chain (HC) and the light chain (LC) should be complementary in order form a functional Retargeting Molecule.
(4) Detargeting mutations can be undertaken as disclosed herein and in publications.

The second chart below cites to exemplary modifications to the above chart based upon the descriptions and sequence examples contained herein:

| | |
|---|---|
| pRC | AAV10 rep and cap genes. see Example 30. |
| pRC-FM | See Examples 3 and 35 to obtain VP1, VP2 and VP3 |
| | proteins fused to a an FM (e.g., tag-type protein). |
| | protein. |
| pHELP | See Example 31 concerning helper genes from herpes |
| | simplex virus (HSV), human papilloma virus (HPV), |
| | bocavirus, and baculovirus. |
| pHC-SCM | See Examples 32, 33 and 35 to obtain catcher-type |
| | sequences. |
| pLC | Comprises an antibody light chain. |

### Example 23—AAV11

Covalently surface modified AAV11 can be made by transfecting a cell with plasmids as set forth below, followed by culturing the transfected cells and then harvesting the covalently surface modified AAV11. The first chart below uses SpyTag-SpyCatcher as specific binding pairs, and Adenovirus Helper Genes.

Modifications are possible and the considerations include:

| Plasmid type | Specifics |
|---|---|
| pGOI | Any gene(s) of interest flanked by AAV ITRs. |
| pRC | AAV 11 rep and cap genes. See Example 30. |
| pRC-FM | pRC-Spy Tag. Provides VP1, VP2 and VP3 proteins |
| | fused to Spy Tag protein. See Examples 33 and 35. |
| pHELP | Comprises one or more adenovirus helper genes. |
| | See Example 31. |
| pHC-SCM | pHC-Spy Catcher. Provides an antibody heavy chain |
| | fused to Spy Catcher. |
| pLC | Comprises an antibody light chain. |

(1) Plasmid pGOI can comprise one or more genes that are desired to be expressed in covalently surface modified AAV7;
(2) Plasmids pRC-FM and pHC-SCM can be modified to use other specific binding pairs. The first member (FM) and the second cognate member (SCM) should be complementary in order to bond.
(3) Plasmids pHC-SCM and pLC can be modified to use other Retargeting Molecules. For example, the heavy chain (HC) and the light chain (LC) should be complementary in order form a functional Retargeting Molecule.
(4) Detargeting mutations can be undertaken as disclosed herein and in publications.

The second chart below cites to exemplary modifications to the above chart based upon the descriptions and sequence examples contained herein:

| | |
|---|---|
| pRC | AAV 11 rep and cap genes. see Example 30. |
| pRC-FM | See Examples 33 and 35 to obtain VP1, VP2 and VP3 |
| | proteins fused to a an FM (e.g., tag-type protein). |
| | protein. |
| pHELP | See Example 31 concerning helper genes from herpes |
| | simplex virus (HSV), human papilloma virus (HPV), |
| | bocavirus, and baculovirus. |
| pHC-SCM | See Examples 32, 33 and 35 to obtain catcher-type |
| | sequences. |
| pLC | Comprises an antibody light chain. |

### Example 24—AAV12

Covalently surface modified AAV12 can be made by transfecting a cell with plasmids as set forth below, followed by culturing the transfected cells and then harvesting the covalently surface modified AAV12. The first chart below uses SpyTag-SpyCatcher as specific binding pairs, and Adenovirus Helper Genes.

Modifications are possible and the considerations include:

| Plasmid type | Specifics |
|---|---|
| pGOI | Any gene(s) of interest flanked by AAV ITRs. |
| pRC | AAV 12 rep and cap genes. See Example 30. |
| pRC-FM | pRC-Spy Tag. Provides VP1, VP2 and VP3 proteins |
| | fused to Spy Tag protein. See Examples 33 and 35. |
| pHELP | Comprises one or more adenovirus helper genes. |
| | See Example 31. |
| pHC-SCM | pHC-Spy Catcher. Provides an antibody heavy chain |
| | fused to Spy Catcher. |
| pLC | Comprises an antibody light chain. |

(1) Plasmid pGOI can comprise one or more genes that are desired to be expressed in covalently surface modified AAV12;
(2) Plasmids pRC-FM and pHC-SCM can be modified to use other specific binding pairs. The first member (FM) and the second cognate member (SCM) should be complementary in order to bond.
(3) Plasmids pHC-SCM and pLC can be modified to use other Retargeting Molecules. For example, the heavy chain (HC) and the light chain (LC) should be complementary in order form a functional Retargeting Molecule.
(4) Detargeting mutations can be undertaken as disclosed herein and in publications.

The second chart below cites to exemplary modifications to the above chart based upon the descriptions and sequence examples contained herein:

| | |
|---|---|
| pRC | AAV 12 rep and cap genes. see Example 30. |
| pRC-FM | See Examples 33 and 35 to obtain VP1, VP2 and VP3 |
| | proteins fused to a an FM (e.g., tag-type protein). |
| | protein. |
| pHELP | See Example 31 concerning helper genes from herpes |
| | simplex virus (HSV), human papilloma virus (HPV), |
| | bocavirus, and baculovirus. |
| pHC-SCM | See Examples 32, 33 and 35 to obtain catcher-type |
| | sequences. |
| pLC | Comprises an antibody light chain. |

### Example 25—AAV13

Covalently surface modified AAV13 can be made by transfecting a cell with plasmids as set forth below, followed by culturing the transfected cells and then harvesting the covalently surface modified AAV13. The first chart below uses SpyTag-SpyCatcher as specific binding pairs, and Adenovirus Helper Genes.

Modifications are possible and the considerations include:

| Plasmid type | Specifics |
|---|---|
| pGOI | Any gene(s) of interest flanked by AAV ITRs. |
| pRC | AAV 13 rep and cap genes. See Example 30. |
| pRC-FM | pRC-Spy Tag. Provides VP1, VP2 and VP3 proteins |
| | fused to Spy Tag protein. See Examples 33 and 35. |
| pHELP | Comprises one or more adenovirus helper genes. |
| | See Example 31. |
| pHC-SCM | pHC-Spy Catcher. Provides an antibody heavy chain |
| | fused to Spy Catcher. |
| pLC | Comprises an antibody light chain. |

(1) Plasmid pGOI can comprise one or more genes that are desired to be expressed in covalently surface modified AAV13;
(2) Plasmids pRC-FM and pHC-SCM can be modified to use other specific binding pairs. The first member (FM) and the second cognate member (SCM) should be complementary in order to bond.
(3) Plasmids pHC-SCM and pLC can be modified to use other Retargeting Molecules. For example, the heavy chain (HC) and the light chain (LC) should be complementary in order form a functional Retargeting Molecule.
(4) Detargeting mutations can be undertaken as disclosed herein and in publications.

The second chart below cites to exemplary modifications to the above chart based upon the descriptions and sequence examples contained herein:

| | |
|---|---|
| pRC | AAV 13 rep and cap genes. See Example 30. |
| pRC-FM | See Examples 33 and 35 to obtain VP1, VP2 and VP3 |
| | proteins fused to a an FM (e.g., tag-type protein). |
| | protein. |
| pHELP | See Example 31 concerning helper genes from herpes |
| | simplex virus (HSV), human papilloma virus (HPV), |
| | bocavirus, and baculovirus. |
| pHC-SCM | See Examples 32, 33 and 35 to obtain catcher-type |
| | sequences. |
| pLC | Comprises an antibody light chain. |

### Example 26—AAV rh10

Covalently surface modified AAV rh10 can be made by transfecting a cell with plasmids as set forth below, followed by culturing the transfected cells and then harvesting the covalently surface modified AAV rh10. The first chart below uses SpyTag-SpyCatcher as specific binding pairs, and Adenovirus Helper Genes.

Modifications are possible and the considerations include:

| Plasmid type | Specifics |
|---|---|
| pGOI | Any gene(s) of interest flanked by AAV ITRs. |
| pRC | AAV rh10 rep and cap genes. See Example 30. |
| pRC-FM | pRC-Spy Tag. Provides VP1, VP2 and VP3 proteins |
| | fused to Spy Tag protein. See Examples 33 and 35. |
| pHELP | Comprises one or more adenovirus helper genes. |
| | See Example 31. |
| pHC-SCM | pHC-Spy Catcher. Provides an antibody heavy chain |
| | fused to Spy Catcher. |
| pLC | Comprises an antibody light chain. |

(1) Plasmid pGOI can comprise one or more genes that are desired to be expressed in covalently surface modified AAV rh10;
(2) Plasmids pRC-FM and pHC-SM can be modified to use other specific binding pairs. The first member (FM) and the second cognate member (SCM) should be complementary in order to bond.
(3) Plasmids pHC-SCM and pLC can be modified to use other Retargeting Molecules. For example, the heavy chain (HO) and the light chain (LO) should be complementary in order form a functional Retargeting Molecule.
(4) Detargeting mutations can be undertaken as disclosed herein and in publications.

The second chart below cites to exemplary modifications to the above chart based upon the descriptions and sequence examples contained herein:

| | |
|---|---|
| pRC | AAV rh10 rep and cap genes. See Example 30. |
| pRC-FM | See Examples 33 and 35 to obtain VP1, VP2 and VP3 |
| | proteins fused to a an FM (e.g., tag-type protein). |
| | protein. |
| pHELP | See Example 31 concerning helper genes from herpes |
| | simplex virus (HSV), human papilloma virus (HPV), |
| | bocavirus, and baculovirus. |
| pHC-SCM | See Examples 32, 33 and 35 to obtain catcher-type |
| | sequences. |
| pLC | Comprises an antibody light chain. |

### Example 27—AAV rh39

Covalently surface modified AAV rh39 can be made by transfecting a cell with plasmids as set forth below, followed by culturing the transfected cells and then harvesting the covalently surface modified AAV rh39. The first chart below uses SpyTag-Spypatcher as specific binding pairs, and Adenovirus Helper Genes.

Modifications are possible and the considerations include:

| Plasmid type | Specifics |
|---|---|
| pGOI | Any gene(s) of interest flanked by AAV ITRs. |
| pRC | AAV rh39 rep and cap genes. See Example 30. |
| pRC-FM | pRC-Spy Tag. Provides VP1, VP2 and VP3 proteins |
| | fused to Spy Tag protein. See Examples 33 and 35. |
| pHELP | Comprises one or more adenovirus helper genes. |
| | See Example 31. |
| pHC-SCM | pHC-Spy Catcher. Provides an antibody heavy chain |
| | fused to Spy Catcher. |
| pLC | Comprises an antibody light chain. |

(1) Plasmid pGOI can comprise one or more genes that are desired to be expressed in covalently surface modified AAV rh39;
(2) Plasmids pRC-FM and pHC-SCM can be modified to use other specific binding pairs. The first member (FM) and the second cognate member (SCM) should be complementary in order to bond.
(3) Plasmids pHC-SCM and pLC can be modified to use other Retargeting Molecules. For example, the heavy chain (HC) and the light chain (LC) should be complementary in order form a functional Retargeting Molecule.
(4) Detargeting mutations can be undertaken as disclosed herein and in publications.

The second chart below cites to exemplary modifications to the above chart based upon the descriptions and sequence examples contained herein:

| | |
|---|---|
| pRC | AAV rh39 rep and cap genes. See Example 30. |
| pRC-FM | See Examples 33 and 35 to obtain VP1, VP2 and VP3 |
| | proteins fused to a an FM (e.g., tag-type protein). |
| | protein. |
| pHELP | See Example 31 concerning helper genes from herpes |
| | simplex virus (HSV), human papilloma virus (HPV), |
| | bocavirus, and baculovirus. |
| pHC-SCM | See Examples 32, 33 and 35 to obtain catcher-type |
| | sequences. |
| pLC | Comprises an antibody light chain. |

### Example 28—AAV rh43

Covalently surface modified AAV rh43 can be made by transfecting a cell with plasmids as set forth below, followed by culturing the transfected cells and then harvesting the covalently surface modified AAV rh43. The first chart below uses SpyTag-SpyCatcher as specific binding pairs, and Adenovirus Helper Genes.

Modifications are possible and the considerations include:

| Plasmid type | Specifics |
|---|---|
| pGOI | Any gene(s) of interest flanked by AAV ITRs. |
| pRC | AAV rh43 rep and cap genes. See Example 30. |
| pRC-FM | pRC-Spy Tag. Provides VP1, VP2 and VP3 proteins |
| | fused to Spy Tag protein. See Examples 33 and 35. |
| pHELP | Comprises one or more adenovirus helper genes. |
| | See Example 31. |
| pHC-SCM | pHC-Spy Catcher. Provides an antibody heavy chain |
| | fused to Spy Catcher. |
| pLC | Comprises an antibody light chain. |

(1) Plasmid pGOI can comprise one or more genes that are desired to be expressed in covalently surface modified AAV rh43;
(2) Plasmids pRC-FM and pHC-SCM can be modified to use other specific binding pairs. The first member (FM) and the second cognate member (SCM) should be complementary in order to bond.
(3) Plasmids pHC-SCM and pLC can be modified to use other Retargeting Molecules. For example, the heavy chain (HC) and the light chain (LC) should be complementary in order form a functional Retargeting Molecule.
(4) Detargeting mutations can be undertaken as disclosed herein and in publications.

The second chart below cites to exemplary modifications to the above chart based upon the descriptions and sequence examples contained herein:

| | |
|---|---|
| pRC | AAV rh43 rep and cap genes. See Example 30. |
| pRC-FM | See Examples 33 and 35 to obtain VP1, VP2 and VP3 |
| | proteins fused to a an FM (e.g., tag-type protein). |
| | protein. |
| pHELP | See Example 31 concerning helper genes from herpes |
| | simplex virus (HSV), human papilloma virus (HPV), |
| | bocavirus, and baculovirus. |
| pHC-SCM | See Examples 32, 33 and 35 to obtain catcher-type |
| | sequences. |
| pLC | Comprises an antibody light chain. |

### Example 29—AAV rh74

Covalently surface modified AAV rh74 can be made by transfecting a cell with plasmids as set forth below, followed by culturing the transfected cells and then harvesting the covalently surface modified AAV rh74. The first chart below uses SpyTag-SpyCatcher as specific binding pairs, and Adenovirus Helper Genes.

Modifications are possible and the considerations include:

| Plasmid type | Specifics |
|---|---|
| pGOI | Any gene(s) of interest flanked by AAV ITRs. |
| pRC | AAV rh74 rep and cap genes. See Example 30. |
| pRC-FM | pRC-Spy Tag. Provides VP1, VP2 and VP3 proteins |
| | fused to Spy Tag protein. See Examples 33 and 35. |
| pHELP | Comprises one or more adenovirus helper genes. |
| | See Example 31. |
| pHC-SCM | pHC-Spy Catcher. Provides an antibody heavy chain |
| | fused to Spy Catcher. |
| pLC | Comprises an antibody light chain. |

(1) Plasmid pGOI can comprise one or more genes that are desired to be expressed in covalently surface modified AAV rh74;
(2) Plasmids pRC-FM and pHC-SCM can be modified to use other specific binding pairs. The first member (FM) and the second cognate member (SCM) should be complementary in order to bond.
(3) Plasmids pHC-SCM and pLC can be modified to use other Retargeting Molecules. For example, the heavy chain (HC) and the light chain (LC) should be complementary in order form a functional Retargeting Molecule.
(4) Detargeting mutations can be undertaken as disclosed herein and in publications.

The second chart below cites to exemplary modifications to the above chart based upon the descriptions and sequence examples contained herein:

| | |
|---|---|
| pRC | AAV rh74 rep and cap genes. See Example 30. |
| pRC-FM | See Examples 33 and 35 to obtain VP1, VP2 and VP3 |
| | proteins fused to a an FM (e.g., tag-type protein). |
| | protein. |
| pHELP | See Example 31 concerning helper genes from herpes |
| | simplex virus (HSV), human papilloma virus (HPV), |
| | bocavirus, and baculovirus. |
| pHC-SCM | See Examples 32, 33 and 35 to obtain catcher-type |
| | sequences. |
| pLC | Comprises an antibody light chain. |

### Sequence Examples

The following examples set forth exemplary sequences, which are optional for use and do not limit the inventions in any manner. Other AAV sequences, helper sequences, Specific Binding Pair sequences, and DNA Barcode sequences are available and accessible. Gene of interest sequences and retargeting molecule sequences can be selected based upon the purpose of the covalently surface modified AAV and the cell/tissue to be targeted.

### Example 30—AAV Polynucleotide Sequences

AAV Rep, Cap and ITR sequences are known in the art. The present inventions are amenable to all AAV serotypes. AAV sequences from various AAV serotypes are set forth below. Many of these sequences are available from the National Center for Biotechnology Information (NCBI).

AAV-1
Full Genome: NC_002077
CapVP1: (SEQ ID NO: 1)
ATGGCTGCCGATGGTTATCTTCCAGATTGGCTCGAGGACAACCTCTCTGAGGGCATTCGCGAGTGGTGGGACTTG
AAACCTGGAGCCCCGAAGCCCAAAGCCAACCAGCAAAAGCAGGACGACGGCCGGGGTCTGGTGCTTCCTGGCTAC
AAGTACCTCGGACCCTTCAACGGACTCGACAAGGGGGAGCCCGTCAACGCGGCGGACGCAGCGGCCCTCGAGCAC
GACAAGGCCTACGACCAGCAGCTCAAAGCGGGTGACAATCCGTACCTGCGGTATAACCACGCCGACGCCGAGTTT
CAGGAGCGTCTGCAAGAAGATACGTCTTTTGGGGGCAACCTCGGGCGAGCAGTCTTCCAGGCCAAGAAGCGGGTT
CTCGAACCTCTCGGTCTGGTTGAGGAAGGCGCTAAGACGGCTCCTGGAAAGAAACGTCCGGTAGAGCAGTCGCCA
CAAGAGCCAGACTCCTCCTCGGGCATCGGCAAGACAGGCCAGCAGCCCGCTAAAAAGAGACTCAATTTTGGTCAG
ACTGGCGACTCAGAGTCAGTCCCCGATCCACAACCTCTCGGAGAACCTCCAGCAACCCCCGCTGCTGTGGGACCT
ACTACAATGGCTTCAGGCGGTGGCGCACCAATGGCAGACAATAACGAAGGCGCCGACGGAGTGGGTAATGCCTCA
GGAAATTGGCATTGCGATTCCACATGGCTGGGCGACAGAGTCATCACCACCAGCACCCGCACCTGGGCCTTGCCC
ACCTACAATAACCACCTCTACAAGCAAATCTCCAGTGCTTCAACGGGGGCCAGCAACGACAACCACTACTTCGGC
TACAGCACCCCCTGGGGGTATTTTGATTTCAACAGATTCCACTGCCACTTTTCACCACGTGACTGGCAGCGACTC
ATCAACAACAATTGGGGATTCCGGCCCAAGAGACTCAACTTCAAACTCTTCAACATCCAAGTCAAGGAGGTCACG
ACGAATGATGGCGTCACAACCATCGCTAATAACCTTACCAGCACGGTTCAAGTCTTCTCGGACTCGGAGTACCAG
CTTCCGTACGTCCTCGGCTCTGCGCACCAGGGCTGCCTCCCTCCGTTCCCGGCGGACGTGTTCATGATTCCGCAA
TACGGCTACCTGACGCTCAACAATGGCAGCCAAGCCGTGGGACGTTCATCCTTTTACTGCCTGGAATATTTCCCT
TCTCAGATGCTGAGAACGGGCAACAACTTTACCTTCAGCTACACCTTTGAGGAAGTGCCTTTCCACAGCAGCTAC
GCGCACAGCCAGAGCCTGGACCGGCTGATGAATCCTCTCATCGACCAATACCTGTATTACCTGAACAGAACTCAA
AATCAGTCCGGAAGTGCCCAAAACAAGGACTTGCTGTTTAGCCGTGGGTCTCCAGCTGGCATGTCTGTTCAGCCC
AAAAACTGGCTACCTGGACCCTGTTATCGGCAGCAGCGCGTTTCTAAAACAAAAACAGACAACAACAACAGCAAT
TTTACCTGGACTGGTGCTTCAAAATATAACCTCAATGGGCGTGAATCCATCATCAACCCTGGCACTGCTATGGCC
TCACACAAAGACGACGAAGACAAGTTCTTTCCCATGAGCGGTGTCATGATTTTTGGAAAAGAGAGCGCCGGAGCT
TCAAACACTGCATTGGACAATGTCATGATTACAGACGAAGAGGAAATTAAAGCCACTAACCCTGTGGCCACCGAA
AGATTTGGGACCGTGGCAGTCAATTTCCAGAGCAGCAGCACAGACCCTGCGACCGGAGATGTGCATGCTATGGGA
GCATTACCTGGCATGGTGTGGCAAGATAGAGACGTGTACCTGCAGGGTCCCATTTGGGCCAAAATTCCTCACACA
GATGGACACTTTCACCCGTCTCCTCTTATGGGCGGCTTTGGACTCAAGAACCCGCCTCCTCAGATCCTCATCAAA
AACACGCCTGTTCCTGCGAATCCTCCGGCGGAGTTTTCAGCTACAAAGTTTGCTTCATTCATCACCCAATACTCC
ACAGGACAAGTGAGTGTGGAAATTGAATGGGAGCTGCAGAAAGAAAACAGCAAGCGCTGGAATCCCGAAGTGCAG
TACACATCCAATTATGCAAAATCTGCCAACGTTGATTTTACTGTGGACAACAATGGACTTTATACTGAGCCTCGC
CCCATTGGCACCCGTTACCTTACCCGTCCCCTGTAA
Rep78: (SEQ ID NO: 2)
ATGCCGGGCTTCTACGAGATCGTGATCAAGGTGCCGAGCGACCTGGACGAGCACCTGCCGGGCATTTCTGACTCG
TTTGTGAGCTGGGTGGCCGAGAAGGAATGGGAGCTGCCCCCGGATTCTGACATGGATCTGAATCTGATTGAGCAG
GCACCCCTGACCGTGGCCGAGAAGCTGCAGCGCGACTTCCTGGTCCAATGGCGCCGCGTGAGTAAGGCCCCGGAG
GCCCTCTTCTTTGTTCAGTTCGAGAAGGGCGAGTCCTACTTCCACCTCCATATTCTGGTGGAGACCACGGGGGTC
AAATCCATGGTGCTGGGCCGCTTCCTGAGTCAGATTAGGGACAAGCTGGTGCAGACCATCTACCGCGGGATCGAG
CCGACCCTGCCCAACTGGTTCGCGGTGACCAAGACGCGTAATGGCGCCGGAGGGGGGAACAAGGTGGTGGACGAG
TGCTACATCCCCAACTACCTCCTGCCCAAGACTCAGCCCGAGCTGCAGTGGGCGTGGACTAACATGGAGGAGTAT
ATAAGCGCCTGTTTGAACCTGGCCGAGCGCAAACGGCTCGTGGCGCAGCACCTGACCCACGTCAGCCAGACCCAG
GAGCAGAACAAGGAGAATCTGAACCCCAATTCTGACGCGCCTGTCATCCGGTCAAAAACCTCCGCGCGCTACATG
GAGCTGGTCGGGTGGCTGGTGGACCGGGGCATCACCTCCGAGAAGCAGTGGATCCAGGAGGACCAGGCCTCGTAC
ATCTCCTTCAACGCCGCTTCCAACTCGCGGTCCCAGATCAAGGCCGCTCTGGACAATGCCGGCAAGATCATGGCG
CTGACCAAATCCGCGCCCGACTACCTGGTAGGCCCCGCTCCGCCCGCGGACATTAAAACCAACCGCATCTACCGC
ATCCTGGAGCTGAACGGCTACGAACCTGCCTACGCCGGCTCCGTCTTTCTCGGCTGGGCCCAGAAAAGGTTCGGG
AAGCGCAACACCATCTGGCTGTTTGGGCCGGCCACCACGGGCAAGACCAACATCGCGGAAGCCATCGCCCACGCC
GTGCCCTTCTACGGCTGCGTCAACTGGACCAATGAGAACTTTCCCTTCAATGATTGCGTCGACAAGATGGTGATC
TGGTGGGAGGAGGGCAAGATGACGGCCAAGGTCGTGGAGTCCGCCAAGGCCATTCTCGGCGGCAGCAAGGTGCGC
GTGGACCAAAAGTGCAAGTCGTCCGCCCAGATCGACCCCACCCCCGTGATCGTCACCTCCAACACCAACATGTGC
GCCGTGATTGACGGGAACAGCACCACCTTCGAGCACCAGCAGCCGTTGCAGGACCGGATGTTCAAATTTGAACTC
ACCCGCCGTCTGGAGCATGACTTTGGCAAGGTGACAAAGCAGGAAGTCAAAGAGTTCTTCCGCTGGGCGCAGGAT
CACGTGACCGAGGTGGCGCATGAGTTCTACGTCAGAAAGGGTGGAGCCAACAAAAGACCCGCCCCCGATGACGCG
GATAAAAGCGAGCCCAAGCGGGCCTGCCCCTCAGTCGCGGATCCATCGACGTCAGACGCGGAAGGAGCTCCGGTG
GACTTTGCCGACAGGTACCAAAACAAATGTTCTCGTCACGCGGGCATGCTTCAGATGCTGTTTCCCTGCAAGACA
TGCGAGAGAATGAATCAGAATTTCAACATTTGCTTCACGCACGGGACGAGAGACTGTTCAGAGTGCTTCCCCGGC
GTGTCAGAATCTCAACCGGTCGTCAGAAAGAGGACGTATCGGAAACTCTGTGCCATTCATCATCTGCTGGGGCGG
GCTCCCGAGATTGCTTGCTCGGCCTGCGATCTGGTCAACGTGGACCTGGATGACTGTGTTTCTGAGCAATAA
AAV-2
Full Genome: NC_001401
Rep78: (SEQ ID NO: 3)
ATGCCGGGGTTTTACGAGATTGTGATTAAGGTCCCCAGCGACCTTGACGAGCATCTGCCCGGCATTTCTGACAGC
TTTGTGAACTGGGTGGCCGAGAAGGAATGGGAGTTGCCGCCAGATTCTGACATGGATCTGAATCTGATTGAGCAG
GCACCCCTGACCGTGGCCGAGAAGCTGCAGCGCGACTTTCTGACGGAATGGCGCCGTGTGAGTAAGGCCCCGGAG
GCCCTTTTCTTTGTGCAATTTGAGAAGGGAGAGAGCTACTTCCACATGCACGTGCTCGTGGAAACCACCGGGGTG
AAATCCATGGTTTTGGGACGTTTCCTGAGTCAGATTCGCGAAAAACTGATTCAGAGAATTTACCGCGGGATCGAG
CCGACTTTGCCAAACTGGTTCGCGGTCACAAAGACCAGAAATGGCGCCGGAGGCGGGAACAAGGTGGTGGATGAG
TGCTACATCCCCAATTACTTGCTCCCCAAAACCCAGCCTGAGCTCCAGTGGGCGTGGACTAATATGGAACAGTAT
TTAAGCGCCTGTTTGAATCTCACGGAGCGTAAACGGTTGGTGGCGCAGCATCTGACGCACGTGTCGCAGACGCAG
GAGCAGAACAAAGAGAATCAGAATCCCAATTCTGATGCGCCGGTGATCAGATCAAAAACTTCAGCCAGGTACATG
GAGCTGGTCGGGTGGCTCGTGGACAAGGGGATTACCTCGGAGAAGCAGTGGATCCAGGAGGACCAGGCCTCATAC
ATCTCCTTCAATGCGGCCTCCAACTCGCGGTCCCAAATCAAGGCTGCCTTGGACAATGCGGGAAAGATTATGAGC
CTGACTAAAACCGCCCCCGACTACCTGGTGGGCCAGCAGCCCGTGGAGGACATTTCCAGCAATCGGATTTATAAA
ATTTTGGAACTAAACGGGTACGATCCCCAATATGCGGCTTCCGTCTTTCTGGGATGGGCCACGAAAAAGTTCGGC
AAGAGGAACACCATCTGGCTGTTTGGGCCTGCAACTACCGGGAAGACCAACATCGCGGAGGCCATAGCCCACACT
GTGCCCTTCTACGGGTGCGTAAACTGGACCAATGAGAACTTTCCCTTCAACGACTGTGTCGACAAGATGGTGATC
TGGTGGGAGGAGGGGAAGATGACCGCCAAGGTCGTGGAGTCGGCCAAAGCCATTCTCGGAGGAAGCAAGGTGCGC
GTGGACCAGAAATGCAAGTCCTCGGCCCAGATAGACCCGACTCCCGTGATCGTCACCTCCAACACCAACATGTGC
GCCGTGATTGACGGGAACTCAACGACCTTCGAACACCAGCAGCCGTTGCAAGACCGGATGTTCAAATTTGAACTC
ACCCGCCGTCTGGATCATGACTTTGGGAAGGTCACCAAGCAGGAAGTCAAAGACTTTTTCCGGTGGGCAAAGGAT
CACGTGGTTGAGGTGGAGCATGAATTCTACGTCAAAAAGGGTGGAGCCAAGAAAAGACCCGCCCCCAGTGACGCA
GATATAAGTGAGCCCAAACGGGTGCGCGAGTCAGTTGCGCAGCCATCGACGTCAGACGCGGAAGCTTCGATCAAC
TACGCAGACAGGTACCAAAACAAATGTTCTCGTCACGTGGGCATGAATCTGATGCTGTTTCCCTGCAGACAATGC
GAGAGAATGAATCAGAATTCAAATATCTGCTTCACTCACGGACAGAAAGACTGTTTAGAGTGCTTTCCCGTGTCA
GAATCTCAACCCGTTTCTGTCGTCAAAAAGGCGTATCAGAAACTGTGCTACATTCATCATATCATGGGAAAGGTG
CCAGACGCTTGCACTGCCTGCGATCTGGTCAATGTGGATTTGGATGACTGCATCTTTGAACAATAA
Rep52: (SEQ ID NO: 4)
ATGGAGCTGGTCGGGTGGCTCGTGGACAAGGGGATTACCTCGGAGAAGCAGTGGATCCAGGAGGACCAGGCCTCA
TACATCTCCTTCAATGCGGCCTCCAACTCGCGGTCCCAAATCAAGGCTGCCTTGGACAATGCGGGAAAGATTATG
AGCCTGACTAAAACCGCCCCCGACTACCTGGTGGGCCAGCAGCCCGTGGAGGACATTTCCAGCAATCGGATTTAT
AAAATTTTGGAACTAAACGGGTACGATCCCCAATATGCGGCTTCCGTCTTTCTGGGATGGGCCACGAAAAAGTTC
GGCAAGAGGAACACCATCTGGCTGTTTGGGCCTGCAACTACCGGGAAGACCAACATCGCGGAGGCCATAGCCCAC
ACTGTGCCCTTCTACGGGTGCGTAAACTGGACCAATGAGAACTTTCCCTTCAACGACTGTGTCGACAAGATGGTG
ATCTGGTGGGAGGAGGGGAAGATGACCGCCAAGGTCGTGGAGTCGGCCAAAGCCATTCTCGGAGGAAGCAAGGTG
CGCGTGGACCAGAAATGCAAGTCCTCGGCCCAGATAGACCCGACTCCCGTGATCGTCACCTCCAACACCAACATG
TGCGCCGTGATTGACGGGAACTCAACGACCTTCGAACACCAGCAGCCGTTGCAAGACCGGATGTTCAAATTTGAA
CTCACCCGCCGTCTGGATCATGACTTTGGGAAGGTCACCAAGCAGGAAGTCAAAGACTTTTTCCGGTGGGCAAAG
GATCACGTGGTTGAGGTGGAGCATGAATTCTACGTCAAAAAGGGTGGAGCCAAGAAAAGACCCGCCCCCAGTGAC
GCAGATATAAGTGAGCCCAAACGGGTGCGCGAGTCAGTTGCGCAGCCATCGACGTCAGACGCGGAAGCTTCGATC
AACTACGCAGACAGGTACCAAAACAAATGTTCTCGTCACGTGGGCATGAATCTGATGCTGTTTCCCTGCAGACAA
TGCGAGAGAATGAATCAGAATTCAAATATCTGCTTCACTCACGGACAGAAAGACTGTTTAGAGTGCTTTCCCGTG
TCAGAATCTCAACCCGTTTCTGTCGTCAAAAAGGCGTATCAGAAACTGTGCTACATTCATCATATCATGGGAAAG
GTGCCAGACGCTTGCACTGCCTGCGATCTGGTCAATGTGGATTTGGATGACTGCATCTTTGAACAATAA
CapVP1: (SEQ ID NO: 5)
ATGGCTGCCGATGGTTATCTTCCAGATTGGCTCGAGGACACTCTCTCTGAAGGAATAAGACAGTGGTGGAAGCTC
AAACCTGGCCCACCACCACCAAAGCCCGCAGAGCGGCATAAGGACGACAGCAGGGGTCTTGTGCTTCCTGGGTAC
AAGTACCTCGGACCCTTCAACGGACTCGACAAGGGAGAGCCGGTCAACGAGGCAGACGCCGCGGCCCTCGAGCAC
GACAAAGCCTACGACCGGCAGCTCGACAGCGGAGACAACCCGTACCTCAAGTACAACCACGCCGACGCGGAGTTT
CAGGAGCGCCTTAAAGAAGATACGTCTTTTGGGGGCAACCTCGGACGAGCAGTCTTCCAGGCGAAAAAGAGGGTT
CTTGAACCTCTGGGCCTGGTTGAGGAACCTGTTAAGACGGCTCCGGGAAAAAAGAGGCCGGTAGAGCACTCTCCT
GTGGAGCCAGACTCCTCCTCGGGAACCGGAAAGGCGGGCCAGCAGCCTGCAAGAAAAAGATTGAATTTTGGTCAG
ACTGGAGACGCAGACTCAGTACCTGACCCCCAGCCTCTCGGACAGCCACCAGCAGCCCCCTCTGGTCTGGGAACT
AATACGATGGCTACAGGCAGTGGCGCACCAATGGCAGACAATAACGAGGGCGCCGACGGAGTGGGTAATTCCTCG
GGAAATTGGCATTGCGATTCCACATGGATGGGCGACAGAGTCATCACCACCAGCACCCGAACCTGGGCCCTGCCC
ACCTACAACAACCACCTCTACAAACAAATTTCCAGCCAATCAGGAGCCTCGAACGACAATCACTACTTTGGCTAC
AGCACCCCTTGGGGGTATTTTGACTTCAACAGATTCCACTGCCACTTTTCACCACGTGACTGGCAAAGACTCATC
AACAACAACTGGGGATTCCGACCCAAGAGACTCAACTTCAAGCTCTTTAACATTCAAGTCAAAGAGGTCACGCAG
AATGACGGTACGACGACGATTGCCAATAACCTTACCAGCACGGTTCAGGTGTTTACTGACTCGGAGTACCAGCTC
CCGTACGTCCTCGGCTCGGCGCATCAAGGATGCCTCCCGCCGTTCCCAGCAGACGTCTTCATGGTGCCACAGTAT
GGATACCTCACCCTGAACAACGGGAGTCAGGCAGTAGGACGCTCTTCATTTTACTGCCTGGAGTACTTTCCTTCT
CAGATGCTGCGTACCGGAAACAACTTTACCTTCAGCTACACTTTTGAGGACGTTCCTTTCCACAGCAGCTACGCT
CACAGCCAGAGTCTGGACCGTCTCATGAATCCTCTCATCGACCAGTACCTGTATTACTTGAGCAGAACAAACACT
CCAAGTGGAACCACCACGCAGTCAAGGCTTCAGTTTTCTCAGGCCGGAGCGAGTGACATTCGGGACCAGTCTAGG
AACTGGCTTCCTGGACCCTGTTACCGCCAGCAGCGAGTATCAAAGACATCTGCGGATAACAACAACAGTGAATAC
TCGTGGACTGGAGCTACCAAGTACCACCTCAATGGCAGAGACTCTCTGGTGAATCCGGGCCCGGCCATGGCAAGC
CACAAGGACGATGAAGAAAAGTTTTTTCCTCAGAGCGGGGTTCTCATCTTTGGGAAGCAAGGCTCAGAGAAAACA
AATGTGGACATTGAAAAGGTCATGATTACAGACGAAGAGGAAATCAGGACAACCAATCCCGTGGCTACGGAGCAG
TATGGTTCTGTATCTACCAACCTCCAGAGAGGCAACAGACAAGCAGCTACCGCAGATGTCAACACACAAGGCGTT
CTTCCAGGCATGGTCTGGCAGGACAGAGATGTGTACCTTCAGGGGCCCATCTGGGCAAAGATTCCACACACGGAC
GGACATTTTCACCCCTCTCCCCTCATGGGTGGATTCGGACTTAAACACCCTCCTCCACAGATTCTCATCAAGAAC
ACCCCGGTACCTGCGAATCCTTCGACCACCTTCAGTGCGGCAAAGTTTGCTTCCTTCATCACACAGTACTCCACG
GGACAGGTCAGCGTGGAGATCGAGTGGGAGCTGCAGAAGGAAAACAGCAAACGCTGGAATCCCGAAATTCAGTAC
ACTTCCAACTACAACAAGTCTGTTAATGTGGACTTTACTGTGGACACTAATGGCGTGTATTCAGAGCCTCGCCCC
ATTGGCACCAGATACCTGACTCGTAATCTGTAA
CapVP2: (SEQ ID NO: 6)
ACGGCTCCGGGAAAAAAGAGGCCGGTAGAGCACTCTCCTGTGGAGCCAGACTCCTCCTCGGGAACCGGAAAGGCG
GGCCAGCAGCCTGCAAGAAAAAGATTGAATTTTGGTCAGACTGGAGACGCAGACTCAGTACCTGACCCCCAGCCT
CTCGGACAGCCACCAGCAGCCCCCTCTGGTCTGGGAACTAATACGATGGCTACAGGCAGTGGCGCACCAATGGCA
GACAATAACGAGGGCGCCGACGGAGTGGGTAATTCCTCGGGAAATTGGCATTGCGATTCCACATGGATGGGCGAC
AGAGTCATCACCACCAGCACCCGAACCTGGGCCCTGCCCACCTACAACAACCACCTCTACAAACAAATTTCCAGC
CAATCAGGAGCCTCGAACGACAATCACTACTTTGGCTACAGCACCCCTTGGGGGTATTTTGACTTCAACAGATTC
CACTGCCACTTTTCACCACGTGACTGGCAAAGACTCATCAACAACAACTGGGGATTCCGACCCAAGAGACTCAAC
TTCAAGCTCTTTAACATTCAAGTCAAAGAGGTCACGCAGAATGACGGTACGACGACGATTGCCAATAACCTTACC
AGCACGGTTCAGGTGTTTACTGACTCGGAGTACCAGCTCCCGTACGTCCTCGGCTCGGCGCATCAAGGATGCCTC
CCGCCGTTCCCAGCAGACGTCTTCATGGTGCCACAGTATGGATACCTCACCCTGAACAACGGGAGTCAGGCAGTA
GGACGCTCTTCATTTTACTGCCTGGAGTACTTTCCTTCTCAGATGCTGCGTACCGGAAACAACTTTACCTTCAGC
TACACTTTTGAGGACGTTCCTTTCCACAGCAGCTACGCTCACAGCCAGAGTCTGGACCGTCTCATGAATCCTCTC
ATCGACCAGTACCTGTATTACTTGAGCAGAACAAACACTCCAAGTGGAACCACCACGCAGTCAAGGCTTCAGTTT
TCTCAGGCCGGAGCGAGTGACATTCGGGACCAGTCTAGGAACTGGCTTCCTGGACCCTGTTACCGCCAGCAGCGA
GTATCAAAGACATCTGCGGATAACAACAACAGTGAATACTCGTGGACTGGAGCTACCAAGTACCACCTCAATGGC
AGAGACTCTCTGGTGAATCCGGGCCCGGCCATGGCAAGCCACAAGGACGATGAAGAAAAGTTTTTTCCTCAGAGC
GGGGTTCTCATCTTTGGGAAGCAAGGCTCAGAGAAAACAAATGTGGACATTGAAAAGGTCATGATTACAGACGAA
GAGGAAATCAGGACAACCAATCCCGTGGCTACGGAGCAGTATGGTTCTGTATCTACCAACCTCCAGAGAGGCAAC
AGACAAGCAGCTACCGCAGATGTCAACACACAAGGCGTTCTTCCAGGCATGGTCTGGCAGGACAGAGATGTGTAC
CTTCAGGGGCCCATCTGGGCAAAGATTCCACACACGGACGGACATTTTCACCCCTCTCCCCTCATGGGTGGATTC
GGACTTAAACACCCTCCTCCACAGATTCTCATCAAGAACACCCCGGTACCTGCGAATCCTTCGACCACCTTCAGT
GCGGCAAAGTTTGCTTCCTTCATCACACAGTACTCCACGGGACAGGTCAGCGTGGAGATCGAGTGGGAGCTGCAG
AAGGAAAACAGCAAACGCTGGAATCCCGAAATTCAGTACACTTCCAACTACAACAAGTCTGTTAATGTGGACTTT
ACTGTGGACACTAATGGCGTGTATTCAGAGCCTCGCCCCATTGGCACCAGATACCTGACTCGTAATCTGTAA
CapVP3: (SEQ ID NO: 7)
ATGGCTACAGGCAGTGGCGCACCAATGGCAGACAATAACGAGGGCGCCGACGGAGTGGGTAATTCCTCGGGAAAT
TGGCATTGCGATTCCACATGGATGGGCGACAGAGTCATCACCACCAGCACCCGAACCTGGGCCCTGCCCACCTAC
AACAACCACCTCTACAAACAAATTTCCAGCCAATCAGGAGCCTCGAACGACAATCACTACTTTGGCTACAGCACC
CCTTGGGGGTATTTTGACTTCAACAGATTCCACTGCCACTTTTCACCACGTGACTGGCAAAGACTCATCAACAAC
AACTGGGGATTCCGACCCAAGAGACTCAACTTCAAGCTCTTTAACATTCAAGTCAAAGAGGTCACGCAGAATGAC
GGTACGACGACGATTGCCAATAACCTTACCAGCACGGTTCAGGTGTTTACTGACTCGGAGTACCAGCTCCCGTAC
GTCCTCGGCTCGGCGCATCAAGGATGCCTCCCGCCGTTCCCAGCAGACGTCTTCATGGTGCCACAGTATGGATAC
CTCACCCTGAACAACGGGAGTCAGGCAGTAGGACGCTCTTCATTTTACTGCCTGGAGTACTTTCCTTCTCAGATG
CTGCGTACCGGAAACAACTTTACCTTCAGCTACACTTTTGAGGACGTTCCTTTCCACAGCAGCTACGCTCACAGC
CAGAGTCTGGACCGTCTCATGAATCCTCTCATCGACCAGTACCTGTATTACTTGAGCAGAACAAACACTCCAAGT
GGAACCACCACGCAGTCAAGGCTTCAGTTTTCTCAGGCCGGAGCGAGTGACATTCGGGACCAGTCTAGGAACTGG
CTTCCTGGACCCTGTTACCGCCAGCAGCGAGTATCAAAGACATCTGCGGATAACAACAACAGTGAATACTCGTGG
ACTGGAGCTACCAAGTACCACCTCAATGGCAGAGACTCTCTGGTGAATCCGGGCCCGGCCATGGCAAGCCACAAG
GACGATGAAGAAAAGTTTTTTCCTCAGAGCGGGGTTCTCATCTTTGGGAAGCAAGGCTCAGAGAAAACAAATGTG
GACATTGAAAAGGTCATGATTACAGACGAAGAGGAAATCAGGACAACCAATCCCGTGGCTACGGAGCAGTATGGT
TCTGTATCTACCAACCTCCAGAGAGGCAACAGACAAGCAGCTACCGCAGATGTCAACACACAAGGCGTTCTTCCA
GGCATGGTCTGGCAGGACAGAGATGTGTACCTTCAGGGGCCCATCTGGGCAAAGATTCCACACACGGACGGACAT
TTTCACCCCTCTCCCCTCATGGGTGGATTCGGACTTAAACACCCTCCTCCACAGATTCTCATCAAGAACACCCCG
GTACCTGCGAATCCTTCGACCACCTTCAGTGCGGCAAAGTTTGCTTCCTTCATCACACAGTACTCCACGGGACAG
GTCAGCGTGGAGATCGAGTGGGAGCTGCAGAAGGAAAACAGCAAACGCTGGAATCCCGAAATTCAGTACACTTCC
AACTACAACAAGTCTGTTAATGTGGACTTTACTGTGGACACTAATGGCGTGTATTCAGAGCCTCGCCCCATTGGC
ACCAGATACCTGACTCGTAATCTGTAA
CapAAP: (SEQ ID NO: 8)
CTGGAGACGCAGACTCAGTACCTGACCCCCAGCCTCTCGGACAGCCACCAGCAGCCCCCTCTGGTCTGGGAACTA
ATACGATGGCTACAGGCAGTGGCGCACCAATGGCAGACAATAACGAGGGCGCCGACGGAGTGGGTAATTCCTCGG
GAAATTGGCATTGCGATTCCACATGGATGGGCGACAGAGTCATCACCACCAGCACCCGAACCTGGGCCCTGCCCA
CCTACAACAACCACCTCTACAAACAAATTTCCAGCCAATCAGGAGCCTCGAACGACAATCACTACTTTGGCTACA
GCACCCCTTGGGGGTATTTTGACTTCAACAGATTCCACTGCCACTTTTCACCACGTGACTGGCAAAGACTCATCA
ACAACAACTGGGGATTCCGACCCAAGAGACTCAACTTCAAGCTCTTTAACATTCAAGTCAAAGAGGTCACGCAGA
ATGACGGTACGACGACGATTGCCAATAACCTTACCAGCACGGTTCAGGTGTTTACTGACTCGGAGTACCAGCTCC
CGTACGTCCTCGGCTCGGCGCATCAAGGATGCCTCCCGCCGTTCCCAGCAGACGTCTTCATGGTGCCACAGTATG
GATACCTCACCCTGA
AAV-3
Full Genome: NC_001729
Rep78: (SEQ ID NO: 9)
ATGCCGGGGTTCTACGAGATTGTCCTGAAGGTCCCGAGTGACCTGGACGAGCGCCTGCCGGGCATTTCTAACTCG
TTTGTTAACTGGGTGGCCGAGAAGGAATGGGACGTGCCGCCGGATTCTGACATGGATCCGAATCTGATTGAGCAG
GCACCCCTGACCGTGGCCGAAAAGCTTCAGCGCGAGTTCCTGGTGGAGTGGCGCCGCGTGAGTAAGGCCCCGGAG
GCCCTCTTTTTTGTCCAGTTCGAAAAGGGGGAGACCTACTTCCACCTGCACGTGCTGATTGAGACCATCGGGGTC
AAATCCATGGTGGTCGGCCGCTACGTGAGCCAGATTAAAGAGAAGCTGGTGACCCGCATCTACCGCGGGGTCGAG
CCGCAGCTTCCGAACTGGTTCGCGGTGACCAAAACGCGAAATGGCGCCGGGGGCGGGAACAAGGTGGTGGACGAC
TGCTACATCCCCAACTACCTGCTCCCCAAGACCCAGCCCGAGCTCCAGTGGGCGTGGACTAACATGGACCAGTAT
TTAAGCGCCTGTTTGAATCTCGCGGAGCGTAAACGGCTGGTGGCGCAGCATCTGACGCACGTGTCGCAGACGCAG
GAGCAGAACAAAGAGAATCAGAACCCCAATTCTGACGCGCCGGTCATCAGGTCAAAAACCTCAGCCAGGTACATG
GAGCTGGTCGGGTGGCTGGTGGACCGCGGGATCACGTCAGAAAAGCAATGGATTCAGGAGGACCAGGCCTCGTAC
ATCTCCTTCAACGCCGCCTCCAACTCGCGGTCCCAGATCAAGGCCGCGCTGGACAATGCCTCCAAGATCATGAGC
CTGACAAAGACGGCTCCGGACTACCTGGTGGGCAGCAACCCGCCGGAGGACATTACCAAAAATCGGATCTACCAA
ATCCTGGAGCTGAACGGGTACGATCCGCAGTACGCGGCCTCCGTCTTCCTGGGCTGGGCGCAAAAGAAGTTCGGG
AAGAGGAACACCATCTGGCTCTTTGGGCCGGCCACGACGGGTAAAACCAACATCGCGGAAGCCATCGCCCACGCC
GTGCCCTTCTACGGCTGCGTAAACTGGACCAATGAGAACTTTCCCTTCAACGATTGCGTCGACAAGATGGTGATC
TGGTGGGAGGAGGGCAAGATGACGGCCAAGGTCGTGGAGAGCGCCAAGGCCATTCTGGGCGGAAGCAAGGTGCGC
GTGGACCAAAAGTGCAAGTCATCGGCCCAGATCGAACCCACTCCCGTGATCGTCACCTCCAACACCAACATGTGC
GCCGTGATTGACGGGAACAGCACCACCTTCGAGCATCAGCAGCCGCTGCAGGACCGGATGTTTGAATTTGAACTT
ACCCGCCGTTTGGACCATGACTTTGGGAAGGTCACCAAACAGGAAGTAAAGGACTTTTTCCGGTGGGCTTCCGAT
CACGTGACTGACGTGGCTCATGAGTTCTACGTCAGAAAGGGTGGAGCTAAGAAACGCCCCGCCTCCAATGACGCG
GATGTAAGCGAGCCAAAACGGGAGTGCACGTCACTTGCGCAGCCGACAACGTCAGACGCGGAAGCACCGGCGGAC
TACGCGGACAGGTACCAAAACAAATGTTCTCGTCACGTGGGCATGAATCTGATGCTTTTTCCCTGTAAAACATGC
GAGAGAATGAATCAAATTTCCAATGTCTGTTTTACGCATGGTCAAAGAGACTGTGGGGAATGCTTCCCTGGAATG
TCAGAATCTCAACCCGTTTCTGTCGTCAAAAAGAAGACTTATCAGAAACTGTGTCCAATTCATCATATCCTGGGA
AGGGCACCCGAGATTGCCTGTTCGGCCTGCGATTTGGCCAATGTGGACTTGGATGACTGTGTTTCTGAGCAATAA
CapVP1: (SEQ ID NO: 10)
ATGGCTGCTGACGGTTATCTTCCAGATTGGCTCGAGGACAACCTTTCTGAAGGCATTCGTGAGTGGTGGGCTCTG
AAACCTGGAGTCCCTCAACCCAAAGCGAACCAACAACACCAGGACAACCGTCGGGGTCTTGTGCTTCCGGGTTAC
AAATACCTCGGACCCGGTAACGGACTCGACAAAGGAGAGCCGGTCAACGAGGCGGACGCGGCAGCCCTCGAACAC
GACAAAGCTTACGACCAGCAGCTCAAGGCCGGTGACAACCCGTACCTCAAGTACAACCACGCCGACGCCGAGTTT
CAGGAGCGTCTTCAAGAAGATACGTCTTTTGGGGGCAACCTTGGCAGAGCAGTCTTCCAGGCCAAAAAGAGGATC
CTTGAGCCTCTTGGTCTGGTTGAGGAAGCAGCTAAAACGGCTCCTGGAAAGAAGGGGGCTGTAGATCAGTCTCCT
CAGGAACCGGACTCATCATCTGGTGTTGGCAAATCGGGCAAACAGCCTGCCAGAAAAAGACTAAATTTCGGTCAG
ACTGGAGACTCAGAGTCAGTCCCAGACCCTCAACCTCTCGGAGAACCACCAGCAGCCCCCACAAGTTTGGGATCT
AATACAATGGCTTCAGGCGGTGGCGCACCAATGGCAGACAATAACGAGGGTGCCGATGGAGTGGGTAATTCCTCA
GGAAATTGGCATTGCGATTCCCAATGGCTGGGCGACAGAGTCATCACCACCAGCACCAGAACCTGGGCCCTGCCC
ACTTACAACAACCATCTCTACAAGCAAATCTCCAGCCAATCAGGAGCTTCAAACGACAACCACTACTTTGGCTAC
AGCACCCCTTGGGGGTATTTTGACTTTAACAGATTCCACTGCCACTTCTCACCACGTGACTGGCAGCGACTCATT
AACAACAACTGGGGATTCCGGCCCAAGAAACTCAGCTTCAAGCTCTTCAACATCCAAGTTAGAGGGGTCACGCAG
AACGATGGCACGACGACTATTGCCAATAACCTTACCAGCACGGTTCAAGTGTTTACGGACTCGGAGTATCAGCTC
CCGTACGTGCTCGGGTCGGCGCACCAAGGCTGTCTCCCGCCGTTTCCAGCGGACGTCTTCATGGTCCCTCAGTAT
GGATACCTCACCCTGAACAACGGAAGTCAAGCGGTGGGACGCTCATCCTTTTACTGCCTGGAGTACTTCCCTTCG
CAGATGCTAAGGACTGGAAATAACTTCCAATTCAGCTATACCTTCGAGGATGTACCTTTTCACAGCAGCTACGCT
CACAGCCAGAGTTTGGATCGCTTGATGAATCCTCTTATTGATCAGTATCTGTACTACCTGAACAGAACGCAAGGA
ACAACCTCTGGAACAACCAACCAATCACGGCTGCTTTTTAGCCAGGCTGGGCCTCAGTCTATGTCTTTGCAGGCC
AGAAATTGGCTACCTGGGCCCTGCTACCGGCAACAGAGACTTTCAAAGACTGCTAACGACAACAACAACAGTAAC
TTTCCTTGGACAGCGGCCAGCAAATATCATCTCAATGGCCGCGACTCGCTGGTGAATCCAGGACCAGCTATGGCC
AGTCACAAGGACGATGAAGAAAAATTTTTCCCTATGCACGGCAATCTAATATTTGGCAAAGAAGGGACAACGGCA
AGTAACGCAGAATTAGATAATGTAATGATTACGGATGAAGAAGAGATTCGTACCACCAATCCTGTGGCAACAGAG
CAGTATGGAACTGTGGCAAATAACTTGCAGAGCTCAAATACAGCTCCCACGACTGGAACTGTCAATCATCAGGGG
GCCTTACCTGGCATGGTGTGGCAAGATCGTGACGTGTACCTTCAAGGACCTATCTGGGCAAAGATTCCTCACACG
GATGGACACTTTCATCCTTCTCCTCTGATGGGAGGCTTTGGACTGAAACATCCGCCTCCTCAAATCATGATCAAA
AATACTCCGGTACCGGCAAATCCTCCGACGACTTTCAGCCCGGCCAAGTTTGCTTCATTTATCACTCAGTACTCC
ACTGGACAGGTCAGCGTGGAAATTGAGTGGGAGCTACAGAAAGAAAACAGCAAACGTTGGAATCCAGAGATTCAG
TACACTTCCAACTACAACAAGTCTGTTAATGTGGACTTTACTGTAGACACTAATGGTGTTTATAGTGAACCTCGC
CCTATTGGAACCCGGTATCTCACACGAAACTTGTGA
AAV-4
Full Genome: NC_001829
Rep78: (SEQ ID NO: 11)
ATGCCGGGGTTCTACGAGATCGTGCTGAAGGTGCCCAGCGACCTGGACGAGCACCTGCCCGGCATTTCTGACTCT
TTTGTGAGCTGGGTGGCCGAGAAGGAATGGGAGCTGCCGCCGGATTCTGACATGGACTTGAATCTGATTGAGCAG
GCACCCCTGACCGTGGCCGAAAAGCTGCAACGCGAGTTCCTGGTCGAGTGGCGCCGCGTGAGTAAGGCCCCGGAG
GCCCTCTTCTTTGTCCAGTTCGAGAAGGGGGACAGCTACTTCCACCTGCACATCCTGGTGGAGACCGTGGGCGTC
AAATCCATGGTGGTGGGCCGCTACGTGAGCCAGATTAAAGAGAAGCTGGTGACCCGCATCTACCGCGGGGTCGAG
CCGCAGCTTCCGAACTGGTTCGCGGTGACCAAGACGCGTAATGGCGCCGGAGGCGGGAACAAGGTGGTGGACGAC
TGCTACATCCCCAACTACCTGCTCCCCAAGACCCAGCCCGAGCTCCAGTGGGCGTGGACTAACATGGACCAGTAT
ATAAGCGCCTGTTTGAATCTCGCGGAGCGTAAACGGCTGGTGGCGCAGCATCTGACGCACGTGTCGCAGACGCAG
GAGCAGAACAAGGAAAACCAGAACCCCAATTCTGACGCGCCGGTCATCAGGTCAAAAACCTCCGCCAGGTACATG
GAGCTGGTCGGGTGGCTGGTGGACCGCGGGATCACGTCAGAAAAGCAATGGATCCAGGAGGACCAGGCGTCCTAC
ATCTCCTTCAACGCCGCCTCCAACTCGCGGTCACAAATCAAGGCCGCGCTGGACAATGCCTCCAAAATCATGAGC
CTGACAAAGACGGCTCCGGACTACCTGGTGGGCCAGAACCCGCCGGAGGACATTTCCAGCAACCGCATCTACCGA
ATCCTCGAGATGAACGGGTACGATCCGCAGTACGCGGCCTCCGTCTTCCTGGGCTGGGCGCAAAAGAAGTTCGGG
AAGAGGAACACCATCTGGCTCTTTGGGCCGGCCACGACGGGTAAAACCAACATCGCGGAAGCCATCGCCCACGCC
GTGCCCTTCTACGGCTGCGTGAACTGGACCAATGAGAACTTTCCGTTCAACGATTGCGTCGACAAGATGGTGATC
TGGTGGGAGGAGGGCAAGATGACGGCCAAGGTCGTAGAGAGCGCCAAGGCCATCCTGGGCGGAAGCAAGGTGCGC
GTGGACCAAAAGTGCAAGTCATCGGCCCAGATCGACCCAACTCCCGTGATCGTCACCTCCAACACCAACATGTGC
GCGGTCATCGACGGAAACTCGACCACCTTCGAGCACCAACAACCACTCCAGGACCGGATGTTCAAGTTCGAGCTC
ACCAAGCGCCTGGAGCACGACTTTGGCAAGGTCACCAAGCAGGAAGTCAAAGACTTTTTCCGGTGGGCGTCAGAT
CACGTGACCGAGGTGACTCACGAGTTTTACGTCAGAAAGGGTGGAGCTAGAAAGAGGCCCGCCCCCAATGACGCA
GATATAAGTGAGCCCAAGCGGGCCTGTCCGTCAGTTGCGCAGCCATCGACGTCAGACGCGGAAGCTCCGGTGGAC
TACGCGGACAGGTACCAAAACAAATGTTCTCGTCACGTGGGTATGAATCTGATGCTTTTTCCCTGCCGGCAATGC
GAGAGAATGAATCAGAATGTGGACATTTGCTTCACGCACGGGGTCATGGACTGTGCCGAGTGCTTCCCCGTGTCA
GAATCTCAACCCGTGTCTGTCGTCAGAAAGCGGACGTATCAGAAACTGTGTCCGATTCATCACATCATGGGGAGG
GCGCCCGAGGTGGCCTGCTCGGCCTGCGAACTGGCCAATGTGGACTTGGATGACTGTGACATGGAACAATAA
CapVP1: (SEQ ID NO: 12)
ATGACTGACGGTTACCTTCCAGATTGGCTAGAGGACAACCTCTCTGAAGGCGTTCGAGAGTGGTGGGCGCTGCAA
CCTGGAGCCCCTAAACCCAAGGCAAATCAACAACATCAGGACAACGCTCGGGGTCTTGTGCTTCCGGGTTACAAA
TACCTCGGACCCGGCAACGGACTCGACAAGGGGGAACCCGTCAACGCAGCGGACGCGGCAGCCCTCGAGCACGAC
AAGGCCTACGACCAGCAGCTCAAGGCCGGTGACAACCCCTACCTCAAGTACAACCACGCCGACGCGGAGTTCCAG
CAGCGGCTTCAGGGCGACACATCGTTTGGGGGCAACCTCGGCAGAGCAGTCTTCCAGGCCAAAAAGAGGGTTCTT
GAACCTCTTGGTCTGGTTGAGCAAGCGGGTGAGACGGCTCCTGGAAAGAAGAGACCGTTGATTGAATCCCCCCAG
CAGCCCGACTCCTCCACGGGTATCGGCAAAAAAGGCAAGCAGCCGGCTAAAAAGAAGCTCGTTTTCGAAGACGAA
ACTGGAGCAGGCGACGGACCCCCTGAGGGATCAACTTCCGGAGCCATGTCTGATGACAGTGAGATGCGTGCAGCA
GCTGGCGGAGCTGCAGTCGAGGGCGGACAAGGTGCCGATGGAGTGGGTAATGCCTCGGGTGATTGGCATTGCGAT
TCCACCTGGTCTGAGGGCCACGTCACGACCACCAGCACCAGAACCTGGGTCTTGCCCACCTACAACAACCACCTC
TACAAGCGACTCGGAGAGAGCCTGCAGTCCAACACCTACAACGGATTCTCCACCCCCTGGGGATACTTTGACTTC
AACCGCTTCCACTGCCACTTCTCACCACGTGACTGGCAGCGACTCATCAACAACAACTGGGGCATGCGACCCAAA
GCCATGCGGGTCAAAATCTTCAACATCCAGGTCAAGGAGGTCACGACGTCGAACGGCGAGACAACGGTGGCTAAT
AACCTTACCAGCACGGTTCAGATCTTTGCGGACTCGTCGTACGAACTGCCGTACGTGATGGATGCGGGTCAAGAG
GGCAGCCTGCCTCCTTTTCCCAACGACGTCTTTATGGTGCCCCAGTACGGCTACTGTGGACTGGTGACCGGCAAC
ACTTCGCAGCAACAGACTGACAGAAATGCCTTCTACTGCCTGGAGTACTTTCCTTCGCAGATGCTGCGGACTGGC
AACAACTTTGAAATTACGTACAGTTTTGAGAAGGTGCCTTTCCACTCGATGTACGCGCACAGCCAGAGCCTGGAC
CGGCTGATGAACCCTCTCATCGACCAGTACCTGTGGGGACTGCAATCGACCACCACCGGAACCACCCTGAATGCC
GGGACTGCCACCACCAACTTTACCAAGCTGCGGCCTACCAACTTTTCCAACTTTAAAAAGAACTGGCTGCCCGGG
CCTTCAATCAAGCAGCAGGGCTTCTCAAAGACTGCCAATCAAAACTACAAGATCCCTGCCACCGGGTCAGACAGT
CTCATCAAATACGAGACGCACAGCACTCTGGACGGAAGATGGAGTGCCCTGACCCCCGGACCTCCAATGGCCACG
GCTGGACCTGCGGACAGCAAGTTCAGCAACAGCCAGCTCATCTTTGCGGGGCCTAAACAGAACGGCAACACGGCC
ACCGTACCCGGGACTCTGATCTTCACCTCTGAGGAGGAGCTGGCAGCCACCAACGCCACCGATACGGACATGTGG
GGCAACCTACCTGGCGGTGACCAGAGCAACAGCAACCTGCCGACCGTGGACAGACTGACAGCCTTGGGAGCCGTG
CCTGGAATGGTCTGGCAAAACAGAGACATTTACTACCAGGGTCCCATTTGGGCCAAGATTCCTCATACCGATGGA
CACTTTCACCCCTCACCGCTGATTGGTGGGTTTGGGCTGAAACACCCGCCTCCTCAAATTTTTATCAAGAACACC
CCGGTACCTGCGAATCCTGCAACGACCTTCAGCTCTACTCCGGTAAACTCCTTCATTACTCAGTACAGCACTGGC
CAGGTGTCGGTGCAGATTGACTGGGAGATCCAGAAGGAGCGGTCCAAACGCTGGAACCCCGAGGTCCAGTTTACC
TCCAACTACGGACAGCAAAACTCTCTGTTGTGGGCTCCCGATGCGGCTGGGAAATACACTGAGCCTAGGGCTATC
GGTACCCGCTACCTCACCCACCACCTGTAA
AAV-5
Full Genome: NC_006152
Rep78: (SEQ ID NO: 13)
ATGGCTACCTTCTATGAAGTCATTGTTCGCGTCCCATTTGACGTGGAGGAACATCTGCCTGGAATTTCTGACAGC
TTTGTGGACTGGGTAACTGGTCAAATTTGGGAGCTGCCTCCAGAGTCAGATTTAAATTTGACTCTGGTTGAACAG
CCTCAGTTGACGGTGGCTGATAGAATTCGCCGCGTGTTCCTGTACGAGTGGAACAAATTTTCCAAGCAGGAGTCC
AAATTCTTTGTGCAGTTTGAAAAGGGATCTGAATATTTTCATCTGCACACGCTTGTGGAGACCTCCGGCATCTCT
TCCATGGTCCTCGGCCGCTACGTGAGTCAGATTCGCGCCCAGCTGGTGAAAGTGGTCTTCCAGGGAATTGAACCC
CAGATCAACGACTGGGTCGCCATCACCAAGGTAAAGAAGGGCGGAGCCAATAAGGTGGTGGATTCTGGGTATATT
CCCGCCTACCTGCTGCCGAAGGTCCAACCGGAGCTTCAGTGGGCGTGGACAAACCTGGACGAGTATAAATTGGCC
GCCCTGAATCTGGAGGAGCGCAAACGGCTCGTCGCGCAGTTTCTGGCAGAATCCTCGCAGCGCTCGCAGGAGGCG
GCTTCGCAGCGTGAGTTCTCGGCTGACCCGGTCATCAAAAGCAAGACTTCCCAGAAATACATGGCGCTCGTCAAC
TGGCTCGTGGAGCACGGCATCACTTCCGAGAAGCAGTGGATCCAGGAAAATCAGGAGAGCTACCTCTCCTTCAAC
TCCACCGGCAACTCTCGGAGCCAGATCAAGGCCGCGCTCGACAACGCGACCAAAATTATGAGTCTGACAAAAAGC
GCGGTGGACTACCTCGTGGGGAGCTCCGTTCCCGAGGACATTTCAAAAAACAGAATCTGGCAAATTTTTGAGATG
AATGGCTACGACCCGGCCTACGCGGGATCCATCCTCTACGGCTGGTGTCAGCGCTCCTTCAACAAGAGGAACACC
GTCTGGCTCTACGGACCCGCCACGACCGGCAAGACCAACATCGCGGAGGCCATCGCCCACACTGTGCCCTTTTAC
GGCTGCGTGAACTGGACCAATGAAAACTTTCCCTTTAATGACTGTGTGGACAAAATGCTCATTTGGTGGGAGGAG
GGAAAGATGACCAACAAGGTGGTTGAATCCGCCAAGGCCATCCTGGGGGGCTCAAAGGTGCGGGTCGATCAGAAA
TGTAAATCCTCTGTTCAAATTGATTCTACCCCTGTCATTGTAACTTCCAATACAAACATGTGTGTGGTGGTGGAT
GGGAATTCCACGACCTTTGAACACCAGCAGCCGCTGGAGGACCGCATGTTCAAATTTGAACTGACTAAGCGGCTC
CCGCCAGATTTTGGCAAGATTACTAAGCAGGAAGTCAAGGACTTTTTTGCTTGGGCAAAGGTCAATCAGGTGCCG
GTGACTCACGAGTTTAAAGTTCCCAGGGAATTGGCGGGAACTAAAGGGGCGGAGAAATCTCTAAAACGCCCACTG
GGTGACGTCACCAATACTAGCTATAAAAGTCTGGAGAAGCGGGCCAGGCTCTCATTTGTTCCCGAGACGCCTCGC
AGTTCAGACGTGACTGTTGATCCCGCTCCTCTGCGACCGCTCAATTGGAATTCAAGGTATGATTGCAAATGTGAC
TATCATGCTCAATTTGACAACATTTCTAACAAATGTGATGAATGTGAATATTTGAATCGGGGCAAAAATGGATGT
ATCTGTCACAATGTAACTCACTGTCAAATTTGTCATGGGATTCCCCCCTGGGAAAAGGAAAACTTGTCAGATTTT
GGGGATTTTGACGATGCCAATAAAGAACAGTAA
CapVP1: (SEQ ID NO: 14)
ATGTCTTTTGTTGATCACCCTCCAGATTGGTTGGAAGAAGTTGGTGAAGGTCTTCGCGAGTTTTTGGGCCTTGAA
GCGGGCCCACCGAAACCAAAACCCAATCAGCAGCATCAAGATCAAGCCCGTGGTCTTGTGCTGCCTGGTTATAAC
TATCTCGGACCCGGAAACGGTCTCGATCGAGGAGAGCCTGTCAACAGGGCAGACGAGGTCGCGCGAGAGCACGAC
ATCTCGTACAACGAGCAGCTTGAGGCGGGAGACAACCCCTACCTCAAGTACAACCACGCGGACGCCGAGTTTCAG
GAGAAGCTCGCCGACGACACATCCTTCGGGGGAAACCTCGGAAAGGCAGTCTTTCAGGCCAAGAAAAGGGTTCTC
GAACCTTTTGGCCTGGTTGAAGAGGGTGCTAAGACGGCCCCTACCGGAAAGCGGATAGACGACCACTTTCCAAAA
AGAAAGAAGGCTCGGACCGAAGAGGACTCCAAGCCTTCCACCTCGTCAGACGCCGAAGCTGGACCCAGCGGATCC
CAGCAGCTGCAAATCCCAGCCCAACCAGCCTCAAGTTTGGGAGCTGATACAATGTCTGCGGGAGGTGGCGGCCCA
TTGGGCGACAATAACCAAGGTGCCGATGGAGTGGGCAATGCCTCGGGAGATTGGCATTGCGATTCCACGTGGATG
GGGGACAGAGTCGTCACCAAGTCCACCCGAACCTGGGTGCTGCCCAGCTACAACAACCACCAGTACCGAGAGATC
AAAAGCGGCTCCGTCGACGGAAGCAACGCCAACGCCTACTTTGGATACAGCACCCCCTGGGGGTACTTTGACTTT
AACCGCTTCCACAGCCACTGGAGCCCCCGAGACTGGCAAAGACTCATCAACAACTACTGGGGCTTCAGACCCCGG
TCCCTCAGAGTCAAAATCTTCAACATTCAAGTCAAAGAGGTCACGGTGCAGGACTCCACCACCACCATCGCCAAC
AACCTCACCTCCACCGTCCAAGTGTTTACGGACGACGACTACCAGCTGCCCTACGTCGTCGGCAACGGGACCGAG
GGATGCCTGCCGGCCTTCCCTCCGCAGGTCTTTACGCTGCCGCAGTACGGTTACGCGACGCTGAACCGCGACAAC
ACAGAAAATCCCACCGAGAGGAGCAGCTTCTTCTGCCTAGAGTACTTTCCCAGCAAGATGCTGAGAACGGGCAAC
AACTTTGAGTTTACCTACAACTTTGAGGAGGTGCCCTTCCACTCCAGCTTCGCTCCCAGTCAGAACCTGTTCAAG
CTGGCCAACCCGCTGGTGGACCAGTACTTGTACCGCTTCGTGAGCACAAATAACACTGGCGGAGTCCAGTTCAAC
AAGAACCTGGCCGGGAGATACGCCAACACCTACAAAAACTGGTTCCCGGGGCCCATGGGCCGAACCCAGGGCTGG
AACCTGGGCTCCGGGGTCAACCGCGCCAGTGTCAGCGCCTTCGCCACGACCAATAGGATGGAGCTCGAGGGCGCG
AGTTACCAGGTGCCCCCGCAGCCGAACGGCATGACCAACAACCTCCAGGGCAGCAACACCTATGCCCTGGAGAAC
ACTATGATCTTCAACAGCCAGCCGGCGAACCCGGGCACCACCGCCACGTACCTCGAGGGCAACATGCTCATCACC
AGCGAGAGCGAGACGCAGCCGGTGAACCGCGTGGCGTACAACGTCGGCGGGCAGATGGCCACCAACAACCAGAGC
TCCACCACTGCCCCCGCGACCGGCACGTACAACCTCCAGGAAATCGTGCCCGGCAGCGTGTGGATGGAGAGGGAC
GTGTACCTCCAAGGACCCATCTGGGCCAAGATCCCAGAGACGGGGGCGCACTTTCACCCCTCTCCGGCCATGGGC
GGATTCGGACTCAAACACCCACCGCCCATGATGCTCATCAAGAACACGCCTGTGCCCGGAAATATCACCAGCTTC
TCGGACGTGCCCGTCAGCAGCTTCATCACCCAGTACAGCACCGGGCAGGTCACCGTGGAGATGGAGTGGGAGCTC
AAGAAGGAAAACTCCAAGAGGTGGAACCCAGAGATCCAGTACACAAACAACTACAACGACCCCCAGTTTGTGGAC
TTTGCCCCGGACAGCACCGGGGAATACAGAACCACCAGACCTATCGGAACCCGATACCTTACCCGACCCCTTTAA
AAV-6
Full Genome: AF028704
Rep78: (SEQ ID NO: 15)
ATGCCGGGGTTTTACGAGATTGTGATTAAGGTCCCCAGCGACCTTGACGAGCATCTGCCCGGCATTTCTGACAGC
TTTGTGAACTGGGTGGCCGAGAAGGAATGGGAGTTGCCGCCAGATTCTGACATGGATCTGAATCTGATTGAGCAG
GCACCCCTGACCGTGGCCGAGAAGCTGCAGCGCGACTTCCTGGTCCAGTGGCGCCGCGTGAGTAAGGCCCCGGAG
GCCCTCTTCTTTGTTCAGTTCGAGAAGGGCGAGTCCTACTTCCACCTCCATATTCTGGTGGAGACCACGGGGGTC
AAATCCATGGTGCTGGGCCGCTTCCTGAGTCAGATTAGGGACAAGCTGGTGCAGACCATCTACCGCGGGATCGAG
CCGACCCTGCCCAACTGGTTCGCGGTGACCAAGACGCGTAATGGCGCCGGAGGGGGGAACAAGGTGGTGGACGAG
TGCTACATCCCCAACTACCTCCTGCCCAAGACTCAGCCCGAGCTGCAGTGGGCGTGGACTAACATGGAGGAGTAT
ATAAGCGCGTGTTTAAACCTGGCCGAGCGCAAACGGCTCGTGGCGCACGACCTGACCCACGTCAGCCAGACCCAG
GAGCAGAACAAGGAGAATCTGAACCCCAATTCTGACGCGCCTGTCATCCGGTCAAAAACCTCCGCACGCTACATG
GAGCTGGTCGGGTGGCTGGTGGACCGGGGCATCACCTCCGAGAAGCAGTGGATCCAGGAGGACCAGGCCTCGTAC
ATCTCCTTCAACGCCGCCTCCAACTCGCGGTCCCAGATCAAGGCCGCTCTGGACAATGCCGGCAAGATCATGGCG
CTGACCAAATCCGCGCCCGACTACCTGGTAGGCCCCGCTCCGCCCGCCGACATTAAAACCAACCGCATTTACCGC
ATCCTGGAGCTGAACGGCTACGACCCTGCCTACGCCGGCTCCGTCTTTCTCGGCTGGGCCCAGAAAAGGTTCGGA
AAACGCAACACCATCTGGCTGTTTGGGCCGGCCACCACGGGCAAGACCAACATCGCGGAAGCCATCGCCCACGCC
GTGCCCTTCTACGGCTGCGTCAACTGGACCAATGAGAACTTTCCCTTCAACGATTGCGTCGACAAGATGGTGATC
TGGTGGGAGGAGGGCAAGATGACGGCCAAGGTCGTGGAGTCCGCCAAGGCCATTCTCGGCGGCAGCAAGGTGCGC
GTGGACCAAAAGTGCAAGTCGTCCGCCCAGATCGATCCCACCCCCGTGATCGTCACCTCCAACACCAACATGTGC
GCCGTGATTGACGGGAACAGCACCACCTTCGAGCACCAGCAGCCGTTGCAGGACCGGATGTTCAAATTTGAACTC
ACCCGCCGTCTGGAGCATGACTTTGGCAAGGTGACAAAGCAGGAAGTCAAAGAGTTCTTCCGCTGGGCGCAGGAT
CACGTGACCGAGGTGGCGCATGAGTTCTACGTCAGAAAGGGTGGAGCCAACAAGAGACCCGCCCCCGATGACGCG
GATAAAAGCGAGCCCAAGCGGGCCTGCCCCTCAGTCGCGGATCCATCGACGTCAGACGCGGAAGGAGCTCCGGTG
GACTTTGCCGACAGGTACCAAAACAAATGTTCTCGTCACGCGGGCATGCTTCAGATGCTGTTTCCCTGCAAAACA
TGCGAGAGAATGAATCAGAATTTCAACATTTGCTTCACGCACGGGACCAGAGACTGTTCAGAATGTTTCCCCGGC
GTGTCAGAATCTCAACCGGTCGTCAGAAAGAGGACGTATCGGAAACTCTGTGCCATTCATCATCTGCTGGGGCGG
GCTCCCGAGATTGCTTGCTCGGCCTGCGATCTGGTCAACGTGGATCTGGATGACTGTGTTTCTGAGCAATAA
CapVP1: (SEQ ID NO: 16)
ATGGCTGCCGATGGTTATCTTCCAGATTGGCTCGAGGACAACCTCTCTGAGGGCATTCGCGAGTGGTGGGACTTG
AAACCTGGAGCCCCGAAACCCAAAGCCAACCAGCAAAAGCAGGACGACGGCCGGGGTCTGGTGCTTCCTGGCTAC
AAGTACCTCGGACCCTTCAACGGACTCGACAAGGGGGAGCCCGTCAACGCGGCGGATGCAGCGGCCCTCGAGCAC
GACAAGGCCTACGACCAGCAGCTCAAAGCGGGTGACAATCCGTACCTGCGGTATAACCACGCCGACGCCGAGTTT
CAGGAGCGTCTGCAAGAAGATACGTCTTTTGGGGGCAACCTCGGGCGAGCAGTCTTCCAGGCCAAGAAGAGGGTT
CTCGAACCTTTTGGTCTGGTTGAGGAAGGTGCTAAGACGGCTCCTGGAAAGAAACGTCCGGTAGAGCAGTCGCCA
CAAGAGCCAGACTCCTCCTCGGGCATTGGCAAGACAGGCCAGCAGCCCGCTAAAAAGAGACTCAATTTTGGTCAG
ACTGGCGACTCAGAGTCAGTCCCCGACCCACAACCTCTCGGAGAACCTCCAGCAACCCCCGCTGCTGTGGGACCT
ACTACAATGGCTTCAGGCGGTGGCGCACCAATGGCAGACAATAACGAAGGCGCCGACGGAGTGGGTAATGCCTCA
GGAAATTGGCATTGCGATTCCACATGGCTGGGCGACAGAGTCATCACCACCAGCACCCGAACATGGGCCTTGCCC
ACCTATAACAACCACCTCTACAAGCAAATCTCCAGTGCTTCAACGGGGGCCAGCAACGACAACCACTACTTCGGC
TACAGCACCCCCTGGGGGTATTTTGATTTCAACAGATTCCACTGCCATTTCTCACCACGTGACTGGCAGCGACTC
ATCAACAACAATTGGGGATTCCGGCCCAAGAGACTCAACTTCAAGCTCTTCAACATCCAAGTCAAGGAGGTCACG
ACGAATGATGGCGTCACGACCATCGCTAATAACCTTACCAGCACGGTTCAAGTCTTCTCGGACTCGGAGTACCAG
TTGCCGTACGTCCTCGGCTCTGCGCACCAGGGCTGCCTCCCTCCGTTCCCGGCGGACGTGTTCATGATTCCGCAG
TACGGCTACCTAACGCTCAACAATGGCAGCCAGGCAGTGGGACGGTCATCCTTTTACTGCCTGGAATATTTCCCA
TCGCAGATGCTGAGAACGGGCAATAACTTTACCTTCAGCTACACCTTCGAGGACGTGCCTTTCCACAGCAGCTAC
GCGCACAGCCAGAGCCTGGACCGGCTGATGAATCCTCTCATCGACCAGTACCTGTATTACCTGAACAGAACTCAG
AATCAGTCCGGAAGTGCCCAAAACAAGGACTTGCTGTTTAGCCGGGGGTCTCCAGCTGGCATGTCTGTTCAGCCC
AAAAACTGGCTACCTGGACCCTGTTACCGGCAGCAGCGCGTTTCTAAAACAAAAACAGACAACAACAACAGCAAC
TTTACCTGGACTGGTGCTTCAAAATATAACCTTAATGGGCGTGAATCTATAATCAACCCTGGCACTGCTATGGCC
TCACACAAAGACGACAAAGACAAGTTCTTTCCCATGAGCGGTGTCATGATTTTTGGAAAGGAGAGCGCCGGAGCT
TCAAACACTGCATTGGACAATGTCATGATCACAGACGAAGAGGAAATCAAAGCCACTAACCCCGTGGCCACCGAA
AGATTTGGGACTGTGGCAGTCAATCTCCAGAGCAGCAGCACAGACCCTGCGACCGGAGATGTGCATGTTATGGGA
GCCTTACCTGGAATGGTGTGGCAAGACAGAGACGTATACCTGCAGGGTCCTATTTGGGCCAAAATTCCTCACACG
GATGGACACTTTCACCCGTCTCCTCTCATGGGCGGCTTTGGACTTAAGCACCCGCCTCCTCAGATCCTCATCAAA
AACACGCCTGTTCCTGCGAATCCTCCGGCAGAGTTTTCGGCTACAAAGTTTGCTTCATTCATCACCCAGTATTCC
ACAGGACAAGTGAGCGTGGAGATTGAATGGGAGCTGCAGAAAGAAAACAGCAAACGCTGGAATCCCGAAGTGCAG
TATACATCTAACTATGCAAAATCTGCCAACGTTGATTTCACTGTGGACAACAATGGACTTTATACTGAGCCTCGC
CCCATTGGCACCCGTTACCTCACCCGTCCCCTGTAA
AAV-7
Full Genome: NC_006260
Rep78: (SEQ ID NO: 17)
ATGCCGGGTTTCTACGAGATCGTGATCAAGGTGCCGAGCGACCTGGACGAGCACCTGCCGGGCATTTCTGACTCG
TTTGTGAACTGGGTGGCCGAGAAGGAATGGGAGCTGCCCCCGGATTCTGACATGGATCTGAATCTGATCGAGCAG
GCACCCCTGACCGTGGCCGAGAAGCTGCAGCGCGACTTCCTGGTCCAATGGCGCCGCGTGAGTAAGGCCCCGGAG
GCCCTGTTCTTTGTTCAGTTCGAGAAGGGCGAGAGCTACTTCCACCTTCACGTTCTGGTGGAGACCACGGGGGTC
AAGTCCATGGTGCTAGGCCGCTTCCTGAGTCAGATTCGGGAGAAGCTGGTCCAGACCATCTACCGCGGGGTCGAG
CCCACGCTGCCCAACTGGTTCGCGGTGACCAAGACGCGTAATGGCGCCGGCGGGGGGAACAAGGTGGTGGACGAG
TGCTACATCCCCAACTACCTCCTGCCCAAGACCCAGCCCGAGCTGCAGTGGGCGTGGACTAACATGGAGGAGTAT
ATAAGCGCGTGTTTGAACCTGGCCGAACGCAAACGGCTCGTGGCGCAGCACCTGACCCACGTCAGCCAGACGCAG
GAGCAGAACAAGGAGAATCTGAACCCCAATTCTGACGCGCCCGTGATCAGGTCAAAAACCTCCGCGCGCTACATG
GAGCTGGTCGGGTGGCTGGTGGACCGGGGCATCACCTCCGAGAAGCAGTGGATCCAGGAGGACCAGGCCTCGTAC
ATCTCCTTCAACGCCGCCTCCAACTCGCGGTCCCAGATCAAGGCCGCGCTGGACAATGCCGGCAAGATCATGGCG
CTGACCAAATCCGCGCCCGACTACCTGGTGGGGCCCTCGCTGCCCGCGGACATTAAAACCAACCGCATCTACCGC
ATCCTGGAGCTGAACGGGTACGATCCTGCCTACGCCGGCTCCGTCTTTCTCGGCTGGGCCCAGAAAAAGTTCGGG
AAGCGCAACACCATCTGGCTGTTTGGGCCCGCCACCACCGGCAAGACCAACATTGCGGAAGCCATCGCCCACGCC
GTGCCCTTCTACGGCTGCGTCAACTGGACCAATGAGAACTTTCCCTTCAACGATTGCGTCGACAAGATGGTGATC
TGGTGGGAGGAGGGCAAGATGACGGCCAAGGTCGTGGAGTCCGCCAAGGCCATTCTCGGCGGCAGCAAGGTGCGC
GTGGACCAAAAGTGCAAGTCGTCCGCCCAGATCGACCCCACCCCCGTGATCGTCACCTCCAACACCAACATGTGC
GCCGTGATTGACGGGAACAGCACCACCTTCGAGCACCAGCAGCCGTTGCAGGACCGGATGTTCAAATTTGAACTC
ACCCGCCGTCTGGAGCACGACTTTGGCAAGGTGACGAAGCAGGAAGTCAAAGAGTTCTTCCGCTGGGCCAGTGAT
CACGTGACCGAGGTGGCGCATGAGTTCTACGTCAGAAAGGGCGGAGCCAGCAAAAGACCCGCCCCCGATGACGCG
GATATAAGCGAGCCCAAGCGGGCCTGCCCCTCAGTCGCGGATCCATCGACGTCAGACGCGGAAGGAGCTCCGGTG
GACTTTGCCGACAGGTACCAAAACAAATGTTCTCGTCACGCGGGCATGATTCAGATGCTGTTTCCCTGCAAAACG
TGCGAGAGAATGAATCAGAATTTCAACATTTGCTTCACACACGGGGTCAGAGACTGTTTAGAGTGTTTCCCCGGC
GTGTCAGAATCTCAACCGGTCGTCAGAAAAAAGACGTATCGGAAACTCTGCGCGATTCATCATCTGCTGGGGCGG
GCGCCCGAGATTGCTTGCTCGGCCTGCGACCTGGTCAACGTGGACCTGGACGACTGCGTTTCTGAGCAATAA
CapVP1: (SEQ ID NO: 18)
ATGGCTGCCGATGGTTATCTTCCAGATTGGCTCGAGGACAACCTCTCTGAGGGCATTCGCGAGTGGTGGGACCTG
AAACCTGGAGCCCCGAAACCCAAAGCCAACCAGCAAAAGCAGGACAACGGCCGGGGTCTGGTGCTTCCTGGCTAC
AAGTACCTCGGACCCTTCAACGGACTCGACAAGGGGGAGCCCGTCAACGCGGCGGACGCAGCGGCCCTCGAGCAC
GACAAGGCCTACGACCAGCAGCTCAAAGCGGGTGACAATCCGTACCTGCGGTATAACCACGCCGACGCCGAGTTT
CAGGAGCGTCTGCAAGAAGATACGTCATTTGGGGGCAACCTCGGGCGAGCAGTCTTCCAGGCCAAGAAGCGGGTT
CTCGAACCTCTCGGTCTGGTTGAGGAAGGCGCTAAGACGGCTCCTGCAAAGAAGAGACCGGTAGAGCCGTCACCT
CAGCGTTCCCCCGACTCCTCCACGGGCATCGGCAAGAAAGGCCAGCAGCCCGCCAGAAAGAGACTCAATTTCGGT
CAGACTGGCGACTCAGAGTCAGTCCCCGACCCTCAACCTCTCGGAGAACCTCCAGCAGCGCCCTCTAGTGTGGGA
TCTGGTACAGTGGCTGCAGGCGGTGGCGCACCAATGGCAGACAATAACGAAGGTGCCGACGGAGTGGGTAATGCC
TCAGGAAATTGGCATTGCGATTCCACATGGCTGGGCGACAGAGTCATTACCACCAGCACCCGAACCTGGGCCCTG
CCCACCTACAACAACCACCTCTACAAGCAAATCTCCAGTGAAACTGCAGGTAGTACCAACGACAACACCTACTTC
GGCTACAGCACCCCCTGGGGGTATTTTGACTTTAACAGATTCCACTGCCACTTCTCACCACGTGACTGGCAGCGA
CTCATCAACAACAACTGGGGATTCCGGCCCAAGAAGCTGCGGTTCAAGCTCTTCAACATCCAGGTCAAGGAGGTC
ACGACGAATGACGGCGTTACGACCATCGCTAATAACCTTACCAGCACGATTCAGGTATTCTCGGACTCGGAATAC
CAGCTGCCGTACGTCCTCGGCTCTGCGCACCAGGGCTGCCTGCCTCCGTTCCCGGCGGACGTCTTCATGATTCCT
CAGTACGGCTACCTGACTCTCAACAATGGCAGTCAGTCTGTGGGACGTTCCTCCTTCTACTGCCTGGAGTACTTC
CCCTCTCAGATGCTGAGAACGGGCAACAACTTTGAGTTCAGCTACAGCTTCGAGGACGTGCCTTTCCACAGCAGC
TACGCACACAGCCAGAGCCTGGACCGGCTGATGAATCCCCTCATCGACCAGTACTTGTACTACCTGGCCAGAACA
CAGAGTAACCCAGGAGGCACAGCTGGCAATCGGGAACTGCAGTTTTACCAGGGCGGGCCTTCAACTATGGCCGAA
CAAGCCAAGAATTGGTTACCTGGACCTTGCTTCCGGCAACAAAGAGTCTCCAAAACGCTGGATCAAAACAACAAC
AGCAACTTTGCTTGGACTGGTGCCACCAAATATCACCTGAACGGCAGAAACTCGTTGGTTAATCCCGGCGTCGCC
ATGGCAACTCACAAGGACGACGAGGACCGCTTTTTCCCATCCAGCGGAGTCCTGATTTTTGGAAAAACTGGAGCA
ACTAACAAAACTACATTGGAAAATGTGTTAATGACAAATGAAGAAGAAATTCGTCCTACTAATCCTGTAGCCACG
GAAGAATACGGGATAGTCAGCAGCAACTTACAAGCGGCTAATACTGCAGCCCAGACACAAGTTGTCAACAACCAG
GGAGCCTTACCTGGCATGGTCTGGCAGAACCGGGACGTGTACCTGCAGGGTCCCATCTGGGCCAAGATTCCTCAC
ACGGATGGCAACTTTCACCCGTCTCCTTTGATGGGCGGCTTTGGACTTAAACATCCGCCTCCTCAGATCCTGATC
AAGAACACTCCCGTTCCCGCTAATCCTCCGGAGGTGTTTACTCCTGCCAAGTTTGCTTCGTTCATCACACAGTAC
AGCACCGGACAAGTCAGCGTGGAAATCGAGTGGGAGCTGCAGAAGGAAAACAGCAAGCGCTGGAACCCGGAGATT
CAGTACACCTCCAACTTTGAAAAGCAGACTGGTGTGGACTTTGCCGTTGACAGCCAGGGTGTTTACTCTGAGCCT
CGCCCTATTGGCACTCGTTACCTCACCCGTAATCTGTAA
AAV-8
Full Genome: NC_006261
Rep78: (SEQ ID NO: 19)
ATGCCGGGCTTCTACGAGATCGTGATCAAGGTGCCGAGCGACCTGGACGAGCACCTGCCGGGCATTTCTGACTCG
TTTGTGAACTGGGTGGCCGAGAAGGAATGGGAGCTGCCCCCGGATTCTGACATGGATCGGAATCTGATCGAGCAG
GCACCCCTGACCGTGGCCGAGAAGCTGCAGCGCGACTTCCTGGTCCAATGGCGCCGCGTGAGTAAGGCCCCGGAG
GCCCTCTTCTTTGTTCAGTTCGAGAAGGGCGAGAGCTACTTTCACCTGCACGTTCTGGTCGAGACCACGGGGGTC
AAGTCCATGGTGCTAGGCCGCTTCCTGAGTCAGATTCGGGAAAAGCTTGGTCCAGACCATCTACCCGCGGGGTCG
AGCCCCACCTTGCCCAACTGGTTCGCGGTGACCAAAGACGCGGTAATGGCGCCGGCGGGGGGGAACAAGGTGGTG
GACGAGTGCTACATCCCCAACTACCTCCTGCCCAAGACTCAGCCCGAGCTGCAGTGGGCGTGGACTAACATGGAG
GAGTATATAAGCGCGTGCTTGAACCTGGCCGAGCGCAAACGGCTCGTGGCGCAGCACCTGACCCACGTCAGCCAG
ACGCAGGAGCAGAACAAGGAGAATCTGAACCCCAATTCTGACGCGCCCGTGATCAGGTCAAAAACCTCCGCGCGC
TATATGGAGCTGGTCGGGTGGCTGGTGGACCGGGGCATCACCTCCGAGAAGCAGTGGATCCAGGAGGACCAGGCC
TCGTACATCTCCTTCAACGCCGCCTCCAACTCGCGGTCCCAGATCAAGGCCGCGCTGGACAATGCCGGCAAGATC
ATGGCGCTGACCAAATCCGCGCCCGACTACCTGGTGGGGCCCTCGCTGCCCGCGGACATTACCCAGAACCGCATC
TACCGCATCCTCGCTCTCAACGGCTACGACCCTGCCTACGCCGGCTCCGTCTTTCTCGGCTGGGCTCAGAAAAAG
TTCGGGAAACGCAACACCATCTGGCTGTTTGGACCCGCCACCACCGGCAAGACCAACATTGCGGAAGCCATCGCC
CACGCCGTGCCCTTCTACGGCTGCGTCAACTGGACCAATGAGAACTTTCCCTTCAATGATTGCGTCGACAAGATG
GTGATCTGGTGGGAGGAGGGCAAGATGACGGCCAAGGTCGTGGAGTCCGCCAAGGCCATTCTCGGCGGCAGCAAG
GTGCGCGTGGACCAAAAGTGCAAGTCGTCCGCCCAGATCGACCCCACCCCCGTGATCGTCACCTCCAACACCAAC
ATGTGCGCCGTGATTGACGGGAACAGCACCACCTTCGAGCACCAGCAGCCTCTCCAGGACCGGATGTTTAAGTTC
GAACTCACCCGCCGTCTGGAGCACGACTTTGGCAAGGTGACAAAGCAGGAAGTCAAAGAGTTCTTCCGCTGGGCC
AGTGATCACGTGACCGAGGTGGCGCATGAGTTTTACGTCAGAAAGGGCGGAGCCAGCAAAAGACCCGCCCCCGAT
GACGCGGATAAAAGCGAGCCCAAGCGGGCCTGCCCCTCAGTCGCGGATCCATCGACGTCAGACGCGGAAGGAGCT
CCGGTGGACTTTGCCGACAGGTACCAAAACAAATGTTCTCGTCACGCGGGCATGCTTCAGATGCTGTTTCCCTGC
AAAACGTGCGAGAGAATGAATCAGAATTTCAACATTTGCTTCACACACGGGGTCAGAGACTGCTCAGAGTGTTTC
CCCGGCGTGTCAGAATCTCAACCGGTCGTCAGAAAGAGGACGTATCGGAAACTCTGTGCGATTCATCATCTGCTG
GGGCGGGCTCCCGAGATTGCTTGCTCGGCCTGCGATCTGGTCAACGTGGACCTGGATGACTGTGTTTCTGAGCAA
TAA
CapVP1: (SEQ ID NO: 20)
ATGGCTGCCGATGGTTATCTTCCAGATTGGCTCGAGGACAACCTCTCTGAGGGCATTCGCGAGTGGTGGGCGCTG
AAACCTGGAGCCCCGAAGCCCAAAGCCAACCAGCAAAAGCAGGACGACGGCCGGGGTCTGGTGCTTCCTGGCTAC
AAGTACCTCGGACCCTTCAACGGACTCGACAAGGGGGAGCCCGTCAACGCGGCGGACGCAGCGGCCCTCGAGCAC
GACAAGGCCTACGACCAGCAGCTGCAGGCGGGTGACAATCCGTACCTGCGGTATAACCACGCCGACGCCGAGTTT
CAGGAGCGTCTGCAAGAAGATACGTCTTTTGGGGGCAACCTCGGGCGAGCAGTCTTCCAGGCCAAGAAGCGGGTT
CTCGAACCTCTCGGTCTGGTTGAGGAAGGCGCTAAGACGGCTCCTGGAAAGAAGAGACCGGTAGAGCCATCACCC
CAGCGTTCTCCAGACTCCTCTACGGGCATCGGCAAGAAAGGCCAACAGCCCGCCAGAAAAAGACTCAATTTTGGT
CAGACTGGCGACTCAGAGTCAGTTCCAGACCCTCAACCTCTCGGAGAACCTCCAGCAGCGCCCTCTGGTGTGGGA
CCTAATACAATGGCTGCAGGCGGTGGCGCACCAATGGCAGACAATAACGAAGGCGCCGACGGAGTGGGTAGTTCC
TCGGGAAATTGGCATTGCGATTCCACATGGCTGGGCGACAGAGTCATCACCACCAGCACCCGAACCTGGGCCCTG
CCCACCTACAACAACCACCTCTACAAGCAAATCTCCAACGGGACATCGGGAGGAGCCACCAACGACAACACCTAC
TTCGGCTACAGCACCCCCTGGGGGTATTTTGACTTTAACAGATTCCACTGCCACTTTTCACCACGTGACTGGCAG
CGACTCATCAACAACAACTGGGGATTCCGGCCCAAGAGACTCAGCTTCAAGCTCTTCAACATCCAGGTCAAGGAG
GTCACGCAGAATGAAGGCACCAAGACCATCGCCAATAACCTCACCAGCACCATCCAGGTGTTTACGGACTCGGAG
TACCAGCTGCCGTACGTTCTCGGCTCTGCCCACCAGGGCTGCCTGCCTCCGTTCCCGGCGGACGTGTTCATGATT
CCCCAGTACGGCTACCTAACACTCAACAACGGTAGTCAGGCCGTGGGACGCTCCTCCTTCTACTGCCTGGAATAC
TTTCCTTCGCAGATGCTGAGAACCGGCAACAACTTCCAGTTTACTTACACCTTCGAGGACGTGCCTTTCCACAGC
AGCTACGCCCACAGCCAGAGCTTGGACCGGCTGATGAATCCTCTGATTGACCAGTACCTGTACTACTTGTCTCGG
ACTCAAACAACAGGAGGCACGGCAAATACGCAGACTCTGGGCTTCAGCCAAGGTGGGCCTAATACAATGGCCAAT
CAGGCAAAGAACTGGCTGCCAGGACCCTGTTACCGCCAACAACGCGTCTCAACGACAACCGGGCAAAACAACAAT
AGCAACTTTGCCTGGACTGCTGGGACCAAATACCATCTGAATGGAAGAAATTCATTGGCTAATCCTGGCATCGCT
ATGGCAACACACAAAGACGACGAGGAGCGTTTTTTTCCCAGTAACGGGATCCTGATTTTTGGCAAACAAAATGCT
GCCAGAGACAATGCGGATTACAGCGATGTCATGCTCACCAGCGAGGAAGAAATCAAAACCACTAACCCTGTGGCT
ACAGAGGAATACGGTATCGTGGCAGATAACTTGCAGCAGCAAAACACGGCTCCTCAAATTGGAACTGTCAACAGC
CAGGGGGCCTTACCCGGTATGGTCTGGCAGAACCGGGACGTGTACCTGCAGGGTCCCATCTGGGCCAAGATTCCT
CACACGGACGGCAACTTCCACCCGTCTCCGCTGATGGGCGGCTTTGGCCTGAAACATCCTCCGCCTCAGATCCTG
ATCAAGAACACGCCTGTACCTGCGGATCCTCCGACCACCTTCAACCAGTCAAAGCTGAACTCTTTCATCACGCAA
TACAGCACCGGACAGGTCAGCGTGGAAATTGAATGGGAGCTGCAGAAGGAAAACAGCAAGCGCTGGAACCCCGAG
ATCCAGTACACCTCCAACTACTACAAATCTACAAGTGTGGACTTTGCTGTTAATACAGAAGGCGTGTACTCTGAA
CCCCGCCCCATTGGCACCCGTTACCTCACCCGTAATCTGTAA
AAV-9
Cap only: AY530579
CapVP1: (SEQ ID NO: 21)
ATGGCTGCCGATGGTTATCTTCCAGATTGGCTCGAGGACAACCTTAGTGAAGGAATTCGCGAGTGGTGGGCTTTG
AAACCTGGAGCCCCTCAACCCAAGGCAAATCAACAACATCAAGACAACGCTCGAGGTCTTGTGCTTCCGGGTTAC
AAATACCTTGGACCCGGCAACGGACTCGACAAGGGGGAGCCGGTCAACGCAGCAGACGCGGCGGCCCTCGAGCAC
GACAAGGCCTACGACCAGCAGCTCAAGGCCGGAGACAACCCGTACCTCAAGTACAACCACGCCGACGCCGAGTTC
CAGGAGCGGCTCAAAGAAGATACGTCTTTTGGGGGCAACCTCGGGCGAGCAGTCTTCCAGGCCAAAAAGAGGCTT
CTTGAACCTCTTGGTCTGGTTGAGGAAGCGGCTAAGACGGCTCCTGGAAAGAAGAGGCCTGTAGAGCAGTCTCCT
CAGGAACCGGACTCCTCCGCGGGTATTGGCAAATCGGGTGCACAGCCCGCTAAAAAGAGACTCAATTTCGGTCAG
ACTGGCGACACAGAGTCAGTCCCAGACCCTCAACCAATCGGAGAACCTCCCGCAGCCCCCTCAGGTGTGGGATCT
CTTACAATGGCTTCAGGTGGTGGCGCACCAGTGGCAGACAATAACGAAGGTGCCGATGGAGTGGGTAGTTCCTCG
GGAAATTGGCATTGCGATTCCCAATGGCTGGGGGACAGAGTCATCACCACCAGCACCCGAACCTGGGCCCTGCCC
ACCTACAACAATCACCTCTACAAGCAAATCTCCAACAGCACATCTGGAGGATCTTCAAATGACAACGCCTACTTC
GGCTACAGCACCCCCTGGGGGTATTTTGACTTCAACAGATTCCACTGCCACTTCTCACCACGTGACTGGCAGCGA
CTCATCAACAACAACTGGGGATTCCGGCCTAAGCGACTCAACTTCAAGCTCTTCAACATTCAGGTCAAAGAGGTT
ACGGACAACAATGGAGTCAAGACCATCGCCAATAACCTTACCAGCACGGTCCAGGTCTTCACGGACTCAGACTAT
CAGCTCCCGTACGTGCTCGGGTCGGCTCACGAGGGCTGCCTCCCGCCGTTCCCAGCGGACGTTTTCATGATTCCT
CAGTACGGGTATCTGACGCTTAATGATGGAAGCCAGGCCGTGGGTCGTTCGTCCTTTTACTGCCTGGAATATTTC
CCGTCGCAAATGCTAAGAACGGGTAACAACTTCCAGTTCAGCTACGAGTTTGAGAACGTACCTTTCCATAGCAGC
TACGCTCACAGCCAAAGCCTGGACCGACTAATGAATCCACTCATCGACCAATACTTGTACTATCTCTCAAAGACT
ATTAACGGTTCTGGACAGAATCAACAAACGCTAAAATTCAGTGTGGCCGGACCCAGCAACATGGCTGTCCAGGGA
AGAAACTACATACCTGGACCCAGCTACCGACAACAACGTGTCTCAACCACTGTGACTCAAAACAACAACAGCGAA
TTTGCTTGGCCTGGAGCTTCTTCTTGGGCTCTCAATGGACGTAATAGCTTGATGAATCCTGGACCTGCTATGGCC
AGCCACAAAGAAGGAGAGGACCGTTTCTTTCCTTTGTCTGGATCTTTAATTTTTGGCAAACAAGGAACTGGAAGA
GACAACGTGGATGCGGACAAAGTCATGATAACCAACGAAGAAGAAATTAAAACTACTAACCCGGTAGCAACGGAG
TCCTATGGACAAGTGGCCACAAACCACCAGAGTGCCCAAGCACAGGCGCAGACCGGCTGGGTTCAAAACCAAGGA
ATACTTCCGGGTATGGTTTGGCAGGACAGAGATGTGTACCTGCAAGGACCCATTTGGGCCAAAATTCCTCACACG
GACGGCAACTTTCACCCTTCTCCGCTGATGGGAGGGTTTGGAATGAAGCACCCGCCTCCTCAGATCCTCATCAAA
AACACACCTGTACCTGCGGATCCTCCAACGGCCTTCAACAAGGACAAGCTGAACTCTTTCATCACCCAGTATTCT
ACTGGCCAAGTCAGCGTGGAGATCGAGTGGGAGCTGCAGAAGGAAAACAGCAAGCGCTGGAACCCGGAGATCCAG
TACACTTCCAACTATTACAAGTCTAATAATGTTGAATTTGCTGTTAATACTGAAGGTGTATATAGTGAACCCCGC
CCCATTGGCACCAGATACCTGACTCGTAATCTGTAA
AAV-10
Partial Genome: AY631965
Rep78: (SEQ ID NO: 22)
ATGCCGGGCTTCTACGAGATCGTGATCAAGGTGCCGAGCGACCTGGACGAGCACCTGCCGGGCATTTCTGACTCG
TTTGTGAACTGGGTGGCCGAGAAGGAATGGGAGCTGCCCCCGGATTCTGACATGGATCGGAATCTGATCGAGCAG
GCACCCCTGACCGTGGCCGAGAAGCTGCAGCGCGACTTCCTGGTCCACTGGCGCCGCGTGAGTAAGGCCCCGGAG
GCCCTCTTCTTTGTTCAGTTCGAGAAGGGCGAGTCCTACTTTCACCTGCACGTTCTGGTCGAGACCACGGGGGTC
AAGTCCATGGTCCTGGGCCGCTTCCTGAGTCAGATCAGAGACAGGCTGGTGCAGACCATCTACCGCGGGGTAGAG
CCCACGCTGCCCAACTGGTTCGCGGTGACCAAGACGCGAAATGGCGCCGGCGGGGGGAACAAGGTGGTGGACGAG
TGCTACATCCCCAACTACCTCCTGCCCAAGACGCAGCCCGAGCTGCAGTGGGCGTGGACTAACATGGAGGAGTAT
ATAAGCGCGTGTCTGAACCTCGCGGAGCGTAAACGGCTCGTGGCGCAGCACCTGACCCACGTCAGCCAGACGCAG
GAGCAGAACAAGGAGAATCTGAACCCGAATTCTGACGCGCCCGTGATCAGGTCAAAAACCTCCGCGCGCTACATG
GAGCTGGTCGGGTGGCTGGTGGACCGGGGCATCACCTCCGAGAAGCAGTGGATCCAGGAGGACCAGGCCTCGTAC
ATCTCCTTCAACGCCGCCTCCAACTCGCGGTCCCAGATCAAGGCCGCGCTGGACAATGCCGGAAAGATCATGGCG
CTGACCAAATCCGCGCCCGACTACCTGGTAGGCCCGTCCTTACCCGCGGACATTAAGGCCAACCGCATCTACCGC
ATCCTGGAGCTCAACGGCTACGACCCCGCCTACGCCGGCTCCGTCTTCCTGGGCTGGGCGCAGAAAAAGTTCGGT
AAAAGGAATACAATTTGGCTGTTCGGGCCCGCCACCACCGGCAAGACCAACATCGCGGAAGCCATCGCCCACGCC
GTGCCCTTCTACGGCTGCGTCAACTGGACCAATGAGAACTTTCCCTTCAACGATTGCGTCGACAAGATGGTGATC
TGGTGGGAGGAGGGCAAGATGACCGCCAAGGTCGTGGAGTCCGCCAAGGCCATTCTGGGCGGAAGCAAGGTGCGC
GTCGACCAAAAGTGCAAGTCCTCGGCCCAGATCGACCCCACGCCCGTGATCGTCACCTCCAACACCAACATGTGC
GCCGTGATCGACGGGAACAGCACCACCTTCGAGCACCAGCAGCCCCTGCAGGACCGCATGTTCAAGTTCGAGCTC
ACCCGCCGTCTGGAGCACGACTTTGGCAAGGTGACCAAGCAGGAAGTCAAAGAGTTCTTCCGCTGGGCTCAGGAT
CACGTGACTGAGGTGACGCATGAGTTCTACGTCAGAAAGGGCGGAGCCACCAAAAGACCCGCCCCCAGTGACGCG
GATATAAGCGAGCCCAAGCGGGCCTGCCCCTCAGTTGCGGAGCCATCGACGTCAGACGCGGAAGCACCGGTGGAC
TTTGCGGACAGGTACCAAAACAAATGTTCTCGTCACGCGGGCATGCTTCAGATGCTGTTTCCCTGCAAGACATGC
GAGAGAATGAATCAGAATTTCAACGTCTGCTTCACGCACGGGGTCAGAGACTGCTCAGAGTGCTTCCCCGGCGCG
TCAGAATCTCAACCTGTCGTCAGAAAAAAGACGTATCAGAAACTGTGCGCGATTCATCATCTGCTGGGGGGGGCA
CCCGAGATTGCGTGTTCGGCCTGCGATCTCGTCAACGTGGACTTGGATGACTGTGTTTCTGAGCAATAA
CapVP1: (SEQ ID NO: 23)
ATGGCTGCTGACGGTTATCTTCCAGATTGGCTCGAGGACAACCTCTCTGAGGGCATTCGCGAGTGGTGGGACCTG
AAACCTGGAGCCCCCAAGCCCAAGGCCAACCAGCAGAAGCAGGACGACGGCCGGGGTCTGGTGCTTCCTGGCTAC
AAGTACCTCGGACCCTTCAACGGACTCGACAAGGGGGAGCCCGTCAACGCGGCGGACGCAGCGGCCCTCGAGCAC
GACAAGGCCTACGACCAGCAGCTCAAAGCGGGTGACAATCCGTACCTGCGGTATAACCACGCCGACGCCGAGTTT
CAGGAGCGTCTGCAAGAAGATACGTCTTTTGGGGGCAACCTCGGGCGAGCAGTCTTCCAGGCCAAGAAGCGGGTT
CTCGAACCTCTCGGTCTGGTTGAGGAAGCTGCTAAGACGGCTCCTGGAAAGAAGAGACCGGTAGAACCGTCACCT
CAGCGTTCCCCCGACTCCTCCACGGGCATCGGCAAGAAAGGCCAGCAGCCCGCTAAAAAGAGACTGAACTTTGGG
CAGACTGGCGAGTCAGAGTCAGTCCCCGACCCTCAACCAATCGGAGAACCACCAGCAGGCCCCTCTGGTCTGGGA
TCTGGTACAATGGCTGCAGGCGGTGGCGCTCCAATGGCAGACAATAACGAAGGCGCCGACGGAGTGGGTAGTTCC
TCAGGAAATTGGCATTGCGATTCCACATGGCTGGGCGACAGAGTCATCACCACCAGCACCCGAACCTGGGCCCTG
CCCACCTACAACAACCACCTCTACAAGCAAATCTCCAACGGGACATCGGGAGGAAGCACCAACGACAACACCTAC
TTCGGCTACAGCACCCCCTGGGGGTATTTTGACTTCAACAGATTCCACTGCCACTTCTCACCACGTGACTGGCAG
CGACTCATCAACAACAACTGGGGATTCCGGCCAAAAAGACTCAGCTTCAAGCTCTTCAACATCCAGGTCAAGGAG
GTCACGCAGAATGAAGGCACCAAGACCATCGCCAATAACCTTACCAGCACGATTCAGGTATTTACGGACTCGGAA
TACCAGCTGCCGTACGTCCTCGGCTCCGCGCACCAGGGCTGCCTGCCTCCGTTCCCGGCGGATGTCTTCATGATT
CCCCAGTACGGCTACCTGACACTGAACAATGGAAGTCAAGCCGTAGGCCGTTCCTCCTTCTACTGCCTGGAATAT
TTTCCATCTCAAATGCTGCGAACTGGAAACAATTTTGAATTCAGCTACACCTTCGAGGACGTGCCTTTCCACAGC
AGCTACGCACACAGCCAGAGCTTGGACCGACTGATGAATCCTCTCATTGACCAGTACCTGTACTACTTATCCAGA
ACTCAGTCCACAGGAGGAACTCAAGGTACCCAGCAATTGTTATTTTCTCAAGCTGGGCCTGCAAACATGTCGGCT
CAGGCCAAGAACTGGCTGCCTGGACCTTGCTACCGGCAGCAGCGAGTCTCCACGACACTGTCGCAAAACAACAAC
AGCAACTTTGCTTGGACTGGTGCCACCAAATATCACCTGAACGGAAGAGACTCTCTGGTGAATCCCGGTGTCGCC
ATGGCAACCCACAAGGACGACGAGGAACGCTTCTTCCCGTCGAGCGGAGTCCTGATGTTTGGAAAACAGGGTGCT
GGAAGAGACAATGTGGACTACAGCAGCGTTATGCTAACAAGCGAAGAAGAAATTAAAACCACTAACCCTGTAGCC
ACAGAACAATACGGCGTGGTGGCTGACAACTTGCAGCAAGCCAATACAGGGCCTATTGTGGGAAATGTCAACAGC
CAAGGAGCCTTACCTGGCATGGTCTGGCAGAACCGAGACGTGTACCTGCAGGGTCCCATCTGGGCCAAGATTCCT
CACACGGACGGCAACTTTCACCCGTCTCCTCTGATGGGCGGCTTTGGACTTAAACACCCGCCTCCACAGATCCTG
ATCAAGAACACGCCGGTACCTGCGGATCCTCCAACAACGTTCAGCCAGGCGAAATTGGCTTCCTTCATCACGCAG
TACAGCACCGGACAGGTCAGCGTGGAAATCGAGTGGGAGCTGCAGAAGGAGAACAGCAAACGCTGGAACCCAGAG
ATTCAGTACACTTCAAACTACTACAAATCTACAAATGTGGACTTTGCTGTCAATACAGAGGGAACTTATTCTGAG
CCTCGCCCCATTGGTACTCGTTATCTGACACGTAATCTGTAA
AAV-11
Partial Genome: AY631966
Rep78: (SEQ ID NO: 24)
ATGCCGGGCTTCTACGAGATCGTGATCAAGGTGCCGAGCGACCTGGACGAGCACCTGCCGGGCATTTCTGACTCG
TTTGTGAACTGGGTGGCCGAGAAGGAATGGGAGCTGCCCCCGGATTCTGACATGGATCGGAATCTGATCGAGCAG
GCACCCCTGACCGTGGCCGAGAAGCTGCAGCGCGACTTCCTGGTCCACTGGCGCCGCGTGAGTAAGGCCCCGGAG
GCCCTCTTCTTTGTTCAGTTCGAGAAGGGCGAGTCCTACTTCCACCTCCACGTTCTCGTCGAGACCACGGGGGTC
AAGTCCATGGTCCTGGGCCGCTTCCTGAGTCAGATCAGAGACAGGCTGGTGCAGACCATCTACCGCGGGGTCGAG
CCCACGCTGCCCAACTGGTTCGCGGTGACCAAGACGCGAAATGGCGCCGGCGGGGGGAACAAGGTGGTGGACGAG
TGCTACATCCCCAACTACCTCCTGCCCAAGACCCAGCCCGAGCTGCAGTGGGCGTGGACTAACATGGAGGAGTAT
ATAAGCGCGTGTCTAAACCTCGCGGAGCGTAAACGGCTCGTGGCGCAGCACCTGACCCACGTCAGCCAGACGCAG
GAGCAGAACAAGGAGAATCTGAACCCGAATTCTGACGCGCCCGTGATCAGGTCAAAAACCTCCGCGCGCTACATG
GAGCTGGTCGGGTGGCTGGTGGACCGGGGCATCACCTCCGAGAAGCAGTGGATCCAGGAGGACCAGGCCTCGTAC
ATCTCCTTCAACGCCGCCTCCAACTCGCGGTCCCAGATCAAGGCCGCGCTGGACAATGCCGGAAAGATCATGGCG
CTGACCAAATCCGCGCCCGACTACCTGGTAGGCCCGTCCTTACCCGCGGACATTAAGGCCAACCGCATCTACCGC
ATCCTGGAGCTCAACGGCTACGACCCCGCCTACGCCGGCTCCGTCTTCCTGGGCTGGGCGCAGAAAAAGTTCGGT
AAACGCAACACCATCTGGCTGTTTGGGCCCGCCACCACCGGCAAGACCAACATCGCGGAAGCCATAGCCCACGCC
GTGCCCTTCTACGGCTGCGTGAACTGGACCAATGAGAACTTTCCCTTCAACGATTGCGTCGACAAGATGGTGATC
TGGTGGGAGGAGGGCAAGATGACCGCCAAGGTCGTGGAGTCCGCCAAGGCCATTCTGGGCGGAAGCAAGGTGCGC
GTGGACCAAAAGTGCAAGTCCTCGGCCCAGATCGACCCCACGCCCGTGATCGTCACCTCCAACACCAACATGTGC
GCCGTGATCGACGGGAACAGCACCACCTTCGAGCACCAGCAGCCGCTGCAGGACCGCATGTTCAAGTTCGAGCTC
ACCCGCCGTCTGGAGCACGACTTTGGCAAGGTGACCAAGCAGGAAGTCAAAGAGTTCTTCCGCTGGGCTCAGGAT
CACGTGACTGAGGTGGCGCATGAGTTCTACGTCAGAAAGGGCGGAGCCACCAAAAGACCCGCCCCCAGTGACGCG
GATATAAGCGAGCCCAAGCGGGCCTGCCCCTCAGTTCCGGAGCCATCGACGTCAGACGCGGAAGCACCGGTGGAC
TTTGCGGACAGGTACCAAAACAAATGTTCTCGTCACGCGGGCATGCTTCAGATGCTGTTTCCCTGCAAGACATGC
GAGAGAATGAATCAGAATTTCAACGTCTGCTTCACGCACGGGGTCAGAGACTGCTCAGAGTGCTTCCCCGGCGCG
TCAGAATCTCAACCCGTCGTCAGAAAAAAGACGTATCAGAAACTGTGCGCGATTCATCATCTGCTGGGGGGGGCA
CCCGAGATTGCGTGTTCGGCCTGCGATCTCGTCAACGTGGACTTGGATGACTGTGTTTCTGAGCAATAA
CapVP1: (SEQ ID NO: 25)
ATGGCTGCTGACGGTTATCTTCCAGATTGGCTCGAGGACAACCTCTCTGAGGGCATTCGCGAGTGGTGGGACCTG
AAACCTGGAGCCCCGAAGCCCAAGGCCAACCAGCAGAAGCAGGACGACGGCCGGGGTCTGGTGCTTCCTGGCTAC
AAGTACCTCGGACCCTTCAACGGACTCGACAAGGGGGAGCCCGTCAACGCGGCGGACGCAGCGGCCCTCGAGCAC
GACAAGGCCTACGACCAGCAGCTCAAAGCGGGTGACAATCCGTACCTGCGGTATAACCACGCCGACGCCGAGTTT
CAGGAGCGTCTGCAAGAAGATACGTCTTTTGGGGGCAACCTCGGGCGAGCAGTCTTCCAGGCCAAGAAGAGGGTA
CTCGAACCTCTGGGCCTGGTTGAAGAAGGTGCTAAAACGGCTCCTGGAAAGAAGAGACCGTTAGAGTCACCACAA
GAGCCCGACTCCTCCTCGGGCATCGGCAAAAAAGGCAAACAACCAGCCAGAAAGAGGCTCAACTTTGAAGAGGAC
ACTGGAGCCGGAGACGGACCCCCTGAAGGATCAGATACCAGCGCCATGTCTTCAGACATTGAAATGCGTGCAGCA
CCGGGCGGAAATGCTGTCGATGCGGGACAAGGTTCCGATGGAGTGGGTAATGCCTCGGGTGATTGGCATTGCGAT
TCCACCTGGTCTGAGGGCAAGGTCACAACAACCTCGACCAGAACCTGGGTCTTGCCCACCTACAACAACCACTTG
TACCTGCGTCTCGGAACAACATCAAGCAGCAACACCTACAACGGATTCTCCACCCCCTGGGGATATTTTGACTTC
AACAGATTCCACTGTCACTTCTCACCACGTGACTGGCAAAGACTCATCAACAACAACTGGGGACTACGACCAAAA
GCCATGCGCGTTAAAATCTTCAATATCCAAGTTAAGGAGGTCACAACGTCGAACGGCGAGACTACGGTCGCTAAT
AACCTTACCAGCACGGTTCAGATATTTGCGGACTCGTCGTATGAGCTCCCGTACGTGATGGACGCTGGACAAGAG
GGGAGCCTGCCTCCTTTCCCCAATGACGTGTTCATGGTGCCTCAATATGGCTACTGTGGCATCGTGACTGGCGAG
AATCAGAACCAAACGGACAGAAACGCTTTCTACTGCCTGGAGTATTTTCCTTCGCAAATGTTGAGAACTGGCAAC
AACTTTGAAATGGCTTACAACTTTGAGAAGGTGCCGTTCCACTCAATGTATGCTCACAGCCAGAGCCTGGACAGA
CTGATGAATCCCCTCCTGGACCAGTACCTGTGGCACTTACAGTCGACTACCTCTGGAGAGACTCTGAATCAAGGC
AATGCAGCAACCACATTTGGAAAAATCAGGAGTGGAGACTTTGCCTTTTACAGAAAGAACTGGCTGCCTGGGCCT
TGTGTTAAACAGCAGAGATTCTCAAAAACTGCCAGTCAAAATTACAAGATTCCTGCCAGCGGGGGCAACGCTCTG
TTAAAGTATGACACCCACTATACCTTAAACAACCGCTGGAGCAACATCGCGCCCGGACCTCCAATGGCCACAGCC
GGACCTTCGGATGGGGACTTCAGTAACGCCCAGCTTATATTCCCTGGACCATCTGTTACCGGAAATACAACAACT
TCAGCCAACAATCTGTTGTTTACATCAGAAGAAGAAATTGCTGCCACCAACCCAAGAGACACGGACATGTTTGGC
CAGATTGCTGACAATAATCAGAATGCTACAACTGCTCCCATAACCGGCAACGTGACTGCTATGGGAGTGCTGCCT
GGCATGGTGTGGCAAAACAGAGACATTTACTACCAAGGGCCAATTTGGGCCAAGATCCCACACGCGGACGGACAT
TTTCATCCTTCACCGCTGATTGGTGGGTTTGGACTGAAACACCCGCCTCCCCAGATATTCATCAAGAACACTCCC
GTACCTGCCAATCCTGCGACAACCTTCACTGCAGCCAGAGTGGACTCTTTCATCACACAATACAGCACCGGCCAG
GTCGCTGTTCAGATTGAATGGGAAATTGAAAAGGAACGCTCCAAACGCTGGAATCCTGAAGTGCAGTTTACTTCA
AACTATGGGAACCAGTCTTCTATGTTGTGGGCTCCTGATACAACTGGGAAGTATACAGAGCCGCGGGTTATTGGC
TCTCGTTATTTGACTAATCATTTGTAA
AAV-12
Partial Genome: DQ813647
Rep78: (SEQ ID NO: 26)
ATGCCGGGGTTCTACGAGGTGGTGATCAAGGTGCCCAGCGACCTGGACGAGCACCTGCCCGGCATTTCTGACTCC
TTTGTGAACTGGGTGGCCGAGAAGGAATGGGAGTTGCCCCCGGATTCTGACATGGATCAGAATCTGATTGAGCAG
GCACCCCTGACCGTGGCCGAGAAGCTGCAGCGCGAGTTCCTGGTGGAATGGCGCCGAGTGAGTAAATTTCTGGAG
GCCAAGTTTTTTGTGCAGTTTGAAAAGGGGGACTCGTACTTTCATTTGCATATTCTGATTGAAATTACCGGCGTG
AAATCCATGGTGGTGGGCCGCTACGTGAGTCAGATTAGGGATAAACTGATCCAGCGCATCTACCGCGGGGTCGAG
CCCCAGCTGCCCAACTGGTTCGCGGTCACAAAGACCCGAAATGGCGCCGGAGGCGGGAACAAGGTGGTGGACGAG
TGCTACATCCCCAACTACCTGCTCCCCAAGGTCCAGCCCGAGCTTCAGTGGGCGTGGACTAACATGGAGGAGTAT
ATAAGCGCCTGTTTGAACCTCGCGGAGCGTAAACGGCTCGTGGCGCAGCACCTGACGCACGTCTCCCAGACCCAG
GAGGGCGACAAGGAGAATCTGAACCCGAATTCTGACGCGCCGGTGATCCGGTCAAAAACCTCCGCCAGGTACATG
GAGCTGGTCGGGTGGCTGGTGGACAAGGGCATCACGTCCGAGAAGCAGTGGATCCAGGAGGACCAGGCCTCGTAC
ATCTCCTTCAACGCGGCCTCCAACTCCCGGTCGCAGATCAAGGCGGCCCTGGACAATGCCTCCAAAATCATGAGC
CTCACCAAAACGGCTCCGGACTATCTCATCGGGCAGCAGCCCGTGGGGGACATTACCACCAACCGGATCTACAAA
ATCCTGGAACTGAACGGGTACGACCCCCAGTACGCCGCCTCCGTCTTTCTCGGCTGGGCCCAGAAAAAGTTTGGA
AAGCGCAACACCATCTGGCTGTTTGGGCCCGCCACCACCGGCAAGACCAACATCGCGGAAGCCATCGCCCACGCG
GTCCCCTTCTACGGCTGCGTCAACTGGACCAATGAGAACTTTCCCTTCAACGACTGCGTCGACAAAATGGTGATT
TGGTGGGAGGAGGGCAAGATGACCGCCAAGGTCGTAGAGTCCGCCAAGGCCATTCTGGGCGGCAGCAAGGTGCGC
GTGGACCAAAAATGCAAGGCCTCTGCGCAGATCGACCCCACCCCCGTGATCGTCACCTCCAACACCAACATGTGC
GCCGTGATTGACGGGAACAGCACCACCTTCGAGCACCAGCAGCCCCTGCAGGACCGGATGTTCAAGTTTGAACTC
ACCCGCCGCCTCGACCACGACTTTGGCAAGGTCACCAAGCAGGAAGTCAAGGACTTTTTCCGGTGGGCGGCTGAT
CACGTGACTGACGTGGCTCATGAGTTTTACGTCACAAAGGGTGGAGCTAAGAAAAGGCCCGCCCCCTCTGACGAG
GATATAAGCGAGCCCAAGCGGCCGCGCGTGTCATTTGCGCAGCCGGAGACGTCAGACGCGGAAGCTCCCGGAGAC
TTCGCCGACAGGTACCAAAACAAATGTTCTCGTCACGCGGGTATGCTGCAGATGCTCTTTCCCTGCAAGACGTGC
GAGAGAATGAATCAGAATTCCAACGTCTGCTTCACGCACGGTCAGAAAGATTGCGGGGAGTGCTTTCCCGGGTCA
GAATCTCAACCGGTTTCTGTCGTCAGAAAAACGTATCAGAAACTGTGCATCCTTCATCAGCTCCGGGGGGCACCC
GAGATCGCCTGCTCTGCTTGCGACCAACTCAACCCCGATTTGGACGATTGCCAATTTGAGCAATAA
CapVP1: (SEQ ID NO: 27)
ATGGCTGCTGACGGTTATCTTCCAGATTGGCTCGAGGACAACCTCTCTGAAGGCATTCGCGAGTGGTGGGCGCTG
AAACCTGGAGCTCCACAACCCAAGGCCAACCAACAGCATCAGGACAACGGCAGGGGTCTTGTGCTTCCTGGGTAC
AAGTACCTCGGACCCTTCAACGGACTCGACAAGGGAGAGCCGGTCAACGAGGCAGACGCCGCGGCCCTCGAGCAC
GACAAGGCCTACGACAAGCAGCTCGAGCAGGGGGACAACCCGTATCTCAAGTACAACCACGCCGACGCCGAGTTC
CAGCAGCGCTTGGCGACCGACACCTCTTTTGGGGGCAACCTCGGGCGAGCAGTCTTCCAGGCCAAAAAGAGGATT
CTCGAGCCTCTGGGTCTGGTTGAAGAGGGCGTTAAAACGGCTCCTGGAAAGAAACGCCCATTAGAAAAGACTCCA
AATCGGCCGACCAACCCGGACTCTGGGAAGGCCCCGGCCAAGAAAAAGCAAAAAGACGGCGAACCAGCCGACTCT
GCTAGAAGGACACTCGACTTTGAAGACTCTGGAGCAGGAGACGGACCCCCTGAGGGATCATCTTCCGGAGAAATG
TCTCATGATGCTGAGATGCGTGCGGCGCCAGGCGGAAATGCTGTCGAGGCGGGACAAGGTGCCGATGGAGTGGGT
AATGCCTCCGGTGATTGGCATTGCGATTCCACCTGGTCAGAGGGCCGAGTCACCACCACCAGCACCCGAACCTGG
GTCCTACCCACGTACAACAACCACCTGTACCTGCGAATCGGAACAACGGCCAACAGCAACACCTACAACGGATTC
TCCACCCCCTGGGGATACTTTGACTTTAACCGCTTCCACTGCCACTTTTCCCCACGCGACTGGCAGCGACTCATC
AACAACAACTGGGGACTCAGGCCGAAATCGATGCGTGTTAAAATCTTCAACATACAGGTCAAGGAGGTCACGACG
TCAAACGGCGAGACTACGGTCGCTAATAACCTTACCAGCACGGTTCAGATCTTTGCGGATTCGACGTATGAACTC
CCATACGTGATGGACGCCGGTCAGGAGGGGAGCTTTCCTCCGTTTCCCAACGACGTCTTTATGGTTCCCCAATAC
GGATACTGCGGAGTTGTCACTGGAAAAAACCAGAACCAGACAGACAGAAATGCCTTTTACTGCCTGGAATACTTT
CCATCCCAAATGCTAAGAACTGGCAACAATTTTGAAGTCAGTTACCAATTTGAAAAAGTTCCTTTCCATTCAATG
TACGCGCACAGCCAGAGCCTGGACAGAATGATGAATCCTTTACTGGATCAGTACCTGTGGCATCTGCAATCGACC
ACTACCGGAAATTCCCTTAATCAAGGAACAGCTACCACCACGTACGGGAAAATTACCACTGGAGACTTTGCCTAC
TACAGGAAAAACTGGTTGCCTGGAGCCTGCATTAAACAACAAAAATTTTCAAAGAATGCCAATCAAAACTACAAG
ATTCCCGCCAGCGGGGGAGACGCCCTTTTAAAGTATGACACGCATACCACTCTAAATGGGCGATGGAGTAACATG
GCTCCTGGACCTCCAATGGCAACCGCAGGTGCCGGGGACTCGGATTTTAGCAACAGCCAGCTGATCTTTGCCGGA
CCCAATCCGAGCGGTAACACGACCACATCTTCAAACAATTTGTTGTTTACCTCAGAAGAGGAGATTGCCACAACA
AACCCACGAGACACGGACATGTTTGGACAGATTGCAGATAATAATCAAAATGCCACCACCGCCCCTCACATCGCT
AACCTGGACGCTATGGGAATTGTTCCCGGAATGGTCTGGCAAAACAGAGACATCTACTACCAGGGCCCTATTTGG
GCCAAGGTCCCTCACACGGACGGACACTTTCACCCTTCGCCGCTGATGGGAGGATTTGGACTGAAACACCCGCCT
CCACAGATTTTCATCAAAAACACCCCCGTACCCGCCAATCCCAATACTACCTTTAGCGCTGCAAGGATTAATTCT
TTTCTGACGCAGTACAGCACCGGACAAGTTGCCGTTCAGATCGACTGGGAAATTCAGAAGGAGCATTCCAAACGC
TGGAATCCCGAAGTTCAATTTACTTCAAACTACGGCACTCAAAATTCTATGCTGTGGGCTCCCGACAATGCTGGC
AACTACCACGAACTCCGGGCTATTGGGTCCCGTTTCCTCACCCACCACTTGTAA
AAV-13
Partial Genome: EU285562
Rep78: (SEQ ID NO: 28)
ATGCCGGGATTCTACGAGATTGTCCTGAAGGTGCCCAGCGACCTGGACGAGCACCTGCCTGGCATTTCTGACTCT
TTTGTAAACTGGGTGGCGGAGAAGGAATGGGAGCTGCCGCCGGATTCTGACATGGATCTGAATCTGATTGAGCAG
GCACCCCTAACCGTGGCCGAAAAGCTGCAACGCGAATTCCTGGTCGAGTGGCGCCGCGTGAGTAAGGCCCCGGAG
GCCCTCTTCTTTGTTCAGTTCGAGAAGGGGGACAGCTACTTCCACCTACACATTCTGGTGGAGACCGTGGGCGTG
AAATCCATGGTGGTGGGCCGCTACGTGAGCCAGATTAAAGAGAAGCTGGTGACCCGCATCTACCGCGGGGTCGAG
CCGCAGCTTCCGAACTGGTTCGCGGTGACCAAGACGCGTAATGGCGCCGGAGGCGGGAACAAGGTGGTGGACGAC
TGCTACATCCCCAACTACCTGCTCCCCAAGACCCAGCCCGAGCTCCAGTGGGCGTGGACTAATATGGACCAGTAT
TTAAGCGCCTGTTTGAATCTCGCGGAGCGTAAACGGCTGGTGGCGCAGCATCTGACGCACGTGTCGCAGACGCAG
GAGCAGAACAAAGAGAACCAGAATCCCAATTCTGACGCGCCGGTGATCAGATCAAAAACCTCCGCGAGGTACATG
GAGCTGGTCGGGTGGCTGGTGGACCGCGGGATCACGTCAGAAAAGCAATGGATCCAGGAGGACCAGGCCTCTTAC
ATCTCCTTCAACGCCGCCTCCAACTCGCGGTCACAAATCAAGGCCGCACTGGACAATGCCTCCAAATTTATGAGC
CTGACAAAAACGGCTCCGGACTACCTGGTGGGAAACAACCCGCCGGAGGACATTACCAGCAACCGGATCTACAAA
ATCCTCGAGATGAACGGGTACGATCCGCAGTACGCGGCCTCCGTCTTCCTGGGCTGGGCGCAAAAGAAGTTCGGG
AAGAGGAACACCATCTGGCTCTTTGGGCCGGCCACGACGGGTAAAACCAACATCGCTGAAGCTATCGCCCACGCC
GTGCCCTTTTACGGCTGCGTGAACTGGACCAATGAGAACTTTCCGTTCAACGATTGCGTCGACAAGATGGTGATC
TGGTGGGAGGAGGGCAAGATGACGGCCAAGGTCGTGGAGTCCGCCAAGGCCATTCTGGGCGGAAGCAAGGTGCGC
GTGGACCAAAAGTGCAAGTCATCGGCCCAGATCGACCCAACTCCCGTCATCGTCACCTCCAACACCAACATGTGC
GCGGTCATCGACGGAAATTCCACCACCTTCGAGCACCAACAACCACTCCAAGACCGGATGTTCAAGTTCGAGCTC
ACCAAGCGCCTGGAGCACGACTTTGGCAAGGTCACCAAGCAGGAAGTCAAGGACTTTTTCCGGTGGGCGTCAGAT
CACGTGACTGAGGTGTCTCACGAGTTTTACGTCAGAAAGGGTGGAGCTAGAAAGAGGCCCGCCCCCAATGACGCA
GATATAAGTGAGCCCAAGCGGGCCTGTCCGTCAGTTGCGCAGCCATCGACGTCAGACGCGGAAGCTCCGGTGGAC
TACGCGGACAGGTACCAAAACAAATGTTCTCGTCACGTGGGCATGAATCTGATGCTTTTTCCCTGCCGGCAATGC
GAGAGAATGAATCAGAATGTGGACATTTGCTTCACGCACGGGGTCATGGACTGTGCCGAGTGCTTCCCCGTGTCA
GAATCTCAACCCGTGTCTGTCGTCAGAAAGCGGACATATCAGAAACTGTGTCCGATTCATCACATCATGGGGAGG
GCGCCCGAGGTGGCTTGTTCGGCCTGCGATCTGGCCAATGTGGACTTGGATGACTGTGACATGGAGCAATAA
CapVP1: (SEQ ID NO: 29)
ATGACTGACGGTTACCTTCCAGATTGGCTAGAGGACAACCTCTCTGAAGGCGTTCGAGAGTGGTGGGCGCTGCAA
CCTGGAGCCCCTAAACCCAAGGCAAATCAACAACATCAGGACAACGCTCGGGGTCTTGTGCTTCCGGGTTACAAA
TACCTCGGACCCGGCAACGGACTTGACAAGGGGGAACCCGTCAACGCAGCGGACGCGGCAGCCCTCGAACACGAC
AAGGCCTACGACCAGCAGCTCAAGGCCGGTGACAACCCCTACCTCAAGTACAACCACGCCGACGCCGAGTTTCAG
GAGCGTCTTCAAGAAGATACGTCTTTTGGGGGCAACCTCGGACGAGCAGTCTTCCAGGCCAAAAAGAGGATCCTT
GAGCCTCTGGGTCTGGTTGAGGAAGCGGCTAAGACGGCTCCTGGAAAAAAGAGACCTGTAGAGCAATCTCCAGCA
GAACCGGACTCCTCTTCGGGCATCGGCAAATCAGGCCAGCAGCCCGCTAGAAAAAGACTGAATTTTGGTCAGACT
GGCGACACAGAGTCAGTCCCAGACCCTCAACCACTCGGACAACCTCCCGCAGCCCCCTCTGGTGTGGGATCTACT
ACAATGGCTTCAGGCGGTGGCGCACCAATGGCAGACAATAACGAGGGTGCCGATGGAGTGGGTAATTCCTCAGGA
AATTGGCATTGCGATTCCCAATGGCTGGGCGACAGAGTCATCACCACCAGCACCCGCACCTGGGCCCTGCCCACC
TACAACAATCACCTCTACAAGCAAATCTCCAGCCAATCAGGAGCCACCAACGACAACCACTACTTTGGCTACAGC
ACCCCCTGGGGGTATTTTGACTTCAACAGATTCCACTGCCACTTTTCACCACGTGACTGGCAAAGACTCATCAAC
AACAACTGGGGATTCCGACCCAAGAGACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCCGAGTACCAGCTCCCG
TACGTCCTCGGCTCGGCGCATCAGGGATGCCTCCCGCCGTTCCCAGCAGACGTCTTCATGGTCCCACAGTATGGA
TACCTCACCCTGAACAACGGGAGTCAGGCGGTAGGACGCTCTTCCTTTTACTGCCTGGAGTACTTTCCTTCTCAG
ATGCTGCGTACTGGAAACAACTTTCAGTTTAGCTACACTTTTGAAGACGTGCCTTTCCACAGCAGCTACGCTCAC
AGCCAAAGTCTGGACCGTCTCATGAATCCTCTGATCGACCAGTACCTGTACTATCTGAACAGGACACAAACAGCC
AGTGGAACTCAGCAGTCTCGGCTACTGTTTAGCCAAGCTGGACCCACCAGTATGTCTCTTCAAGCTAAAAACTGG
CTGCCTGGACCTTGCTACAGACAGCAGCGTCTGTCAAAGCAGGCAAACGACAACAACAACAGCAACTTTCCCTGG
ACTGGTGCCACCAAATATCATCTGAATGGCCGGGACTCATTGGTGAACCCGGGCCCTGCTATGGCCAGTCACAAG
GATGACAAAGAAAAGTTTTTCCCCATGCATGGAACCCTGATATTTGGTAAAGAAGGAACAAATGCCAACAACGCG
GATTTGGAAAATGTCATGATTACAGATGAAGAAGAAATCCGCACCACCAATCCCGTGGCTACGGAGCAGTACGGG
ACTGTGTCAAATAATTTGCAAAACTCAAACGCTGGTCCAACTACTGGAACTGTCAATCACCAAGGAGCGTTACCT
GGTATGGTGTGGCAGGATCGAGACGTGTACCTGCAGGGACCCATTTGGGCCAAGATTCCTCACACCGATGGACAC
TTTCATCCTTCTCCACTGATGGGAGGTTTTGGGCTCAAACACCCGCCTCCTCAGATCATGATCAAAAACACTCCC
GTTCCAGCCAATCCTCCCACAAACTTTAGTGCGGCAAAGTTTGCTTCCTTCATCACACAGTACTCCACGGGGCAG
GTCAGCGTGGAGATCGAGTGGGAGCTGCAGAAGGAGAACAGCAAACGCTGGAATCCCGAAATTCAGTACACTTCC
AACTACAACAAATCTGTTAATGTGGACTTTACTGTGGACACTAATGGTGTGTATTCAGAGCCTCGCCCCATTGGC
ACCAGATACCTGACTCGTAATCTGTAA
AAV rh10 (SEQ ID NO: 30)
GenBank: AY243015.1-Non-human primate Adeno-associated virus isolate
AAVrh. 10 capsid protein (VP1) gene, complete cds

| | |
|---|---|
| 1 | ATGGCTGCCG ATGGTTATCT TCCAGATTGG CTCGAGGACA ACCTCTCTGA GGGCATTCGC |
| 61 | GAGTGGTGGG ACTTGAAACC TGGAGCCCCG AAACCCAAAG CCAACCAGCA AAAGCAGGAC |
| 121 | GACGGCCGGG GTCTGGTGCT TCCTGGCTAC AAGTACCTCG GACCCTTCAA CGGACTCGAC |
| 181 | AAGGGGGAGC CCGTCAACGC GGCGGACGCA GCGGCCCTCG AGCACGACAA GGCCTACGAC |
| 241 | CAGCAGCTCA AAGCGGGTGA CAATCCGTAC CTGCGGTATA ACCACGCCGA CGCCGAGTTT |
| 301 | CAGGAGCGTC TGCAAGAAGA TACGTCTTTT GGGGGCAACC TCGGGCGAGC AGTCTTCCAG |
| 361 | GCCAAGAAGC GGGTTCTCGA ACCTCTCGGT CTGGTTGAGG AAGGCGCTAA GACGGCTCCT |
| 421 | GGAAAGAAGA GACCGGTAGA GCCATCACCC CAGCGTTCTC CAGACTCCTC TACGGGCATC |
| 481 | GGCAAGAAAG GCCAGCAGCC CGCGAAAAAG AGACTCAACT TTGGGCAGAC TGGCGACTCA |
| 541 | GAGTCAGTGC CCGACCCTCA ACCAATCGGA GAACCCCCCG CAGGCCCCTC TGGTCTGGGA |
| 601 | TCTGGTACAA TGGCTGCAGG CGGTGGCGCT CCAATGGCAG ACAATAACGA AGGCGCCGAC |
| 661 | GGAGTGGGTA GTTCCTCAGG AAATTGGCAT TGCGATTCCA CATGGCTGGG CGACAGAGTC |
| 721 | ATCACCACCA GCACCCGAAC CTGGGCCCTC CCCACCTACA ACAACCACCT CTACAAGCAA |
| 781 | ATCTCCAACG GGACTTCGGG AGGAAGCACC AACGACAACA CCTACTTCGG CTACAGCACC |
| 841 | CCCTGGGGGT ATTTTGACTT TAACAGATTC CACTGCCACT TCTCACCACG TGACTGGCAG |
| 901 | CGACTCATCA ACAACAACTG GGGATTCCGG CCCAAGAGAC TCAACTTCAA GCTCTTCAAC |
| 961 | ATCCAGGTCA AGGAGGTCAC GCAGAATGAA GGCACCAAGA CCATCGCCAA TAACCTTACC |
| 1021 | AGCACGATTC AGGTCTTTAC GGACTCGGAA TACCAGCTCC CGTACGTCCT CGGCTCTGCG |
| 1081 | CACCAGGGCT GCCTGCCTCC GTTCCCGGCG GACGTCTTCA TGATTCCTCA GTACGGGTAC |
| 1141 | CTGACTCTGA ACAATGGCAG TCAGGCCGTG GGCCGTTCCT CCTTCTACTG CCTGGAGTAC |
| 1201 | TTTCCTTCTC AAATGCTGAG AACGGGCAAC AACTTTGAGT TCAGCTACCA GTTTGAGGAC |
| 1261 | GTGCCTTTTC ACAGCAGCTA CGCGCACAGC CAAAGCCTGG ACCGGCTGAT GAACCCCCTC |
| 1321 | ATCGACCAGT ACCTGTACTA CCTGTCTCGG ACTCAGTCCA CGGGAGGTAC CGCAGGAACT |
| 1381 | CAGCAGTTGC TATTTTCTCA GGCCGGGCCT AATAACATGT CGGCTCAGGC CAAAAACTGG |
| 1441 | CTACCCGGGC CCTGCTACCG GCAGCAACGC GTCTCCACGA CACTGTCGCA AAATAACAAC |
| 1501 | AGCAACTTTG CCTGGACCGG TGCCACCAAG TATCATCTGA ATGGCAGAGA CTCTCTGGTA |
| 1561 | AATCCCGGTG TCGCTATGGC AACCCACAAG GACGACGAAG AGCGATTTTT TCCGTCCAGC |
| 1621 | GGAGTCTTAA TGTTTGGGAA ACAGGGAGCT GGAAAAGACA ACGTGGACTA TAGCAGCGTT |
| 1681 | ATGCTAACCA GTGAGGAAGA AATTAAAACC ACCAACCCAG TGGCCACAGA ACAGTACGGC |
| 1741 | GTGGTGGCCG ATAACCTGCA ACAGCAAAAC GCCGCTCCTA TTGTAGGGGC CGTCAACAGT |
| 1801 | CAAGGAGCCT TACCTGGCAT GGTCTGGCAG AACCGGGACG TGTACCTGCA GGGTCCTATC |
| 1861 | TGGGCCAAGA TTCCTCACAC GGACGGAAAC TTTCATCCCT CGCCGCTGAT GGGAGGCTTT |
| 1921 | GGACTGAAAC ACCCGCCTCC TCAGATCCTG ATTAAGAATA CACCTGTTCC CGCGGATCCT |
| 1981 | CCAACTACCT TCAGTCAAGC TAAGCTGGCG TCGTTCATCA CGCAGTACAG CACCGGACAG |
| 2041 | GTCAGCGTGG AAATTGAATG GGAGCTGCAG AAAGAAAACA GCAAACGCTG GAACCCAGAG |
| 2101 | ATTCAATACA CTTCCAACTA CTACAAATCT ACAAATGTGG ACTTTGCTGT TAACACAGAT |
| 2161 | GGCACTTATT CTGAGCCTCG CCCCATCGGC ACCCGTTACC TCACCCGTAA TCTGTAA |

AAV rh39: (SEQ ID NO: 31)
GENBANK: EU368921.1 ADENO-ASSOCIATED VIRUS ISOLATE RH. 39 CAPSID PROTEIN VP1
GENE, PARTIAL CDS

| | |
|---|---|
| 1 | ATGGCTGCCG ATGGTTATCT TCCAGATTGG CTCGAGGACA ACCTCTCTGA GGGCATTCGC |
| 61 | GAGTGGTGGG CGCTGAAACC TGGAGCCCCG AAGCCCAAAG CCAACCAGCA AAAGCAGGAC |
| 121 | GACGGCCGGG GTCTGGTGCT TCCTGGCTAC AAGTACCTCG GACCCTTCAA CGGACTCGAC |
| 181 | AAGGGGGAGC CCGTCAACGC GGCGGACGCA GCGGCCCTCG AGCACGACAA GGCCTACGAC |
| 241 | CAGCAGCTCA AAGCGGGTGA CAATCCGTAC CTGCGGTATA ACCACGCCGA CGCCGAGTTT |
| 301 | CAGGAGCGTC TGCAAGAAGA TACGTCTTTT GGGGGCAACC TCGGGCGAGC AGTCTTCCAG |
| 361 | GCCAAGAAGC GGGTTCTCGA ACCTCTCGGT CTGGTTGAGG AAGCTGCTAA GACGGCTCCT |
| 421 | GGAAAGAAGA GACCGGTAGA ACCGTCACCT CAGCGTTCCC CCGACTCCTC CACGGGCATC |
| 481 | GGCAAGAAAG GCCAGCAGCC CGCTAAAAAG AGACTGAACT TTGGTCAGAC TGGCGACTCA |
| 541 | GAGTCAGTCC CCGACCCTCA ACCAATCGGA GAACCACCAG CAGGCCCCTC TGGTCTGGGA |
| 601 | TCTGGTACAA TGGCTGCAGG CGGTGGCGCT CCAATGGCAG ACAATAACGA AGGCGCCGAC |
| 661 | GGAGTGGGTA GTTCCTCAGG AAATTGGCAT TGCGATTCCA CATGGCTGGG CGACAGAGTC |
| 721 | ATCACCACCA GCACCCGAAC CTGGGCCCTG CCCACCTACA ACAACCACCT CTACAAGCAA |
| 781 | ATATCCAATG GGACATCGGG AGGAAGCACC AACGACAACA CCTACTTCGG CTACAGCACC |
| 841 | CCCTGGGGGT ATTTTGACTT CAACAGATTC CACTGCCACT TCTCACCACG TGACTGGCAG |
| 901 | CGACTCATCA ACAACAACTG GGGATTCCGG CCAAAAAGAC TCAGCTTCAA GCTCTTCAAC |
| 961 | ATCCAGGTCA AGGAGGTCAC GCAGAATGAA GGCACCAAGA CCATCGCCAA TAACCTTACC |
| 1021 | AGCACGATTC AGGTATTTAC GGACTCGGAA TACCAGCTGC CGTACGTCCT CGGCTCCGCG |
| 1081 | CACCAGGGCT GCCTGCCTCC GTTCCCGGCG GACGTCTTCA TGATTCCCCA GTACGGCTAC |
| 1141 | CTTACACTGA ACAATGGAAG TCAAGCCGTA GGCCGTTCCT CCTTCTACTG CCTGGAATAT |
| 1201 | TTTCCATCTC AAATGCTGCG AACTGGAAAC AATTTTGAAT TCAGCTACAC CTTCGAGGAC |
| 1261 | GTGCCTTTCC ACAGCAGCTA CGCACACAGC CAGAGCTTGG ACCGACTGAT GAATCCTCTC |
| 1321 | ATCGACCAGT ACCTGTACTA CTTATCCAGA ACTCAGTCCA CAGGAGGAAC TCAAGGTACC |
| 1381 | CAGCAATTGT TATTTTCTCA AGCTGGGCCT GCAAACATGT CGGCTCAGGC TAAGAACTGG |
| 1441 | CTACCTGGAC CTTGCTACCG GCAGCAGCGA GTCTCTACGA CACTGTCGCA AAACAACAAC |
| 1501 | AGCAACTTTG CTTGGACTGG TGCCACCAAA TATCACCTGA ACGGAAGAGA CTCTTTGGTA |
| 1561 | AATCCCGGTG TCGCCATGGC AACCCACAAG GACGACGAGG AACGCTTCTT CCCGTCGAGT |
| 1621 | GGAGTCCTGA TGTTTGGAAA ACAGGGTGCT GGAAGAGACA ATGTGGACTA CAGCAGCGTT |
| 1681 | ATGCTAACCA GCGAAGAAGA AATTAAAACC ACTAACCCTG TAGCCACAGA ACAATACGGT |
| 1741 | GTGGTGGCTG ATAACTTGCA GCAAACCAAT ACGGGGCCTA TTGTGGGAAA TGTCAACAGC |
| 1801 | CAAGGAGCCT TACCTGGCAT GGTCTGGCAG AACCGAGACG TGTACCTGCA GGGTCCCATC |
| 1861 | TGGGCCAAGA TTCCTCACAC GGACGGCAAC TTCCACCCTT CACCGCTAAT GGGAGGATTT |
| 1921 | GGACTGAAGC ACCCACCTCC TCAGATCCTG ATCAAGAACA CGCCGGTACC TGCGGATCCT |
| 1981 | CCAACAACGT TCAGCCAGGC GAAATTGGCT TCCTTCATTA CGCAGTACAG CACCGGACAG |
| 2041 | GTCAGCGTGG AAATCGAGTG GGAGCTGCAG AAGGAGAACA GCAAACGCTG GAACCCAGAG |
| 2101 | ATTCAGTACA CTTCAAACTA CTACAAATCT ACAAATGTGG ACTTTGCTGT CAATACAGAG |
| 2161 | GGAACTTATT CTGAGCCTCG CCCCATTGGT ACTCGTTACC TCACCCGTAA TCTG |

AAV rh43: (SEQ ID NO: 32)
GENBANK: JA400153.1 AAV serotype, clone rh. 43

| | |
|---|---|
| 1 | ATGGCTGCCG ATGGTTATCT TCCAGATTGG CTCGAGGACA ACCTCTCTGA GGGCATTCGC |
| 61 | GAGTGGTGGG ACTTGAAACC TGGAGCCCCG AAACCCAAAG CCAACCAGCA AAAGCAGGAC |
| 121 | GACGGCCGGG GCCTGGTGCT TCCTGGCTAC AAGTACCTCG GACCCTTCAA CGGACTCGAC |
| 181 | AAGGGGGAGC CCGTCAACGC GGCGGACGCA GCGGCCCTCG AGCACGACAA GGCCTACGAC |
| 241 | CAGCAGCTCG AAGCGGGTGA CAATCCGTAC CTGCGGTATA ACCACGCCGA CGCCGAGTTT |
| 301 | CAGGAGCGTC TGCAAGAAGA TACGTCTTTT GGGGGCAACC TCGGGCGAGC AGTCTTCCAG |
| 361 | GCCAAGAAGC GGGTTCTCGA ACCTCTCGGT CTGGTTGAGG AAGGCGCTAA GACGGCTCCT |
| 421 | GGAAAGAAGA GACCAGTAGA GCAGTCACCC CAAGAACCAG ACTCCTCCTC GGGCATCGGC |
| 481 | AAGAAAGGCC AACAGCCCGC CAGAAAAAGA CTCAATTTTG GCCAGACTGG CGACTCAGAG |
| 541 | TCAGTTCCAG ACCCTCAACC TCTCGGAGAA CCTCCAGCAG CGCCCTCTGG TGTGGGACCT |
| 601 | AATACAATGG CTGCAGGCGG TGGCGCACCA ATGGCAGACA ATAACGAAGG CGCCGACGGA |
| 661 | GTGGGTAGTT CCTCGGGAAA TTGGCATTGC GATTCCACAT GGCTGGGCGA CAGAGTCATC |
| 721 | ACCACCAGCA CCCGAACCTG GGCCCTGCCC ACCTACAACA ACCACCTCTA CAAGCAAATC |
| 781 | TCCAACGGGA CATCGGGAGG AGCCACCAAC GACAACACCT ACTTCGGCTA CAGCACCCCC |
| 841 | TGGGGGTATT TTGACTTTAA CAGATTCCAC TGCCACTTTT CACCACGTGA CTGGCAGCGA |
| 901 | CTCATCAACA ACAACTGGGG ATTCCGGCCC AAGAGACTCA GCTTCAAGCT CTTCAACATC |
| 961 | CAGGTCAAGG AGGTCACGCA GAATGAAGGC ACCAAGACCA TCGCCAATAA CCTCACCAGC |
| 1021 | ACCATCCAGG TGTTTACGGA CTCGGAGTAC CAGCTGCCGT ACGTTCTCGG CTCTGCCCAC |
| 1081 | CAGGGCTGCC TGCCTCCGTT CCCGGCGGAC GTGTTCATGA TTCCCCAGTA CGGCTACCTA |
| 1141 | ACACTCAACA ACGGTAGTCA GGCCGTGGGA CGCTCCTCCT TCTACTGCCT GGAATACTTT |
| 1201 | CCTTCGCAGA TGCTGAGAAC CGGCAACAAC TTCCAGTTTA CTTACACCTT CGAGGACGTG |
| 1261 | CCTTTCCACA GCAGCTACGC CCACAGCCAG AGCTTGGACC GGCTGATGAA TCCTCTGATT |
| 1321 | GACCAGTACC TGTACTACTT GTCTCGGACT CAAACAACAG GAGGCACGGC AAATACGCAG |
| 1381 | ACTCTGGGCT TCAGCCAAGG TGGGCCTAAT ACAATGGCCA ATCAGGCAAA GAACTGGCTG |
| 1441 | CCAGGACCCT GTTACCGCCA ACAACGCGTC TCAACGACAA CCGGGCAAAA CAACAATAGC |
| 1501 | AACTTTGCCT GGACTGCTGG GACCAAATAC CATCTGAATG GAAGAAATTC ATTGGCTAAT |
| 1561 | CCTGGCATCG CTATGGCAAC ACACAAAGAC GACGAGGAGC GTTTTTTCCC AGTAACGGGA |
| 1621 | TCCTGTTTTT GGCAACAAAA TGCTGCCAGA GACAATGCGG ATTACAGCGA TGTCATGCTC |
| 1681 | ACCAGCGAGG AAGAAATCAA AACCACTAAC CCTGTGGCTA CAGAGGAATA CGGTATCGTG |
| 1741 | GCAGATAACT TGCAGCAGCA AAACACGGCT CCTCAAATTG GAACTGTCAA CAGCCAGGGG |
| 1801 | GCCTTACCCG GTATGGTCTG GCAGAACCGG GACGTGTACC TGCAGGGTCC CATCTGGGCC |
| 1861 | AAGATTCCTC ACACGGACGG CAACTTCCAC CCGTCTCCGC TGATGGGCGG CTTTGGCCTG |
| 1921 | AAACATCCTC CGCCTCAGAT CCTGATCAAG AACACGCCTG TACCTGCGGA TCCTCCGACC |
| 1981 | ACCTTCAACC AGTCAAAGCT GAACTCTTTC ATCACGCAAT ACAGCACCGG ACAGGTCAGC |
| 2041 | GTGGAAATTG AATGGGAGCT ACAGAAGGAA AACAGCAAGC GCTGGAACCC CGAGATCCAG |
| 2101 | TACACCTCCA ACTACTACAA ATCTACAAGT GTGGACTTTG CTGTTAATAC AGAAGGCGTG |
| 2161 | TACTCTGAAC CCCGCCCCAT TGGCACCCGT TACCTCACCC GTAATCTGTA A |

AAVrh. 74: (SEQ ID NO: 33)
Nucleotide sequence encoding AAV rh74 capsid protein:
ATGGCTGCCGATGGTTATCTTCCAGATTGGCTCGAGGACAACCTCTCTGAGGGCATTCGCGAGTGGTGGGACCTGAA
ACCTGGAGCCCCGAAACCCAAAGCCAACCAGCAAAAGCAGGACAACGGCCGGGGTCTGGTGCTTCCTGGCTACAAGT
ACCTCGGACCCTTCAACGGACTCGACAAGGGGGAGCCCGTCAACGCGGCGGACGCAGCGGCCCTCGAGCACGACAAG
GCCTACGACCAGCAGCTCCAAGCGGGTGACAATCCGTACCTGCGGTATAATCACGCCGACGCCGAGTTTCAGGAGCG
TCTGCAAGAAGATACGTCTTTTGGGGGCAACCTCGGGCGCGCAGTCTTCCAGGCCAAAAAGCGGGTTCTCGAACCTC
TGGGCCTGGTTGAATCGCCGGTTAAGACGGCTCCTGGAAAGAAGAGACCGGTAGAGCCATCACCCCAGCGCTCTCCA
GACTCCTCTACGGGCATCGGCAAGAAAGGCCAGCAGCCCGCAAAAAAGAGACTCAATTTTGGGCAGACTGGCGACTC
AGAGTCAGTCCCCGACCCTCAACCAATCGGAGAACCACCAGCAGGCCCCTCTGGTCTGGGATCTGGTACAATGGCTG
CAGGCGGTGGCGCTCCAATGGCAGACAATAACGAAGGCGCCGACGGAGTGGGTAGTTCCTCAGGAAATTGGCATTGC
GATTCCACATGGCTGGGCGACAGAGTCATCACCACCAGCACCCGCACCTGGGCCCTGCCCACCTACAACAACCACCT
CTACAAGCAAATCTCCAACGGGACCTCGGGAGGAAGCACCAACGACAACACCTACTTCGGCTACAGCACCCCCTGGG
GGTATTTTGACTTCAACAGATTCCACTGCCACTTTTCACCACGTGACTGGCAGCGACTCATCAACAACAACTGGGGA
TTCCGGCCCAAGAGGCTCAACTTCAAGCTCTTCAACATCCAAGTCAAGGAGGTCACGCAGAATGAAGGCACCAAGAC
CATCGCCAATAACCTTACCAGCACGATTCAGGTCTTTACGGACTCGGAATACCAGCTCCCGTACGTGCTCGGCTCGG
CGCACCAGGGCTGCCTGCCTCCGTTCCCGGCGGACGTCTTCATGATTCCTCAGTACGGGTACCTGACTCTGAACAAT
GGCAGTCAGGCTGTGGGCCGGTCGTCCTTCTACTGCCTGGAGTACTTTCCTTCTCAAATGCTGAGAACGGGCAACAA
CTTTGAATTCAGCTACAACTTCGAGGACGTGCCCTTCCACAGCAGCTACGCGCACAGCCAGAGCCTGGACCGGCTGA
TGAACCCTCTCATCGACCAGTACTTGTACTACCTGTCCCGGACTCAAAGCACGGGCGGTACTGCAGGAACTCAGCAG
TTGCTATTTTCTCAGGCCGGGCCTAACAACATGTCGGCTCAGGCCAAGAACTGGCTACCCGGTCCCTGCTACCGGCA
GCAACGCGTCTCCACGACACTGTCGCAGAACAACAACAGCAACTTTGCCTGGACGGGTGCCACCAAGTATCATCTGA
ATGGCAGAGACTCTCTGGTGAATCCTGGCGTTGCCATGGCTACCCACAAGGACGACGAAGAGCGATTTTTTCCATCC
AGCGGAGTCTTAATGTTTGGGAAACAGGGAGCTGGAAAAGACAACGTGGACTATAGCAGCGTGATGCTAACCAGCGA
GGAAGAAATAAAGACCACCAACCCAGTGGCCACAGAACAGTACGGCGTGGTGGCCGATAACCTGCAACAGCAAAACG
CCGCTCCTATTGTAGGGGCCGTCAATAGTCAAGGAGCCTTACCTGGCATGGTGTGGCAGAACCGGGACGTGTACCTG
CAGGGTCCCATCTGGGCCAAGATTCCTCATACGGACGGCAACTTTCATCCCTCGCCGCTGATGGGAGGCTTTGGACT
GAAGCATCCGCCTCCTCAGATCCTGATTAAAAACACACCTGTTCCCGCCGATCCTCCGACCACCTTCAATCAGGCCA
AGCTGGCTTCTTTCATCACGCAGTACAGTACCGGTCAGGTCAGCGTGGAGATCGAGTGGGAGCTGCAGAAGGAGAAC
AGCAAACGCTGGAACCCAGAGATTCAGTACACTTCCAACTACTACAAATCTACAAATGTGGACTTTGCTGTCAATAC
TGAGGGTACTTATTCCGAGCCTCGCCCCATTGGCACCCGTTACCTCACCCGTAATCTGTAA
AAV2 7M8: AAV2 7m8 is characterized by a 10-amino acid peptide (SEQ ID NO: 34)
LALGETTRPA
ITR Sequence (SEQ ID NO: 35)
CCTGCAGGCAGCTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTCCT
Rep2 Sequence-Contains Rep78 and Rep52 (start codon underlined) (SEQ ID
NO: 36)
ATGCCGGGGTTTTACGAGATTGTGATTAAGGTCCCCAGCGACCTTGACGAGCATCTGCCCGGCATTTCTGACAGC
TTTGTGAACTGGGTGGCCGAGAAGGAATGGGAGTTGCCGCCAGATTCTGACATGGATCTGAATCTGATTGAGCAG
GCACCCCTGACCGTGGCCGAGAAGCTGCAGCGCGACTTTCTGACGGAATGGCGCCGTGTGAGTAAGGCCCCGGAG
GCCCTTTTCTTTGTGCAATTTGAGAAGGGAGAGAGCTACTTCCACATGCACGTGCTCGTGGAAACCACCGGGGTG
AAATCCATGGTTTTGGGACGTTTCCTGAGTCAGATTCGCGAAAAACTGATTCAGAGAATTTACCGCGGGATCGAG
CCGACTTTGCCAAACTGGTTCGCGGTCACAAAGACCAGAAATGGCGCCGGAGGCGGGAACAAGGTGGTGGATGAG
TGCTACATCCCCAATTACTTGCTCCCCAAAACCCAGCCTGAGCTCCAGTGGGCGTGGACTAATATGGAACAGTAT
TTAAGCGCCTGTTTGAATCTCACGGAGCGTAAACGGTTGGTGGCGCAGCATCTGACGCACGTGTCGCAGACGCAG
GAGCAGAACAAAGAGAATCAGAATCCCAATTCTGATGCGCCGGTGATCAGATCAAAAACTTCAGCCAGGTACATG
GAGCTGGTCGGGTGGCTCGTGGACAAGGGGATTACCTCGGAGAAGCAGTGGATCCAGGAGGACCAGGCCTCATAC
ATCTCCTTCAATGCGGCCTCCAACTCGCGGTCCCAAATCAAGGCTGCCTTGGACAATGCGGGAAAGATTATGAGC
CTGACTAAAACCGCCCCCGACTACCTGGTGGGCCAGCAGCCCGTGGAGGACATTTCCAGCAATCGGATTTATAAA
ATTTTGGAACTAAACGGGTACGATCCCCAATATGCGGCTTCCGTCTTTCTGGGATGGGCCACGAAAAAGTTCGGC
AAGAGGAACACCATCTGGCTGTTTGGGCCTGCAACTACCGGGAAGACCAACATCGCGGAGGCCATAGCCCACACT
GTGCCCTTCTACGGGTGCGTAAACTGGACCAATGAGAACTTTCCCTTCAACGACTGTGTCGACAAGATGGTGATC
TGGTGGGAGGAGGGGAAGATGACCGCCAAGGTCGTGGAGTCGGCCAAAGCCATTCTCGGAGGAAGCAAGGTGCGC
GTGGACCAGAAATGCAAGTCCTCGGCCCAGATAGACCCGACTCCCGTGATCGTCACCTCCAACACCAACATGTGC
GCCGTGATTGACGGGAACTCAACGACCTTCGAACACCAGCAGCCGTTGCAAGACCGGATGTTCAAATTTGAACTC
ACCCGCCGTCTGGATCATGACTTTGGGAAGGTCACCAAGCAGGAAGTCAAAGACTTTTTCCGGTGGGCAAAGGAT
CACGTGGTTGAGGTGGAGCATGAATTCTACGTCAAAAAGGGTGGAGCCAAGAAAAGACCCGCCCCCAGTGACGCA
GATATAAGTGAGCCCAAACGGGTGCGCGAGTCAGTTGCGCAGCCATCGACGTCAGACGCGGAAGCTTCGATCAAC
TACGCAGACAGGTACCAAAACAAATGTTCTCGTCACGTGGGCATGAATCTGATGCTGTTTCCCTGCAGACAATGC
GAGAGAATGAATCAGAATTCAAATATCTGCTTCACTCACGGACAGAAAGACTGTTTAGAGTGCTTTCCCGTGTCA
GAATCTCAACCCGTTTCTGTCGTCAAAAAGGCGTATCAGAAACTGTGCTACATTCATCATATCATGGGAAAGGTG
CCAGACGCTTGCACTGCCTGCGATCTGGTCAATGTGGATTTGGATGACTGCATCTTTGAACAATAA
Cap2 Sequence-contains sequentially VP1, VP2, AAP, VP3 (start codons
underlined) (SEQ ID NO: 37)
ATGGCTGCCGATGGTTATCTTCCAGATTGGCTCGAGGACACTCTCTCTGAAGGAATAAGACAGTGGTGGAAGCTC
AAACCTGGCCCACCACCACCAAAGCCCGCAGAGCGGCATAAGGACGACAGCAGGGGTCTTGTGCTTCCTGGGTAC
AAGTACCTCGGACCCTTCAACGGACTCGACAAGGGAGAGCCGGTCAACGAGGCAGACGCCGCGGCCCTCGAGCAC
GACAAAGCCTACGACCGGCAGCTCGACAGCGGAGACAACCCGTACCTCAAGTACAACCACGCCGACGCGGAGTTT
CAGGAGCGCCTTAAAGAAGATACGTCTTTTGGGGGCAACCTCGGACGAGCAGTCTTCCAGGCGAAAAAGAGGGTT
CTTGAACCTCTGGGCCTGGTTGAGGAACCTGTTAAGACGGCTCCGGGAAAAAAGAGGCCGGTAGAGCACTCTCCT
GTGGAGCCAGACTCCTCCTCGGGAACCGGAAAGGCGGGCCAGCAGCCTGCAAGAAAAAGATTGAATTTTGGTCAG
ACTGGAGACGCAGACTCAGTACCTGACCCCCAGCCTCTCGGACAGCCACCAGCAGCCCCCTCTGGTCTGGGAACT
AATACGATGGCTACAGGCAGTGGCGCACCAATGGCAGACAATAACGAGGGCGCCGACGGAGTGGGTAATTCCTCG
GGAAATTGGCATTGCGATTCCACATGGATGGGCGACAGAGTCATCACCACCAGCACCCGAACCTGGGCCCTGCCC
ACCTACAACAACCACCTCTACAAACAAATTTCCAGCCAATCAGGAGCCTCGAACGACAATCACTACTTTGGCTAC
AGCACCCCTTGGGGGTATTTTGACTTCAACAGATTCCACTGCCACTTTTCACCACGTGACTGGCAAAGACTCATC
AACAACAACTGGGGATTCCGACCCAAGAGACTCAACTTCAAGCTCTTTAACATTCAAGTCAAAGAGGTCACGCAG
AATGACGGTACGACGACGATTGCCAATAACCTTACCAGCACGGTTCAGGTGTTTACTGACTCGGAGTACCAGCTC
CCGTACGTCCTCGGCTCGGCGCATCAAGGATGCCTCCCGCCGTTCCCAGCAGACGTCTTCATGGTGCCACAGTAT
GGATACCTCACCCTGAACAACGGGAGTCAGGCAGTAGGACGCTCTTCATTTTACTGCCTGGAGTACTTTCCTTCT
CAGATGCTGCGTACCGGAAACAACTTTACCTTCAGCTACACTTTTGAGGACGTTCCTTTCCACAGCAGCTACGCT
CACAGCCAGAGTCTGGACCGTCTCATGAATCCTCTCATCGACCAGTACCTGTATTACTTGAGCAGAACAAACACT
CCAAGTGGAACCACCACGCAGTCAAGGCTTCAGTTTTCTCAGGCCGGAGCGAGTGACATTCGGGACCAGTCTAGG
AACTGGCTTCCTGGACCCTGTTACCGCCAGCAGCGAGTATCAAAGACATCTGCGGATAACAACAACAGTGAATAC
TCGTGGACTGGAGCTACCAAGTACCACCTCAATGGCAGAGACTCTCTGGTGAATCCGGGCCCGGCCATGGCAAGC
CACAAGGACGATGAAGAAAAGTTTTTTCCTCAGAGCGGGGTTCTCATCTTTGGGAAGCAAGGCTCAGAGAAAACA
AATGTGGACATTGAAAAGGTCATGATTACAGACGAAGAGGAAATCAGGACAACCAATCCCGTGGCTACGGAGCAG
TATGGTTCTGTATCTACCAACCTCCAGAGAGGCAACAGACAAGCAGCTACCGCAGATGTCAACACACAAGGCGTT
CTTCCAGGCATGGTCTGGCAGGACAGAGATGTGTACCTTCAGGGGCCCATCTGGGCAAAGATTCCACACACGGAC
GGACATTTTCACCCCTCTCCCCTCATGGGTGGATTCGGACTTAAACACCCTCCTCCACAGATTCTCATCAAGAAC
ACCCCGGTACCTGCGAATCCTTCGACCACCTTCAGTGCGGCAAAGTTTGCTTCCTTCATCACACAGTACTCCACG
GGACAGGTCAGCGTGGAGATCGAGTGGGAGCTGCAGAAGGAAAACAGCAAACGCTGGAATCCCGAAATTCAGTAC
ACTTCCAACTACAACAAGTCTGTTAATGTGGACTTTACTGTGGACACTAATGGCGTGTATTCAGAGCCTCGCCCC
ATTGGCACCAGATACCTGACTCGTAATCTGTAA
Cap5 Sequence-contains sequentially VP1, VP2, AAP, VP3
underlined) (SEQ ID NO: 38)
ATGGCTTTTGTTGATCACCCTCCAGATTGGTTGGAAGAAGTTGGTGAAGGTCTTCGCGAGTTTTTGGGCCTTGAA
GCGGGCCCACCGAAACCAAAACCCAATCAGCAGCATCAAGATCAAGCCCGTGGTCTTGTGCTGCCTGGTTATAAC
TATCTCGGACCCGGAAACGGTCTCGATCGAGGAGAGCCTGTCAACAGGGCAGACGAGGTCGCGCGAGAGCACGAC
ATCTCGTACAACGAGCAGCTTGAGGCGGGAGACAACCCCTACCTCAAGTACAACCACGCGGACGCCGAGTTTCAG
GAGAAGCTCGCCGACGACACATCCTTCGGGGGAAACCTCGGAAAGGCAGTCTTTCAGGCCAAGAAAAGGGTTCTC
GAACCTTTTGGCCTGGTTGAAGAGGGTGCTAAGACGGCCCCTACCGGAAAGCGGATAGACGACCACTTTCCAAAA
AGAAAGAAGGCTCGGACCGAAGAGGACTCCAAGCCTTCCACCTCGTCAGACGCCGAAGCTGGACCCAGCGGATCC
CAGCAGCTGCAAATCCCAGCCCAACCAGCCTCAAGTTTGGGAGCTGATACAATGTCTGCGGGAGGTGGCGGCCCA
TTGGGCGACAATAACCAAGGTGCCGATGGAGTGGGCAATGCCTCGGGAGATTGGCATTGCGATTCCACGTGGATG
GGGGACAGAGTCGTCACCAAGTCCACCCGAACCTGGGTGCTGCCCAGCTACAACAACCACCAGTACCGAGAGATC
AAAAGCGGCTCCGTCGACGGAAGCAACGCCAACGCCTACTTTGGATACAGCACCCCCTGGGGGTACTTTGACTTT
AACCGCTTCCACAGCCACTGGAGCCCCCGAGACTGGCAAAGACTCATCAACAACTACTGGGGCTTCAGACCCCGG
TCCCTCAGAGTCAAAATCTTCAACATTCAAGTCAAAGAGGTCACGGTGCAGGACTCCACCACCACCATCGCCAAC
AACCTCACCTCCACCGTCCAAGTGTTTACGGACGACGACTACCAGCTGCCCTACGTCGTCGGCAACGGGACCGAG
GGATGCCTGCCGGCCTTCCCTCCGCAGGTCTTTACGCTGCCGCAGTACGGTTACGCGACGCTGAACCGCGACAAC
ACAGAAAATCCCACCGAGAGGAGCAGCTTCTTCTGCCTAGAGTACTTTCCCAGCAAGATGCTGAGAACGGGCAAC
AACTTTGAGTTTACCTACAACTTTGAGGAGGTGCCCTTCCACTCCAGCTTCGCTCCCAGTCAGAACCTCTTCAAG
CTGGCCAACCCGCTGGTGGACCAGTACTTGTACCGCTTCGTGAGCACAAATAACACTGGCGGAGTCCAGTTCAAC
AAGAACCTGGCCGGGAGATACGCCAACACCTACAAAAACTGGTTCCCGGGGCCCATGGGCCGAACCCAGGGCTGG
AACCTGGGCTCCGGGGTCAACCGCGCCAGTGTCAGCGCCTTCGCCACGACCAATAGGATGGAGCTCGAGGGCGCG
AGTTACCAGGTGCCCCCGCAGCCGAACGGCATGACCAACAACCTCCAGGGCAGCAACACCTATGCCCTGGAGAAC
ACTATGATCTTCAACAGCCAGCCGGCGAACCCGGGCACCACCGCCACGTACCTCGAGGGCAACATGCTCATCACC
AGCGAGAGCGAGACGCAGCCGGTGAACCGCGTGGCGTACAACGTCGGCGGGCAGATGGCCACCAACAACCAGAGC
TCCACCACTGCCCCCGCGACCGGCACGTACAACCTCCAGGAAATCGTGCCCGGCAGCGTGTGGATGGAGAGGGAC
GTGTACCTCCAAGGACCCATCTGGGCCAAGATCCCAGAGACGGGGGCGCACTTTCACCCCTCTCCGGCCATGGGC
GGATTCGGACTCAAACACCCACCGCCCATGATGCTCATCAAGAACACGCCTGTGCCCGGAAATATCACCAGCTTC
TCGGACGTGCCCGTCAGCAGCTTCATCACCCAGTACAGCACCGGGCAGGTCACCGTGGAGATGGAGTGGGAGCTC
AAGAAGGAAAACTCCAAGAGGTGGAACCCAGAGATCCAGTACACAAACAACTACAACGACCCCCAGTTTGTGGAC
TTTGCCCCGGACAGCACCGGGGAATACAGAAGCACCAGACCTATCGGAACCCGATACCTTACCCGACCCCTTTAA

### Example 31—Adenovirus Polynucleotide Sequences

Adenovirus (Ad) polynucleotides can be selected from any serotype, and representative polynucleotides are exemplified below.

```
E2A Full Sequence
(SEQ ID NO: 39)
  CGACCGCACCCTGTGACGAAAGCCGCCCGCAAGCTGCGCCCCTGAGTTAGTCATCTGAACTTCGGCCTGGGCGT
CTCTGGGAAGTACCACAGTGGTGGGAGCGGGACTTTCCTGGTACACCAGGGCAGCGGGCCAACTACGGGGATTAA
GGTTATTACGAGGTGTGGTGGTAATAGCCGCCTGTTCGAGGAGAATTCGGTTTCGGTGGGCGCGGATTCCGTTGA
CCCGGGATATCATGTGGGGTCCCGCGCTCATGTAGTTTATTCGGGTTGAGTAGTCTTGGGCAGCTCCAGCCGCAA
GTCCCATTTGTGGCTGGTAACTCCACATGTAGGGCGTGGGAATTTCCTTGCTCATAATGGCGCTGACGACAGGTG
CTGGCGCCGGGTGTGGCCGCTGGAGATGACGTAGTTTTCGCGCTTAAATTTGAGAAAGGGCGCGAAACTAGTCCT
TAAGAGTCAGCGCGCAGTATTTGCTGAAGAGAGCCTCCGCGTCTTCCAGCGTGCGCCGAAGCTGATCTTCGCTTT
TGTGATACAGGCAGCTGCGGGTGAGGGAGCGCAGAGACCTGTTTTTTATTTTCAGCTCTTGTTCTTGGCCCCTGC
TTTGTTGAAATATAGCATACAGAGTGGGAAAAATCCTATTTCTAAGCTCGCGGGTCGATACGGGTTCGTTGGGCG
CCAGACGCAGCGCTCCTCCTCCTGCTGCTGCCGCCGCTGTGGATTTCTTGGGCTTTGTCAGAGTCTTGCTATCCG
GTCGCCTTTGCTTCTGTGTGACCGCTGCTGTTGCTGCCGCTGCCGCTGCCGCCGGTGCAGTAGGGGCTGTAGAGA
TGACGGTAGTAATGCAGGATGTTACGGGGGAAGGCCACGCCGTGATGGTAGAGAAGAAAGCGGCGGGCGAAGGAG
ATGTTGCCCCCACAGTCTTGCAAGCAAGCAACTATGGCGTTCTTGTGCCCGCGCCACGAGCGGTAGCCTTGGCGC
TGTTGTTGCTCTTGGGCTAACGGCGGCGGCTGCTTAGACTTACCGGCCCTGGTTCCAGTGGTGTCCCATCTACGG
TTGGGTCGGCGAACAGGCAGTGCCGGCGGCGCCTGAGGAGCGGAGGTTGTAGCGATGCTGGGAACGGTTGCCAAT
TTCTGGGGCGCCGGCGAGGGGAATGCGACCGAGGGTGACGGTGTTTCGTCTGACACCTCTTCGGCCTCGGAAGCT
TCGTCTAGGCTGTCCCAGTCTTCCATCATCTCCTCCTCCTCGTCCAAAACCTCCTCTGCCTGACTGTCCCAGTAT
TCCTCCTCGTCCGTGGGTGGCGGCGGCGGCAGCTGCAGCTTCTTTTTGGGTGCCATCCTGGGAAGCAAGGGCCCG
CGGCTGCTGATAGGGCTGCGGCGGCGGGGGGATTGGGTTGAGCTCCTCGCCGGACTGGGGGTCCAGGTAAACCCC
CCGTCCCTTTCGTAGCAGAAACTCTTGGCGGGCTTTGTTGATGGCTTGCAATTGGCCAAGGATGTGGCCCTGGGT
AATGACGCAGGCGGTAAGCTCCGCATTTGGCGGGCGGGATTGGTCTTCGTAGAACCTAATCTCGTGGGCGTGGTA
GTCCTCAGGTACAAATTTGCGAAGGTAAGCCGACGTCCACAGCCCCGGAGTGAGTTTCAACCCCGGAGCCGCGGA
CTTTTCGTCAGGCGAGGGACCCTGCAGCTCAAAGGTACCGATAATTTGACTTTCGCTAAGCAGTTGCGAATTGCA
GACCAGGGAGCGGTGCGGGGTGCATAGGTTGCAGCGACAGTGACACTCCAGTAGGCCGTCACCGCTCACGTCTTC
CATGATGTCGGAGTGGTAGGCAAGGTAGTTGGCTAGCTGCAGAAGGTAGCAGTGACCCCAAAGCGGCGGAGGGCA
TTCACGGTACTTAATGGGCACAAAGTCGCTAGGAAGCGCACAGCAGGTGGCGGGCAGAATTCCTGAACGCTCTAG
GATAAAGTTCCTAAAGTTTTGCAACATGCTTTGACTGGTGAAGTCTGGCAGACCCTGTTGCAGGGTTTTAAGCAG
GCGTTCGGGGAAGATAATGTCCGCCAGGTGCGCGGCCACGGAGCGCTCGTTGAAGGCCGTCCATAGGTCCTTCAA
GTTTTGCTTTAGCAGCTTCTGCAGCTCCTTTAGGTTGCGCTCCTCCAGGCATTGCTGCCACACGCCCATGGCCGT
TTGCCAGGTGTAGCACAGAAATAAGTAAACGCAGTCGCGGACGTAGTCGCGGCGCGCCTCGCCCTTGAGCGTGGA
ATGAAGCACGTTTTGCCCGAGGCGGTTTTCGTGCAAAATTCCAAGGTAGGAGACCAGGTTGCAGAGCTCCACGTT
GGAAATTTTGCAGGCCTGGCGCACGTAGCCCTGGCGAAAGGTGTAGTGCAACGTTTCCTCTAGCTTGCGCTGCAT
CTCCGGGTCAGCAAAGAACCGCTGCATGCACTCAAGCTCCACGGTAACAAGCACTGCGGCCATCATTAGCTTGCG
TCGCTCCTCCAAGTCGGCAGGCTCGCGCGTCTCAAGCCAGCGCGCCAGCTGCTCATCGCCAACTGCGGGTAGGCC
CTCCTCGGTTTGTTCTTGCAAGTTTGCATCCCTCTCCAGGGGTCGTGCACGGCGCACGATCAGCTCGCTCATGAC
TGTGCTCATAACCTTGGGGGGTAGGTTAAGTGCCGGGTAGGCAAAGTGGGTGACCTCGATGCTGCGTTTCAGCAC
GGCTAGGCGCGCGTTGTCACCCTCAAGTTCCACCAGCACTCCACAGTGACTTTCATTTTCGCTGTTTTCTTGTTG
CAGAGCGTTTGCCGCGCGTTTCTCGTCGCGTCCAAGACCCTCAAAGATTTTTGGCACTTCGTCGAGCGAGGCGAT
ATCAGGTATGACAGCGCCCTGCCGCAAGGCCAGCTGCTTGTCCGCTCGGCTGCGGTTGGCACGGCAGGATAGGGG
TATCTTGCAGTTTTGGAAAAAGATGTGATAGGTGGCAAGCACCTCTGGCACGGCAAATACGGGGTAGAAGTTGAG
GCGCGGGTTGGGCTCGCATGTGCCGTTTTCTTGGCGTTTGGGGGGTACGCGCGGTGAGAACAGGTGGCGTTCGTA
GGCAAGGCTGACATCCGCTATGGCGAGGGGCACATCGCTGCGCTCTTGCAACGCGTCGCAGATAATGGCGCACTG
GCGCTGCAGATGCTTCAACAGCACGTCGTCTCCCACATCTAGGTAGTCGCCATGCCTTTGGTCCCCCCGCCCGAC
TTGTTCCTCGTTTGCCTCTGCGTCGTCCTGGTCTTGCTTTTTATCCTCTGTTGGTACTGAGCGATCCTCGTCGTC
TTCGCTTACAAAACCTGGGTCCTGCTCGATAATCACTTCCTCCTCCTCAAGCGGGGGTGCCTCGACGGGGAAGGT
GGTAGGCGCGTTGGCGGCATCGGTGGAGGCGGTGGTGGCGAACTCAAAGGGGGCGGTTAGGCTGTCCTCCTTCTC
GACTGACTCCATGATCTTTTTCTGCCTATAGGAGAAGGAAATGGCCAGTCGGGAAGAGGAGCAGCGCGAAACCAC
CCCCGAGCGCGGACGCGGTGCGGCGCGACGTCCACCAACCATGGAGGACGTGTCGTCCCCGTCGCCGTCGCCGCC
GCCTCCCCGCGCGCCCCCAAAAAAGCGGCTGAGGCGGCGTCTCGAGTCCGAGGACGAAGAAGACTCGTCACAAGA
TGCGCTGGTGCCGCGCACACCCAGCCCGCGGCCATCGACCTCGACGGCGGATTTGGCCATTGCGTCCAAAAAGAA
AAAGAAGCGCCCCTCTCCCAAGCCCGAGCGCCCGCCATCCCCAGAGGTGATCGTGGACAGCGAGGAAGAAAGAGA
AGATGTGGCGCTACAAATGGTGGGTTTCAGCAACCCACCGGTGCTAATCAAGCACGGCAAGGGAGGTAAGCGCAC
GGTGCGGCGGCTGAATGAAGACGACCCAGTGGCGCGGGGTATGCGGACGCAAGAGGAAAAGGAAGAGTCCAGTGA
AGCGGAAAGTGAAAGCACGGTGATAAACCCGCTGAGCCTGCCGATCGTGTCTGCGTGGGAGAAGGGCATGGAGGC
TGCGCGCGCGTTGATGGACAAGTACCACGTGGATAACGATCTAAAGGCAAACTTCAAGCTACTGCCTGACCAAGT
GGAAGCTCTGGCGGCCGTATGCAAGACCTGGCTAAACGAGGAGCACCGCGGGTTGCAGCTGACCTTCACCAGCAA
CAAGACCTTTGTGACGATGATGGGGCGATTCCTGCAGGCGTACCTGCAGTCGTTTGCAGAGGTAACCTACAAGCA
CCACGAGCCCACGGGCTGCGCGTTGTGGCTGCACCGCTGCGCTGAGATCGAAGGCGAGCTTAAGTGTCTACACGG
GAGCATTATGATAAATAAGGAGCACGTGATTGAAATGGATGTGACGAGCGAAAACGGGCAGCGCGCGCTGAAGGA
GCAGTCTAGCAAGGCCAAGATCGTGAAGAACCGGTGGGGCCGAAATGTGGTGCAGATCTCCAACACCGACGCAAG
GTGCTGCGTGCATGACGCGGCCTGTCCGGCCAATCAGTTTTCCGGCAAGTCTTGCGGCATGTTCTTCTCTGAAGG
CGCAAAGGCTCAGGTGGCTTTTAAGCAGATCAAGGCTTTCATGCAGGCGCTGTATCCTAACGCCCAGACCGGGCA
CGGTCACCTTCTGATGCCACTACGGTGCGAGTGCAACTCAAAGCCTGGGCATGCACCCTTTTTGGGAAGGCAGCT
ACCAAAGTTGACTCCGTTCGCCCTGAGCAACGCGGAGGACCTGGACGCGGATCTGATCTCCGACAAGAGCGTGCT
GGCCAGCGTGCACCACCCGGCGCTGATAGTGTTCCAGTGCTGCAACCCTGTGTATCGCAACTCGCGCGCGCAGGG
CGGAGGCCCCAACTGCGACTTCAAGATATCGGCGCCCGACCTGCTAAACGCGTTGGTGATGGTGCGCAGCCTGTG
GAGTGAAAACTTCACCGAGCTGCCGCGGATGGTTGTGCCTGAGTTTAAGTGGAGCACTAAACACCAGTATCGCAA
CGTGTCCCTGCCAGTGGCGCATAGCGATGCGCGGCAGAACCCCTTTGATTTTTAAACGGCGCAGACGGCAAGGGT
GGGGGGTAAATAATCACCCGAGAGTGTACAAATAAAAACATTTGCCTTTATTGAAAGTGTCTCCTAGTACATTAT
TTTTACATGTTTTTCAAGTGACAAAAAGAAGTGGCGCTCCTAATCTGCGCACTGTGGCTGCGGAAGTAGGGCGAG
TGGCGCTCCAGGAAGCTGTAGAGCTGTTCCTGGTTGCGACGCAGGGTGGGCTGTACCTGGGGACTGTTAAGCATG
GAGTTGGGTACC
E2A ORF Sequence
(SEQ ID NO: 40)
ATGGCCAGTCGGGAAGAGGAGCAGCGCGAAACCACCCCCGAGCGCGGACGCGGTGCGGCGCGACGTCCACCAACC
ATGGAGGACGTGTCGTCCCCGTCGCCGTCGCCGCCGCCTCCCCGCGCGCCCCCAAAAAAGCGGCTGAGGCGGCGT
CTCGAGTCCGAGGACGAAGAAGACTCGTCACAAGATGCGCTGGTGCCGCGCACACCCAGCCCGCGGCCATCGACC
TCGACGGCGGATTTGGCCATTGCGTCCAAAAAGAAAAAGAAGCGCCCCTCTCCCAAGCCCGAGCGCCCGCCATCC
CCAGAGGTGATCGTGGACAGCGAGGAAGAAAGAGAAGATGTGGCGCTACAAATGGTGGGTTTCAGCAACCCACCG
GTGCTAATCAAGCACGGCAAGGGAGGTAAGCGCACGGTGCGGCGGCTGAATGAAGACGACCCAGTGGCGCGGGGT
ATGCGGACGCAAGAGGAAAAGGAAGAGTCCAGTGAAGCGGAAAGTGAAAGCACGGTGATAAACCCGCTGAGCCTG
CCGATCGTGTCTGCGTGGGAGAAGGGCATGGAGGCTGCGCGCGCGTTGATGGACAAGTACCACGTGGATAACGAT
CTAAAGGCAAACTTCAAGCTACTGCCTGACCAAGTGGAAGCTCTGGCGGCCGTATGCAAGACCTGGCTAAACGAG
GAGCACCGCGGGTTGCAGCTGACCTTCACCAGCAACAAGACCTTTGTGACGATGATGGGGCGATTCCTGCAGGCG
TACCTGCAGTCGTTTGCAGAGGTAACCTACAAGCACCACGAGCCCACGGGCTGCGCGTTGTGGCTGCACCGCTGC
GCTGAGATCGAAGGCGAGCTTAAGTGTCTACACGGGAGCATTATGATAAATAAGGAGCACGTGATTGAAATGGAT
GTGACGAGCGAAAACGGGCAGCGCGCGCTGAAGGAGCAGTCTAGCAAGGCCAAGATCGTGAAGAACCGGTGGGGC
CGAAATGTGGTGCAGATCTCCAACACCGACGCAAGGTGCTGCGTGCATGACGCGGCCTGTCCGGCCAATCAGTTT
TCCGGCAAGTCTTGCGGCATGTTCTTCTCTGAAGGCGCAAAGGCTCAGGTGGCTTTTAAGCAGATCAAGGCTTTC
ATGCAGGCGCTGTATCCTAACGCCCAGACCGGGCACGGTCACCTTCTGATGCCACTACGGTGCGAGTGCAACTCA
AAGCCTGGGCATGCACCCTTTTTGGGAAGGCAGCTACCAAAGTTGACTCCGTTCGCCCTGAGCAACGCGGAGGAC
CTGGACGCGGATCTGATCTCCGACAAGAGCGTGCTGGCCAGCGTGCACCACCCGGCGCTGATAGTGTTCCAGTGC
TGCAACCCTGTGTATCGCAACTCGCGCGCGCAGGGCGGAGGCCCCAACTGCGACTTCAAGATATCGGCGCCCGAC
CTGCTAAACGCGTTGGTGATGGTGCGCAGCCTGTGGAGTGAAAACTTCACCGAGCTGCCGCGGATGGTTGTGCCT
GAGTTTAAGTGGAGCACTAAACACCAGTATCGCAACGTGTCCCTGCCAGTGGCGCATAGCGATGCGCGGCAGAAC
CCCTTTGATTTTTAA
E4 Full Sequence
(SEQ ID NO: 41)
CCCGGGCGTTTTAGGGCGGAGTAACTTGCATGTATTGGGAATTGTAGTTTTTTTAAAATGGGAAGTGACGTATCG
TGGGAAAACGGAAGTGAAGATTTGAGGAAGTTGTGGGTTTTTTGGCTTTCGTTTCTGGGCGTAGGTTCGCGTGCG
GTTTTCTGGGTGTTTTTTGTGGACTTTAACCGTTACGTCATTTTTTAGTCCTATATATACTCGCTCTGTACTTGG
CCCTTTTTACACTGTGACTGATTGAGCTGGTGCCGTGTCGAGTGGTGTTTTTTAATAGGTTTTTTTACTGGTAAG
GCTGACTGTTATGGCTGCCGCTGTGGAAGCGCTGTATGTTGTTCTGGAGCGGGAGGGTGCTATTTTGCCTAGGCA
GGAGGGTTTTTCAGGTGTTTATGTGTTTTTCTCTCCTATTAATTTTGTTATACCTCCTATGGGGGCTGTAATGTT
GTCTCTACGCCTGCGGGTATGTATTCCCCCGGGCTATTTCGGTCGCTTTTTAGCACTGACCGATGTTAACCAACC
TGATGTGTTTACCGAGTCTTACATTATGACTCCGGACATGACCGAGGAACTGTCGGTGGTGCTTTTTAATCACGG
TGACCAGTTTTTTTACGGTCACGCCGGCATGGCCGTAGTCCGTCTTATGCTTATAAGGGTTGTTTTTCCTGTTGT
AAGACAGGCTTCTAATGTTTAAATGTTTTTTTTTTTGTTATTTTATTTTGTGTTTAATGCAGGAACCCGCAGACA
TGTTTGAGAGAAAAATGGTGTCTTTTTCTGTGGTGGTTCCGGAACTTACCTGCCTTTATCTGCATGAGCATGACT
ACGATGTGCTTGCTTTTTTGCGCGAGGCTTTGCCTGATTTTTTGAGCAGCACCTTGCATTTTATATCGCCGCCCA
TGCAACAAGCTTACATAGGGGCTACGCTGGTTAGCATAGCTCCGAGTATGCGTGTCATAATCAGTGTGGGTTCTT
TTGTCATGGTTCCTGGCGGGGAAGTGGCCGCGCTGGTCCGTGCAGACCTGCACGATTATGTTCAGCTGGCCCTGC
GAAGGGACCTACGGGATCGCGGTATTTTTGTTAATGTTCCGCTTTTGAATCTTATACAGGTCTGTGAGGAACCTG
AATTTTTGCAATCATGATTCGCTGCTTGAGGCTGAAGGTGGAGGGCGCTCTGGAGCAGATTTTTACAATGGCCGG
ACTTAATATTCGGGATTTGCTTAGAGACATATTGATAAGGTGGCGAGATGAAAATTATTTGGGCATGGTTGAAGG
TGCTGGAATGTTTATAGAGGAGATTCACCCTGAAGGGTTTAGCCTTTACGTCCACTTGGACGTGAGGGCAGTTTG
CCTTTTGGAAGCCATTGTGCAACATCTTACAAATGCCATTATCTGTTCTTTGGCTGTAGAGTTTGACCACGCCAC
CGGAGGGGAGCGCGTTCACTTAATAGATCTTCATTTTGAGGTTTTGGATAATCTTTTGGAATAAAAAAAAAAAAA
CATGGTTCTTCCAGCTCTTCCCGCTCCTCCCGTGTGTGACTCGCAGAACGAATGTGTAGGTTGGCTGGGTGTGGC
TTATTCTGCGGTGGTGGATGTTATCAGGGCAGCGGCGCATGAAGGAGTTTACATAGAACCCGAAGCCAGGGGGCG
CCTGGATGCTTTGAGAGAGTGGATATACTACAACTACTACACAGAGCGAGCTAAGCGACGAGACCGGAGACGCAG
ATCTGTTTGTCACGCCCGCACCTGGTTTTGCTTCAGGAAATATGACTACGTCCGGCGTTCCATTTGGCATGACAC
TACGACCAACACGATCTCGGTTGTCTCGGCGCACTCCGTACAGTAGGGATCGCCTACCTCCTTTTGAGACAGAGA
CCCGCGCTACCATACTGGAGGATCATCCGCTGCTGCCCGAATGTAACACTTTGACAATGCACAACGTGAGTTACG
TGCGAGGTCTTCCCTGCAGTGTGGGATTTACGCTGATTCAGGAATGGGTTGTTCCCTGGGATATGGTTCTGACGC
GGGAGGAGCTTGTAATCCTGAGGAAGTGTATGCACGTGTGCCTGTGTTGTGCCAACATTGATATCATGACGAGCA
TGATGATCCATGGTTACGAGTCCTGGGCTCTCCACTGTCATTGTTCCAGTCCCGGTTCCCTGCAGTGCATAGCCG
GCGGGCAGGTTTTGGCCAGCTGGTTTAGGATGGTGGTGGATGGCGCCATGTTTAATCAGAGGTTTATATGGTACC
GGGAGGTGGTGAATTACAACATGCCAAAAGAGGTAATGTTTATGTCCAGCGTGTTTATGAGGGGTCGCCACTTAA
TCTACCTGCGCTTGTGGTATGATGGCCACGTGGGTTCTGTGGTCCCCGCCATGAGCTTTGGATACAGCGCCTTGC
ACTGTGGGATTTTGAACAATATTGTGGTGCTGTGCTGCAGTTACTGTGCTGATTTAAGTGAGATCAGGGTGCGCT
GCTGTGCCCGGAGGACAAGGCGTCTCATGCTGCGGGCGGTGCGAATCATCGCTGAGGAGACCACTGCCATGTTGT
ATTCCTGCAGGACGGAGCGGCGGCGGCAGCAGTTTATTCGCGCGCTGCTGCAGCACCACCGCCCTATCCTGATGC
ACGATTATGACTCTACCCCCATGTAGGCGTGGACTTCCCCTTCGCCGCCCGTTGAGCAACCGCAAGTTGGACAGC
AGCCTGTGGCTCAGCAGCTGGACAGCGACATGAACTTAAGCGAGCTGCCCGGGGAGTTTATTAATATCACTGATG
AGCGTTTGGCTCGACAGGAAACCGTGTGGAATATAACACCTAAGAATATGTCTGTTACCCATGATATGATGCTTT
TTAAGGCCAGCCGGGGAGAAAGGACTGTGTACTCTGTGTGTTGGGAGGGAGGTGGCAGGTTGAATACTAGGGTTC
TGTGAGTTTGATTAAGGTACGGTGATCAATATAAGCTATGTGGTGGTGGGGCTATACTACTGAATGAAAAATGAC
TTGAAATTTTCTGCAATTGAAAAATAAACACGTTGAAACATAACATGCAACAGGTTCACGATTCTTTATTCCTGG
GCAATGTAGGAGAAGGTGTAAGAGTTGGTAGCAAAAGTTTCAGTGGTGTATTTTCCACTTTCCCAGGACCATGTA
AAAGACATAGAGTAAGTGCTTACCTCGCTAGTTTCTGTGGATTCACTAGAA
E4 Orf6 Sequence
(SEQ ID NO: 42)
ATGACTACGTCCGGCGTTCCATTTGGCATGACACTACGACCAACACGATCTCGGTTGTCTCGGCGCACTCCGTAC
AGTAGGGATCGCCTACCTCCTTTTGAGACAGAGACCCGCGCTACCATACTGGAGGATCATCCGCTGCTGCCCGAA
TGTAACACTTTGACAATGCACAACGTGAGTTACGTGCGAGGTCTTCCCTGCAGTGTGGGATTTACGCTGATTCAG
GAATGGGTTGTTCCCTGGGATATGGTTCTGACGCGGGAGGAGCTTGTAATCCTGAGGAAGTGTATGCACGTGTGC
CTGTGTTGTGCCAACATTGATATCATGACGAGCATGATGATCCATGGTTACGAGTCCTGGGCTCTCCACTGTCAT
TGTTCCAGTCCCGGTTCCCTGCAGTGCATAGCCGGCGGGCAGGTTTTGGCCAGCTGGTTTAGGATGGTGGTGGAT
GGCGCCATGTTTAATCAGAGGTTTATATGGTACCGGGAGGTGGTGAATTACAACATGCCAAAAGAGGTAATGTTT
ATGTCCAGCGTGTTTATGAGGGGTCGCCACTTAATCTACCTGCGCTTGTGGTATGATGGCCACGTGGGTTCTGTG
GTCCCCGCCATGAGCTTTGGATACAGCGCCTTGCACTGTGGGATTTTGAACAATATTGTGGTGCTGTGCTGCAGT
TACTGTGCTGATTTAAGTGAGATCAGGGTGCGCTGCTGTGCCCGGAGGACAAGGCGTCTCATGCTGCGGGCGGTG
CGAATCATCGCTGAGGAGACCACTGCCATGTTGTATTCCTGCAGGACGGAGCGGCGGCGGCAGCAGTTTATTCGC
GCGCTGCTGCAGCACCACCGCCCTATCCTGATGCACGATTATGACTCTACCCCCATGTAG
VA Sequence (VA transcripts I and II are underlined)
(SEQ ID NO: 43)
CGTAATCCGTAGATGTACCTGGACATCCAGGTGATGCCGGCGGCGGTGGTGGAGGCGCGCGGAAAGTCGCGGAC
GCGGTTCCAGATGTTGCGCAGCGGCAAAAAGTGCTCCATGGTCGGGACGCTCTGGCCGGTGAGGCGTGCGCAGTC
GTTGACGCTCTAGACCGTGCAAAAGGAGAGCCTGTAAGCGGGCACTCTTCCGTGGTCTGGTGGATAAATTCGCAA
GGGTATCATGGCGGACGACCGGGGTTCGAACCCCGGATCCGGCCGTCCGCCGTGATCCATGCGGTTACCGCCCGC
GTGTCGAACCCAGGTGTGCGACGTCAGACAACGGGGGAGCGCTCCTTTTGGCTTCCTTCCAGGCGCGGCGGCTGC
TGCGCTAGCTTTTTTGGCCACTGGCCGCGCGCGGCGTAAGCGGTTAGGCTGGAAAGCGAAAGCATTAAGTGGCTC
GCTCCCTGTAGCCGGAGGGTTATTTTCCAAGGGTTGAGTCGCAGGACCCCCGGTTCGAGTCTCGGGCCGGCCGGA
CTGCGGCGAACGGGGGTTTGCCTCCCCGTCATGCAAGACCCCGCTTGCAAATTCCTCCGGAAACAGGGACGAGCC
  CCTTTTTTGCTTTTCCCAGATGCATCCGGTGCTGCGGCAGATGCGCCCCCCTCCTCAGCAGCGGCAAGAGCAAGA
GCAGCGGCAGACATGCAGGGCACCCTCCCCTTCTCCTACCGCGTCAGGAGGGGCAACATCCTACATCGA
Sequences for E1A and E1B are both contained within Accession AY339865.1
Ad5 E1A
Two proteins can be transcribed, a 32 kDa protein (first accession number)
and a 27 kDa protein (second accession number). These are both splice
variants from the transcript:
Accession 1: AAQ19284.1
Accession 2: AAQ19285.1
(SEQ ID NO: 44)
ATGAGACATATTATCTGCCACGGAGGTGTTATTACCGAAGAAATGGCCGCCAGTCTTTTGGACCAGCTGATCGAA
GAGGTACTGGCTGATAATCTTCCACCTCCTAGCCATTTTGAACCACCTACCCTTCACGAACTGTATGATTTAGAC
GTGACGGCCCCCGAAGATCCCAACGAGGAGGCGGTTTCGCAGATTTTTCCCGACTCTGTAATGTTGGCGGTGCAG
GAAGGGATTGACTTACTCACTTTTCCGCCGGCGCCCGGTTCTCCGGAGCCGCCTCACCTTTCCCGGCAGCCCGAG
CAGCCGGAGCAGAGAGCCTTGGGTCCGGTTTCTATGCCAAACCTTGTACCGGAGGTGATCGATCTTACCTGCCAC
GAGGCTGGCTTTCCACCCAGTGACGACGAGGATGAAGAGGGTGAGGAGTTTGTGTTAGATTATGTGGAGCACCCC
GGGCACGGTTGCAGGTCTTGTCATTATCACCGGAGGAATACGGGGGACCCAGATATTATGTGTTCGCTTTGCTAT
ATGAGGACCTGTGGCATGTTTGTCTACAGTCCTGTGTCTGAACCTGAGCCTGAGCCCGAGCCAGAACCGGAGCCT
GCAAGACCTACCCGCCGTCCTAAAATGGCGCCTGCTATCCTGAGACGCCCGACATCACCTGTGTCTAGAGAATGC
AATAGTAGTACGGATAGCTGTGACTCCGGTCCTTCTAACACACCTCCTGAGATACACCCGGTGGTCCCGCTGTGC
CCCATTAAACCAGTTGCCGTGAGAGTTGGTGGGCGTCGCCAGGCTGTGGAATGTATCGAGGACTTGCTTAACGAG
CCTGGGCAACCTTTGGACTTGAGCTGTAAACGCCCCAGGCCATAA
(SEQ ID NO: 45)
ATGAGACATATTATCTGCCACGGAGGTGTTATTACCGAAGAAATGGCCGCCAGTCTTTTGGACCAGCTGATCGAA
GAGGTACTGGCTGATAATCTTCCACCTCCTAGCCATTTTGAACCACCTACCCTTCACGAACTGTATGATTTAGAC
GTGACGGCCCCCGAAGATCCCAACGAGGAGGCGGTTTCGCAGATTTTTCCCGACTCTGTAATGTTGGCGGTGCAG
GAAGGGATTGACTTACTCACTTTTCCGCCGGCGCCCGGTTCTCCGGAGCCGCCTCACCTTTCCCGGCAGCCCGAG
CAGCCGGAGCAGAGAGCCTTGGGTCCGGTTTCTATGCCAAACCTTGTACCGGAGGTGATCGATCTTACCTGCCAC
GAGGCTGGCTTTCCACCCAGTGACGACGAGGATGAAGAGGGTCCTGTGTCTGAACCTGAGCCTGAGCCCGAGCCA
GAACCGGAGCCTGCAAGACCTACCCGCCGTCCTAAAATGGCGCCTGCTATCCTGAGACGCCCGACATCACCTGTG
TCTAGAGAATGCAATAGTAGTACGGATAGCTGTGACTCCGGTCCTTCTAACACACCTCCTGAGATACACCCGGTG
GTCCCGCTGTGCCCCATTAAACCAGTTGCCGTGAGAGTTGGTGGGCGTCGCCAGGCTGTGGAATGTATCGAGGAC
TTGCTTAACGAGCCTGGGCAACCTTTGGACTTGAGCTGTAAACGCCCCAGGCCATAA
Ad5 E1B_19K
Accession: AAQ19286.1
(SEQ ID NO: 46)
ATGGAGGCTTGGGAGTGTTTGGAAGATTTTTCTGCTGTGCGTAACTTGCTGGAACAGAGCTCTAACAGTACCTCT
TGGTTTTGGAGGTTTCTGTGGGGCTCATCCCAGGCAAAGTTAGTCTGCAGAATTAAGGAGGATTACAAGTGGGAA
TTTGAAGAGCTTTTGAAATCCTGTGGTGAGCTGTTTGATTCTTTGAATCTGGGTCACCAGGCGCTTTTCCAAGAG
AAGGTCATCAAGACTTTGGATTTTTCCACACCGGGGCGCGCTGCGGCTGCTGTTGCTTTTTTGAGTTTTATAAAG
GATAAATGGAGCGAAGAAACCCATCTGAGCGGGGGGTACCTGCTGGATTTTCTGGCCATGCATCTGTGGAGAGCG
GTTGTGAGACACAAGAATCGCCTGCTACTGTTGTCTTCCGTCCGCCCGGCGATAATACCGACGGAGGAGCAGCAG
CAGCAGCAGGAGGAAGCCAGGCGGCGGCGGCAGGAGCAGAGCCCATGGAACCCGAGAGCCGGCCTGGACCCTCGG
GAATGA
Ad5 E1B_55K
Accession: AAQ19287.1
(SEQ ID NO: 47)
ATGGAGCGAAGAAACCCATCTGAGCGGGGGGTACCTGCTGGATTTTCTGGCCATGCATCTGTGGAGAGCGGTTGT
GAGACACAAGAATCGCCTGCTACTGTTGTCTTCCGTCCGCCCGGCGATAATACCGACGGAGGAGCAGCAGCAGCA
GCAGGAGGAAGCCAGGCGGCGGCGGCAGGAGCAGAGCCCATGGAACCCGAGAGCCGGCCTGGACCCTCGGGAATG
AATGTTGTACAGGTGGCTGAACTGTATCCAGAACTGAGACGCATTTTGACAATTACAGAGGATGGGCAGGGGCTA
AAGGGGGTAAAGAGGGAGCGGGGGGCTTGTGAGGCTACAGAGGAGGCTAGGAATCTAGCTTTTAGCTTAATGACC
AGACACCGTCCTGAGTGTATTACTTTTCAACAGATCAAGGATAATTGCGCTAATGAGCTTGATCTGCTGGCGCAG
AAGTATTCCATAGAGCAGCTGACCACTTACTGGCTGCAGCCAGGGGATGATTTTGAGGAGGCTATTAGGGTATAT
GCAAAGGTGGCACTTAGGCCAGATTGCAAGTACAAGATCAGCAAACTTGTAAATATCAGGAATTGTTGCTACATT
TCTGGGAACGGGGCCGAGGTGGAGATAGATACGGAGGATAGGGTGGCCTTTAGATGTAGCATGATAAATATGTGG
CCGGGGGTGCTTGGCATGGACGGGGTGGTTATTATGAATGTAAGGTTTACTGGCCCCAATTTTAGCGGTACGGTT
TTCCTGGCCAATACCAACCTTATCCTACACGGTGTAAGCTTCTATGGGTTTAACAATACCTGTGTGGAAGCCTGG
ACCGATGTAAGGGTTCGGGGCTGTGCCTTTTACTGCTGCTGGAAGGGGGTGGTGTGTCGCCCCAAAAGCAGGGCT
TCAATTAAGAAATGCCTCTTTGAAAGGTGTACCTTGGGTATCCTGTCTGAGGGTAACTCCAGGGTGCGCCACAAT
GTGGCCTCCGACTGTGGTTGCTTCATGCTAGTGAAAAGCGTGGCTGTGATTAAGCATAACATGGTATGTGGCAAC
TGCGAGGACAGGGCCTCTCAGATGCTGACCTGCTCGGACGGCAACTGTCACCTGCTGAAGACCATTCACGTAGCC
AGCCACTCTCGCAAGGCCTGGCCAGTGTTTGAGCATAACATACTGACCCGCTGTTCCTTGCATTTGGGTAACAGG
AGGGGGGTGTTCCTACCTTACCAATGCAATTTGAGTCACACTAAGATATTGCTTGAGCCCGAGAGCATGTCCAAG
GTGAACCTGAACGGGGTGTTTGACATGACCATGAAGATCTGGAAGGTGCTGAGGTACGATGAGACCCGCACCAGG
TGCAGACCCTGCGAGTGTGGCGGTAAACATATTAGGAACCAGCCTGTGATGCTGGATGTGACCGAGGAGCTGAGG
CCCGATCACTTGGTGCTGGCCTGCACCCGCGCTGAGTTTGGCTCTAGCGATGAAGATACAGATTGA
Sequences for E2A and E4A are both contained within Accession MN088492
Ad5 E2A orf:
Accession: QHX41645.1
(SEQ ID NO: 48)
ATGGCCAGTCGGGAAGAGGAGCAGCGCGAAACCACCCCCGAGCGCGGACGCGGTGCGGCGCGACGTCCACCAACC
ATGGAGGACGTGTCGTCCCCGTCGCCGTCGCCGCCGCCTCCCCGCGCGCCCCCAAAAAAGCGGCTGAGGCGGCGT
CTCGAGTCCGAGGACGAAGAAGACTCGTCACAAGATGCGCTGGTGCCGCGCACACCCAGCCCGCGGCCATCGACC
TCGACGGCGGATTTGGCCATTGCGTCCAAAAAGAAAAAGAAGCGCCCCTCTCCCAAGCCCGAGCGCCCGCCATCC
CCAGAGGTGATCGTGGACAGCGAGGAAGAAAGAGAAGATGTGGCGCTACAAATGGTGGGTTTCAGCAACCCACCG
GTGCTAATCAAGCACGGCAAGGGAGGTAAGCGCACGGTGCGGCGGCTGAATGAAGACGACCCAGTGGCGCGGGGT
ATGCGGACGCAAGAGGAAAAGGAAGAGTCCAGTGAAGCGGAAAGTGAAAGCACGGTGATAAACCCGCTGAGCCTG
CCGATCGTGTCTGCGTGGGAGAAGGGCATGGAGGCTGCGCGCGCGTTGATGGACAAGTACCACGTGGATAACGAT
CTAAAGGCAAACTTCAAGCTACTGCCTGACCAAGTGGAAGCTCTGGCGGCCGTATGCAAGACCTGGCTAAACGAG
GAGCACCGCGGGTTGCAGCTGACCTTCACCAGCAACAAGACCTTTGTGACGATGATGGGGCGATTCCTGCAGGCG
TACCTGCAGTCGTTTGCAGAGGTAACCTACAAGCACCACGAGCCCACGGGCTGCGCGTTGTGGCTGCACCGCTGC
GCTGAGATCGAAGGCGAGCTTAAGTGTCTACACGGGAGCATTATGATAAATAAGGAGCACGTGATTGAAATGGAT
GTGACGAGCGAAAACGGGCAGCGCGCGCTGAAGGAGCAGTCTAGCAAGGCCAAGATCGTGAAGAACCGGTGGGGC
CGAAATGTGGTGCAGATCTCCAACACCGACGCAAGGTGCTGCGTGCATGACGCGGCCTGTCCGGCCAATCAGTTT
TCCGGCAAGTCTTGCGGCATGTTCTTCTCTGAAGGCGCAAAGGCTCAGGTGGCTTTTAAGCAGATCAAGGCTTTC
ATGCAGGCGCTGTATCCTAACGCCCAGACCGGGCACGGTCACCTTCTGATGCCACTACGGTGCGAGTGCAACTCA
AAGCCTGGGCATGCACCCTTTTTGGGAAGGCAGCTACCAAAGTTGACTCCGTTCGCCCTGAGCAACGCGGAGGAC
CTGGACGCGGATCTGATCTCCGACAAGAGCGTGCTGGCCAGCGTGCACCACCCGGCGCTGATAGTGTTCCAGTGC
TGCAACCCTGTGTATCGCAACTCGCGCGCGCAGGGCGGAGGCCCCAACTGCGACTTCAAGATATCGGCGCCCGAC
CTGCTAAACGCGTTGGTGATGGTGCGCAGCCTGTGGAGTGAAAACTTCACCGAGCTGCCGCGGATGGTTGTGCCT
GAGTTTAAGTGGAGCACTAAACACCAGTATCGCAACGTGTCCCTGCCAGTGGCGCATAGCGATGCGCGGCAGAAC
CCCTTTGATTTTTAA
Ad5 E4A:
Two proteins are present in this ORF. The first is a splice variant
contained within the ORF. The second is a non-spliced transcript present in
the ORF. Accession 1: QHX41659.1
Accession 2: QHX41660.1
(SEQ ID NO: 49)
ATGACTACGTCCGGCGTTCCATTTGGCATGACACTACGACCAACACGATCTCGGTTGTCTCGGCGCACTCCGTAC
AGTAGGGATCGCCTACCTCCTTTTGAGACAGAGACCCGCGCTACCATACTGGAGGATCATCCGCTGCTGCCCGAA
TGTAACACTTTGACAATGCACAACGCGTGGACTTCCCCTTCGCCGCCCGTTGAGCAACCGCAAGTTGGACAGCAG
CCTGTGGCTCAGCAGCTGGACAGCGACATGAACTTAAGCGAGCTGCCCGGGGAGTTTATTAATATCACTGATGAG
CGTTTGGCTCGACAGGAAACCGTGTGGAATATAACACCTAAGAATATGTCTGTTACCCATGATATGATGCTTTTT
AAGGCCAGCCGGGGAGAAAGGACTGTGTACTCTGTGTGTTGGGAGGGAGGTGGCAGGTTGAATACTAGGGTTCTG
TGA
(SEQ ID NO: 50)
ATGACTACGTCCGGCGTTCCATTTGGCATGACACTACGACCAACACGATCTCGGTTGTCTCGGCGCACTCCGTAC
AGTAGGGATCGCCTACCTCCTTTTGAGACAGAGACCCGCGCTACCATACTGGAGGATCATCCGCTGCTGCCCGAA
TGTAACACTTTGACAATGCACAACGTGAGTTACGTGCGAGGTCTTCCCTGCAGTGTGGGATTTACGCTGATTCAG
GAATGGGTTGTTCCCTGGGATATGGTTCTGACGCGGGAGGAGCTTGTAATCCTGAGGAAGTGTATGCACGTGTGC
CTGTGTTGTGCCAACATTGATATCATGACGAGCATGATGATCCATGGTTACGAGTCCTGGGCTCTCCACTGTCAT
TGTTCCAGTCCCGGTTCCCTGCAGTGCATAGCCGGCGGGCAGGTTTTGGCCAGCTGGTTTAGGATGGTGGTGGAT
GGCGCCATGTTTAATCAGAGGTTTATATGGTACCGGGAGGTGGTGAATTACAACATGCCAAAAGAGGTAATGTTT
ATGTCCAGCGTGTTTATGAGGGGTCGCCACTTAATCTACCTGCGCTTGTGGTATGATGGCCACGTGGGTTCTGTG
GTCCCCGCCATGAGCTTTGGATACAGCGCCTTGCACTGTGGGATTTTGAACAATATTGTGGTGCTGTGCTGCAGT
TACTGTGCTGATTTAAGTGAGATCAGGGTGCGCTGCTGTGCCCGGAGGACAAGGCGTCTCATGCTGCGGGCGGTG
CGAATCATCGCTGAGGAGACCACTGCCATGTTGTATTCCTGCAGGACGGAGCGGCGGCGGCAGCAGTTTATTCGC
GCGCTGCTGCAGCACCACCGCCCTATCCTGATGCACGATTATGACTCTACCCCCATGTAG
Ad5 VA:
Accession: AF369965.1
(SEQ ID NO: 51)
TCGATGTAGGATGTTGCCCCTCCTGACGCGGTAGGAGAAGGGGAGGGTGCCCTGCATGTCTGCCGCTGCTCTTGC
TCTTGCCGCTGCTGAGGAGGGGGGCGCATCTGCCGCAGCACCGGATGCATCTGGGAAAAGCAAAAAAGGGGCTCG
TCCCTGTTTCCGGAGGAATTTGCAAGCGGGGTCTTGCATGACGGGGAGGCAAACCCCCGTTCGCCGCAGTCCGGC
CGGCCCGAGACTCGAACCGGGGGTCCTGCGACTCAACCCTTGGAAAATAACCCTCCGGCTACAGGGAGCGAGCCA
CTTAATGCTTTCGCTTTCCAGCCTAACCGCTTACGCCGCGCGCGGCCAGTGGCCAAAAAAGCTAGCGCAGCAGCC
GCCGCGCCTGGAAGGAAGCCAAAAGGAGCGCTCCCCCGTTGTCTGACGTCGCACACCTGGGTTCGACACGCGGGC
GGTAACCGCATGGATCACGGCGGACGGCCGGATCCGGGGTTCGAACCCCGGTCGTCCGCCATGATACCCTTGCGA
ATTTATCCACCAGACCACGGAAGAGTGCCCGCTTACAGGCTCTCCTTTTGCACGGTCTAGAGCGTCAACGACTGC
GCACGCCTCACCGGCCAGAGCGTCCCGACCATGGAGCACTTTTTGCCGCTGCGCAACATCTGGAACCGCGTCCGC
GACTTTCCGCGCGCCTCCACCACCGCCGCCGGCATCACCTGGATGTCCAGGTACATCTACGGATTACG
```

### Example 32—Specific Binding Pair Polynucleotide Sequences

Sequences can be found in one or more of these publications, for example, in WO2022/234276A1, WO2022129547A1, WO2011/098772, WO2016/193746, WO2018/197854, WO2018/189517 or Li et al., J. Mol. Biol. 426, 309-317 (2014); or can be designed or obtained by methods known in the art, for example, as described in Zakeri et al, 2012, and in Zakeri et al, 2010.

SpyTag Polynucleotide Sequence
(SEQ ID NO: 52)
GCCCACATCGTGATGGTGGACGCCTACAAGCCGACGAAG
SpyCatcher Polynucleotide Sequence
(SEQ ID NO: 53)
GTGGATACCCTGTCCGGACTGAGCAGTGAGCAAGGCCAGTCCGGAGATATGACAATTGAAGAAGATAGCGCCACC
CATATTAAATTCTCCAAAAGAGATGAGGACGGCAAAGAGCTGGCTGGAGCAACAATGGAGCTGAGAGATTCCTCT
GGAAAGACTATTAGTACATGGATCTCTGATGGCCAAGTGAAAGATTTCTATCTGTATCCAGGAAAGTACACATTT
GTCGAAACCGCTGCACCAGACGGATATGAGGTGGCTACAGCTATTACCTTTACAGTGAATGAGCAAGGACAGGTG
ACTGTTAATGGCAAAGCTACTAAAGGAGACGCTCATATTTAA

### Example 33—SpyTag Peptide Sequence

(SEQ ID NO: 54)
AHIVMVDAYKPTK

### Example 34—DNA Barcode Sequences

(SEQ ID NO: 55)
CACATATCAGAGTGCGACACACAGACTGTGAG
(SEQ ID NO: 56)
ACACATCTCGTGAGAGCACGCACACACGCGCG
(SEQ ID NO: 57)
CACTCGACTCTCGCGTCATATATATCAGCTGT
(SEQ ID NO: 58)
TCTGTATCTCTATGTGACAGTCGAGCGCTGCG
(SEQ ID NO: 59)
ACACACGCGAGACAGAACGCGCTATCTCAGAG
(SEQ ID NO: 60)
CTATACGTATATCTATACACTAGATCGCGTGT
(SEQ ID NO: 61)
CTCTCGCATACGCGAGCTCACTACGCGCGCGT
(SEQ ID NO: 62)
CGCATGACACGTGTGTCATAGAGAGATAGTAT
(SEQ ID NO: 63)
CACACGCGCGCTATATTCACGTGCTCACTGTG
(SEQ ID NO: 64)
ACACACTCTATCAGATCACGACACGACGATGT
(SEQ ID NO: 65)
CTATACATAGTGATGTCACTCACGTGTGATAT
(SEQ ID NO: 66)
CAGAGAGATATCTCTGCATGTAGAGCAGAGAG
(SEQ ID NO: 67)
CGCGACACGCTCGCGCCACAGAGACACGCACA
(SEQ ID NO: 68)
CTCACACTCTCTCACACTCTGCTCTGACTCTC
(SEQ ID NO: 69)
TATATATGTCTATAGATCTCTCTATCGCGCTC
(SEQ ID NO: 70)
GATGTCTGAGTGTGTGGAGACTAGAGATAGTG
(SEQ ID NO: 71)
TCTCGTCGCAGTCTCTATGTGTATATAGATAT
(SEQ ID NO: 72)
GCGCGCGCACTCTCTGGAGACACGTCGCACAC
(SEQ ID NO: 73)
ACACATATCGCACTACGTGTGTCTCGATGCGC
(SEQ ID NO: 74)
CGCACACATAGATACATGTCATATGAGAGTGT
(SEQ ID NO: 75)
TCTCGCGCGTGCACGCCTCGCTCGACGAGCGC
(SEQ ID NO: 76)
TATAGAGCTCTACATAGCTGAGACGACGCGCG
(SEQ ID NO: 77)
ACATATCGTACTCTCTGATATATCGAGTATAT
(SEQ ID NO: 78)
TGTCATGTGTACACACGTGTGCACTCACACTC
(SEQ ID NO: 79)
ACACGTGTGCTCTCTCGATATACGCGAGAGAG
(SEQ ID NO: 80)
CGTGTCTAGCGCGCGCGTGTGAGATATATATC
(SEQ ID NO: 81)
CTCACGTACGTCACACGCGCACGCACTACAGA
(SEQ ID NO: 82)
CACACGAGATCTCATCAGACACACACGCACAT
(SEQ ID NO: 83)
GACGAGCGTCTGAGAGTGTGTCTCTGAGAGTA
(SEQ ID NO: 84)
CACACGCACTGAGATAGATGAGTATAGACACA
(SEQ ID NO: 85)
GCTGTGTGTGCTCGTCTCTCAGATAGTCTATA
(SEQ ID NO: 86)
ACACGCATGACACACTTATATACAGAGTCGAG
(SEQ ID NO: 87)
GCGCTCTCTCACATACTATATGCTCTGTGTGA
(SEQ ID NO: 88)
CTCTATATATCTCGTCAGAGAGCTCTCTCATC
(SEQ ID NO: 89)
GCGAGAGTGAGACGCATGCTCTCGTGTACTGT
(SEQ ID NO: 90)
AGCGCTGCGACACGCGAGACGCGAGCGCGTAG
(SEQ ID NO: 91)
GCGTGTGTCGAGTGTATGTACGCTCTCTATAT
(SEQ ID NO: 92)
TAGAGAGCGTCGCGTGGTGCACTCGCGCTCTC
(SEQ ID NO: 93)
TATCTCTCGAGTCGCGCTCACACATACACGTC
(SEQ ID NO: 94)
ATAGTACACTCTGTGTTATCTCTGTAGAGTCT
(SEQ ID NO: 95)
GATATATATGTGTGTAGTGACACACAGAGCAC
(SEQ ID NO: 96)
ATATGACATACACGCACGTCTCTCGTCTGTGC
(SEQ ID NO: 97)
ACACAGTAGAGCGAGCGTCGCGCATAGAGCGC
(SEQ ID NO: 98)
CTATCTAGCACTCACACGTGTCACTCTGCGTG
(SEQ ID NO: 99)
CGCGCGAGTATCTCGTAGCACACATATAGCGC
(SEQ ID NO: 100)
GTATATATATACGTCTTCTCACGAGAGCGCAC
(SEQ ID NO: 101)
TAGATGCGAGAGTAGAATAGCGACATCTCTCT
(SEQ ID NO: 102)
GCACGATGTCAGCGCGTGTGCTCTCTACACAG

### Example 35—Additional Specific Binding Pairs

Additional binding pairs are reported. See, for example, WO2022/234276A1, WO2011/098772, WO2022129547A1, WO2016/193746, WO2018/197854, WO2018/189517 or Li et al., J. Mol. Biol. 426, 309-317 (2014).

| Peptide partner | Exemplary Sequence |
|---|---|
| SpyCatcher (SEQ | VDTLSGLSSEQGQSGDMTIEEDSATHIKFSKRDEDGKELAGATMELRDSSGK |
| ID NO. 103) | TISTWISDGQVKDFYLYPGKYTFVETAAPDGYEVATAITFTVNEQGQVTVNG |
| | KATKGDAHI |
| SpyCatcher ΔN1 | DSATHIKFSKRDEDGKELAGATMELRDSSGKTISTWISDGQVKDFYLYPGKY |
| (SEQ ID NO. 104) | TFVETAAPDGYEVATAITFTVNEQGQVTVNGKATKGDAHI |
| SpyCatcher002 | VTTLSGLSGEQGPSGDMTTEEDSATHIKFSKRDEDGRELAGATMELRDSSGK |
| (SEQ ID NO. 105) | TISTWISDGHVKDFYLYPGKYTFVETAAPDGYEVATAITFTVNEQGQVTVNG |
| | EATKGDAHT |
| Spycatcher003 | VTTLSGLSGEQGPSGDMTTEEDSATHIKFSKRDEDGRELAGATMELRDSSGK |
| (SEQ ID NO. 106) | TISTWISDGHVKDFYLYPGKYTFVETAAPDGYEVATPIEFTVNEDGQVTVDG |
| | EATEGDAHT |
| SpyCatcher ΔN1 | DSATHIKFSKRDEDGKELAGATMELRDSSGKTISTWISDGQVKDFYLYPGKY |
| ΔC2 (SEQ ID | TFVETAAPDGYEVATAITFTVNEQGQVTVNG |
| NO. 107) | |
| SnoopCatcher | KPLRGAVFSLQKQHPDYPDIYGAIDQNGTYQNVRTGEDGKLIFKNLSDGKYR |
| (SEQ ID NO. 108) | LFENSEPAGYKPVQNKPIVAFQIVNGEVRDVTSIVPQDIPATYEFTNGKHYI |
| | TNEPIPPK |
| Spyligase (SEQ | DYDGQSGDGKELAGATMELRDSSGKTISTWISDGQVKDFYLYPGKYTFVETA |
| ID NO. 109) | APDGYEVATAITFTVNEQGQVTVNGKATKGGSGGSGGSGEDSATHI* |
| SdyCatcher (SEQ | LSGETGQSGNTTIEEDSTTHVKESKRDANGKELAGAMIELRNLSGQTIQSWI |
| ID NO. 110) | SDGTVKVFYLMPGTYQFVETAAPEGYELAAPITFTIDEKGQIWVDS |
| RrgACatcher (SEQ | KLGDIEFIKVNKNDKKPLRGAVFSLQKQHPDYPDIYGAIDQNGTYQNVRTGE |
| ID NO. 111) | DGKLTFKNLSDGKYRLFENSEPAGYKPVQNKPIVAFQIVNGEVRDVTSIVPQ |
| Dogcatcher V1 | KLGEIEFIKVDKIDKKPLRGAVFSLQKQHPDYPDIYGAIDQNGTYQDVRTGE |
| (SEQ ID NO. 112) | DGKLIFTNLSDGKYRLIENSEPPGYKPVQNKPIVSFRIVDGEVRDVTSIVPQ |
| Dogcatcher V2 | KIGEIEFIKVDKIDKKPLRGAVESLQKQHPDYPDIYGAIDQNGTYQDVRTGE |
| (SEQ ID NO. 113) | DGKLTFTNLSDGKYRLFENSEPPGYKPVQNKPIVAFQIVDGEVRDVTSIVPQ |
| PsCsCatcher (SEQ | EQDVVFSKVNVAGEEIAGAKIQLKDAQGQVVHSWTSKAGQSETVKLKAGTYT |
| ID NO. 114) | FHEASAPTGYLAVTDITFEVDVQGKVTVKDANGNGVKAD |
| PilinC (SEQ ID | ATTVHGETVVNGAKITVTKNLDLVNSNALIPNTDFTFKIEPDTTVNEDGNKE |
| NO. 115) | KGVALNTPMTKVTYTNSDKGGSNTKTAEFDFSEVTFEKPGVYYYKVTEEKID |
| | KVPGVSYDTTSYTVQVHVLWNEEQQKPVATYIVGYKEGSKVPIQFKNSIDST |
| | TLTVKKKVSGTGGDRSKDENFGLTLKANQYYKASEKVMIEKTTKGGQAPVQT |
| | EASIDQLYHFTLKDGESIKVINLPVGVDYVVTEDDYKSEKYTTNVEVSPQDG |
| | AVKNIAGNSTEQETSTDKDMTI |
| QueenCatcher | IDTMSGLSGETGQSGNTTIEEDSTTHVKFSKRDSNGKELAGAMIELRNLSGQ |
| (SEQ ID NO. 116) | TIQSWVSDGTVKDFYLMPGTYQFVETAAPEGYELAAPITFTVNEQGQVTVNG |
| | KATKGDAHI |
| Kat I (SEQ ID | DTMSGLSGETGQSGNTTIEEDSTTHVKFSKRDSNGKELAGAMIELRNLSGQT |
| NO. 117) | IQSWVSDGTVKDFYLMPGTYQFVETAAPEGYELAAPITETVQDNGEVQIQGK |
| | ATRGDVPI |
| Mooncake (SEQ ID | IDTMSGLSGETGQSGNITIEEDSTTHVKFSKRDSNGKELAGAMIELRNLSGQ |
| NO. 118) | TIQSWVSDGTVKDFYLMPGTYQFVETAAPEGYELAAPITFTVQDNGEVIIQG |
| | RLTRGDVHI |
| SpyTag002 (SEQ | VPTIVMVDAYKRYK |
| ID NO. 119) | |
| SpyTag003 (SEQ | RGVPHIVMVDAYKRYK |
| ID NO. 120) | |
| SnoopTag Jr (SEQ | KLGSIEFIKVNK |
| ID NO. 121) | |
| DogTag (SEQ ID | DIPATYEFTDGKHYITNEPIPPK |
| NO. 122) | |
| PsCsTag (SEQ ID | GNKLTVTDQAAPS |
| NO. 123) | |
| RrgATag (SEQ ID | DIPATYEFTNDKHYITNEP |
| NO. 124) | |
| lsopepTag (SEQ | TDKDMTITFIN KKDAE |
| ID NO. 125) | |
| Clib9 (SEQ ID | RGVPTIVMVDCYKRYK |
| NO. 126) | |
| Rum2Tag (SEQ ID | GTPIVIMVDEAKPSLP |
| NO. 127) | |
| Rum3Tag (SEQ ID | GNPLIVMIDEAEQKEI |
| NO. 128) | |
| Rum4Tag (SEQ ID | AGGIIVMKDNTTKVSI |
| NO. 129) | |
| Rum5Tag (SEQ ID | GNPIVTMIDDATLVKI |
| NO. 130) | |
| Rum6Tag (SEQ ID | GNSTITMVDDTTKVHI |
| NO. 131) | |
| Rum7Tag (SEQ ID | GTPLIVMVDDTTKVEI |
| NO. 132) | |
| SpyLigase: KTag | ATHIKFSKRD |
| (SEQ ID NO. 133) | |

Nucleotide and amino acid sequences for specific binding partners have been published or can be designed or obtained by methods known in the art, for example, as described in Zakeri et a, 2012, and in Zakeri et al, 2010 See, for example:

| Tag/Catcher | |
|---|---|
| Peptide Sequence | Corresponding DNA Sequence |
| SpyTag (SEQ ID NO. 134) | (SEQ ID NO. 135) |
| AHIVMVDAYKPTK | GCTCACATCG TGATGGTGGA CGCTTACAAG CCCACCAAG |
| SdyTag (SEQ ID NO. 136) | (SEQ ID NO. 137) |
| DPIVMIDNDKPIT | GACCCCATCG TGATGATCGA CAACGACAAG CCCATCACC |
| SnoopTag (SEQ ID NO. 138) | (SEQ ID NO. 139) |
| KLGDIEFIKVNK | AAACTGGGTG ATATTGAATT TATTAAAGTT AATAAA |
| PhoTag (SEQ ID NO. 140) | (SEQ ID NO. 141) |
| LVTGTAHIVMVDNYKPIVETGD | CTGGTTACCG GCACCGCACA TATTGTTATG GTTGATAACT |
| | ATAAGCCGAT CGTGGAAACC GGTGAT |
| EntTag (SEQ ID NO. 142) | (SEQ ID NO. 143) |
| NTIVMVDKLKEVPPT | ACCGAAGTTA GCGGTAATAC CATTGTGATG GTGGATAAAC |
| | TGAAAGAAGT TCCGCCTACC |
| RumTag (SEQ ID NO. 144) | (SEQ ID NO. 145) |
| SENGNPLIVMVDDTTKVKIS | AGCGAAAACG GCAACCCGCT GATTGTGATG GTGGATGATA |
| | CCACCAAAGT GAAAATTAGC |
| Rum2Tag (SEQ ID NO. 146) | (SEQ ID NO. 147) |
| GTPIVIMVDEAKPSLPD | GGCACCCCGA TTGTGATTAT GGTGGATGAA GCGAAACCGA |
| | GCCTGCCGGA T |
| Rum3Tag (SEQ ID NO. 148) | (SEQ ID NO. 149) |
| GNPLIVMIDEAEQKEIP | GGCAACCCGC TGATTGTGAT GATTGATGAA GCGGAACAGA |
| | AAGAAATTCC G |
| Rum4Tag (SEQ ID NO. 150) | (SEQ ID NO. 151) |
| AGGIIVMKDNTIKVSIS | GCGGGCGGCA TTATTGTGAT GAAAGATAAC ACCACCAAAG |
| | TGAGCATTAG C |
| Rum5Tag (SEQ ID NO. 152) | (SEQ ID NO. 153) |
| GNPIVTMIDDAILVKIS | GGCAACCCGA TTGTGACCAT GATTGATGAT GCGACCCTGG |
| | TGAAAATT |
| Rum6Tag (SEQ ID NO. 154) | (SEQ ID NO. 155) |
| GNSTITMVDDTIKVHIT | GGCAACAGCA CCATTACCAT GGTGGATGAT ACCACCAAAG |
| | TGCATATTAC C |
| Rum7Tag (SEQ ID NO. 156) | (SEQ ID NO. 157) |
| GTPLIVMVDDTTKVEIS | GGCACCCCGC TGATTGTGAT GGTGGATGAT ACCACCAAAG |
| | TGGAAATTAG C |
| RumTrunkTag D9N (SEQ ID NO. | (SEQ ID NO. 159) |
| 158) | GGTAATCCGC TGATTGTGAT GGTGAATGAT ACCACCAAAG |
| GNPLIVMVNDTTKVK | TGAAA |
| RumTrunkTag tag (SEQ ID NO. | (SEQ ID NO. 161) |
| 160) | GGCAACCCGC TGATTGTGAT GGTGGATGAT ACCACCAAAG |
| GNPLIVMVDDTTKVK | TGAAA |
| KTag/BacTag (SEQ ID NO. 162) | (SEQ ID NO. 163) |
| NEKVTGQFEIVKVDANDKTK | GGTCAGTTCG AAATTGTTAA AGTTGATGCA AACGATAAAA |
| | CTAAA |
| Bac2Tag (SEQ ID NO. 164) | (SEQ ID NO. 165) |
| SKSLGQFEIVKVDAQDKTK | AGCAAAAGCC TGGGCCAGTT TGAAATTGTG AAAGTGGATG |
| | CGCAGGATAA AACCAAA |
| Bac3Tag (SEQ ID NO. 166) | (SEQ ID NO. 167) |
| LGQFEIVKVDSQDKTK | CTGGGCCAGT TTGAAATTGT TAAAGTTGAT AGCCAGGATA |
| | AAACCAAA |
| Bac4Tag (SEQ ID NO. 168) | (SEQ ID NO. 169) |
| VTGQFEIVKVDAEDKTR | GTTACCGGTC AGTTTGAAAT CGTTAAAGTT GATGCCGAAG |
| | ATAAGACCCG T |
| Bac5Tag (SEQ ID NO. 170) | (SEQ ID NO. 171) |
| EKVMGQFEIMKVDANDKTK | GAAAAAGTGA TGGGCCAGTT CGAAATCATG AAAGTTGATG |
| | CCAACGACAA GACCAAA |
| Cpe0147 esterbond | (SEQ ID NO. 173) |
| forming tag (SEQ ID NO. 172) | GACACCAAGC AGGTGGTGAA GCACGAGGAC AAGAACGACA |
| DTKQVVKHEDKNDKAQTLVVEKP | AGGCCCAGAC CCTGGTGGTG GAGAAGCCC |
| SpyCatcher (SEQ ID NO. 174) | (SEQ ID NO. 175) |
| GAMVDTLSGLSSEQGQSGDMTIEEDS | GGTGCAATGG TTGATACCCT GAGCGGTCTG AGCAGCGAAC |
| ATHIKESKRDEDGKELAGATMELRDS | AGGGTCAGAG CGGTGATATG ACCATTGAAG AAGATAGCGC |
| SGKTISTWISDGQVKDFYLYPGKYTF | AACCCACATC AAATTCAGCA AACGTGATGA AGATGGTAAA |
| VETAAPDGYEVATAITFTVNEQGQVT | GAACTGGCAG GCGCAACAAT GGAACTGCGT GATAGCAGCG |
| | GTAAAACCAT TAGCACCTGG ATTAGTGATG GTCAGGTGAA |
| | AGATTTTTAT CTGTACCCTG GCAAATACAC CTTTGTTGAA |
| | ACCGCAGCAC CGGATGGTTA TGAAGTTGCA ACCGCAATTA |
| | CCTTTACCGT TAATGAACAG GGCCAGGTTA CCGTGAATGG |
| | TAAAGCAACC AAAGGTGATG CACATATT |
| SdyCatcher (SEQ ID NO. 176) | (SEQ ID NO. 177) |
| IDTMSGLSGETGQSGNTTIEEDSTTH | ATGGGTATTG ATACCATGAG CGGTCTGAGC GGTGAAACCG |
| VKFSKRDSNGKELAGAMIELRNLSGQ | GTCAGAGCGG TAATACCACC ATTGAAGAGG ATAGCACCAC |
| TIQSWVSDGTVKDFYLMPGTYQFVET | ACATGTGAAA TTCAGCAAAC GCGATGCAAA CGGCAAAGAA |
| AAPEGYELAAPIIFTIDEKGQIWVDS | CTGGCAGGCG CAATGATTGA ACTGCGTAAT CTGAGTGGTC |
| | AGACCATTCA GAGCTGGGTT AGTGATGGCA CCGTTAAAGA |
| | TTTTTATCTG ATGCCTGGCA CCTATCAGTT TGTTGAAACC |
| | GCAGCACCGG AAGGTTATGA GCTGGCAGCA CCGATTACCT |
| | TTACCATTGA TGAAAAAGGT CAGATTTGGG TTGATAGC |
| SnoopCatcher (SEQ ID NO. 178) | (SEQ ID NO. 179) |
| SSGLVPRGSHMKPLRGAVESLOKQHP | AGCAGCGGCC TGGTGCCGCG CGGCAGCCAT ATGAAGCCGC |
| DYPDIYGAIDQNGTYQNVRTGEDGKL | TGCGTGGTGC CGTGTTTAGC CTGCAGAAAC AGCATCCCGA |
| TFKNLSDGKYRLFENSEPAGYKPVQN | CTATCCCGAT ATCTATGGCG CGATTGATCA GAATGGGACC |
| KPIVAFQIVNGEVRDVTSIVPQDIPA | TATCAAAATG TGCGTACCGG CGAAGATGGT AAACTGACCT |
| TYEFTNGKHYITNEPIPPK | TTAAGAATCT GAGCGATGGC AAATATCGCC TGTTTGAAAA |
| | TAGCGAACCC GCTGGCTATA AACCGGTGCA GAATAAGCCG |
| | ATTGTGGCGT TTCAGATTGT GAATGGCGAA GTGCGTGATG |
| | TGACCAGCAT TGTGCCGCAG GATATTCCGG CTACATATGA |
| | ATTTACCAAC GGTAAACATT ATATCACCAA TGAACCGATA |
| | CCGCCGAAA |
| FimP domain 3 (SEQ ID NO. 180) | (SEQ ID NO. 181) |
| GSLSKYGKVILTKTGTDDLADKTKYN | GGTAGCCTGA GCAAATATGG TAAAGTGATT CTGACCAAAA |
| GAQFQVYECTKTASGATLRDSDPSTQ | CCGGCACCGA TGATCTGGCA GATAAAACCA AATATAACGG |
| TVDPLTIGGEKTFTTAGQGTVEINYL | TGCACAGTTT CAGGTGTATG AATGTACCAA AACAGCAAGC |
| RANDYVNGAKKDQLTDEDYYCLVETK | GGTGCAACCC TGCGTGATAG CGATCCGAGC ACACAGACCG |
| APEGYNLQADPLPERVLAEKAEKKA | TTGATCCGCT GACCATTGGT GGTGAAAAAA CCTTTACCAC |
| | CGCAGGTCAG GGCACCGTTG AAATTAATTA TCTGCGTGCC |
| | AATGATTATG TGAACGGTGC AAAAAAAGAT CAGCTGACCG |
| | ATGAAGATTA TTACTGTCTG GTTGAAACCA AAGCACCGGA |
| | AGGTTATAAT CTGCAGGCAG ATCCGCTGCC GTTTCGTGTT |
| | CTGGCCGAAA AAGCAGAAAA AAAAGCC |
| ancillary pilin domain 2 (SEQ | (SEQ ID NO. 183) |
| ID NO. 182) | GGTAGCACCA CCAAAGTGAA ACTGATTAAA GTTGATCAGG |
| GSTTKVKLIKVDQDHNRLEGVGFKLV | ATCACAATCG TCTGGAAGGT GTTGGTTTTA AACTGGTTAG |
| SVARDVSAAAVPLIGEYRYSSSGQVG | CGTTGCACGT GATGTTAGCG CAGCAGCAGT TCCGCTGATT |
| RTLYTDKNGEIFVTNLPLGNYRFKEV | GGTGAATATC GTTATAGCAG CAGCGGTCAG GTTGGTCGTA |
| EPLAGYAVTTLDTDVQLVDHQLVT | CCCTGTATAC CGATAAAAAT GGCGAAATTT TCGTTACCAA |
| | TCTGCCGCTG GGTAACTATC GTTTTAAAGA AGTTGAACCG |
| | CTGGCAGGTT ATGCAGTTAC CACACTGGAT ACCGATGTTC |
| | AGCTGGTTGA TCATCAGCTG GTGACC |
| ancillary pilin domain 3 (SEQ | (SEQ ID NO. 185) |
| ID NO. 184) | CCGCGTGGTA ATGTTGATTT TATGAAAGTT GATGGTCGCA |
| PRGNVDFMKVDGRTNTSLQGAMEKVM | CCAATACCAG CCTGCAGGGT GCAATGTTTA AAGTGATGAA |
| KEESGHYTPVLQNGKEVVVTSGKDGR | AGAAGAAAGC GGTCACTATA CACCGGTGCT GCAGAATGGT |
| FRVEGLEYGTYYLWELQAPTGYVQLT | AAAGAAGTTG TTGTTACCAG CGGTAAAGAT GGTCGTTTTC |
| SPVSFTIGKDTRKELV | GTGTTGAAGG TCTGGAATAT GGCACCTATT ATCTGTGGGA |
| | ACTGCAGGCA CCGACCGGTT ATGTTCAGCT GACCAGTCCG |
| | GTTAGTTTTA CCATTGGCAA AGATACCCGT AAAGAACTGG |
| | TG |
| SpaD Domain 3 (SEQ ID NO. 186) | (SEQ ID NO. 187) |
| VVTYHGKLKVVKKDGKEAGKVLKGAE | GTTGTTACCT ATCATGGTAA ACTGAAAGTG GTGAAAAAAG |
| FELYQCTSAAVLGKGPLTVDGVKKWT | ACGGTAAAGA GGCAGGCAAA GTTCTGAAAG GTGCAGAATT |
| TGDDGTFTIDGLHVTDFEDGKEAAPA | TGAACTGTAT CAGTGTACCA GCGCAGCAGT TTTAGGTAAA |
| TKKFCLKETKAPAGYALPDPNVTEIE | GGTCCGCTGA CCGTTGATGG TGTGAAAAAA TGGACCACCG |
| FTRAKISEKDKFEGDDEVT | GTGATGATGG CACCTTTACC ATTGATGGTC TGCATGTTAC |
| | CGATTTTGAA GATGGTAAAG AAGCCGCACC GGCAACCAAA |
| | AAATTCTGTC TGAAAGAAAC CAAAGCACCG GCAGGTTATG |
| | CACTGCCTGA TCCGAATGTG ACCGAAATTG AATTTACCCG |
| | TGCAAAAATC AGCGAGAAAG ATAAATTTGA AGGCGACGAT |
| | GAAGTGACC |
| Pilin subunit (SpaA) domain 2 | (SEQ ID NO. 189) |
| (SEQ ID NO. 188) | AGCACCAATG ATACCACCAC ACAGAATGTT GTTCTGACCA |
| STNDTTTQNVVLTKYGEDKDVTAIDR | AATATGGCTT CGATAAAGAT GTTACCGCAA TTGATCGTGC |
| ATDQIWIGDGAKPLOGVDFTIYNVTA | AACCGATCAG ATTTGGACCG GTGATGGTGC AAAACCGCTG |
| NYWASPKDYKGSFDSAPVAATGTIND | CAGGGTGTTG ATTTTACCAT TTATAACGTG ACCGCCAATT |
| KGQLTQALPIQSKDASGKTRAAVYLF | ATTGGGCAAG CCCGAAAGAT TATAAAGGCA GCTTTGATAG |
| HETNPRAGYNTSADFWLTLPAKAAAD | CGCACCGGTT GCAGCCACCG GTACAACAAA TGATAAAGGC |
| GNVY | CAGCTGACCC AGGCACTGCC GATTCAGAGC AAAGATGCAA |
| | GCGGTAAAAC CCGTGCAGCA GTTTACCTGT TTCACGAAAC |
| | CAATCCGCGT GCAGGTTATA ATACCAGCGC AGATTTTTGG |
| | CTGACCCTGC CTGCAAAAGC AGCAGCAGAT GGTAATGTTT |
| | AT |
| Pilin subunit (SpaA) domain 3 | (SEQ ID NO. 191) |
| (SEQ ID NO. 190) | ACCACCTATG AACGTACCTT TGTTAAAAAA GACGCCGAAA |
| TTYERTFVKKDAETKEVLEGAGFKIS | CCAAAGAAGT TCTGGAAGGC GCAGGCTTTA AAATCAGCAA |
| NSDGKFLKLTDKDGQSVSIGEGFIDV | TAGTGATGGC AAATTCCTGA AACTGACCGA TAAAGATGGT |
| LANNYRLTWVAESDATVFTSDKSGKF | CAGAGCGTTA GCATTGGTGA AGGTTTTATT GATGTTCTGG |
| GLNGFADNTTTYTAVETNVPDGYDAA | CCAATAACTA TCGTCTGACC TGGGTTGCAG AAAGTGATGC |
| ANTDEKADNS | AACCGTTTTT ACCAGCGATA AAAGCGGCAA ATTTGGTCTG |
| | AATGGTTTTG CAGATAATAC CACCACCTAT ACCGCAGTTG |
| | AAACCAATGT TCCGGATGGT TATGATGCAG CAGCAAACAC |
| | CGATTTCAAA GCCGATAATA GC |
| Surface protein Spb1 domain 3 | (SEQ ID NO. 193) |
| (SEQ ID NO. 192) | GGTCAGATTA CCATCAAAAA AATCGATGGT AGCACCAAAG |
| GQITIKKIDGSTKASLQGAIFVLKNA | CAAGCCTGCA GGGTGCAATT TTTGTTCTGA AAAATGCAAC |
| TGQFLNENDTNNVEWGTEANATEYTT | CGGTCAGTTC CTGAATTTTA ACGATACCAA TAATGTTGAA |
| GADGIITITGLKEGTYYLVEKKAPLG | TGGGGCACCG AAGCAAATGC CACCGAATAT ACCACCGGTG |
| YNLLDNSQKVILGDGATDTTNSDNLL | CAGATGGTAT TATTACCATT ACCGGTCTGA AAGAAGGCAC |
| VNP | CTATTACCTG GTTGAAAAAA AAGCACCGCT GGGTTATAAT |
| | CTGCTGGATA ATTCACAGAA AGTGATTTTA GGTGATGGTG |
| | CAACCGATAC CACCAATAGC GATAACCTGC TGGTTAATCC |
| | G |
| PsCsCatcher (SEQ ID NO. 194) | (SEQ ID NO. 195) |
| EQDVVFSKVNVAGEEIAGAKIQLKDA | GAACAGGATG TTGTGTTTAG CAAAGTTAAT GTTGCCGGTG |
| QGQVVHSWTSKAGQSETVKLKAGTYT | AAGAAATTGC GGGTGCAAAA ATCCAGCTGA AAGATGCACA |
| FHEASAPTGYLAVTDITFEVDVQGKV | GGGTCAAGTT GTTCATAGCT GGACCAGCAA AGCAGGTCAG |
| TVKDANGNGVKAD | AGCGAAACCG TTAAACTGAA AGCAGGCACC TATACCTTTC |
| | ATGAAGCAAG CGCACCGACC GGTTATCTGG CAGTTACCGA |
| | TATTACCTTT GAAGTTGATG TTCAGGGTAA AGTGACCGTT |
| | AAAGATGCAA ATGGTAATGG TGTGAAAGCC GAC |
| RgA Catcher (SEQ ID NO. 196) | (SEQ ID NO. 197) |
| KLGDIEFIKVNKNDKKPLRGAVESLQ | AAACTGGGTG ATATTGAGTT CATCAAAGTG AACAAAAACG |
| KQHPDYPDIYGAIDQNGTYQNVRTGE | ATAAAAAACC GCTGCGTGGT GCAGTTTTTA GCCTGCAGAA |
| DGKLTFKNLSDGKYRLFENSEPAGYK | ACAGCATCCG GATTACCCGG ATATTTATGG TGCAATTGAT |
| PVQNKPIVAFQIVNGEVRDVTSIVPQ | CAGAATGGCA CCTATCAGAA TGTTCGTACC GGTGAAGATG |
| | GTAAACTGAC CTTTAAAAAC CTGAGCGACG GTAAATATCG |
| | CCTGTTTGAA AATAGCGAAC CGGCAGGTTA TAAACCGGTT |
| | CAGAATAAAC CGATTGTGGC CTTTCAGATT GTTAATGGTG |
| | AAGTTCGTGA TGTGACCAGC ATTGTTCCGC AG |
| Major Pilin SpaD Domain 1 (SEQ | (SEQ ID NO. 199) |
| ID NO. 198) | GGTAGCGAAC GTAAAGGTAG TCTGACCCTG CATAAAAAGA |
| GSERKGSLTLHKKKGAESEKRATGKE | AAGGTGCAGA AAGCGAAAAA CGTGCAACCG GTAAAGAAAT |
| MDDVAGEPLNGVTFKITKLNEDLQNG | GGATGATGTT GCCGGTGAAC CGCTGAATGG TGTTACCTTT |
| DWAKFPKTAADAKGHETSTTKEVETS | AAAATCACCA AACTGAACTT CGATCTGCAG AATGGTGATT |
| GNGTAVFDNLDLGIYLVEETKAPDGI | GGGCAAAATT TCCGAAAACC GCAGCAGATG CAAAAGGTCA |
| VTGAPFIVSIPMVNEASDAWNYNVVA | TGAAACCAGC ACCACCAAAG AAGTGGAAAC CAGCGGTAAT |
| | GGCACCGCAG TTTTTGATAA TCTGGATCTG GGTATTTACC |
| | TGGTGGAAGA AACCAAAGCA CCGGATGGTA TTGTTACAGG |
| | TGCACCGTTT ATTGTTAGCA TTCCGATGGT TAATGAAGCA |
| | AGTGATGCCT GGAATTATAA CGTTGTTGCA |
| Cpe0147 esther-forming split- | (SEQ ID NO. 201) |
| protein pair (SEQ ID NO. 200) | AACCTGCCCG AGGTGAAGGA CGGCACCCTG AGGACCACCG |
| NLPEVKDGTLRTTVIADGVNGSSEKE | TGATCGCCGA CGGCGTGAAC GGCAGCAGCG AGAAGGAGGC |
| ALVSFENSKDGVDVKDTINYEGLVAN | CCTGGTGAGC TTCGAGAACA GCAAGGACGG CGTGGACGTG |
| QNYTLTGTLMHVKADGSLEEIATKTT | AAGGACACCA TCAACTACGA GGGCCTGGTG GCCAACCAGA |
| NVTAGENGNGTWGLDFGNQKLQVGEK | ACTACACCCT GACCGGCACC CTGATGCACG TGAAGGCCGA |
| YVVFENAESVENLIDTDKDYNLDTKQ | CGGCAGCCTG GAGGAGATCG CCACCAAGAC CACCAACGTG |
| VVKHEDKNDKAQTLVVEKP | ACCGCCGGCG AGAACGGCAA CGGCACCTGG GGCCTGGACT |
| | TCGGCAACCA GAAGCTGCAG GTGGGCGAGA AGTACGTGGT |
| | GTTCGAGAAC GCCGAGAGCG TGGAGAACCT GATCGACACC |
| | GACAAGGACT ACAACCTGGA CACCAAGCAG GTGGTGAAGC |
| | ACGAGGACAA GAACGACAAG GCCCAGACCC TGGTGGTGGA |
| | GAAGCCC |

### Example 36—Herpes Simplex Virus (HSV) Polynucleotide Sequences

HSV polynucleotides can be selected from any serotype, and representative polynucleotides are exemplified below. Weindler and Heilbronn 1991 (Weindler, Friedrich W., and R. E. G. I. N. E. Heilbronn. “A subset of herpes simplex virus replication genes provides helper functions for productive adeno-associated virus replication.” Journal of virology 65.5 (1991): 2476-2483); Ward et al. 2001 (Ward et al. Rep-dependent initiation of adeno-associated virus type 2 DNA replication by a herpes simplex virus type 1 replication complex in a reconstituted system. J. Virol. 2001; 75:10250-10258); Herpes Clément et al. 2009 (Clément, Nathalie, David R. Knop, and Barry J. Byrne. “Large-scale adeno-associated viral vector production using a herpesvirus-based system enables manufacturing for clinical studies.” Human gene therapy 20.8 (2009): 796-806); and Meier et al. 2020 (Meier, Anita F., Cornel Fraefel, and Michael Seyffert. “The Interplay between Adeno-Associated Virus and Its Helper Viruses.” Viruses 12.6 (2020)) disclose seven HSV replication genes (UL5, UL8, UL9, UL29, UL30, UL42, and UL52) that led to productive AAV replication, of which HSV-1 helicase-primase complex (HP; UL5/UL8/UL52) and the single-strand DNA binding protein ICP8 (gene UL29) is sufficient to restore AAV progeny production. HSV replication gene (UL5, UL8, UL52, UL29, UL9, UL30, and UL42) sequences as available at the GenBank are listed below:

UL5 helicase-primase helicase subunit [Human alphaherpesvirus 1 (Herpes simplex virus type 1)] Gene ID: 2703420. NCBI Reference Sequence: NC_001806.2.

| (SEQ ID NO. 202) | |
|---|---|
| 1 | atggcggcgg ccgggggga gcgccagcta gacggacaga aacccggccc gccgcacctt |
| 61 | cagcaacccg gggaccgacc agccgttcca gggagggccg aggccttttt aaattttacg |
| 121 | tctatgcacg gggtgcagcc aatccttaag cgcatccgag agctctcgca acaacagctc |
| 181 | gacggagcgc aagtgcccca tctgcagtgg ttccgggacg tggcggcctt agagtccccc |
| 241 | gcaggcctgc ccctcaggga gtttccgttc gcggtgtatc ttatcaccgg caacgctggc |
| 301 | tccggaaaga gcacgtgcgt gcagacaatc aacgaggtct tggactgtgt ggtgacgggc |
| 361 | gccacgcgca ttgcggccca aaacatgtac gccaaactct cgggcgcctt tctcagccga |
| 421 | cccatcaaca ccatctttca tgaatttggg tttcgcggga atcacgtcca ggcccaactg |
| 481 | ggacagtacc cgtacaccct gaccagcaac cccgcctcgc tggaggacct gcagcgacga |
| 541 | gatctgacgt actactggga ggtgattttg gacctcacga agcgcgccct ggccgcctcc |
| 601 | gggggcgagg agttgcggaa cgagtttcgc gccctggccg ccctggaacg gaccctgggg |
| 661 | ttggccgagg gcgccctgac gcggttggcc ccggccaccc acggggcgct gccggccttt |
| 721 | acccgcagca acgtgatcgt catcgacgag gccgggctcc ttgggcgtca cctcctcacg |
| 781 | gccgtggtgt attgctggtg gatgattaac gccctgtacc acacccccca gtacgcggcc |
| 841 | cgcctgcggc ccgtgttggt gtgtgtgggc tcgccgacgc agacggcgtc cctggagtcg |
| 901 | accttcgagc accagaaact gcggtgttcc gtccgccaga gcgagaacgt gctcacgtac |
| 961 | ctcatctgca accgcacgct gcgcgagtac gcccgcctct cgtatagctg ggccattttt |
| 1021 | attaacaaca aacggtgcgt cgagcacgag ttcggtaacc tcatgaaggt gctggagtac |
| 1081 | ggcctgccca tcaccgagga gcacatgcag ttcgtggatc gcttcgtcgt cccggaaaac |
| 1141 | tacatcacca accccgccaa cctccccggc tggacgcggc tgttctcctc ccacaaagag |
| 1201 | gtgagcgcgt acatggccaa gctccacgcc tacctgaagg tgacccgtga gggggagttc |
| 1261 | gtcgtgttca ccctccccgt gcttacgttc gtgtcggtca aggagtttga cgaataccga |
| 1321 | cggctgacac accagcccgg cctgacgatt gaaaagtggc tcacggccaa cgccagccgc |
| 1381 | atcaccaact actcgcagag ccaggaccag gacgcggggc acatgcgctg cgaggtgcac |
| 1441 | agcaaacagc agctggtcgt ggcccgcaac gacgtcactt acgtcctcaa cagccagatc |
| 1501 | gcggtgaccg cgcgcctgcg aaaactggtt tttgggttta gtgggacgtt ccgggccttc |
| 1561 | gaggcagtgt tgcgtgacga cagctttgta aagactcagg gggagacttc ggtggagttt |
| 1621 | gcctacaggt tcctgtcgcg gctcatattt agcgggctta tctcctttta caactttctg |
| 1681 | cagcgcccgg gcctggatgc gacccagagg accctcgcct acgcccgcat gggagaacta |
| 1741 | acggcggaga ttctgtctct gcgccccaaa tcttcggggg tgccgacgca ggcgtcggta |
| 1801 | atggccgacg caggcgcccc cggcgagcgt gcgtttgatt ttaagcaact ggggccgcgg |
| 1861 | gacgggggcc cggacgattt tcccgacgac gacctcgacg ttattttcgc ggggctggac |
| 1921 | gaacaacagc tcgacgtgtt ttactgccac tacacccccg gggaaccgga gaccaccgcc |
| 1981 | gccgttcaca cccagtttgc gctgctgaag cgggccttcc tcgggagatt ccgaatcctc |
| 2041 | caagagctct tcggggaggc atttgaagtc gcccccttta gcacgtacgt ggacaacgtt |
| 2101 | atcttccggg gctgcgagat gctgaccggc tcgccgcgcg gggggctgat gtccgtcgcc |
| 2161 | ctgcagacgg acaattatac gctcatggga tacacgtacg cacgggtgtt tgcctttgcg |
| 2221 | gacgagctgc ggaggcggca cgcgacggcc aacgtggccg agttactgga agaggccccc |
| 2281 | ctgccttacg tggtcttgcg ggaccaacac ggcttcatgt ccgtcgtcaa caccaacatc |
| 2341 | agcgagtttg tcgagtccat tgactctacg gagctggcca tggccataaa cgccgactac |
| 2401 | ggcatcagct ccaagcttgc catgaccatc acgcgctccc agggccttag cctggacaag |
| 2461 | gtcgccatct gctttacgcc cggcaacctg cgcctcaaca gcgcgtacgt ggccatgtcc |
| 2521 | cgcaccacct cctccgaatt ccttcgcatg aacttaaatc cgctccggga gcgccacgag |
| 2581 | cgcgatgacg tcattagtga gcacatacta tcggctctgc gcgatccgaa cgtggtcatt |
| 2641 | gtctattaac ccgccgtccc cttacagttc caccgaaccc ggcccggggg actcactacc |
| 2701 | caccgcgaga tgtccaatcc acagacgacc atcgcgtata gcctatgcca cgccagggcc |
| 2761 | tcgctgacca gcgcactgcc cgacgccgcg caggtggtgc atgtttttga gtacggcacc |
| 2821 | cgcgcgatca tggtacgggg ccgggagcgc caggaccgcc tgccgcgcgg aggcgttgtt |
| 2881 | atccagcaca cccccattgg gctgttggtg attatcgact gtcgcgccga attttgtgcc |
| 2941 | taccgcttta taggccggga cagcaaccag aagctcgaac gcgggtggga cgcccatatg |
| 3001 | tacgcgtatc cgttcgactc ctgggtcagc tcctcgcgcg gcgaaagcgc ccggagcgcc |
| 3061 | acggccggca ttttgaccgt ggtctggacc gcggacacca tttacatcac tgcaaccatt |
| 3121 | tacgggtcgc ccccagagga gacgccaggc gcggcacacg gggtgggcgc cgcgcctcca |
| 3181 | cccccgacaa ccgcctgccc cgggacggcc gagtttctcc agcccaccgc ggacctgctg |
| 3241 | gtagaggtgc tgcgggagat tcaactgagc cccgccctgg aatacgcaga caaacttttg |
| 3301 | gggtcctagg atcccggccg gatcgcgctc gtcacccgac actgaaatgc cccccccccc |
| 3361 | ttgcgggcgg tccattaaa |

UL8 helicase-primase subunit [Human alphaherpesvirus 1 (Herpes simplex virus type 1)] Gene ID: 2703432. NCBI Reference Sequence: NC_001806.2.

| (SEQ ID NO. 203) | |
|---|---|
| 1 | atggacaccg cagatatcgt gtgggtggag gagagcgtca gcgccattac cctttacgcg |
| 61 | gtatggctgc ccccccgcgc tcgcgagtac ttccacgccc tggtgtattt tgtatgtcgc |
| 121 | aacgccgcag gggagggtcg cgcgcgcttt gcggaggtct ccgtcaccgc gacggagctg |
| 181 | cgggatttct acggctccgc ggacgtctcc gtccaggccg tcgtggcggc cgcccgcgcc |
| 241 | gcgacgacgc cggccgcctc cccgctggag cccctggaga acccgactct gtggcgggcg |
| 301 | ctgtacgcgt gcgtcctggc ggccctggag cgccagaccg ggccggtggc cctgttcgcc |
| 361 | ccgctgcgta tcggctcgga cccacgcacg ggactggtgg tgaaagttga gagagcgtcg |
| 421 | tggggcccgc ccgccgcccc tcgcgccgct ctcctggtcg cggaggccaa cattgacatc |
| 481 | gaccctatgg ccctggcggc gcgcgttgcc gagcatcccg acgcgcggct ggcgtgggcg |
| 541 | cgcctggcgg ccattcgcga caccccccag tgcgcgtccg ccgcttcgct gaccgttaac |
| 601 | atcaccaccg gaaccgcgct atttgcgcgc gaataccaga ctcttgcgtt tccgccgatc |
| 661 | aagaaggagg gcgcgttcgg ggacctggtc gaggtgtgcg aggtgggcct gcggccacgc |
| 721 | gggcacccgc aacgagtcac ggcacgggtg ctgctgcccc gcgattacga ctactttgta |
| 781 | agcgccggcg agaagttctc cgcgccggcg ctcgtcgccc ttttccggca gtggcatacc |
| 841 | acggtccacg ccgcccccgg ggccctggcc cccgtctttg cctttctggg gcccgagttt |
| 901 | gaggtccggg ggggacccgt cccgtacttt gccgtcctgg ggtttccggg ttggcccacg |
| 961 | ttcaccgtgc cggccacggc cgagtcggca cgggacctgg tgcgcggggc cgcggccgct |
| 1021 | tacgccgcgc tcctgggggc ctggcccgcg gtgggggcca gggtcgtcct ccccccgcga |
| 1081 | gcctggcccg gcgtggcctc ggcggcagcc ggatgcctcc tgcccgcggt gcgggaggcg |
| 1141 | gtggcgcggt ggcatcccgc cactaaaatc atccaactgt tagacccgcc cgcggccgtc |
| 1201 | gggcccgtct ggacggcgcg gttttgcttc cccggacttc gcgcccagct cctggcggcc |
| 1261 | ctggccgacc tcggggggag cgggctggcg gacccccacg gccggacggg cctagcaaga |
| 1321 | ctggacgcgc tggtggtggc cgctccctca gagccctggg ccggggccgt cttggagcgc |
| 1381 | ctggtcccgg acacgtgcaa cgcctgccct gcgctgcggc agctcctggg tggggtaatg |
| 1441 | gccgccgtct gcctgcagat cgaggagacg gccagctcgg tgaagttcgc ggtctgcggg |
| 1501 | ggcgatgggg gtgcgttctg gggtgtcttt aacgtggacc cccaagacgc ggatgcggct |
| 1561 | tccggggtga tcgaggacgc ccggcgggcc atcgagacgg ccgtgggagc cgtgcttagg |
| 1621 | gccaacggcc tccggctgcg gcacccactg tgcctggccc tcgagggcgt ctacacccac |
| 1681 | gcagtcgcct ggagccaggc gggagtgtgg ttctggaact cccgcgacaa cactgaccat |
| 1741 | cttgggggat ttcctctccg cgggcccgcg tacaccacgg cggcaggggt cgtacgcgac |
| 1801 | acgctgcgac gggtcctggg cctgacaacg gcatgcgtgc cggaggagga cgcactcacg |
| 1861 | gcccggggcc ttatggagga cgcctgcgac cgccttatct tggacgcgtt taataaacgg |
| 1921 | ttggacgcgg agtactggag cgttcgggtg tccccctttg aggccagcga ccccttgccc |
| 1981 | cccactgcct tccgcggcgg cgccttgctg gacgcagagc actactggcg gcgcgtcgtg |
| 2041 | cgtgtctgtc ccggaggcgg ggagtcggtc ggcgtccccg tcgatctata cccgcggccc |
| 2101 | cttgtgctcc cccccgtgga ctgcgctcat cacctgcgcg aaatcctgcg cgagattgag |
| 2161 | ttggtgttta ccggggtgct ggcgggagta tggggcgagg gggggaagtt tgtgtatccc |
| 2221 | tttgacgaca agatgtcgtt tctgtttgcc tgagtttgac caataaa |

UL52 helicase-primase primase subunit [Human alphaherpesvirus 1 (Herpes simplex virus type 1)]. Gene ID: 2703423. NCBI Reference Sequence: NC_001806.2.

| (SEQ ID NO. 204) | |
|---|---|
| 1 | atggggcagg aagacgggaa ccgcggggag aggcgggcgg ccgggactcc cgtggaggtg |
| 61 | accgcgcttt atgcgaccga cgggtgcgtt attacctctt cgatcgccct cctcacaaac |
| 121 | tctctactgg gggccgagcc ggtttatata ttcagctacg acgcatacac gcacgatggc |
| 181 | cgtgccgacg ggcccacgga gcaagacagg ttcgaagaga gtcgggcgct ctaccaagcg |
| 241 | tcgggcgggc taaatggcga ctccttccga gtaacctttt gtttattggg gacggaagtg |
| 301 | ggtgggaccc accaggcccg cgggcgaacc cgacccatgt tcgtctgtcg cttcgagcga |
| 361 | gcggacgacg tcgccgcgct acaggacgcc ctggcgcacg ggaccccgct acaaccggac |
| 421 | cacatcgccg ccaccctgga cgcggaggcc acgttcgcgc tgcatgcgaa catgatcctg |
| 481 | gctctcaccg tggccatcaa caacgccagc ccccgcaccg gacgcgacgc cgccgcggcg |
| 541 | cagtatgatc agggcgcgtc cctacgctcg ctcgtggggc gcacgtccct gggacaacgc |
| 601 | ggccttacca cgctatacgt ccaccacgag gtgcgcgtgc ttgccgcgta ccgcagggcg |
| 661 | tattatggaa gcgcgcagag tcccttctgg tttcttagca aattcgggcc ggacgaaaaa |
| 721 | agcctggtgc tcaccactcg gtactacctg cttcaggccc agcgtctggg gggcgcgggg |
| 781 | gccacgtacg acctgcaggc catcaaggac atctgcgcca cctacgcgat tccccacgcc |
| 841 | ccccgccccg acaccgtcag cgctgcgtcc ctgacctcgt ttgccgccat cacgcggttc |
| 901 | tgttgcacga gccagtacgc ccgcggggcc gcggcggccg ggtttccgct ttacgtggag |
| 961 | cgccgtattg cggccgacgt ccgcgagacc agtgcgctgg agaagttcat aacccacgat |
| 1021 | cgcagttgcc tgcgcgtgtc cgaccgtgaa ttcattacgt acatctacct ggcccatttt |
| 1081 | gagtgtttca gccccccgcg cctagccacg catcttcggg ccgtgacgac ccacgacccc |
| 1141 | aaccccgcgg ccagcacgga gcagccctcg cccctgggca gggaggccgt ggaacaattt |
| 1201 | ttttgtcacg tgcgcgccca actgaatatc ggggagtacg tcaaacacaa cgtgaccccc |
| 1261 | cgggagaccg tcctggatgg cgatacggcc aaggcctacc tgcgcgctcg cacgtacgcg |
| 1321 | cccggggccc tgacgcccgc ccccgcgtat tgcggggccg tggactccgc caccaaaatg |
| 1381 | atggggcgtt tggcggacgc cgaaaagctc ctggtccccc gcgggtggcc cgcgtttgcg |
| 1441 | cccgccagtc ccggggagga cacggcgggc ggcacgccgc ccccacagac ctgcggaatt |
| 1501 | gtcaagcgcc tcctgagact ggccgccacg gaacagcagg gccccacacc cccggcgatc |
| 1561 | gcggcgctta tccgtaatgc ggcggtgcag actcccctgc ccgtctaccg gatatccatg |
| 1621 | gtccccacgg gacaggcatt tgccgcgctg gcctgggacg actgggcccg cataacgcgg |
| 1681 | gacgctcgcc tggccgaagc ggtcgtgtcc gccgaagcgg cggcgcaccc cgaccacggc |
| 1741 | gcgctgggca ggcggctcac ggatcgcatc cgcgcccagg gccccgtgat gccccctggc |
| 1801 | ggcctggatg ccggggggca gatgtacgtg aatcgcaacg agatattcaa cggcgcgctg |
| 1861 | gcaatcacaa acatcatcct ggatctcgac atcgccctga aggagcccgt cccctttcgc |
| 1921 | cggctccacg aggccctggg ccactttagg cgcggggctc tggctgcggt tcagctcctg |
| 1981 | tttcccgcgg cccgcgtgga ccccgacgca tatccctgtt attttttcaa aagcgcatgt |
| 2041 | cggcccggcc cggcgtccgt gggttccggc agcggactcg gcaacgacga cgacggggac |
| 2101 | tggtttccct gctacgacga cgccggtgat gaggagtggg cggaggaccc gggcgccatg |
| 2161 | gacacatccc acgatccccc ggacgacgag gttgcctact ttgacctgtg ccacgaagtc |
| 2221 | ggccccacgg cggaacctcg cgaaacggat tcgcccgtgt gttcctgcac cgacaagatc |
| 2281 | ggactgcggg tgtgcatgcc cgtccccgcc ccgtacgtcg tccacggttc tctaacgatg |
| 2341 | cggggggtgg cacgggtcat ccagcaggcg gtgctgttgg accgagattt tgtggaggcc |
| 2401 | atcgggagct acgtaaaaaa cttcctgttg atcgatacgg gggtgtacgc ccacggccac |
| 2461 | agcctgcgct tgccgtattt tgccaaaatc gcccccgacg ggcctgcgtg cggaaggctg |
| 2521 | ctgccagtgt ttgtgatccc ccccgcctgc aaagacgttc cggcgtttgt cgccgcgcac |
| 2581 | gccgacccgc ggcgcttcca ttttcacgcc ccgcccacct atctcgcttc cccccgggag |
| 2641 | atccgtgtcc tgcacagcct gggtggggac tatgtgagct tctttgaaag gaaggcgtcc |
| 2701 | cgcaacgcgc tggaacactt tgggcgacgc gagaccctga cggaggtcct gggtcggtac |
| 2761 | aacgtacagc cggatgcggg ggggaccgtc gaggggttcg catcggaact gctggggcgg |
| 2821 | atagtcgcgt gcatcgaaac ccactttccc gaacacgccg gcgaatatca ggccgtatcc |
| 2881 | gtccggcggg ccgtcagtaa ggacgactgg gtcctcctac agctagtccc cgttcgcggt |
| 2941 | accctgcagc aaagcctgtc gtgtctgcgc tttaagcacg gccgggcgag tcgcgccacg |
| 3001 | gcgcggacat tcgtcgcgct gagcgtcggg gccaacaacc gcctgtgcgt gtccttgtgt |
| 3061 | cagcagtgct ttgccgccaa atgcgacagc aaccgcctgc acacgctgtt taccattgac |
| 3121 | gccggcacgc catgctcgcc gtccgttccc tgcagcacct ctcaaccgtc gtcttgataa |
| 3181 | cggcgtacgg cctcgtgctc gtgtggtaca ccgtcttcgg tgccagtccg ctgcaccgat |
| 3241 | gtatttacgc ggtacgcccc accggcacca acaacgacac cgccctcgtg tggatgaaaa |
| 3301 | tgaaccagac cctattgttt ctgggggccc cgacgcaccc ccccaacggg ggctggcgca |
| 3361 | accacgccca tatctgctac gccaatctta tcgcgggtag ggtcgtgccc ttccaggtcc |
| 3421 | cacctgacgc catgaatcgt cggatcatga acgtccacga ggcagttaac tgtctggaga |
| 3481 | ccctatggta cacacgggtg cgtctggtgg tcgtagggtg gttcctgtat ctggcgttcg |
| 3541 | tcgccctcca ccaacgccga tgtatgtttg gcgtcgtgag tcccgcccac aagatggtgg |
| 3601 | ccccggccac ctacctcttg aactacgcag gccgcatcgt atcgagcgtg ttcctgcagt |
| 3661 | acccctacac gaaaattacc cgcctgctct gcgagctgtc ggtccagcgg caaaacctgg |
| 3721 | ttcagttgtt tgagacggac ccggtcacct tcttgtacca ccgccccgcc atcggggtca |
| 3781 | tcgtaggctg cgagttgatg ctacgctttg tggccgtggg tctcatcgtc ggcaccgctt |
| 3841 | tcatatcccg gggggcatgt gcgatcacat accccctgtt tctgaccatc accacctggt |
| 3901 | gttttgtctc caccatcggc ctgacagagc tgtattgtat tctgcggcgg ggcccggccc |
| 3961 | ccaagaacgc agacaaggcc gccgccccgg ggcgatccaa ggggctgtcg ggcgtctgcg |
| 4021 | ggcgctgctg ttccatcatc ctctcgggca tcgcagtgcg attgtgttat atcgccgtgg |
| 4081 | tggccggggt ggtgctcgtg gcgcttcact acgagcagga gatccagagg cgcctgtttg |
| 4141 | atgtatgacg tcacatccag gccggcggaa accgtaacgg catatgcaaa ttggaaactg |
| 4201 | tcctgtcttg gggcccaccc acccgacgcg tcatatgcaa atgaaaatcg gtcccccgag |
| 4261 | gccacgtgta gcctggatcc caacgacccc gcccatgggt cccaattggc cgtcccgtta |
| 4321 | ccaagaccaa cccagccagc gtatccaccc ccgcccgggt ccccgcggaa gcggaacggg |
| 4381 | gtatgtgata tgctaattaa a |

UL29 (ICP8) single-stranded DNA-binding protein [Human alphaherpesvirus 1 (Herpes simplex virus type 1)] Gene ID: 2703458. NCBI Reference Sequence: NC_001806.2.

| (SEQ ID NO. 205) | |
|---|---|
| 1 | atggagacaa agcccaagac ggcaaccacc atcaaggtcc cccccgggcc cctgggatac |
| 61 | gtgtacgctc gcgcgtgtcc gtccgaaggc atcgagcttc tggcgttact gtcggcacgc |
| 121 | agcggcgatt ccgacgtcgc cgtggcgccc ctggtcgtgg gcctgaccgt ggagagcggc |
| 181 | tttgaggcca acgtggccgt ggtcgtgggt tctcgcacga cggggctcgg gggtaccgcg |
| 241 | gtgtccctga aactgacgcc ctcgcactac agctcgtccg tgtacgtctt tcacggcggc |
| 301 | cggcacctgg accccagcac ccaggccccg aacctgacgc gactttgcga gcgggcacgc |
| 361 | cgccattttg gcttttcgga ctacaccccc cggcccggcg acctcaaaca cgagacgacg |
| 421 | ggggaggcgc tgtgtgagcg cctcggcctg gacccggacc gcgccctcct gtatctggtc |
| 481 | gttaccgagg gcttcaagga ggccgtgtgc atcaacaaca cctttctgca cctgggaggc |
| 541 | tcggacaagg taaccatagg cggggcggag gtgcaccgca tacccgtgta cccgttgcag |
| 601 | ctgttcatgc cggattttag ccgtgtcatc gcagagccgt tcaacgccaa ccaccgatcg |
| 661 | atcggggaga attttaccta cccgcttccg ttttttaacc gccccctcaa ccgcctcctg |
| 721 | ttcgaggcgg tcgtgggacc cgccgccgtg gcactgcgat gccgaaacgt ggacgccgtg |
| 781 | gcccgcgcgg ccgcccacct ggcgtttgac gaaaaccacg agggcgccgc cctccccgcc |
| 841 | gacattacgt tcacggcctt cgaagccagc cagggtaaga ccccgcgggg cgggcgcgac |
| 901 | ggcggcggca agggcccggc gggcgggttc gaacagcgcc tggcctccgt catggccgga |
| 961 | gacgccgccc tggccctcga gtctatcgtg tcgatggccg tctttgacga gccgcccacc |
| 1021 | gacatctccg cgtggccgct gttcgagggc caggacacgg ccgcggcccg cgccaacgcc |
| 1081 | gtcggggcgt acctggcgcg cgccgcggga ctcgtggggg ccatggtatt tagcaccaac |
| 1141 | tcggccctcc atctcaccga ggtggacgac gccggcccgg cggacccaaa ggaccacagc |
| 1201 | aaaccctcct tttaccgctt cttcctcgtg cccgggaccc acgtggcggc caacccacag |
| 1261 | gtggaccgcg agggacacgt ggtgcccggg ttcgagggtc ggcccaccgc gcccctcgtc |
| 1321 | ggcggaaccc aggaatttgc cggcgagcac ctggccatgc tgtgtgggtt ttccccggcg |
| 1381 | ctgctggcca agatgctgtt ttacctggag cgctgcgacg gcggcgtgat cgtcgggcgc |
| 1441 | caggagatgg acgtgtttcg atacgtcgcg gactccaacc agaccgacgt gccctgtaac |
| 1501 | ctatgcacct tcgacacgcg ccacgcctgc gtacacacga cgctcatgcg cctccgggcg |
| 1561 | cgccatccaa agttcgccag cgccgcccgc ggagccatcg gcgtcttcgg gaccatgaac |
| 1621 | agcatgtaca gcgactgcga cgtgctggga aactacgccg ccttctcggc cctgaagcgc |
| 1681 | gcggacggat ccgagaccgc ccggaccatc atgcaggaga cgtaccgcgc ggcgaccgag |
| 1741 | cgcgtcatgg ccgaactcga gaccctgcag tacgtggacc aggcggtccc cacggccatg |
| 1801 | gggcggctgg agaccatcat caccaaccgc gaggccctgc atacggtggt gaacaacgtc |
| 1861 | aggcaggtcg tggaccgcga ggtggagcag ctgatgcgca acctggtgga ggggaggaac |
| 1921 | ttcaagtttc gcgacggtct gggcgaggcc aaccacgcca tgtccctgac gctggacccg |
| 1981 | tacgcgtgcg ggccgtgccc cctgcttcag cttctcgggc ggcgatccaa cctcgccgtg |
| 2041 | taccaggacc tggccctgag tcagtgccac ggggtgttcg ccgggcagtc ggtcgagggg |
| 2101 | cgcaactttc gcaatcaatt ccaaccggtg ctgcggcggc gcgtgatgga catgtttaac |
| 2161 | aacgggtttc tgtcggccaa aacgctgacg gtcgcgctct cggagggggc ggctatctgc |
| 2221 | gcccccagcc taacggccgg ccagacggcc cccgccgaga gcagcttcga gggcgacgtt |
| 2281 | gcccgcgtga ccctggggtt tcccaaggag ctgcgcgtca agagccgcgt gttgttcgcg |
| 2341 | ggcgcgagcg ccaacgcgtc cgaggccgcc aaggcgcggg tcgccagcct ccagagcgcc |
| 2401 | taccagaagc ccgacaagcg cgtggacatc ctcctcggac cgctgggctt tctgctgaag |
| 2461 | cagttccacg cggccatctt ccccaacggc aagcccccgg ggtccaacca gccgaacccg |
| 2521 | cagtggttct ggacggccct ccaacgcaac cagcttcccg cccggctcct gtcgcgcgag |
| 2581 | gacatcgaga ccatcgcgtt cattaaaaag ttttccctgg actacggcgc gataaacttt |
| 2641 | attaacctgg cccccaacaa cgtgagcgag ctggcgatgt actacatggc aaaccagatt |
| 2701 | ctgcggtact gcgatcactc gacatacttc atcaacaccc ttacggccat catcgcgggg |
| 2761 | tcccgccgtc cccccagcgt gcaggctgcg gccgcgtggt ccgcgcaggg cggggcgggc |
| 2821 | ctggaggccg gggcccgcgc gctgatggac gccgtggacg cgcatccggg cgcgtggacg |
| 2881 | tccatgttcg ccagctgcaa cctgctgcgg cccgtcatgg cggcgcgccc catggtcgtg |
| 2941 | ttggggttga gcatcagcaa gtactacggc atggccggca acgaccgtgt gtttcaggcc |
| 3001 | gggaactggg ccagcctgat gggcggcaaa aacgcgtgcc cgctccttat ttttgaccgc |
| 3061 | acccgcaagt tcgtcctggc ctgtccccgg gccgggtttg tgtgcgcggc ctcaagcctc |
| 3121 | ggcggcggag cgcacgaaag ctcgctgtgc gagcagctcc ggggcattat ctccgagggc |
| 3181 | ggggcggccg tcgccagtag cgtgttcgtg gcgaccgtga aaagcctggg gccccgcacc |
| 3241 | cagcagctgc agatcgagga ctggctggcg ctcctggagg acgagtacct aagcgaggag |
| 3301 | atgatggagc tgaccgcgcg tgccctggag cgcggcaacg gcgagtggtc gacggacgcg |
| 3361 | gccctggagg tggcgcacga ggccgaggcc ctagtcagcc aactcggcaa cgccggggag |
| 3421 | gtgtttaact ttggggattt tggctgcgag gacgacaacg cgacgccgtt cggcggcccg |
| 3481 | ggggccccgg gaccggcatt tgccggccgc aaacgggcgt tccacgggga tgacccgttt |
| 3541 | ggggaggggc cccccgacaa aaagggagac ctgacgttgg atatgctgtg aggggttggg |
| 3601 | gggtggggga acctagggcg gggcggggaa tgtgtgtaaa ataaa |

UL9 DNA replication origin-binding helicase [Human alphaherpesvirus 1 (Herpes simplex virus type 1)] Gene ID: 2703434. NCBI Reference Sequence: NC_001806.2.

| (SEQ ID NO. 206) | |
|---|---|
| 1 | atgcctttcg tggggggcgc ggagtcggga gatcctctgg gggccgggcg tcccattggg |
| 61 | gacgacgagt gcgaacagta cacgtcgagc gtatcgctag cgcggatgtt gtacgggggg |
| 121 | gatttggccg aatgggtgcc ccgggttcac ccgaaaacaa cgatcgagcg gcagcagcac |
| 181 | ggacccgtca ccttccccaa cgcgagcgcc ccgacggcca ggtgcgtgac tgtggtccgc |
| 241 | gcgccaatgg ggtcgggaaa aactaccgcg ctgatccgct ggctgcggga agcgatccac |
| 301 | tctccggaca cgagtgtgct cgtcgtctcc tgtcgtcgga gttttaccca gaccctagcg |
| 361 | acgcggttcg ctgagtcagg cctggtcgac tttgtcacct acttctcatc caccaattac |
| 421 | attatgaacg accgcccctt ccaccgactt atcgtccagg tggaaagcct tcatcgcgtg |
| 481 | ggccccaacc ttctgaacaa ctacgacgtc ctcgttctgg acgaggttat gtcgacgctg |
| 541 | ggccagctct attcgccaac gatgcagcaa ctgggccgcg tggatgcgtt aatgctacgc |
| 601 | ctgctgcgca cctgtcctcg gatcatcgcc atggacgcaa ccgccaacgc gcagttggtg |
| 661 | gacttcctgt gcggtctccg gggcgaaaaa aacgtgcatg tggtggtcgg cgagtacgcc |
| 721 | atgcccgggt tttcggcgcg ccggtgcctg tttctcccgc gtctggggac cgagctcctg |
| 781 | caggctgccc tgcgcccgcc cgggccgccg agcggcccgt ctccggacgc ctctccggac |
| 841 | gcccgggggg ccacgttctt tggggagctg gaagcgcgcc ttggcggggg cgataacatc |
| 901 | tgcatttttt cgtcgacggt ctccttcgcg gagatcgtgg cccggttctg ccgtcagttt |
| 961 | acggaccgcg tgctgttgct tcactcgctc acccccctcg gggacgtgac cacgtggggc |
| 1021 | caataccgcg tggttatata cacgacggtc gtaaccgtgg gcctcagctt cgatcccctg |
| 1081 | cactttgatg gcatgttcgc ctacgtgaaa cccatgaact acggaccgga catggtgtcc |
| 1141 | gtgtaccagt ccctgggacg ggtgcgcacc ctccgcaagg gggagctact gatttacatg |
| 1201 | gacggctccg gggcgcgctc ggagcccgtc tttacgccca tgctccttaa tcacgtggtc |
| 1261 | agttcctgcg gccagtggcc cgcgcagttc tcccaggtca caaacctgct gtgtcgccgg |
| 1321 | ttcaaggggc gctgtgacgc gtcggcatgc gacacgtcgc tggggcgggg gtcgcgcatc |
| 1381 | tacaacaaat tccgttacaa acactacttt gagagatgca cgctggcgtg tctctcggac |
| 1441 | agccttaaca tccttcacat gctgctgacc ctaaactgca tacgcgtgcg cttctgggga |
| 1501 | cacgacgata ccctgacccc aaaggacttc tgtctgtttt tgcggggcgt acatttcgac |
| 1561 | gccctcaggg cccagcgcga tctacgggag ctgcggtgcc gggatcccga ggcgtcgctg |
| 1621 | ccggcccagg ccgccgagac ggaggaggtg ggtcttttcg tcgaaaaata cctccggtcc |
| 1681 | gatgtcgcgc cggcggaaat tgtcgcgctc atgcgcaacc tcaacagcct gatgggacgc |
| 1741 | acgcggttta tttacctggc gttgctggag gcctgtctcc gcgttcccat ggccacccgc |
| 1801 | agcagcgcca tatttcggcg gatctatgac cactacgcca cgggcgtcat ccccacgatc |
| 1861 | aacgtcaccg gagagctgga gctcgtggcc ctgcccccca ccctgaacgt aacccccgtc |
| 1921 | tgggagctgt tgtgcctgtg cagcaccatg gccgcgcgcc tgcattggga ctcggcggcc |
| 1981 | gggggatctg ggaggacctt cggccccgat gacgtgctgg acctactgac cccccactac |
| 2041 | gaccgctaca tgcagctggt gttcgaactg ggccactgta acgtaaccga cggacttctg |
| 2101 | ctctcggagg aagccgtcaa gcgcgtcgcc gacgccctaa gcggctgtcc cccgcgcggg |
| 2161 | tccgttagcg agacggacca cgcggtggcg ctgttcaaga taatctgggg cgaactgttt |
| 2221 | ggcgtgcaga tggccaaaag cacgcagacg tttcccgggg cggggcgcgt taaaaacctc |
| 2281 | accaaacaga caatcgtggg gttgttggac gcccaccaca tcgaccacag cgcctgccgg |
| 2341 | acccacaggc agctgtacgc cctgcttatg gcccacaagc gggagtttgc gggcgcgcgc |
| 2401 | ttcaagctac gcgtgcccgc gtgggggcgc tgtttgcgca cgcactcatc cagcgccaac |
| 2461 | cccaacgctg acatcatcct ggaggcggcg ctgtcggagc tccccaccga ggcctggccc |
| 2521 | atgatgcagg gggcggtgaa ctttagcacc ctataagtct cgggaccgca ctcgttcggt |
| 2581 | acgtggtcgt ccgcggaccg gcggcgctgt tgccggaacg caccgagggg ccaagttggc |
| 2641 | ccccggaccc gggccgtttc ccacccccac cccaacccca aaaaccgccc cccccccgtc |
| 2701 | accggtttcc gcgacccacc gggcccggcc aggcacggca gcatgggacc cacagaccgc |
| 2761 | ccgtgatcct taggggccgt gcgatggaca ccgcagatat cgtgtgggtg gaggagagcg |
| 2821 | tcagcgccat taccctttac gcggtatggc tgcccccccg cgctcgcgag tacttccacg |
| 2881 | ccctggtgta ttttgtatgt cgcaacgccg caggggaggg tcgcgcgcgc tttgcggagg |
| 2941 | tctccgtcac cgcgacggag ctgcgggatt tctacggctc cgcggacgtc tccgtccagg |
| 3001 | ccgtcgtggc ggccgcccgc gccgcgacga cgccggccgc ctccccgctg gagcccctgg |
| 3061 | agaacccgac tctgtggcgg gcgctgtacg cgtgcgtcct ggcggccctg gagcgccaga |
| 3121 | ccgggccggt ggccctgttc gccccgctgc gtatcggctc ggacccacgc acgggactgg |
| 3181 | tggtgaaagt tgagagagcg tcgtggggcc cgcccgccgc ccctcgcgcc gctctcctgg |
| 3241 | tcgcggaggc caacattgac atcgacccta tggccctggc ggcgcgcgtt gccgagcatc |
| 3301 | ccgacgcgcg gctggcgtgg gcgcgcctgg cggccattcg cgacaccccc cagtgcgcgt |
| 3361 | ccgccgcttc gctgaccgtt aacatcacca ccggaaccgc gctatttgcg cgcgaatacc |
| 3421 | agactcttgc gtttccgccg atcaagaagg agggcgcgtt cggggacctg gtcgaggtgt |
| 3481 | gcgaggtggg cctgcggcca cgcgggcacc cgcaacgagt cacggcacgg gtgctgctgc |
| 3541 | cccgcgatta cgactacttt gtaagcgccg gcgagaagtt ctccgcgccg gcgctcgtcg |
| 3601 | cccttttccg gcagtggcat accacggtcc acgccgcccc cggggccctg gcccccgtct |
| 3661 | ttgcctttct ggggcccgag tttgaggtcc gggggggacc cgtcccgtac tttgccgtcc |
| 3721 | tggggtttcc gggttggccc acgttcaccg tgccggccac ggccgagtcg gcacgggacc |
| 3781 | tggtgcgcgg ggccgcggcc gcttacgccg cgctcctggg ggcctggccc gcggtggggg |
| 3841 | ccagggtcgt cctccccccg cgagcctggc ccggcgtggc ctcggcggca gccggatgcc |
| 3901 | tcctgcccgc ggtgcgggag gcggtggcgc ggtggcatcc cgccactaaa atcatccaac |
| 3961 | tgttagaccc gcccgcggcc gtcgggcccg tctggacggc gcggttttgc ttccccggac |
| 4021 | ttcgcgccca gctcctggcg gccctggccg acctcggggg gagcgggctg gcggaccccc |
| 4081 | acggccggac gggcctagca agactggacg cgctggtggt ggccgctccc tcagagccct |
| 4141 | gggccggggc cgtcttggag cgcctggtcc cggacacgtg caacgcctgc cctgcgctgc |
| 4201 | ggcagctcct gggtggggta atggccgccg tctgcctgca gatcgaggag acggccagct |
| 4261 | cggtgaagtt cgcggtctgc gggggcgatg ggggtgcgtt ctggggtgtc tttaacgtgg |
| 4321 | acccccaaga cgcggatgcg gcttccgggg tgatcgagga cgcccggcgg gccatcgaga |
| 4381 | cggccgtggg agccgtgctt agggccaacg gcctccggct gcggcaccca ctgtgcctgg |
| 4441 | ccctcgaggg cgtctacacc cacgcagtcg cctggagcca ggcgggagtg tggttctgga |
| 4501 | actcccgcga caacactgac catcttgggg gatttcctct ccgcgggccc gcgtacacca |
| 4561 | cggcggcagg ggtcgtacgc gacacgctgc gacgggtcct gggcctgaca acggcatgcg |
| 4621 | tgccggagga ggacgcactc acggcccggg gccttatgga ggacgcctgc gaccgcctta |
| 4681 | tcttggacgc gtttaataaa cggttggacg cggagtactg gagcgttcgg gtgtccccct |
| 4741 | ttgaggccag cgaccccttg ccccccactg ccttccgcgg cggcgccttg ctggacgcag |
| 4801 | agcactactg gcggcgcgtc gtgcgtgtct gtcccggagg cggggagtcg gtcggcgtcc |
| 4861 | ccgtcgatct atacccgcgg ccccttgtgc tcccccccgt ggactgcgct catcacctgc |
| 4921 | gcgaaatcct gcgcgagatt gagttggtgt ttaccggggt gctggcggga gtatggggcg |
| 4981 | agggggggaa gtttgtgtat ccctttgacg acaagatgtc gtttctgttt gcctgagttt |
| 5041 | gaccaataaa |

UL30 DNA polymerase catalytic subunit [Human alphaherpesvirus 1 (Herpes simplex virus type 1)] Gene ID: 2703462. NCBI Reference Sequence: NC_001806.2.

| (SEQ ID NO. 207) | |
|---|---|
| 1 | atgttttccg gtggcggcgg cccgctgtcc cccggaggaa agtcggcggc cagggcggcg |
| 61 | tccgggtttt ttgcgcccgc cggccctcgc ggagccagcc ggggaccccc gccttgtttg |
| 121 | aggcaaaact tttacaaccc ctacctcgcc ccagtcggga cgcaacagaa gccgaccggg |
| 181 | ccaacccagc gccatacgta ctatagcgaa tgcgatgaat ttcgattcat cgccccgcgg |
| 241 | gtgctggacg aggatgcccc cccggagaag cgcgccgggg tgcacgacgg tcacctcaag |
| 301 | cgcgccccca aggtgtactg cgggggggac gagcgcgacg tcctccgcgt cgggtcgggc |
| 361 | ggcttctggc cgcggcgctc gcgcctgtgg ggcggcgtgg accacgcccc ggcggggttc |
| 421 | aaccccaccg tcaccgtctt tcacgtgtac gacatcctgg agaacgtgga gcacgcgtac |
| 481 | ggcatgcgcg cggcccagtt ccacgcgcgg tttatggacg ccatcacacc gacggggacc |
| 541 | gtcatcacgc tcctgggcct gactccggaa ggccaccggg tggccgttca cgtttacggc |
| 601 | acgcggcagt acttttacat gaacaaggag gaggtcgaca ggcacctaca atgccgcgcc |
| 661 | ccacgagatc tctgcgagcg catggccgcg gccctgcgcg agtccccggg cgcgtcgttc |
| 721 | cgcggcatct ccgcggacca cttcgaggcg gaggtggtgg agcgcaccga cgtgtactac |
| 781 | tacgagacgc gccccgctct gttttaccgc gtctacgtcc gaagcgggcg cgtgctgtcg |
| 841 | tacctgtgcg acaacttctg cccggccatc aagaagtacg agggtggggt cgacgccacc |
| 901 | acccggttca tcctggacaa ccccgggttc gtcaccttcg gctggtaccg tctcaaaccg |
| 961 | ggccggaaca acacgctagc ccagccgcgg gccccgatgg ccttcgggac atccagcgac |
| 1021 | gtcgagttta actgtacggc ggacaacctg gccatcgagg ggggcatgag cgacctaccg |
| 1081 | gcatacaagc tcatgtgctt cgatatcgaa tgcaaggcgg ggggggagga cgagctggcc |
| 1141 | tttccggtgg ccgggcaccc ggaggacctg gtcatccaga tatcctgtct gctctacgac |
| 1201 | ctgtccacca ccgccctgga gcacgtcctc ctgttttcgc tcggttcctg cgacctcccc |
| 1261 | gaatcccacc tgaacgagct ggcggccagg ggcctgccca cgcccgtggt tctggaattc |
| 1321 | gacagcgaat tcgagatgct gttggccttc atgacccttg tgaaacagta cggccccgag |
| 1381 | ttcgtgaccg ggtacaacat catcaacttc gactggccct tcttgctggc caagctgacg |
| 1441 | gacatttaca aggtccccct ggacgggtac ggccgcatga acggccgggg cgtgtttcgc |
| 1501 | gtgtgggaca taggccagag ccacttccag aagcgcagca agataaaggt gaacggcatg |
| 1561 | gtgaacatcg acatgtacgg gattataacc gacaagatca agctctcgag ctacaagctc |
| 1621 | aacgccgtgg ccgaagccgt cctgaaggac aagaagaagg acctgagcta tcgcgacatc |
| 1681 | cccgcctact acgccgccgg gcccgcgcaa cgcggggtga tcggcgagta ctgcatacag |
| 1741 | gattccctgc tggtgggcca gctgtttttt aagtttttgc cccatctgga gctctcggcc |
| 1801 | gtcgcgcgct tggcgggtat taacatcacc cgcaccatct acgacggcca gcagatccgc |
| 1861 | gtctttacgt gcctgctgcg cctggccgac cagaagggct ttattctgcc ggacacccag |
| 1921 | gggcgattta ggggcgccgg gggggaggcg cccaagcgtc cggccgcagc ccgggaggac |
| 1981 | gaggagcggc cagaggagga gggggaggac gaggacgaac gcgaggaggg cgggggcgag |
| 2041 | cgggagccgg agggcgcgcg ggagaccgcc ggcaggcacg tggggtacca gggggccagg |
| 2101 | gtccttgacc ccacttccgg gtttcacgtg aaccccgtgg tggtgttcga ctttgccagc |
| 2161 | ctgtacccca gcatcatcca ggcccacaac ctgtgcttca gcacgctctc cctgagggcc |
| 2221 | gacgcagtgg cgcacctgga ggcgggcaag gactacctgg agatcgaggt gggggggcga |
| 2281 | cggctgttct tcgtcaaggc tcacgtgcga gagagcctcc tcagcatcct cctgcgggac |
| 2341 | tggctcgcca tgcgaaagca gatccgctcg cggattcccc agagcagccc cgaggaggcc |
| 2401 | gtgctcctgg acaagcagca ggccgccatc aaggtcgtgt gtaactcggt gtacgggttc |
| 2461 | acgggagtgc agcacggact cctgccgtgc ctgcacgttg ccgcgacggt gacgaccatc |
| 2521 | ggccgcgaga tgctgctcgc gacccgcgag tacgtccacg cgcgctgggc ggccttcgaa |
| 2581 | cagctcctgg ccgatttccc ggaggcggcc gacatgcgcg cccccgggcc ctattccatg |
| 2641 | cgcatcatct acggggacac ggactccatc tttgtgctgt gccgcggcct cacggccgcc |
| 2701 | gggctgacgg ccgtgggcga caagatggcg agccacatct cgcgcgcgct gtttctgccc |
| 2761 | cccatcaaac tcgagtgcga aaagacgttc accaagctgc tgctgatcgc caagaaaaag |
| 2821 | tacatcggcg tcatctacgg gggtaagatg ctcatcaagg gcgtggatct ggtgcgcaaa |
| 2881 | aacaactgcg cgtttatcaa ccgcacctcc agggccctgg tcgacctgct gttttacgac |
| 2941 | gataccgtct ccggagcggc cgccgcgtta gccgagcgcc ccgcggagga gtggctggcg |
| 3001 | cgacccctgc ccgagggact gcaggcgttc ggggccgtcc tcgtagacgc ccatcggcgc |
| 3061 | atcaccgacc cggagaggga catccaggac tttgtcctca ccgccgaact gagcagacac |
| 3121 | ccgcgcgcgt acaccaacaa gcgcctggcc cacctgacgg tgtattacaa gctcatggcc |
| 3181 | cgccgcgcgc aggtcccgtc catcaaggac cggatcccgt acgtgatcgt ggcccagacc |
| 3241 | cgcgaggtag aggagacggt cgcgcggctg gccgccctcc gcgagctaga cgccgccgcc |
| 3301 | ccaggggacg agcccgcccc ccccgcggcc ctgccctccc cggccaagcg cccccgggag |
| 3361 | acgccgtcgc ctgccgaccc cccgggaggc gcgtccaagc cccgcaagct gctggtgtcc |
| 3421 | gagctggccg aggatcccgc atacgccatt gcccacggcg tcgccctgaa cacggactat |
| 3481 | tacttctccc acctgttggg ggcggcgtgc gtgacattca aggccctgtt tgggaataac |
| 3541 | gccaagatca ccgagagtct gttaaaaagg tttattcccg aagtgtggca ccccccggac |
| 3601 | gacgtggccg cgcggctccg gaccgcaggg ttcggggcgg tgggtgccgg cgctacggcg |
| 3661 | gaggaaactc gtcgaatgtt gcatagagcc tttgatactc tagcatgagc cccccgtcga |
| 3721 | agctgatgtc cctcatttta caataaa |

UL42 DNA polymerase processivity subunit [Human alphaherpesvirus 1 (Herpes simplex virus type 1)] Gene ID: 24271471. NCBI Reference Sequence: NC_001806.2.

| (SEQ ID NO. 208) | |
|---|---|
| 1 | atgacggatt cccctggcgg tgtggccccc gcctcccccg tggaggacgc gtcggacgcg |
| 61 | tccctcgggc agccggagga gggggcgccc tgccaggtgg tcctgcaggg cgccgaactt |
| 121 | aatggaatcc tacaggcgtt tgccccgctg cgcacgagcc ttctggactc gcttctggtt |
| 181 | atgggcgacc ggggcatcct tatccataac acgatctttg gggagcaggt gttcctgccc |
| 241 | ctggaacact cgcaattcag tcggtatcgc tggcgcggac ccacggcggc gttcctgtct |
| 301 | ctcgtggacc agaagcgctc cctcctgagc gtgtttcgcg ccaaccagta cccggaccta |
| 361 | cgtcgggtgg agttggcgat cacgggccag gccccgtttc gcacgctggt tcagcgcata |
| 421 | tggacgacga cgtccgacgg cgaggccgtt gagctagcca gcgagacgct gatgaagcgc |
| 481 | gaactgacga gctttgtggt gctggttccc cagggaaccc ccgacgttca gttgcgcctg |
| 541 | acgaggccgc agctcaccaa ggtccttaac gcgaccgggg ccgatagtgc cacgcccacc |
| 601 | acgttcgagc tcggggttaa cggcaaattt tccgtgttca ccacgagtac ctgcgtcacc |
| 661 | tttgctgccc gcgaggaggg cgtgtcgtcc agcaccagca cccaggtcca gatcctgtcc |
| 721 | aacgcgctca ccaaggcggg ccaggcggcc gccaacgcca agacggtgta cggggaaaat |
| 781 | acccatcgca ccttctctgt ggtcgtcgac gattgcagca tgcgggcggt gctccggcga |
| 841 | ctgcaggtcg gcgggggcac cctcaagttc ttcctcacga cccccgtccc cagtctgtgc |
| 901 | gtcaccgcca ccggtcccaa cgcggtatcg gcggtatttc tcctgaaacc ccagaagatt |
| 961 | tgcctggact ggctgggtca tagccagggg tctccttcag ccgggagctc ggcctcccgg |
| 1021 | gcctctggga gcgagccaac agacagccag gactccgcgt cggacgcggt cagccacggc |
| 1081 | gatccggaag acctcgatgg cgctgcccgg gcgggagagg cgggggcctt gcatgcctgt |
| 1141 | ccgatgccgt cgtcgaccac gcgggtcact cccacgacca agcgggggcg ctcggggggc |
| 1201 | gaggatgcgc gcgcggacac ggccctaaag aaacctaaga cggggtcgcc caccgcaccc |
| 1261 | ccgcccgcag atccagtccc cctggacacg gaggacgact ccgatgcggc ggacgggacg |
| 1321 | gcggcccgtc ccgccgctcc agacgcccgg agcggaagcc gttacgcgtg ttactttcgc |
| 1381 | gacctcccga ccggagaagc aagccccggc gccttctccg ccttccgggg gggcccccaa |
| 1441 | accccgtatg gttttggatt cccctgacgg ggcggggcct tggcggccgc ccaactctcg |
| 1501 | caccatcccg ggttaatgta aataaa |

### Example 37—Human Papilloma Virus (HPV) Helper Polynucleotide Sequences

HPV polynucleotides can be selected from any serotype, and representative polynucleotides are exemplified below. Meier et al. 2020; Cao et al. 2012 (Cao, M., et al. “HPV-16 E1, E2 and E6 each complement the Ad5 helper gene set, increasing rAAV2 and wt AAV2 production.” Gene therapy 19.4 (2012): 418-424); You et al. 2006 (You, Hong, et al. “Multiple human papillomavirus genes affect the adeno-associated virus life cycle.” Virology 344.2 (2006): 532-540); and Ogston et al. 2000 (Ogston, P.; Raj, K.; Beard, P. Productive Replication of Adeno-Associated Virus Can Occur in Human Papillomavirus Type 16 (HPV-16) Episome-Containing Keratinocytes and Is Augmented by the HPV-16 E2 Protein. J. Virol. 2000, 74, 3494-3504) disclose four HPV early genes E1, E2, E6 and E7, of which E1 shows the highest helping activity, E2 and E6 with intermediate helper activity and E7 with little effect or possibly a slight decrease in cap expression. The three HPV genes (E1, E2, and E6) are unable to stimulate significant rAAV replication in HEK293 cells when used alone. However, when used in conjunction (complementation) with the standard Ad5 helper gene set, E1, E2 and E6 are each capable of significantly boosting rAAV DNA replication and virus particle yield. HPV early gene (E1, E2, E6 and E7) sequences as disclosed at the GenBank are listed below:

E1 replication protein E1 [Human papillomavirus type 16] Gene ID: 1489075. NCBI Reference Sequence: NC_001526.4.

| (SEQ ID NO. 209) | |
|---|---|
| 1 | atggctgatc ctgcaggtac caatggggaa gagggtacgg gatgtaatgg atggttttat |
| 61 | gtagaggctg tagtggaaaa aaaaacaggg gatgctatat cagatgacga gaacgaaaat |
| 121 | gacagtgata caggtgaaga tttggtagat tttatagtaa atgataatga ttatttaaca |
| 181 | caggcagaaa cagagacagc acatgcgttg tttactgcac aggaagcaaa acaacataga |
| 241 | gatgcagtac aggttctaaa acgaaagtat ttgggtagtc cacttagtga tattagtgga |
| 301 | tgtgtagaca ataatattag tcctagatta aaagctatat gtatagaaaa acaaagtaga |
| 361 | gctgcaaaaa ggagattatt tgaaagcgaa gacagcgggt atggcaatac tgaagtggaa |
| 421 | actcagcaga tgttacaggt agaagggcgc catgagactg aaacaccatg tagtcagtat |
| 481 | agtggtggaa gtgggggtgg ttgcagtcag tacagtagtg gaagtggggg agagggtgtt |
| 541 | agtgaaagac acactatatg ccaaacacca cttacaaata ttttaaatgt actaaaaact |
| 601 | agtaatgcaa aggcagcaat gttagcaaaa tttaaagagt tatacggggt gagtttttca |
| 661 | gaattagtaa gaccatttaa aagtaataaa tcaacgtgtt gcgattggtg tattgctgca |
| 721 | tttggactta cacccagtat agctgacagt ataaaaacac tattacaaca atattgttta |
| 781 | tatttacaca ttcaaagttt agcatgttca tggggaatgg ttgtgttact attagtaaga |
| 841 | tataaatgtg gaaaaaatag agaaacaatt gaaaaattgc tgtctaaact attatgtgtg |
| 901 | tctccaatgt gtatgatgat agagcctcca aaattgcgta gtacagcagc agcattatat |
| 961 | tggtataaaa caggtatatc aaatattagt gaagtgtatg gagacacgcc agaatggata |
| 1021 | caaagacaaa cagtattaca acatagtttt aatgattgta catttgaatt atcacagatg |
| 1081 | gtacaatggg cctacgataa tgacatagta gacgatagtg aaattgcata taaatatgca |
| 1141 | caattggcag acactaatag taatgcaagt gcctttctaa aaagtaattc acaggcaaaa |
| 1201 | attgtaaagg attgtgcaac aatgtgtaga cattataaac gagcagaaaa aaaacaaatg |
| 1261 | agtatgagtc aatggataaa atatagatgt gatagggtag atgatggagg tgattggaag |
| 1321 | caaattgtta tgtttttaag gtatcaaggt gtagagttta tgtcattttt aactgcatta |
| 1381 | aaaagatttt tgcaaggcat acctaaaaaa aattgcatat tactatatgg tgcagctaac |
| 1441 | acaggtaaat cattatttgg tatgagttta atgaaatttc tgcaagggtc tgtaatatgt |
| 1501 | tttgtaaatt ctaaaagcca tttttggtta caaccattag cagatgccaa aataggtatg |
| 1561 | ttagatgatg ctacagtgcc ctgttggaac tacatagatg acaatttaag aaatgcattg |
| 1621 | gatggaaatt tagtttctat ggatgtaaag catagaccat tggtacaact aaaatgccct |
| 1681 | ccattattaa ttacatctaa cattaatgct ggtacagatt ctaggtggcc ttatttacat |
| 1741 | aatagattgg tggtgtttac atttcctaat gagtttccat ttgacgaaaa cggaaatcca |
| 1801 | gtgtatgagc ttaatgataa gaactggaaa tcctttttct caaggacgtg gtccagatta |
| 1861 | agtttgcacg aggacgagga caaggaaaac gatggagact ctttgccaac gtttaaatgt |
| 1921 | gtgtcaggac aaaatactaa cacattatga |

E2 regulatory protein E2 [Human papillomavirus type 16] Gene ID: 1489080. NCBI Reference Sequence: NC_001526.4.

| (SEQ ID NO. 210) | |
|---|---|
| 1 | atggagactc tttgccaacg tttaaatgtg tgtcaggaca aaatactaac acattatgaa |
| 61 | aatgatagta cagacctacg tgaccatata gactattgga aacacatgcg cctagaatgt |
| 121 | gctatttatt acaaggccag agaaatggga tttaaacata ttaaccacca ggtggtgcca |
| 181 | acactggctg tatcaaagaa taaagcatta caagcaattg aactgcaact aacgttagaa |
| 241 | acaatatata actcacaata tagtaatgaa aagtggacat tacaagacgt tagccttgaa |
| 301 | gtgtatttaa ctgcaccaac aggatgtata aaaaaacatg gatatacagt ggaagtgcag |
| 361 | tttgatggag acatatgcaa tacaatgcat tatacaaact ggacacatat atatatttgt |
| 421 | gaagaagcat cagtaactgt ggtagagggt caagttgact attatggttt atattatgtt |
| 481 | catgaaggaa tacgaacata ttttgtgcag tttaaagatg atgcagaaaa atatagtaaa |
| 541 | aataaagtat gggaagttca tgcgggtggt caggtaatat tatgtcctac atctgtgttt |
| 601 | agcagcaacg aagtatcctc tcctgaaatt attaggcagc acttggccaa ccaccccgcc |
| 661 | gcgacccata ccaaagccgt cgccttgggc accgaagaaa cacagacgac tatccagcga |
| 721 | ccaagatcag agccagacac cggaaacccc tgccacacca ctaagttgtt gcacagagac |
| 781 | tcagtggaca gtgctccaat cctcactgca tttaacagct cacacaaagg acggattaac |
| 841 | tgtaatagta acactacacc catagtacat ttaaaaggtg atgctaatac tttaaaatgt |
| 901 | ttaagatata gatttaaaaa gcattgtaca ttgtatactg cagtgtcgtc tacatggcat |
| 961 | tggacaggac ataatgtaaa acataaaagt gcaattgtta cacttacata tgatagtgaa |
| 1021 | tggcaacgtg accaattttt gtctcaagtt aaaataccaa aaactattac agtgtctact |
| 1081 | ggatttatgt ctatatga |

E6 protein E6*;transforming protein E6 [Human papillomavirus type 16] Gene ID: 1489078. NCBI Reference Sequence: NC_001526.4.

| (SEQ ID NO. 211) | |
|---|---|
| 1 | atgcaccaaa agagaactgc aatgtttcag gacccacagg agcgacccag aaagttacca |
| 61 | cagttatgca cagagctgca aacaactata catgatataa tattagaatg tgtgtactgc |
| 121 | aagcaacagt tactgcgacg tgaggtatat gactttgctt ttcgggattt atgcatagta |
| 181 | tatagagatg ggaatccata tgctgtatgt gataaatgtt taaagtttta ttctaaaatt |
| 241 | agtgagtata gacattattg ttatagtttg tatggaacaa cattagaaca gcaatacaac |
| 301 | aaaccgttgt gtgatttgtt aattaggtgt attaactgtc aaaagccact gtgtcctgaa |
| 361 | gaaaagcaaa gacatctgga caaaaagcaa agattccata atataagggg tcggtggacc |
| 421 | ggtcgatgta tgtcttgttg cagatcatca agaacacgta gagaaaccca gctgtaa |

E7 transforming protein E7 [Human papillomavirus type 16] Gene ID: 1489079. NCBI Reference Sequence: NC_001526.4.

| (SEQ ID NO. 212) | |
|---|---|
| 1 | atgcatggag atacacctac attgcatgaa tatatgttag atttgcaacc agagacaact |
| 61 | gatctctact gttatgagca attaaatgac agctcagagg aggaggatga aatagatggt |
| 121 | ccagctggac aagcagaacc ggacagagcc cattacaata ttgtaacctt ttgttgcaag |
| 181 | tgtgactcta cgcttcggtt gtgcgtacaa agcacacacg tagacattcg tactttggaa |
| 241 | gacctgttaa tgggcacact aggaattgtg tgccccatct gttctcagaa accataa |

### Example 38—Bocavirus 1 and Baculovirus Helper Polynucleotide Sequences

Bocavirus polynucleotides can be selected from any serotype, and representative polynucleotides as disclosed at the GenBank are exemplified below. Meier et al 2020; Wang, Zekun, et al. 2017 (Wang, Zekun, et al. “Human bocavirus 1 is a novel helper for adeno-associated virus replication.” Journal of virology 91.18 (2017): 10-1128); Guido, Marcello, et al. 2016 (Guido, Marcello, et al. “Human bocavirus: current knowledge and future challenges.” World journal of gastroenterology 22.39 (2016): 8684); and Ning, Kang, et al. 2022 (Ning, Kang, et al. “The small nonstructural protein NP1 of human bocavirus 1 directly interacts with Ku70 and RPA70 and facilitates viral DNA replication.” PLoS pathogens 18.6 (2022): el 010578) disclose that human bocavirus 1 (HBoV1) NS2 (but not NS4), NP1, and BocaSR were required for AAV2 DNA replication and progeny virion formation. Novel small NS proteins (NS2, NS3 and NS4) have been identified in HBoV1, which contain the predictive domains of NS1 activities. HBoV1 expresses one large nonstructural protein (NS1), four small nonstructural proteins (NS2, NS3, NS4, and NP1), one small noncoding RNA (bocavirus-encoded small RNA, BocaSR), and three viral capsid proteins (VP1, VP2, and VP3) from a single precursor mRNA (pre-mRNA) via alternative splicing. NS1, NP1, and BocaSR are essential for DNA replication of HBoV1.

| HBOV1 NP1 | |
|---|---|
| (SEQ ID NO. 213) | |
| 1 | atgacgaaga tgagctcagg gaatatgaaa gacaagcatc gctcctacaa aagaaaaggg |
| 61 | agtccagaaa gaggggagag gaagagacac tggcagacaa ctcatcacag gagcaggagc |
| 121 | cgcagcccga tccgacacag tggggagaga ggctcgggct catatcatca ggaacaccca |
| 181 | atcagccacc tattgtcttg cactgcttcg aagacctcag accaagtgat gaagacgagg |
| 241 | gagagtacat cggggaaaaa agacaataga acaaatccat acactgtatt cagtcaacac |
| 301 | agagcttcca atcctgaagc tccagggtgg tgtgggttct actggcactc tactcgcatt |
| 401 | gctagagatg gtactaattc aatctttaat gaaatgaaac aacagtttca acagctacaa |
| 421 | attgataata aaataggatg ggataacact agagaactat tgtttaatca aaagaaaaca |
| 481 | ctagatcaaa aatacagaaa tatgttctgg cactttagaa ataactctga ttgtgaaaga |
| 541 | tgtaattact gggatgatgt gtaccgtaga cacttagcta atgtttcctc acagacagaa |
| 601 | gcagacgaga taactgacga ggaaatgctt tctgctgctg aaagcatgga agcagatgcc |
| 661 | tccaattaag agacagccta gagggtgggt gctgcctgga tacagatatc ttgggc |
| HBOV1 NS1 | |
| (SEQ ID NO. 214) | |
| 1 | gccggcagac atattggatt ccaagatggc gtctgtacaa ccacgtcaca tataaaataa |
| 61 | taaatattca caaggaggag tggttatatg atgtaatcca taaccactcc caggaaatga |
| 121 | cgtatgatag ccaatcagaa ttaagtatta aacctatata agctgctgca cttcctgatt |
| 181 | caatcagact gcatccggtc tccggcgagt gaacatctct ggaaaaagct ccacgcttgt |
| 241 | ggtgagtcta ctatggcttt caatcctcct gtgattagag ctttttctca acctgctttt |
| 301 | acttatgtct tcaaatttcc atatccacaa tggaaagaaa aagaatggct gcttcatgca |
| 361 | cttttagctc atggaactga acaatctatg atacaattaa gaaactgcgc ttctcatccg |
| 421 | gatgaagaca taatccgtga tgacttgctt atttctttag aagatcgcca ttttggggct |
| 481 | gttctctgca aggctgttta catggcaaca actactctca tgtcacacaa acaaaggaat |
| 541 | atgtttcctc gttgtgacat catagttcag tctgagctag gagagaaaaa cttacactgc |
| 601 | catattatag ttgggggaga aggactaagc aagaggaatg ctaaatcatc ctgtgctcag |
| 661 | ttctatggtt taatactagc tgagataatt caacgctgca aatctcttct ggctacacgt |
| 721 | ccttttgaac ctgaggaggc tgacatattt cacactctaa aaaaggctga gcgagaggca |
| 781 | tggggtggag ttactggcgg caacatgcag atccttcaat atagagatcg cagaggagac |
| 841 | cttcatgcac aaacagtgga tcctcttcgc ttcttcaaaa actacctttt acctaaaaat |
| 901 | agatgtattt catcttacag caaacctgat gtttgtactt ctcctgacaa ctggttcatt |
| 961 | ttagctgaaa aaacttactc tcacactctt attaacgggc tgccgcttcc agaacattac |
| 1021 | agaaaaaact accacgcaac cctagataac gaagtcattc cagggcctca agcaatggcc |
| 1081 | tatggaggac gtggtccgtg ggaacatctt cctgaggtag gagatcagcg cctagctgcg |
| 1141 | tcttctgtta gcactactta taaacctaac aaaaaagaaa aacttatgct aaacttgcta |
| 1201 | gacaaatgta aagagctaaa tctattagtt tatgaagact tagtagctaa ttgtcctgaa |
| 1261 | ctactcctta tgcttgaagg tcaaccagga ggggcacgcc ttatagaaca agtcttgggc |
| 1321 | atgcaccata ttaatgtttg ttctaacttt acagctctca catatctttt tcatctacat |
| 1381 | cctgttactt cgcttgactc agacaataaa gctttacagc ttttgttgat tcaaggctat |
| 1441 | aatcctctag ccgttggtca cgccctgtgc tgtgtcctga acaaacaatt cgggaaacaa |
| 1501 | aacactgttt gcttttacgg gcctgcctca acaggtaaaa caaatatggc caaggcaatc |
| 1561 | gtccaaggga ttagacttta tgggtgtgtt aatcatttga acaaaggatt tgtatttaat |
| 1621 | gactgcagac aacgcctagt tgtttggtgg gaggagtgct taatgcacca ggattgggtg |
| 1681 | gaacctgcaa agtgtatctt gggcgggaca gaatgcagaa ttgacgtcaa gcatagagac |
| 1741 | agtgtacttt taactcaaac acctgtaatt atatccacta accacgatat ctacgcggtt |
| 1801 | gttggtggca attctgtttc tcatgttcac gcggctccat taaaagaaag agtgattcag |
| 1861 | ctaaatttta tgaaacaact tcctcaaaca tttggagaaa tcactgctac tgagattgca |
| 1921 | gctcttctac agtggtgttt caatgagtac gactgtactc tgacaggatt taaacaaaaa |
| 1981 | tggaatttag acaaaattcc aaactcattt cctcttgggg tcctttgtcc tactcattca |
| 2041 | caggacttta cacttcacga aaacggatac tgcactgatt gcggtggtta ccttcctcat |
| 2101 | agtgctgaca attctatgta cactgatcgc gcaagcgaaa ctagcacagg agacatcaca |
| 2161 | ccaagtaagt aaatacgcat gcgcaagtaa ttcttttact ttcacttcgc tatttttacc |
| 2221 | aatttttact tttaggtgac ttgggggatt cggacggaga agacaccgag cctgagacat |
| 2281 | cgcaagtgga ctattgtcca cccaagaaac gtcgtctaac tgctccagca agtcctccaa |
| 2341 | actcacctgc gagctctgta agtactatta ctttctttaa cacttggcac gcacagccac |
| 2401 | gtgacgaaga tgagctcagg gaatatgaaa gacaagcatc gctcctacaa aagaaaaggg |
| 2461 | agtccagaaa gaggggagag gaagagacac tggcagacaa ctcatcacag gagcaggagc |
| 2521 | cgcagcccga tccgacacag tggggagaga ggctcgggct catatcatca ggaacaccca |
| 2581 | atcagccacc tatcgtcttg cactgcttcg aagacctcag accaagtgat gaagacgagg |
| 2641 | gaaagtacat cggggaaaaa agacaataga acaaatccat acactgtatt cagtcaacac |
| 2701 | agagcttcca atcctgaagc tccagggtgg tgtgggttct actggcactc tactcgcatt |
| 2761 | gctagagatg gtactaattc aatctttaat gaaatgaaac aacagtttca acagctacaa |
| 2821 | attgataata aaataggatg ggataacact agagaactat tgtttaatca aaagaaaaca |
| 2881 | ctagatcaaa aatacagaaa tatgttctgg cactttagaa ataactctga ttgtgaaaga |
| 2941 | tgtaattact gggatgatgt gtaccgtaga cacttagcta atgtttcctc acagacagaa |
| 3001 | gcagacgaga taactgacga ggaaatgctt tctgctgctg aaagcatgga agcagatgcc |
| 3061 | tccaattaag agacagccta gagggtgggt gctgcctgga tacagatatc ttgggccatt |
| 3121 | taatccactt gataacggtg aacctgtaaa taacgctgat cgcgctgctc aattacatga |
| 3181 | tcacgcctac tctgaactaa taaagagtgg taaaaatcca tacctgtatt tcaataaagc |
| 3241 | tgatgaaaaa ttcattgatg atctaaaaga cgattggtca attggtggaa ttattggatc |
| 3301 | cagttttttt aaaataaagc gcgccgtggc tcctgctttg ggaaataaag agagagccca |
| 3361 | aaaaagacac ttttactttg ctaactcaaa taaaggtgca aaaaaaacaa aaaaaagtga |
| 3421 | acctaaacca ggaacctcaa aaatgtctga cactgacatt caagaccaac aacctgatac |
| 3481 | tgtggacgca ccacaaaaca cctcaggggg aggaacagga agtattggag gaggaaaagg |
| 3541 | atctggtgtg gggatttcca ctggagggtg ggtcggaggt tctcactttt cagacaaata |
| 3601 | tgtggttact aaaaacacaa gacaatttat aaccacaatt caaaatggtc acctctacaa |
| 3661 | aacagaggcc attgaaacaa caaaccaaag tggaaaatca cagcgctgcg tcacaactcc |
| 3721 | atggacatac tttaacttta atcaatacag ctgtcacttc tcaccacagg attggcagcg |
| 3781 | ccttacaaat gaatataagc gcttcagacc taaagcaatg caagtaaaaa tttacaactt |
| 3841 | gcaaataaaa caaatacttt caaatggtgc tgacacaaca tacaacaatg acctcacagc |
| 3901 | tggcgttcac atcttttgtg atggagagca tgcttaccca aatgcatctc atccatggga |
| 3961 | tgaggacgtc atgcctgatc ttccatacaa gacctggaaa ctttttcaat atggatatat |
| 4021 | tcctattgaa aatgaactcg cagatcttga tggaaatgca gctggaggca atgctacaga |
| 4081 | aaaagcactt ctgtatcaga tgcctttttt tctacttgaa aacagtgacc accaagtact |
| 4141 | tagaactggt gagagcactg aatttacttt taactttgac tgtgaatggg ttaacaatga |
| 4201 | aagagcatac attcctcctg gactaatgtt taatccaaaa gtcccaacaa gaagagttca |
| 4261 | gtacataaga caaaacggaa gcacagcagc cagcacaggc agaattcagc catactcaaa |
| 4321 | accaacaagc tggatgacag gacctggcct gctcagtgca caaagagtag gaccacagtc |
| 4381 | atcagacact gctccattca tggtttgcac taacccagaa ggaacacaca taaacacagg |
| 4441 | tgctgcagga tttggatctg gctttgatcc tccaagcgga tgtctggcac caactaacct |
| 4501 | agaatacaaa cttcagtggt accagacacc agaaggaaca ggaaataatg gaaacataat |
| 4561 | tgcaaaccca tcactctcaa tgcttagaga ccaactccta tacaaaggaa accagaccac |
| 4621 | atacaatcta gtgggggaca tatggatgtt tccaaatcaa gtctgggaca gatttcctat |
| 4681 | caccagagaa aatccaatct ggtgcaaaaa accaagggct gacaaacaca caatcatgga |
| 4741 | tccatttgat ggatcaattg caatggatca tcctccaggc actattttta taaaaatggc |
| 4801 | aaaaattcca gttccaactg cctcaaatgc agactcatac ctaaacatat actgtactgg |
| 4861 | acaagtcagc tgtgaaattg tatgggaggt agaaagatac gcaacaaaga actggcgtcc |
| 4921 | agaaagaaga catactgcac tcgggatgtc actgggagga gaaagcaact acacgcctac |
| 4981 | ataccacgtg gatccaacag gagcatacat ccagcccacg tcatatgatc agtgtatgcc |
| 5041 | agtaaaaaca aacatcaata aagtgttgta atcttataag cctctttttt gcttctgctt |
| 5101 | acaagttcct cctcaatgga caagcggaaa gtgaagggtg actgtagtcc tgagctcatg |
| 5161 | ggttcaagac cacagcccga tggtagtggt gttaccgtct cgaacctagc cgacagccct |
| 5221 | tgtacattgt ggggggagct gttttgtttg cttatgcaat cgcgaaactc tatatctttt |
| 5281 | aatgtgttgt tgttgtaca |

It is to be understood that the description, specific examples and data, while indicating exemplary embodiments, are given by way of illustration and are not intended to limit the present inventions. Various changes and modifications within the present inventions, including combining embodiments in whole and in part, will become apparent to the skilled artisan from the discussion, disclosure and data contained herein, and thus are considered part of the inventions.

1. A method of producing a covalently surface modified adeno-associated virus (AAV), wherein the method comprises the steps of (A) transfecting a cell with:
(i) a plasmid comprising a gene of interest flanked by AAV inverted terminal repeats;
(ii) a plasmid comprising an AAV rep gene and an AAV cap gene;
(iii) a plasmid comprising AAV rep and cap genes and a polynucleotide sequence encoding a first member of a specific binding pair;
(iv) a plasmid comprising one or more helper polynucleotide sequences;
(v) a plasmid comprising a polynucleotide sequence encoding a first portion of a retargeting molecule and a polynucleotide encoding a second cognate member of the specific binding pair; and
(vi) a plasmid comprising a polynucleotide encoding a second portion of a retargeting molecule;
(B) culturing the transfected cell to allow expression of plasmids (i) to (vi) and assembly of proteins to form covalently surface modified adeno-associated virus; and
(C) harvesting the covalently surface modified adeno-associated virus.

2. The method according to claim 1, wherein the cell is a mammalian cell, wherein the mammalian cell is a human cell, and, wherein the human cell is a HEK 293 cell.

3.-5. (canceled)

6. The method according to claim 2, wherein the HEK 293 cell is an adherent HEK 293 cell.

7. The method according to claim 1, wherein the retargeting molecule is a monoclonal antibody, wherein the first portion of the retargeting molecule is an antibody heavy chain or an antibody light chain, and wherein the second portion of the retargeting molecule is an antibody light chain or an antibody heavy chain.

8.-11. (canceled)

12. The method according to claim 1, wherein the specific binding pair is SpyTag-SpyCatcher, wherein the first member of a specific binding pair is SpyTag peptide.

13. (canceled)

14. The method according to claim 7, wherein the first member of a specific binding pair is SpyTag peptide, and the polynucleotide encoding the SpyTag peptide is inserted into the AAV cap gene to encode recombinant capsid proteins.

15. The method according to claim 14, wherein the cell expresses at least one recombinant capsid protein selected from the group consisting of a recombinant VP1 protein comprising a SpyTag amino acid sequence, a recombinant VP2 protein comprising a SpyTag amino acid sequence, and a recombinant VP3 protein comprising a SpyTag amino acid sequence.

16. The method according to claim 12, wherein the second cognate member of the specific binding pair is a SpyCatcher protein.

17. The method according to claim 1, wherein VP1 protein is mutated in a galactose binding domain to detarget liver cells, wherein a detargeting mutation is at least one selected from the group consisting of N272A and W503A.

18. (canceled)

19. The method according to claim 1, wherein the covalently surface modified adeno-associated virus comprises
a plurality of first members bound to second cognate members, wherein the second cognate members also are bound to retargeting molecules; and a gene of interest.

20. The method according to claim 19, wherein the first members are SpyTag peptides and second cognate members at SpyCatcher proteins, wherein the retargeting molecules are antibodies, antibody fragments or antibody derivatives.

21. (canceled)

22. The method according to claim 1, further comprising the step of (D) purifying the covalently surface modified adeno-associated virus using depth filtration followed by single-pass tangential flow filtration, wherein the depth filtration does not require an endonuclease.

23. (canceled)

24. The method according to claim 22, further comprising the step of (E) purifying the covalently surface modified adeno-associated virus using affinity chromatography followed by ionic exchange chromatography.

25. The method according to claim 1, wherein the helper polynucleotide sequences encode adenovirus E4, adenovirus E2, and VA RNA.

26. The method according to claim 1, wherein one or more retargeting molecules can bind to one or more targets.

27. A covalently surface modified adeno-associated virus comprising a plurality of first members bound to second cognate members, wherein the second cognate members also are bound to retargeting molecules, and a gene of interest, wherein the covalently surface modified adeno-associated virus is made by a method according to claim 1.

28.-52. (canceled)

53. An AAV preparation comprising the covalently surface modified adeno-associated virus of claim 27.

54. A biologic drug product comprising the covalently surface modified adeno-associated virus of claim 27.

55. A method of screening retargeting molecules for production of a covalently surface modified adeno-associated virus species, wherein the method comprises the steps of:
(I) providing (A) a first plurality of nucleic acids encoding retargeting molecules that are different from one another and (B) a second plurality of DNA barcodes that are different from one another, wherein each individual DNA barcode of the second plurality is assigned to an individual covalently surface modified adeno-associated virus comprising a retargeting molecule of the first plurality for creating a covalently surface modified adeno-associated virus species;
(II) producing covalently surface modified adeno-associated virus species by
(A) transfecting a cell with:
(i) a plasmid comprising a polynucleotide that comprises a DNA barcode, wherein the polynucleotide that comprises the DNA barcode is flanked by AAV inverted terminal repeats;
(ii) a plasmid comprising an AAV rep gene and an AAV cap gene;
(iii) a plasmid comprising AAV rep and cap genes and a polynucleotide sequence encoding a first member of a specific binding pair;
(iv) a plasmid comprising one or more helper sequences;
(v) a plasmid comprising a polynucleotide sequence encoding a first portion of a retargeting molecule of the first plurality and a polynucleotide encoding a second cognate member of the specific binding pair; and
(vi) a plasmid comprising a polynucleotide encoding a second portion of a retargeting molecule of the first plurality;
(B) culturing the transfected cell to allow expression of plasmids (i) to (vi) and assembly of proteins to form a covalently surface modified adeno-associated virus species;
(C) harvesting the covalently surface modified adeno-associated virus species, wherein the covalently surface modified adeno-associated virus species will become part of a covalently surface modified adeno-associated virus library;
(D) repeating steps (A) to (C) to produce each covalently surface modified adeno-associated virus species to form the covalently surface modified adeno-associated viruses library; and
(E) detecting each DNA barcode of the second plurality in order to screen each species to determine a property.

56. The method of claim 55, wherein a species is selected based upon a property, wherein the property is genomic titer.

57. (canceled)

58. The method according to claim 55, wherein the retargeting molecule is an Fc-containing protein, and wherein the Fc-containing protein is a monoclonal antibody.

59. (canceled)

60. The method according to claim 58, wherein the monoclonal antibody is a multispecific antibody, such as a bispecific antibody or a trispecific antibody.

61. The method according to claim 58, wherein the Fc-containing protein is an Fc-fusion protein, wherein the Fc-fusion protein is a receptor-Fc-fusion protein, and wherein the receptor-Fc-fusion protein is a trap protein.

62.-63. (canceled)

64. The method according to claim 55, wherein the retargeting molecule is selected from the group consisting of an fab, f(ab′), f(Ab′)2, single chain antibody and a mini-trap protein.

65. The method according to claim 55, wherein the helper polynucleotide sequences encode adenovirus E4, adenovirus E2, and VA RNA.

66. A method of screening covalently surface modified adeno-associated virus species by screening retargeting molecules for production of the covalently surface modified adeno-associated virus species according to claim 1.

67.-68. (canceled)


SEQUENCE LISTING (214 sequences)

SEQ ID NO: 1 - DNA, 2211 bp, unidentified
  misc_feature 1..2211: note = Adeno associated virus 1
  source 1..2211: mol_type = genomic DNA
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 2 - DNA, 1872 bp, unidentified
  misc_feature 1..1872: note = Adeno associated virus 1
  source 1..1872: mol_type = genomic DNA
atgccgggcttctacgagatcgtgatcaaggtgccgagcgacctggacgagcacctgccgggcatttctgactcgtttgtgagctgggtggccgagaaggaatgggagctgcccccggattctgacatggatctgaatctgattgagcaggcacccctgaccgtggccgagaagctgcagcgcgacttcctggtccaatggcgccgcgtgagtaaggccccggaggccctcttctttgttcagttcgagaagggcgagtcctacttccacctccatattctggtggagaccacgggggtcaaatccatggtgctgggccgcttcctgagtcagattagggacaagctggtgcagaccatctaccgcgggatcgagccgaccctgcccaactggttcgcggtgaccaagacgcgtaatggcgccggaggggggaacaaggtggtggacgagtgctacatccccaactacctcctgcccaagactcagcccgagctgcagtgggcgtggactaacatggaggagtatataagcgcctgtttgaacctggccgagcgcaaacggctcgtggcgcagcacctgacccacgtcagccagacccaggagcagaacaaggagaatctgaaccccaattctgacgcgcctgtcatccggtcaaaaacctccgcgcgctacatggagctggtcgggtggctggtggaccggggcatcacctccgagaagcagtggatccaggaggaccaggcctcgtacatctccttcaacgccgcttccaactcgcggtcccagatcaaggccgctctggacaatgccggcaagatcatggcgctgaccaaatccgcgcccgactacctggtaggccccgctccgcccgcggacattaaaaccaaccgcatctaccgcatcctggagctgaacggctacgaacctgcctacgccggctccgtctttctcggctgggcccagaaaaggttcgggaagcgcaacaccatctggctgtttgggccggccaccacgggcaagaccaacatcgcggaagccatcgcccacgccgtgcccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagccgttgcaggaccggatgttcaaatttgaactcacccgccgtctggagcatgactttggcaaggtgacaaagcaggaagtcaaagagttcttccgctgggcgcaggatcacgtgaccgaggtggcgcatgagttctacgtcagaaagggtggagccaacaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaagacatgcgagagaatgaatcagaatttcaacatttgcttcacgcacgggacgagagactgttcagagtgcttccccggcgtgtcagaatctcaaccggtcgtcagaaagaggacgtatcggaaactctgtgccattcatcatctgctggggcgggctcccgagattgcttgctcggcctgcgatctggtcaacgtggacctggatgactgtgtttctgagcaataa

SEQ ID NO: 3 - DNA, 1866 bp, unidentified
  misc_feature 1..1866: note = Adeno associated virus 2
  source 1..1866: mol_type = genomic DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 4 - DNA, 1194 bp, unidentified
  misc_feature 1..1194: note = Adeno associated virus 2
  source 1..1194: mol_type = genomic DNA
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 5 - DNA, 2208 bp, unidentified
  misc_feature 1..2208: note = Adeno associated virus 2
  source 1..2208: mol_type = genomic DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 6 - DNA, 1797 bp, unidentified
  misc_feature 1..1797: note = Adeno associated virus 2
  source 1..1797: mol_type = genomic DNA
acggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 7 - DNA, 1602 bp, unidentified
  misc_feature 1..1602: note = Adeno associated virus 2
  source 1..1602: mol_type = genomic DNA
atggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 8 - DNA, 615 bp, unidentified
  misc_feature 1..615: note = Adeno associated virus 2
  source 1..615: mol_type = genomic DNA
ctggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctga

SEQ ID NO: 9 - DNA, 1875 bp, unidentified
  misc_feature 1..1875: note = Adeno associated virus 3
  source 1..1875: mol_type = genomic DNA
atgccggggttctacgagattgtcctgaaggtcccgagtgacctggacgagcgcctgccgggcatttctaactcgtttgttaactgggtggccgagaaggaatgggacgtgccgccggattctgacatggatccgaatctgattgagcaggcacccctgaccgtggccgaaaagcttcagcgcgagttcctggtggagtggcgccgcgtgagtaaggccccggaggccctcttttttgtccagttcgaaaagggggagacctacttccacctgcacgtgctgattgagaccatcggggtcaaatccatggtggtcggccgctacgtgagccagattaaagagaagctggtgacccgcatctaccgcggggtcgagccgcagcttccgaactggttcgcggtgaccaaaacgcgaaatggcgccgggggcgggaacaaggtggtggacgactgctacatccccaactacctgctccccaagacccagcccgagctccagtgggcgtggactaacatggaccagtatttaagcgcctgtttgaatctcgcggagcgtaaacggctggtggcgcagcatctgacgcacgtgtcgcagacgcaggagcagaacaaagagaatcagaaccccaattctgacgcgccggtcatcaggtcaaaaacctcagccaggtacatggagctggtcgggtggctggtggaccgcgggatcacgtcagaaaagcaatggattcaggaggaccaggcctcgtacatctccttcaacgccgcctccaactcgcggtcccagatcaaggccgcgctggacaatgcctccaagatcatgagcctgacaaagacggctccggactacctggtgggcagcaacccgccggaggacattaccaaaaatcggatctaccaaatcctggagctgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtaaactggaccaatgagaactttcccttcaacgattgcgtcgacaagatggtgatctggtgggaggagggcaagatgacggccaaggtcgtggagagcgccaaggccattctgggcggaagcaaggtgcgcgtggaccaaaagtgcaagtcatcggcccagatcgaacccactcccgtgatcgtcacctccaacaccaacatgtgcgccgtgattgacgggaacagcaccaccttcgagcatcagcagccgctgcaggaccggatgtttgaatttgaacttacccgccgtttggaccatgactttgggaaggtcaccaaacaggaagtaaaggactttttccggtgggcttccgatcacgtgactgacgtggctcatgagttctacgtcagaaagggtggagctaagaaacgccccgcctccaatgacgcggatgtaagcgagccaaaacgggagtgcacgtcacttgcgcagccgacaacgtcagacgcggaagcaccggcggactacgcggacaggtaccaaaacaaatgttctcgtcacgtgggcatgaatctgatgctttttccctgtaaaacatgcgagagaatgaatcaaatttccaatgtctgttttacgcatggtcaaagagactgtggggaatgcttccctggaatgtcagaatctcaacccgtttctgtcgtcaaaaagaagacttatcagaaactgtgtccaattcatcatatcctgggaagggcacccgagattgcctgttcggcctgcgatttggccaatgtggacttggatgactgtgtttctgagcaataa

SEQ ID NO: 10 - DNA, 2211 bp, unidentified
  misc_feature 1..2211: note = Adeno associated virus 3
  source 1..2211: mol_type = genomic DNA
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagggggctgtagatcagtctcctcaggaaccggactcatcatctggtgttggcaaatcgggcaaacagcctgccagaaaaagactaaatttcggtcagactggagactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttagaggggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactggaactgtcaatcatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtga

SEQ ID NO: 11 - DNA, 1872 bp, unidentified
  misc_feature 1..1872: note = Adeno associated virus 4
  source 1..1872: mol_type = genomic DNA
atgccggggttctacgagatcgtgctgaaggtgcccagcgacctggacgagcacctgcccggcatttctgactcttttgtgagctgggtggccgagaaggaatgggagctgccgccggattctgacatggacttgaatctgattgagcaggcacccctgaccgtggccgaaaagctgcaacgcgagttcctggtcgagtggcgccgcgtgagtaaggccccggaggccctcttctttgtccagttcgagaagggggacagctacttccacctgcacatcctggtggagaccgtgggcgtcaaatccatggtggtgggccgctacgtgagccagattaaagagaagctggtgacccgcatctaccgcggggtcgagccgcagcttccgaactggttcgcggtgaccaagacgcgtaatggcgccggaggcgggaacaaggtggtggacgactgctacatccccaactacctgctccccaagacccagcccgagctccagtgggcgtggactaacatggaccagtatataagcgcctgtttgaatctcgcggagcgtaaacggctggtggcgcagcatctgacgcacgtgtcgcagacgcaggagcagaacaaggaaaaccagaaccccaattctgacgcgccggtcatcaggtcaaaaacctccgccaggtacatggagctggtcgggtggctggtggaccgcgggatcacgtcagaaaagcaatggatccaggaggaccaggcgtcctacatctccttcaacgccgcctccaactcgcggtcacaaatcaaggccgcgctggacaatgcctccaaaatcatgagcctgacaaagacggctccggactacctggtgggccagaacccgccggaggacatttccagcaaccgcatctaccgaatcctcgagatgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtgaactggaccaatgagaactttccgttcaacgattgcgtcgacaagatggtgatctggtgggaggagggcaagatgacggccaaggtcgtagagagcgccaaggccatcctgggcggaagcaaggtgcgcgtggaccaaaagtgcaagtcatcggcccagatcgacccaactcccgtgatcgtcacctccaacaccaacatgtgcgcggtcatcgacggaaactcgaccaccttcgagcaccaacaaccactccaggaccggatgttcaagttcgagctcaccaagcgcctggagcacgactttggcaaggtcaccaagcaggaagtcaaagactttttccggtgggcgtcagatcacgtgaccgaggtgactcacgagttttacgtcagaaagggtggagctagaaagaggcccgcccccaatgacgcagatataagtgagcccaagcgggcctgtccgtcagttgcgcagccatcgacgtcagacgcggaagctccggtggactacgcggacaggtaccaaaacaaatgttctcgtcacgtgggtatgaatctgatgctttttccctgccggcaatgcgagagaatgaatcagaatgtggacatttgcttcacgcacggggtcatggactgtgccgagtgcttccccgtgtcagaatctcaacccgtgtctgtcgtcagaaagcggacgtatcagaaactgtgtccgattcatcacatcatggggagggcgcccgaggtggcctgctcggcctgcgaactggccaatgtggacttggatgactgtgacatggaacaataa

SEQ ID NO: 12 - DNA, 2205 bp, unidentified
  misc_feature 1..2205: note = Adeno associated virus 4
  source 1..2205: mol_type = genomic DNA
atgactgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgcggagttccagcagcggcttcagggcgacacatcgtttgggggcaacctcggcagagcagtcttccaggccaaaaagagggttcttgaacctcttggtctggttgagcaagcgggtgagacggctcctggaaagaagagaccgttgattgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatgcgtgcagcagctggcggagctgcagtcgagggcggacaaggtgccgatggagtgggtaatgcctcgggtgattggcattgcgattccacctggtctgagggccacgtcacgaccaccagcaccagaacctgggtcttgcccacctacaacaaccacctctacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtgatggatgcgggtcaagagggcagcctgcctccttttcccaacgacgtctttatggtgccccagtacggctactgtggactggtgaccggcaacacttcgcagcaacagactgacagaaatgccttctactgcctggagtactttccttcgcagatgctgcggactggcaacaactttgaaattacgtacagttttgagaaggtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcccgggccttcaatcaagcagcagggcttctcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggctgaaacacccgcctcctcaaatttttatcaagaacaccccggtacctgcgaatcctgcaacgaccttcagctctactccggtaaactccttcattactcagtacagcactggccaggtgtcggtgcagattgactgggagatccagaaggagcggtccaaacgctggaaccccgaggtccagtttacctccaactacggacagcaaaactctctgttgtgggctcccgatgcggctgggaaatacactgagcctagggctatcggtacccgctacctcacccaccacctgtaa

SEQ ID NO: 13 - DNA, 1833 bp, unidentified
  misc_feature 1..1833: note = Adeno associated virus 5
  source 1..1833: mol_type = genomic DNA
atggctaccttctatgaagtcattgttcgcgtcccatttgacgtggaggaacatctgcctggaatttctgacagctttgtggactgggtaactggtcaaatttgggagctgcctccagagtcagatttaaatttgactctggttgaacagcctcagttgacggtggctgatagaattcgccgcgtgttcctgtacgagtggaacaaattttccaagcaggagtccaaattctttgtgcagtttgaaaagggatctgaatattttcatctgcacacgcttgtggagacctccggcatctcttccatggtcctcggccgctacgtgagtcagattcgcgcccagctggtgaaagtggtcttccagggaattgaaccccagatcaacgactgggtcgccatcaccaaggtaaagaagggcggagccaataaggtggtggattctgggtatattcccgcctacctgctgccgaaggtccaaccggagcttcagtgggcgtggacaaacctggacgagtataaattggccgccctgaatctggaggagcgcaaacggctcgtcgcgcagtttctggcagaatcctcgcagcgctcgcaggaggcggcttcgcagcgtgagttctcggctgacccggtcatcaaaagcaagacttcccagaaatacatggcgctcgtcaactggctcgtggagcacggcatcacttccgagaagcagtggatccaggaaaatcaggagagctacctctccttcaactccaccggcaactctcggagccagatcaaggccgcgctcgacaacgcgaccaaaattatgagtctgacaaaaagcgcggtggactacctcgtggggagctccgttcccgaggacatttcaaaaaacagaatctggcaaatttttgagatgaatggctacgacccggcctacgcgggatccatcctctacggctggtgtcagcgctccttcaacaagaggaacaccgtctggctctacggacccgccacgaccggcaagaccaacatcgcggaggccatcgcccacactgtgcccttttacggctgcgtgaactggaccaatgaaaactttccctttaatgactgtgtggacaaaatgctcatttggtgggaggagggaaagatgaccaacaaggtggttgaatccgccaaggccatcctggggggctcaaaggtgcgggtcgatcagaaatgtaaatcctctgttcaaattgattctacccctgtcattgtaacttccaatacaaacatgtgtgtggtggtggatgggaattccacgacctttgaacaccagcagccgctggaggaccgcatgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctcaattggaattcaaggtatgattgcaaatgtgactatcatgctcaatttgacaacatttctaacaaatgtgatgaatgtgaatatttgaatcggggcaaaaatggatgtatctgtcacaatgtaactcactgtcaaatttgtcatgggattcccccctgggaaaaggaaaacttgtcagattttggggattttgacgatgccaataaagaacagtaa

SEQ ID NO: 14 - DNA, 2175 bp, unidentified
  misc_feature 1..2175: note = Adeno associated virus 5
  source 1..2175: mol_type = genomic DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 15 - DNA, 1872 bp, unidentified
  misc_feature 1..1872: note = Adeno associated virus 6
  source 1..1872: mol_type = genomic DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgacttcctggtccagtggcgccgcgtgagtaaggccccggaggccctcttctttgttcagttcgagaagggcgagtcctacttccacctccatattctggtggagaccacgggggtcaaatccatggtgctgggccgcttcctgagtcagattagggacaagctggtgcagaccatctaccgcgggatcgagccgaccctgcccaactggttcgcggtgaccaagacgcgtaatggcgccggaggggggaacaaggtggtggacgagtgctacatccccaactacctcctgcccaagactcagcccgagctgcagtgggcgtggactaacatggaggagtatataagcgcgtgtttaaacctggccgagcgcaaacggctcgtggcgcacgacctgacccacgtcagccagacccaggagcagaacaaggagaatctgaaccccaattctgacgcgcctgtcatccggtcaaaaacctccgcacgctacatggagctggtcgggtggctggtggaccggggcatcacctccgagaagcagtggatccaggaggaccaggcctcgtacatctccttcaacgccgcctccaactcgcggtcccagatcaaggccgctctggacaatgccggcaagatcatggcgctgaccaaatccgcgcccgactacctggtaggccccgctccgcccgccgacattaaaaccaaccgcatttaccgcatcctggagctgaacggctacgaccctgcctacgccggctccgtctttctcggctgggcccagaaaaggttcggaaaacgcaacaccatctggctgtttgggccggccaccacgggcaagaccaacatcgcggaagccatcgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgatcccacccccgtgatcgtcacctccaacaccaacatgtgcgccgtgattgacgggaacagcaccaccttcgagcaccagcagccgttgcaggaccggatgttcaaatttgaactcacccgccgtctggagcatgactttggcaaggtgacaaagcaggaagtcaaagagttcttccgctgggcgcaggatcacgtgaccgaggtggcgcatgagttctacgtcagaaagggtggagccaacaagagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacatgcgagagaatgaatcagaatttcaacatttgcttcacgcacgggaccagagactgttcagaatgtttccccggcgtgtcagaatctcaaccggtcgtcagaaagaggacgtatcggaaactctgtgccattcatcatctgctggggcgggctcccgagattgcttgctcggcctgcgatctggtcaacgtggatctggatgactgtgtttctgagcaataa

SEQ ID NO: 16 - DNA, 2211 bp, unidentified
  misc_feature 1..2211: note = Adeno associated virus 6
  source 1..2211: mol_type = genomic DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 17 - DNA, 1872 bp, unidentified
  misc_feature 1..1872: note = Adeno associated virus 7
  source 1..1872: mol_type = genomic DNA
atgccgggtttctacgagatcgtgatcaaggtgccgagcgacctggacgagcacctgccgggcatttctgactcgtttgtgaactgggtggccgagaaggaatgggagctgcccccggattctgacatggatctgaatctgatcgagcaggcacccctgaccgtggccgagaagctgcagcgcgacttcctggtccaatggcgccgcgtgagtaaggccccggaggccctgttctttgttcagttcgagaagggcgagagctacttccaccttcacgttctggtggagaccacgggggtcaagtccatggtgctaggccgcttcctgagtcagattcgggagaagctggtccagaccatctaccgcggggtcgagcccacgctgcccaactggttcgcggtgaccaagacgcgtaatggcgccggcggggggaacaaggtggtggacgagtgctacatccccaactacctcctgcccaagacccagcccgagctgcagtgggcgtggactaacatggaggagtatataagcgcgtgtttgaacctggccgaacgcaaacggctcgtggcgcagcacctgacccacgtcagccagacgcaggagcagaacaaggagaatctgaaccccaattctgacgcgcccgtgatcaggtcaaaaacctccgcgcgctacatggagctggtcgggtggctggtggaccggggcatcacctccgagaagcagtggatccaggaggaccaggcctcgtacatctccttcaacgccgcctccaactcgcggtcccagatcaaggccgcgctggacaatgccggcaagatcatggcgctgaccaaatccgcgcccgactacctggtggggccctcgctgcccgcggacattaaaaccaaccgcatctaccgcatcctggagctgaacgggtacgatcctgcctacgccggctccgtctttctcggctgggcccagaaaaagttcgggaagcgcaacaccatctggctgtttgggcccgccaccaccggcaagaccaacattgcggaagccatcgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagccgttgcaggaccggatgttcaaatttgaactcacccgccgtctggagcacgactttggcaaggtgacgaagcaggaagtcaaagagttcttccgctgggccagtgatcacgtgaccgaggtggcgcatgagttctacgtcagaaagggcggagccagcaaaagacccgcccccgatgacgcggatataagcgagcccaagcgggcctgcccctcagtcgcggatccatcgacgtcagacgcggaaggagctccggtggactttgccgacaggtaccaaaacaaatgttctcgtcacgcgggcatgattcagatgctgtttccctgcaaaacgtgcgagagaatgaatcagaatttcaacatttgcttcacacacggggtcagagactgtttagagtgtttccccggcgtgtcagaatctcaaccggtcgtcagaaaaaagacgtatcggaaactctgcgcgattcatcatctgctggggcgggcgcccgagattgcttgctcggcctgcgacctggtcaacgtggacctggacgactgcgtttctgagcaataa

SEQ ID NO: 18 - DNA, 2214 bp, unidentified
  misc_feature 1..2214: note = Adeno associated virus 7
  source 1..2214: mol_type = genomic DNA
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctgcaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctgtaa

SEQ ID NO: 19 - DNA, 1878 bp, unidentified
  misc_feature 1..1878: note = Adeno associated virus 8
  source 1..1878: mol_type = genomic DNA
atgccgggcttctacgagatcgtgatcaaggtgccgagcgacctggacgagcacctgccgggcatttctgactcgtttgtgaactgggtggccgagaaggaatgggagctgcccccggattctgacatggatcggaatctgatcgagcaggcacccctgaccgtggccgagaagctgcagcgcgacttcctggtccaatggcgccgcgtgagtaaggccccggaggccctcttctttgttcagttcgagaagggcgagagctactttcacctgcacgttctggtcgagaccacgggggtcaagtccatggtgctaggccgcttcctgagtcagattcgggaaaagcttggtccagaccatctacccgcggggtcgagccccaccttgcccaactggttcgcggtgaccaaagacgcggtaatggcgccggcgggggggaacaaggtggtggacgagtgctacatccccaactacctcctgcccaagactcagcccgagctgcagtgggcgtggactaacatggaggagtatataagcgcgtgcttgaacctggccgagcgcaaacggctcgtggcgcagcacctgacccacgtcagccagacgcaggagcagaacaaggagaatctgaaccccaattctgacgcgcccgtgatcaggtcaaaaacctccgcgcgctatatggagctggtcgggtggctggtggaccggggcatcacctccgagaagcagtggatccaggaggaccaggcctcgtacatctccttcaacgccgcctccaactcgcggtcccagatcaaggccgcgctggacaatgccggcaagatcatggcgctgaccaaatccgcgcccgactacctggtggggccctcgctgcccgcggacattacccagaaccgcatctaccgcatcctcgctctcaacggctacgaccctgcctacgccggctccgtctttctcggctgggctcagaaaaagttcgggaaacgcaacaccatctggctgtttggacccgccaccaccggcaagaccaacattgcggaagccatcgcccacgccgtgcccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagcctctccaggaccggatgtttaagttcgaactcacccgccgtctggagcacgactttggcaaggtgacaaagcaggaagtcaaagagttcttccgctgggccagtgatcacgtgaccgaggtggcgcatgagttttacgtcagaaagggcggagccagcaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacgtgcgagagaatgaatcagaatttcaacatttgcttcacacacggggtcagagactgctcagagtgtttccccggcgtgtcagaatctcaaccggtcgtcagaaagaggacgtatcggaaactctgtgcgattcatcatctgctggggcgggctcccgagattgcttgctcggcctgcgatctggtcaacgtggacctggatgactgtgtttctgagcaataa

SEQ ID NO: 20 - DNA, 2217 bp, unidentified
  misc_feature 1..2217: note = Adeno associated virus 8
  source 1..2217: mol_type = genomic DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 21 - DNA, 2211 bp, unidentified
  misc_feature 1..2211: note = Adeno associated virus 9
  source 1..2211: mol_type = genomic DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 22 - DNA, 1869 bp, unidentified
  misc_feature 1..1869: note = Adeno associated virus 10
  source 1..1869: mol_type = genomic DNA
atgccgggcttctacgagatcgtgatcaaggtgccgagcgacctggacgagcacctgccgggcatttctgactcgtttgtgaactgggtggccgagaaggaatgggagctgcccccggattctgacatggatcggaatctgatcgagcaggcacccctgaccgtggccgagaagctgcagcgcgacttcctggtccactggcgccgcgtgagtaaggccccggaggccctcttctttgttcagttcgagaagggcgagtcctactttcacctgcacgttctggtcgagaccacgggggtcaagtccatggtcctgggccgcttcctgagtcagatcagagacaggctggtgcagaccatctaccgcggggtagagcccacgctgcccaactggttcgcggtgaccaagacgcgaaatggcgccggcggggggaacaaggtggtggacgagtgctacatccccaactacctcctgcccaagacgcagcccgagctgcagtgggcgtggactaacatggaggagtatataagcgcgtgtctgaacctcgcggagcgtaaacggctcgtggcgcagcacctgacccacgtcagccagacgcaggagcagaacaaggagaatctgaacccgaattctgacgcgcccgtgatcaggtcaaaaacctccgcgcgctacatggagctggtcgggtggctggtggaccggggcatcacctccgagaagcagtggatccaggaggaccaggcctcgtacatctccttcaacgccgcctccaactcgcggtcccagatcaaggccgcgctggacaatgccggaaagatcatggcgctgaccaaatccgcgcccgactacctggtaggcccgtccttacccgcggacattaaggccaaccgcatctaccgcatcctggagctcaacggctacgaccccgcctacgccggctccgtcttcctgggctgggcgcagaaaaagttcggtaaaaggaatacaatttggctgttcgggcccgccaccaccggcaagaccaacatcgcggaagccatcgcccacgccgtgcccttctacggctgcgtcaactggaccaatgagaactttcccttcaacgattgcgtcgacaagatggtgatctggtgggaggagggcaagatgaccgccaaggtcgtggagtccgccaaggccattctgggcggaagcaaggtgcgcgtcgaccaaaagtgcaagtcctcggcccagatcgaccccacgcccgtgatcgtcacctccaacaccaacatgtgcgccgtgatcgacgggaacagcaccaccttcgagcaccagcagcccctgcaggaccgcatgttcaagttcgagctcacccgccgtctggagcacgactttggcaaggtgaccaagcaggaagtcaaagagttcttccgctgggctcaggatcacgtgactgaggtgacgcatgagttctacgtcagaaagggcggagccaccaaaagacccgcccccagtgacgcggatataagcgagcccaagcgggcctgcccctcagttgcggagccatcgacgtcagacgcggaagcaccggtggactttgcggacaggtaccaaaacaaatgttctcgtcacgcgggcatgcttcagatgctgtttccctgcaagacatgcgagagaatgaatcagaatttcaacgtctgcttcacgcacggggtcagagactgctcagagtgcttccccggcgcgtcagaatctcaacctgtcgtcagaaaaaagacgtatcagaaactgtgcgcgattcatcatctgctggggcgggcacccgagattgcgtgttcggcctgcgatctcgtcaacgtggacttggatgactgtgtttctgagcaataa

SEQ ID NO: 23 - DNA, 2217 bp, unidentified
  misc_feature 1..2217: note = Adeno associated virus 10
  source 1..2217: mol_type = genomic DNA
atggctgctgacggttatcttccagattggctcgaggacaacctctctgagggcattcgcgagtggtgggacctgaaacctggagcccccaagcccaaggccaaccagcagaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaagctgctaagacggctcctggaaagaagagaccggtagaaccgtcacctcagcgttcccccgactcctccacgggcatcggcaagaaaggccagcagcccgctaaaaagagactgaactttgggcagactggcgagtcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctgcccacctacaacaaccacctctacaagcaaatctccaacgggacatcgggaggaagcaccaacgacaacacctacttcggctacagcaccccctgggggtattttgacttcaacagattccactgccacttctcaccacgtgactggcagcgactcatcaacaacaactggggattccggccaaaaagactcagcttcaagctcttcaacatccaggtcaaggaggtcacgcagaatgaaggcaccaagaccatcgccaataaccttaccagcacgattcaggtatttacggactcggaataccagctgccgtacgtcctcggctccgcgcaccagggctgcctgcctccgttcccggcggatgtcttcatgattccccagtacggctacctgacactgaacaatggaagtcaagccgtaggccgttcctccttctactgcctggaatattttccatctcaaatgctgcgaactggaaacaattttgaattcagctacaccttcgaggacgtgcctttccacagcagctacgcacacagccagagcttggaccgactgatgaatcctctcattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggaagagactctctggtgaatcccggtgtcgccatggcaacccacaaggacgacgaggaacgcttcttcccgtcgagcggagtcctgatgtttggaaaacagggtgctggaagagacaatgtggactacagcagcgttatgctaacaagcgaagaagaaattaaaaccactaaccctgtagccacagaacaatacggcgtggtggctgacaacttgcagcaagccaatacagggcctattgtgggaaatgtcaacagccaaggagccttacctggcatggtctggcagaaccgagacgtgtacctgcagggtcccatctgggccaagattcctcacacggacggcaactttcacccgtctcctctgatgggcggctttggacttaaacacccgcctccacagatcctgatcaagaacacgccggtacctgcggatcctccaacaacgttcagccaggcgaaattggcttccttcatcacgcagtacagcaccggacaggtcagcgtggaaatcgagtgggagctgcagaaggagaacagcaaacgctggaacccagagattcagtacacttcaaactactacaaatctacaaatgtggactttgctgtcaatacagagggaacttattctgagcctcgccccattggtactcgttatctgacacgtaatctgtaa

SEQ ID NO: 24 - DNA, 1869 bp, unidentified
  misc_feature 1..1869: note = Adeno associated virus 11
  source 1..1869: mol_type = genomic DNA
atgccgggcttctacgagatcgtgatcaaggtgccgagcgacctggacgagcacctgccgggcatttctgactcgtttgtgaactgggtggccgagaaggaatgggagctgcccccggattctgacatggatcggaatctgatcgagcaggcacccctgaccgtggccgagaagctgcagcgcgacttcctggtccactggcgccgcgtgagtaaggccccggaggccctcttctttgttcagttcgagaagggcgagtcctacttccacctccacgttctcgtcgagaccacgggggtcaagtccatggtcctgggccgcttcctgagtcagatcagagacaggctggtgcagaccatctaccgcggggtcgagcccacgctgcccaactggttcgcggtgaccaagacgcgaaatggcgccggcggggggaacaaggtggtggacgagtgctacatccccaactacctcctgcccaagacccagcccgagctgcagtgggcgtggactaacatggaggagtatataagcgcgtgtctaaacctcgcggagcgtaaacggctcgtggcgcagcacctgacccacgtcagccagacgcaggagcagaacaaggagaatctgaacccgaattctgacgcgcccgtgatcaggtcaaaaacctccgcgcgctacatggagctggtcgggtggctggtggaccggggcatcacctccgagaagcagtggatccaggaggaccaggcctcgtacatctccttcaacgccgcctccaactcgcggtcccagatcaaggccgcgctggacaatgccggaaagatcatggcgctgaccaaatccgcgcccgactacctggtaggcccgtccttacccgcggacattaaggccaaccgcatctaccgcatcctggagctcaacggctacgaccccgcctacgccggctccgtcttcctgggctgggcgcagaaaaagttcggtaaacgcaacaccatctggctgtttgggcccgccaccaccggcaagaccaacatcgcggaagccatagcccacgccgtgcccttctacggctgcgtgaactggaccaatgagaactttcccttcaacgattgcgtcgacaagatggtgatctggtgggaggagggcaagatgaccgccaaggtcgtggagtccgccaaggccattctgggcggaagcaaggtgcgcgtggaccaaaagtgcaagtcctcggcccagatcgaccccacgcccgtgatcgtcacctccaacaccaacatgtgcgccgtgatcgacgggaacagcaccaccttcgagcaccagcagccgctgcaggaccgcatgttcaagttcgagctcacccgccgtctggagcacgactttggcaaggtgaccaagcaggaagtcaaagagttcttccgctgggctcaggatcacgtgactgaggtggcgcatgagttctacgtcagaaagggcggagccaccaaaagacccgcccccagtgacgcggatataagcgagcccaagcgggcctgcccctcagttccggagccatcgacgtcagacgcggaagcaccggtggactttgcggacaggtaccaaaacaaatgttctcgtcacgcgggcatgcttcagatgctgtttccctgcaagacatgcgagagaatgaatcagaatttcaacgtctgcttcacgcacggggtcagagactgctcagagtgcttccccggcgcgtcagaatctcaacccgtcgtcagaaaaaagacgtatcagaaactgtgcgcgattcatcatctgctggggcgggcacccgagattgcgtgttcggcctgcgatctcgtcaacgtggacttggatgactgtgtttctgagcaataa

SEQ ID NO: 25 - DNA, 2202 bp, unidentified
  misc_feature 1..2202: note = Adeno associated virus 11
  source 1..2202: mol_type = genomic DNA
atggctgctgacggttatcttccagattggctcgaggacaacctctctgagggcattcgcgagtggtgggacctgaaacctggagccccgaagcccaaggccaaccagcagaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggtactcgaacctctgggcctggttgaagaaggtgctaaaacggctcctggaaagaagagaccgttagagtcaccacaagagcccgactcctcctcgggcatcggcaaaaaaggcaaacaaccagccagaaagaggctcaactttgaagaggacactggagccggagacggaccccctgaaggatcagataccagcgccatgtcttcagacattgaaatgcgtgcagcaccgggcggaaatgctgtcgatgcgggacaaggttccgatggagtgggtaatgcctcgggtgattggcattgcgattccacctggtctgagggcaaggtcacaacaacctcgaccagaacctgggtcttgcccacctacaacaaccacttgtacctgcgtctcggaacaacatcaagcagcaacacctacaacggattctccaccccctggggatattttgacttcaacagattccactgtcacttctcaccacgtgactggcaaagactcatcaacaacaactggggactacgaccaaaagccatgcgcgttaaaatcttcaatatccaagttaaggaggtcacaacgtcgaacggcgagactacggtcgctaataaccttaccagcacggttcagatatttgcggactcgtcgtatgagctcccgtacgtgatggacgctggacaagaggggagcctgcctcctttccccaatgacgtgttcatggtgcctcaatatggctactgtggcatcgtgactggcgagaatcagaaccaaacggacagaaacgctttctactgcctggagtattttccttcgcaaatgttgagaactggcaacaactttgaaatggcttacaactttgagaaggtgccgttccactcaatgtatgctcacagccagagcctggacagactgatgaatcccctcctggaccagtacctgtggcacttacagtcgactacctctggagagactctgaatcaaggcaatgcagcaaccacatttggaaaaatcaggagtggagactttgccttttacagaaagaactggctgcctgggccttgtgttaaacagcagagattctcaaaaactgccagtcaaaattacaagattcctgccagcgggggcaacgctctgttaaagtatgacacccactataccttaaacaaccgctggagcaacatcgcgcccggacctccaatggccacagccggaccttcggatggggacttcagtaacgcccagcttatattccctggaccatctgttaccggaaatacaacaacttcagccaacaatctgttgtttacatcagaagaagaaattgctgccaccaacccaagagacacggacatgtttggccagattgctgacaataatcagaatgctacaactgctcccataaccggcaacgtgactgctatgggagtgctgcctggcatggtgtggcaaaacagagacatttactaccaagggccaatttgggccaagatcccacacgcggacggacattttcatccttcaccgctgattggtgggtttggactgaaacacccgcctccccagatattcatcaagaacactcccgtacctgccaatcctgcgacaaccttcactgcagccagagtggactctttcatcacacaatacagcaccggccaggtcgctgttcagattgaatgggaaattgaaaaggaacgctccaaacgctggaatcctgaagtgcagtttacttcaaactatgggaaccagtcttctatgttgtgggctcctgatacaactgggaagtatacagagccgcgggttattggctctcgttatttgactaatcatttgtaa

SEQ ID NO: 26 - DNA, 1866 bp, unidentified
  misc_feature 1..1866: note = Adeno associated virus 12
  source 1..1866: mol_type = genomic DNA
atgccggggttctacgaggtggtgatcaaggtgcccagcgacctggacgagcacctgcccggcatttctgactcctttgtgaactgggtggccgagaaggaatgggagttgcccccggattctgacatggatcagaatctgattgagcaggcacccctgaccgtggccgagaagctgcagcgcgagttcctggtggaatggcgccgagtgagtaaatttctggaggccaagttttttgtgcagtttgaaaagggggactcgtactttcatttgcatattctgattgaaattaccggcgtgaaatccatggtggtgggccgctacgtgagtcagattagggataaactgatccagcgcatctaccgcggggtcgagccccagctgcccaactggttcgcggtcacaaagacccgaaatggcgccggaggcgggaacaaggtggtggacgagtgctacatccccaactacctgctccccaaggtccagcccgagcttcagtgggcgtggactaacatggaggagtatataagcgcctgtttgaacctcgcggagcgtaaacggctcgtggcgcagcacctgacgcacgtctcccagacccaggagggcgacaaggagaatctgaacccgaattctgacgcgccggtgatccggtcaaaaacctccgccaggtacatggagctggtcgggtggctggtggacaagggcatcacgtccgagaagcagtggatccaggaggaccaggcctcgtacatctccttcaacgcggcctccaactcccggtcgcagatcaaggcggccctggacaatgcctccaaaatcatgagcctcaccaaaacggctccggactatctcatcgggcagcagcccgtgggggacattaccaccaaccggatctacaaaatcctggaactgaacgggtacgacccccagtacgccgcctccgtctttctcggctgggcccagaaaaagtttggaaagcgcaacaccatctggctgtttgggcccgccaccaccggcaagaccaacatcgcggaagccatcgcccacgcggtccccttctacggctgcgtcaactggaccaatgagaactttcccttcaacgactgcgtcgacaaaatggtgatttggtgggaggagggcaagatgaccgccaaggtcgtagagtccgccaaggccattctgggcggcagcaaggtgcgcgtggaccaaaaatgcaaggcctctgcgcagatcgaccccacccccgtgatcgtcacctccaacaccaacatgtgcgccgtgattgacgggaacagcaccaccttcgagcaccagcagcccctgcaggaccggatgttcaagtttgaactcacccgccgcctcgaccacgactttggcaaggtcaccaagcaggaagtcaaggactttttccggtgggcggctgatcacgtgactgacgtggctcatgagttttacgtcacaaagggtggagctaagaaaaggcccgccccctctgacgaggatataagcgagcccaagcggccgcgcgtgtcatttgcgcagccggagacgtcagacgcggaagctcccggagacttcgccgacaggtaccaaaacaaatgttctcgtcacgcgggtatgctgcagatgctctttccctgcaagacgtgcgagagaatgaatcagaattccaacgtctgcttcacgcacggtcagaaagattgcggggagtgctttcccgggtcagaatctcaaccggtttctgtcgtcagaaaaacgtatcagaaactgtgcatccttcatcagctccggggggcacccgagatcgcctgctctgcttgcgaccaactcaaccccgatttggacgattgccaatttgagcaataa

SEQ ID NO: 27 - DNA, 2229 bp, unidentified
  misc_feature 1..2229: note = Adeno associated virus 12
  source 1..2229: mol_type = genomic DNA
atggctgctgacggttatcttccagattggctcgaggacaacctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtatctcaagtacaaccacgccgacgccgagttccagcagcgcttggcgaccgacacctcttttgggggcaacctcgggcgagcagtcttccaggccaaaaagaggattctcgagcctctgggtctggttgaagagggcgttaaaacggctcctggaaagaaacgcccattagaaaagactccaaatcggccgaccaacccggactctgggaaggccccggccaagaaaaagcaaaaagacggcgaaccagccgactctgctagaaggacactcgactttgaagactctggagcaggagacggaccccctgagggatcatcttccggagaaatgtctcatgatgctgagatgcgtgcggcgccaggcggaaatgctgtcgaggcgggacaaggtgccgatggagtgggtaatgcctccggtgattggcattgcgattccacctggtcagagggccgagtcaccaccaccagcacccgaacctgggtcctacccacgtacaacaaccacctgtacctgcgaatcggaacaacggccaacagcaacacctacaacggattctccaccccctggggatactttgactttaaccgcttccactgccacttttccccacgcgactggcagcgactcatcaacaacaactggggactcaggccgaaatcgatgcgtgttaaaatcttcaacatacaggtcaaggaggtcacgacgtcaaacggcgagactacggtcgctaataaccttaccagcacggttcagatctttgcggattcgacgtatgaactcccatacgtgatggacgccggtcaggaggggagctttcctccgtttcccaacgacgtctttatggttccccaatacggatactgcggagttgtcactggaaaaaaccagaaccagacagacagaaatgccttttactgcctggaatactttccatcccaaatgctaagaactggcaacaattttgaagtcagttaccaatttgaaaaagttcctttccattcaatgtacgcgcacagccagagcctggacagaatgatgaatcctttactggatcagtacctgtggcatctgcaatcgaccactaccggaaattcccttaatcaaggaacagctaccaccacgtacgggaaaattaccactggagactttgcctactacaggaaaaactggttgcctggagcctgcattaaacaacaaaaattttcaaagaatgccaatcaaaactacaagattcccgccagcgggggagacgcccttttaaagtatgacacgcataccactctaaatgggcgatggagtaacatggctcctggacctccaatggcaaccgcaggtgccggggactcggattttagcaacagccagctgatctttgccggacccaatccgagcggtaacacgaccacatcttcaaacaatttgttgtttacctcagaagaggagattgccacaacaaacccacgagacacggacatgtttggacagattgcagataataatcaaaatgccaccaccgcccctcacatcgctaacctggacgctatgggaattgttcccggaatggtctggcaaaacagagacatctactaccagggccctatttgggccaaggtccctcacacggacggacactttcacccttcgccgctgatgggaggatttggactgaaacacccgcctccacagattttcatcaaaaacacccccgtacccgccaatcccaatactacctttagcgctgcaaggattaattcttttctgacgcagtacagcaccggacaagttgccgttcagatcgactgggaaattcagaaggagcattccaaacgctggaatcccgaagttcaatttacttcaaactacggcactcaaaattctatgctgtgggctcccgacaatgctggcaactaccacgaactccgggctattgggtcccgtttcctcacccaccacttgtaa

SEQ ID NO: 28 - DNA, 1872 bp, unidentified
  misc_feature 1..1872: note = Adeno associated virus 13
  source 1..1872: mol_type = genomic DNA
atgccgggattctacgagattgtcctgaaggtgcccagcgacctggacgagcacctgcctggcatttctgactcttttgtaaactgggtggcggagaaggaatgggagctgccgccggattctgacatggatctgaatctgattgagcaggcacccctaaccgtggccgaaaagctgcaacgcgaattcctggtcgagtggcgccgcgtgagtaaggccccggaggccctcttctttgttcagttcgagaagggggacagctacttccacctacacattctggtggagaccgtgggcgtgaaatccatggtggtgggccgctacgtgagccagattaaagagaagctggtgacccgcatctaccgcggggtcgagccgcagcttccgaactggttcgcggtgaccaagacgcgtaatggcgccggaggcgggaacaaggtggtggacgactgctacatccccaactacctgctccccaagacccagcccgagctccagtgggcgtggactaatatggaccagtatttaagcgcctgtttgaatctcgcggagcgtaaacggctggtggcgcagcatctgacgcacgtgtcgcagacgcaggagcagaacaaagagaaccagaatcccaattctgacgcgccggtgatcagatcaaaaacctccgcgaggtacatggagctggtcgggtggctggtggaccgcgggatcacgtcagaaaagcaatggatccaggaggaccaggcctcttacatctccttcaacgccgcctccaactcgcggtcacaaatcaaggccgcactggacaatgcctccaaatttatgagcctgacaaaaacggctccggactacctggtgggaaacaacccgccggaggacattaccagcaaccggatctacaaaatcctcgagatgaacgggtacgatccgcagtacgcggcctccgtcttcctgggctgggcgcaaaagaagttcgggaagaggaacaccatctggctctttgggccggccacgacgggtaaaaccaacatcgctgaagctatcgcccacgccgtgcccttttacggctgcgtgaactggaccaatgagaactttccgttcaacgattgcgtcgacaagatggtgatctggtgggaggagggcaagatgacggccaaggtcgtggagtccgccaaggccattctgggcggaagcaaggtgcgcgtggaccaaaagtgcaagtcatcggcccagatcgacccaactcccgtcatcgtcacctccaacaccaacatgtgcgcggtcatcgacggaaattccaccaccttcgagcaccaacaaccactccaagaccggatgttcaagttcgagctcaccaagcgcctggagcacgactttggcaaggtcaccaagcaggaagtcaaggactttttccggtgggcgtcagatcacgtgactgaggtgtctcacgagttttacgtcagaaagggtggagctagaaagaggcccgcccccaatgacgcagatataagtgagcccaagcgggcctgtccgtcagttgcgcagccatcgacgtcagacgcggaagctccggtggactacgcggacaggtaccaaaacaaatgttctcgtcacgtgggcatgaatctgatgctttttccctgccggcaatgcgagagaatgaatcagaatgtggacatttgcttcacgcacggggtcatggactgtgccgagtgcttccccgtgtcagaatctcaacccgtgtctgtcgtcagaaagcggacatatcagaaactgtgtccgattcatcacatcatggggagggcgcccgaggtggcttgttcggcctgcgatctggccaatgtggacttggatgactgtgacatggagcaataa

SEQ ID NO: 29 - DNA, 2202 bp, unidentified
  misc_feature 1..2202: note = Adeno associated virus 13
  source 1..2202: mol_type = genomic DNA
atgactgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggacttgacaagggggaacccgtcaacgcagcggacgcggcagccctcgaacacgacaaggcctacgaccagcagctcaaggccggtgacaacccctacctcaagtacaaccacgccgacgccgagtttcaggagcgtcttcaagaagatacgtcttttgggggcaacctcggacgagcagtcttccaggccaaaaagaggatccttgagcctctgggtctggttgaggaagcggctaagacggctcctggaaaaaagagacctgtagagcaatctccagcagaaccggactcctcttcgggcatcggcaaatcaggccagcagcccgctagaaaaagactgaattttggtcagactggcgacacagagtcagtcccagaccctcaaccactcggacaacctcccgcagccccctctggtgtgggatctactacaatggcttcaggcggtggcgcaccaatggcagacaataacgagggtgccgatggagtgggtaattcctcaggaaattggcattgcgattcccaatggctgggcgacagagtcatcaccaccagcacccgcacctgggccctgcccacctacaacaatcacctctacaagcaaatctccagccaatcaggagccaccaacgacaaccactactttggctacagcaccccctgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactccgagtaccagctcccgtacgtcctcggctcggcgcatcagggatgcctcccgccgttcccagcagacgtcttcatggtcccacagtatggatacctcaccctgaacaacgggagtcaggcggtaggacgctcttccttttactgcctggagtactttccttctcagatgctgcgtactggaaacaactttcagtttagctacacttttgaagacgtgcctttccacagcagctacgctcacagccaaagtctggaccgtctcatgaatcctctgatcgaccagtacctgtactatctgaacaggacacaaacagccagtggaactcagcagtctcggctactgtttagccaagctggacccaccagtatgtctcttcaagctaaaaactggctgcctggaccttgctacagacagcagcgtctgtcaaagcaggcaaacgacaacaacaacagcaactttccctggactggtgccaccaaatatcatctgaatggccgggactcattggtgaacccgggccctgctatggccagtcacaaggatgacaaagaaaagtttttccccatgcatggaaccctgatatttggtaaagaaggaacaaatgccaacaacgcggatttggaaaatgtcatgattacagatgaagaagaaatccgcaccaccaatcccgtggctacggagcagtacgggactgtgtcaaataatttgcaaaactcaaacgctggtccaactactggaactgtcaatcaccaaggagcgttacctggtatggtgtggcaggatcgagacgtgtacctgcagggacccatttgggccaagattcctcacaccgatggacactttcatccttctccactgatgggaggttttgggctcaaacacccgcctcctcagatcatgatcaaaaacactcccgttccagccaatcctcccacaaactttagtgcggcaaagtttgcttccttcatcacacagtactccacggggcaggtcagcgtggagatcgagtgggagctgcagaaggagaacagcaaacgctggaatcccgaaattcagtacacttccaactacaacaaatctgttaatgtggactttactgtggacactaatggtgtgtattcagagcctcgccccattggcaccagatacctgactcgtaatctgtaa

SEQ ID NO: 30 - DNA, 2217 bp, unidentified
  misc_feature 1..2217: note = Non human primate Adeno associated virus
  source 1..2217: mol_type = unassigned DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaa

SEQ ID NO: 31 - DNA, 2214 bp, unidentified
  source 1..2214: mol_type = genomic DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaagctgctaagacggctcctggaaagaagagaccggtagaaccgtcacctcagcgttcccccgactcctccacgggcatcggcaagaaaggccagcagcccgctaaaaagagactgaactttggtcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctgcccacctacaacaaccacctctacaagcaaatatccaatgggacatcgggaggaagcaccaacgacaacacctacttcggctacagcaccccctgggggtattttgacttcaacagattccactgccacttctcaccacgtgactggcagcgactcatcaacaacaactggggattccggccaaaaagactcagcttcaagctcttcaacatccaggtcaaggaggtcacgcagaatgaaggcaccaagaccatcgccaataaccttaccagcacgattcaggtatttacggactcggaataccagctgccgtacgtcctcggctccgcgcaccagggctgcctgcctccgttcccggcggacgtcttcatgattccccagtacggctaccttacactgaacaatggaagtcaagccgtaggccgttcctccttctactgcctggaatattttccatctcaaatgctgcgaactggaaacaattttgaattcagctacaccttcgaggacgtgcctttccacagcagctacgcacacagccagagcttggaccgactgatgaatcctctcatcgaccagtacctgtactacttatccagaactcagtccacaggaggaactcaaggtacccagcaattgttattttctcaagctgggcctgcaaacatgtcggctcaggctaagaactggctacctggaccttgctaccggcagcagcgagtctctacgacactgtcgcaaaacaacaacagcaactttgcttggactggtgccaccaaatatcacctgaacggaagagactctttggtaaatcccggtgtcgccatggcaacccacaaggacgacgaggaacgcttcttcccgtcgagtggagtcctgatgtttggaaaacagggtgctggaagagacaatgtggactacagcagcgttatgctaaccagcgaagaagaaattaaaaccactaaccctgtagccacagaacaatacggtgtggtggctgataacttgcagcaaaccaatacggggcctattgtgggaaatgtcaacagccaaggagccttacctggcatggtctggcagaaccgagacgtgtacctgcagggtcccatctgggccaagattcctcacacggacggcaacttccacccttcaccgctaatgggaggatttggactgaagcacccacctcctcagatcctgatcaagaacacgccggtacctgcggatcctccaacaacgttcagccaggcgaaattggcttccttcattacgcagtacagcaccggacaggtcagcgtggaaatcgagtgggagctgcagaaggagaacagcaaacgctggaacccagagattcagtacacttcaaactactacaaatctacaaatgtggactttgctgtcaatacagagggaacttattctgagcctcgccccattggtactcgttacctcacccgtaatctg

SEQ ID NO: 32 - DNA, 2211 bp, unidentified
  source 1..2211: mol_type = genomic DNA
atggctgccgatggttatcttccagattggctcgaggacaacctctctgagggcattcgcgagtggtgggacttgaaacctggagccccgaaacccaaagccaaccagcaaaagcaggacgacggccggggcctggtgcttcctggctacaagtacctcggacccttcaacggactcgacaagggggagcccgtcaacgcggcggacgcagcggccctcgagcacgacaaggcctacgaccagcagctcgaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccagtagagcagtcaccccaagaaccagactcctcctcgggcatcggcaagaaaggccaacagcccgccagaaaaagactcaattttggccagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgttttttcccagtaacgggatcctgtttttggcaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctacagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 33 - DNA, 2217 bp, unidentified
  source 1..2217: mol_type = genomic DNA
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgccgatcctccgaccaccttcaatcaggccaagctggcttctttcatcacgcagtacagtaccggtcaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 34 - protein, 10 aa, unidentified
LALGETTRPA

SEQ ID NO: 35 - DNA, 141 bp, unidentified
  misc_feature 1..141: note = ITR Sequence
  source 1..141: mol_type = unassigned DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 36 - DNA, 1866 bp, unidentified; identical to SEQ ID NO: 3
  misc_feature 1..1866: note = Rep2 Sequence Contains Rep78 and Rep52
  source 1..1866: mol_type = unassigned DNA

SEQ ID NO: 37 - DNA, 2208 bp, unidentified; identical to SEQ ID NO: 5
  misc_feature 1..2208: note = Cap2 Sequence contains sequentially VP1, VP2, AAP, VP3
  source 1..2208: mol_type = unassigned DNA

SEQ ID NO: 38 - DNA, 2175 bp, unidentified
  misc_feature 1..2175: note = Cap5 Sequence contains sequentially VP1, VP2, AAP, VP3
  source 1..2175: mol_type = unassigned DNA
atggcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaagcaccagacctatcggaacccgataccttacccgacccctttaa

SEQ ID NO: 39 - DNA, 5336 bp, unidentified
  misc_feature 1..5336: note = E2A Full Sequence
  source 1..5336: mol_type = unassigned DNA
cgaccgcaccctgtgacgaaagccgcccgcaagctgcgcccctgagttagtcatctgaacttcggcctgggcgtctctgggaagtaccacagtggtgggagcgggactttcctggtacaccagggcagcgggccaactacggggattaaggttattacgaggtgtggtggtaatagccgcctgttcgaggagaattcggtttcggtgggcgcggattccgttgacccgggatatcatgtggggtcccgcgctcatgtagtttattcgggttgagtagtcttgggcagctccagccgcaagtcccatttgtggctggtaactccacatgtagggcgtgggaatttccttgctcataatggcgctgacgacaggtgctggcgccgggtgtggccgctggagatgacgtagttttcgcgcttaaatttgagaaagggcgcgaaactagtccttaagagtcagcgcgcagtatttgctgaagagagcctccgcgtcttccagcgtgcgccgaagctgatcttcgcttttgtgatacaggcagctgcgggtgagggagcgcagagacctgttttttattttcagctcttgttcttggcccctgctttgttgaaatatagcatacagagtgggaaaaatcctatttctaagctcgcgggtcgatacgggttcgttgggcgccagacgcagcgctcctcctcctgctgctgccgccgctgtggatttcttgggctttgtcagagtcttgctatccggtcgcctttgcttctgtgtgaccgctgctgttgctgccgctgccgctgccgccggtgcagtaggggctgtagagatgacggtagtaatgcaggatgttacgggggaaggccacgccgtgatggtagagaagaaagcggcgggcgaaggagatgttgcccccacagtcttgcaagcaagcaactatggcgttcttgtgcccgcgccacgagcggtagccttggcgctgttgttgctcttgggctaacggcggcggctgcttagacttaccggccctggttccagtggtgtcccatctacggttgggtcggcgaacaggcagtgccggcggcgcctgaggagcggaggttgtagcgatgctgggaacggttgccaatttctggggcgccggcgaggggaatgcgaccgagggtgacggtgtttcgtctgacacctcttcggcctcggaagcttcgtctaggctgtcccagtcttccatcatctcctcctcctcgtccaaaacctcctctgcctgactgtcccagtattcctcctcgtccgtgggtggcggcggcggcagctgcagcttctttttgggtgccatcctgggaagcaagggcccgcggctgctgatagggctgcggcggcggggggattgggttgagctcctcgccggactgggggtccaggtaaaccccccgtccctttcgtagcagaaactcttggcgggctttgttgatggcttgcaattggccaaggatgtggccctgggtaatgacgcaggcggtaagctccgcatttggcgggcgggattggtcttcgtagaacctaatctcgtgggcgtggtagtcctcaggtacaaatttgcgaaggtaagccgacgtccacagccccggagtgagtttcaaccccggagccgcggacttttcgtcaggcgagggaccctgcagctcaaaggtaccgataatttgactttcgctaagcagttgcgaattgcagaccagggagcggtgcggggtgcataggttgcagcgacagtgacactccagtaggccgtcaccgctcacgtcttccatgatgtcggagtggtaggcaaggtagttggctagctgcagaaggtagcagtgaccccaaagcggcggagggcattcacggtacttaatgggcacaaagtcgctaggaagcgcacagcaggtggcgggcagaattcctgaacgctctaggataaagttcctaaagttttgcaacatgctttgactggtgaagtctggcagaccctgttgcagggttttaagcaggcgttcggggaagataatgtccgccaggtgcgcggccacggagcgctcgttgaaggccgtccataggtccttcaagttttgctttagcagcttctgcagctcctttaggttgcgctcctccaggcattgctgccacacgcccatggccgtttgccaggtgtagcacagaaataagtaaacgcagtcgcggacgtagtcgcggcgcgcctcgcccttgagcgtggaatgaagcacgttttgcccgaggcggttttcgtgcaaaattccaaggtaggagaccaggttgcagagctccacgttggaaattttgcaggcctggcgcacgtagccctggcgaaaggtgtagtgcaacgtttcctctagcttgcgctgcatctccgggtcagcaaagaaccgctgcatgcactcaagctccacggtaacaagcactgcggccatcattagcttgcgtcgctcctccaagtcggcaggctcgcgcgtctcaagccagcgcgccagctgctcatcgccaactgcgggtaggccctcctcggtttgttcttgcaagtttgcatccctctccaggggtcgtgcacggcgcacgatcagctcgctcatgactgtgctcataaccttggggggtaggttaagtgccgggtaggcaaagtgggtgacctcgatgctgcgtttcagcacggctaggcgcgcgttgtcaccctcaagttccaccagcactccacagtgactttcattttcgctgttttcttgttgcagagcgtttgccgcgcgtttctcgtcgcgtccaagaccctcaaagatttttggcacttcgtcgagcgaggcgatatcaggtatgacagcgccctgccgcaaggccagctgcttgtccgctcggctgcggttggcacggcaggataggggtatcttgcagttttggaaaaagatgtgataggtggcaagcacctctggcacggcaaatacggggtagaagttgaggcgcgggttgggctcgcatgtgccgttttcttggcgtttggggggtacgcgcggtgagaacaggtggcgttcgtaggcaaggctgacatccgctatggcgaggggcacatcgctgcgctcttgcaacgcgtcgcagataatggcgcactggcgctgcagatgcttcaacagcacgtcgtctcccacatctaggtagtcgccatgcctttggtccccccgcccgacttgttcctcgtttgcctctgcgtcgtcctggtcttgctttttatcctctgttggtactgagcgatcctcgtcgtcttcgcttacaaaacctgggtcctgctcgataatcacttcctcctcctcaagcgggggtgcctcgacggggaaggtggtaggcgcgttggcggcatcggtggaggcggtggtggcgaactcaaagggggcggttaggctgtcctccttctcgactgactccatgatctttttctgcctataggagaaggaaatggccagtcgggaagaggagcagcgcgaaaccacccccgagcgcggacgcggtgcggcgcgacgtccaccaaccatggaggacgtgtcgtccccgtcgccgtcgccgccgcctccccgcgcgcccccaaaaaagcggctgaggcggcgtctcgagtccgaggacgaagaagactcgtcacaagatgcgctggtgccgcgcacacccagcccgcggccatcgacctcgacggcggatttggccattgcgtccaaaaagaaaaagaagcgcccctctcccaagcccgagcgcccgccatccccagaggtgatcgtggacagcgaggaagaaagagaagatgtggcgctacaaatggtgggtttcagcaacccaccggtgctaatcaagcacggcaagggaggtaagcgcacggtgcggcggctgaatgaagacgacccagtggcgcggggtatgcggacgcaagaggaaaaggaagagtccagtgaagcggaaagtgaaagcacggtgataaacccgctgagcctgccgatcgtgtctgcgtgggagaagggcatggaggctgcgcgcgcgttgatggacaagtaccacgtggataacgatctaaaggcaaacttcaagctactgcctgaccaagtggaagctctggcggccgtatgcaagacctggctaaacgaggagcaccgcgggttgcagctgaccttcaccagcaacaagacctttgtgacgatgatggggcgattcctgcaggcgtacctgcagtcgtttgcagaggtaacctacaagcaccacgagcccacgggctgcgcgttgtggctgcaccgctgcgctgagatcgaaggcgagcttaagtgtctacacgggagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcatgacgcggcctgtccggccaatcagttttccggcaagtcttgcggcatgttcttctctgaaggcgcaaaggctcaggtggcttttaagcagatcaaggctttcatgcaggcgctgtatcctaacgcccagaccgggcacggtcaccttctgatgccactacggtgcgagtgcaactcaaagcctgggcatgcaccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaaacggcgcagacggcaagggtggggggtaaataatcacccgagagtgtacaaataaaaacatttgcctttattgaaagtgtctcctagtacattatttttacatgtttttcaagtgacaaaaagaagtggcgctcctaatctgcgcactgtggctgcggaagtagggcgagtggcgctccaggaagctgtagagctgttcctggttgcgacgcagggtgggctgtacctggggactgttaagcatggagttgggtacc

SEQ ID NO: 40 - DNA, 1590 bp, unidentified
  misc_feature 1..1590: note = E2A ORF Sequence
  source 1..1590: mol_type = unassigned DNA
atggccagtcgggaagaggagcagcgcgaaaccacccccgagcgcggacgcggtgcggcgcgacgtccaccaaccatggaggacgtgtcgtccccgtcgccgtcgccgccgcctccccgcgcgcccccaaaaaagcggctgaggcggcgtctcgagtccgaggacgaagaagactcgtcacaagatgcgctggtgccgcgcacacccagcccgcggccatcgacctcgacggcggatttggccattgcgtccaaaaagaaaaagaagcgcccctctcccaagcccgagcgcccgccatccccagaggtgatcgtggacagcgaggaagaaagagaagatgtggcgctacaaatggtgggtttcagcaacccaccggtgctaatcaagcacggcaagggaggtaagcgcacggtgcggcggctgaatgaagacgacccagtggcgcggggtatgcggacgcaagaggaaaaggaagagtccagtgaagcggaaagtgaaagcacggtgataaacccgctgagcctgccgatcgtgtctgcgtgggagaagggcatggaggctgcgcgcgcgttgatggacaagtaccacgtggataacgatctaaaggcaaacttcaagctactgcctgaccaagtggaagctctggcggccgtatgcaagacctggctaaacgaggagcaccgcgggttgcagctgaccttcaccagcaacaagacctttgtgacgatgatggggcgattcctgcaggcgtacctgcagtcgtttgcagaggtaacctacaagcaccacgagcccacgggctgcgcgttgtggctgcaccgctgcgctgagatcgaaggcgagcttaagtgtctacacgggagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcatgacgcggcctgtccggccaatcagttttccggcaagtcttgcggcatgttcttctctgaaggcgcaaaggctcaggtggcttttaagcagatcaaggctttcatgcaggcgctgtatcctaacgcccagaccgggcacggtcaccttctgatgccactacggtgcgagtgcaactcaaagcctgggcatgcaccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaa

SEQ ID NO: 41 - DNA, 3201 bp, unidentified
  misc_feature 1..3201: note = E4 Full Sequence
  source 1..3201: mol_type = unassigned DNA
cccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaa

SEQ ID NO: 42 - DNA, 885 bp, unidentified
  misc_feature 1..885: note = E4 Orf6 Sequence
  source 1..885: mol_type = unassigned DNA
atgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtag

SEQ ID NO: 43 - DNA, 743 bp, unidentified
  misc_feature 1..743: note = VA Sequence
  source 1..743: mol_type = unassigned DNA
cgtaatccgtagatgtacctggacatccaggtgatgccggcggcggtggtggaggcgcgcggaaagtcgcggacgcggttccagatgttgcgcagcggcaaaaagtgctccatggtcgggacgctctggccggtgaggcgtgcgcagtcgttgacgctctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatcctacatcga

SEQ ID NO: 44 - DNA, 870 bp, unidentified
  misc_feature 1..870: note = Human mastadenovirus C
  source 1..870: mol_type = genomic DNA
atgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataa

SEQ ID NO: 45 - DNA, 732 bp, unidentified
  misc_feature 1..732: note = Human mastadenovirus C
  source 1..732: mol_type = genomic DNA
atgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataa

SEQ ID NO: 46 - DNA, 531 bp, unidentified
  misc_feature 1..531: note = Human mastadenovirus C
  source 1..531: mol_type = genomic DNA
atggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatga

SEQ ID NO: 47 - DNA, 1491 bp, unidentified
  misc_feature 1..1491: note = Human mastadenovirus C
  source 1..1491: mol_type = genomic DNA
atggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattga

SEQ ID NO: 48 - DNA, 1590 bp, unidentified; identical to SEQ ID NO: 40
  misc_feature 1..1590: note = Human mastadenovirus C
  source 1..1590: mol_type = genomic DNA

SEQ ID NO: 49 - DNA, 453 bp, unidentified
  misc_feature 1..453: note = Human mastadenovirus C
  source 1..453: mol_type = genomic DNA
atgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtga

SEQ ID NO: 50 - DNA, 885 bp, unidentified; identical to SEQ ID NO: 42
  misc_feature 1..885: note = Human mastadenovirus C
  source 1..885: mol_type = genomic DNA

SEQ ID NO: 51 - DNA, 743 bp, synthetic construct
  misc_feature 1..743: note = Synthetic
  source 1..743: mol_type = other DNA
tcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacg

SEQ ID NO: 52 - DNA, 39 bp, synthetic construct
  misc_feature 1..39: note = Synthetic
  source 1..39: mol_type = other DNA
gcccacatcgtgatggtggacgcctacaagccgacgaag

SEQ ID NO: 53 - DNA, 342 bp, synthetic construct
  misc_feature 1..342: note = Synthetic
  source 1..342: mol_type = other DNA
gtggataccctgtccggactgagcagtgagcaaggccagtccggagatatgacaattgaagaagatagcgccacccatattaaattctccaaaagagatgaggacggcaaagagctggctggagcaacaatggagctgagagattcctctggaaagactattagtacatggatctctgatggccaagtgaaagatttctatctgtatccaggaaagtacacatttgtcgaaaccgctgcaccagacggatatgaggtggctacagctattacctttacagtgaatgagcaaggacaggtgactgttaatggcaaagctactaaaggagacgctcatatttaa

SEQ ID NO: 54 - protein, 13 aa, synthetic construct
  REGION 1..13: note = Synthetic
AHIVMVDAYKPTK

SEQ ID NO: 55 - DNA, 32 bp, synthetic construct
  misc_feature 1..32: note = Synthetic
  source 1..32: mol_type = other DNA
cacatatcagagtgcgacacacagactgtgag

SEQ ID NO: 56 - DNA, 32 bp, synthetic construct
  misc_feature 1..32: note = Synthetic
  source 1..32: mol_type = other DNA
acacatctcgtgagagcacgcacacacgcgcg

SEQ ID NO: 57 - DNA, 32 bp, synthetic construct
  misc_feature 1..32: note = Synthetic
  source 1..32: mol_type = other DNA
cactcgactctcgcgtcatatatatcagctgt

SEQ ID NO: 58 - DNA, 32 bp, synthetic construct
  misc_feature 1..32: note = Synthetic
  source 1..32: mol_type = other DNA
tctgtatctctatgtgacagtcgagcgctgcg

SEQ ID NO: 59 - DNA, 32 bp, synthetic construct
  misc_feature 1..32: note = Synthetic
  source 1..32: mol_type = other DNA
acacacgcgagacagaacgcgctatctcagag

SEQ ID NO: 60 - DNA, 32 bp, synthetic construct
  misc_feature 1..32: note = Synthetic
  source 1..32: mol_type = other DNA
ctatacgtatatctatacactagatcgcgtgt

SEQ ID NO: 61 - DNA, 32 bp, synthetic construct
  misc_feature 1..32: note = Synthetic
  source 1..32: mol_type = other DNA
ctctcgcatacgcgagctcactacgcgcgcgt

SEQ ID NO: 62 - DNA, 32 bp, synthetic construct
  misc_feature 1..32: note = Synthetic
  source 1..32: mol_type = other DNA
cgcatgacacgtgtgtcatagagagatagtat

SEQ ID NO: 63 - DNA, 32 bp, synthetic construct
  misc_feature 1..32: note = Synthetic
  source 1..32: mol_type = other DNA
cacacgcgcgctatattcacgtgctcactgtg

SEQ ID NO: 64 - DNA, 32 bp, synthetic construct
  misc_feature 1..32: note = Synthetic
  source 1..32: mol_type = other DNA
acacactctatcagatcacgacacgacgatgt

SEQ ID NO: 65 - DNA, 32 bp, synthetic construct
  misc_feature 1..32: note = Synthetic
  source 1..32: mol_type = other DNA
ctatacatagtgatgtcactcacgtgtgatat

SEQ ID NO: 66 - DNA, 32 bp, synthetic construct
  misc_feature 1..32: note = Synthetic
  source 1..32: mol_type = other DNA
cagagagatatctctgcatgtagagcagagag

SEQ ID NO: 67 - DNA, 32 bp, synthetic construct
  misc_feature 1..32: note = Synthetic
  source 1..32: mol_type = other DNA
cgcgacacgctcgcgccacagagacacgcaca

SEQ ID NO: 68 - DNA, 32 bp, synthetic construct
  misc_feature 1..32: note = Synthetic
  source 1..32: mol_type = other DNA
ctcacactctctcacactctgctctgactctc

SEQ ID NO: 69 - DNA, 32 bp, synthetic construct
  misc_feature 1..32: note = Synthetic
  source 1..32: mol_type = other DNA
tatatatgtctatagatctctctatcgcgctc

SEQ ID NO: 70 - DNA, 32 bp, synthetic construct
  misc_feature 1..32: note = Synthetic
  source 1..32: mol_type = other DNA
gatgtctgagtgtgtggagactagagatagtg

SEQ ID NO: 71 - DNA, 32 bp, synthetic construct
  misc_feature 1..32: note = Synthetic
  source 1..32: mol_type = other DNA
tctcgtcgcagtctctatgtgtatatagatat

SEQ ID NO: 72 - DNA, 32 bp, synthetic construct
  misc_feature 1..32: note = Synthetic
  source 1..32: mol_type = other DNA
gcgcgcgcactctctggagacacgtcgcacac

SEQ ID NO: 73 - DNA, 32 bp, synthetic construct
  misc_feature 1..32: note = Synthetic
  source 1..32: mol_type = other DNA
acacatatcgcactacgtgtgtctcgatgcgc

SEQ ID NO: 74 - DNA, 32 bp, synthetic construct
  misc_feature 1..32: note = Synthetic
  source 1..32: mol_type = other DNA
cgcacacatagatacatgtcatatgagagtgt

SEQ ID NO: 75 - DNA, 32 bp, synthetic construct
  misc_feature 1..32: note = Synthetic
  source 1..32: mol_type = other DNA
tctcgcgcgtgcacgcctcgctcgacgagcgc

SEQ ID NO: 76 - DNA, 32 bp, synthetic construct
  misc_feature 1..32: note = Synthetic
  source 1..32: mol_type = other DNA
tatagagctctacatagctgagacgacgcgcg

SEQ ID NO: 77 - DNA, 32 bp, synthetic construct
  misc_feature 1..32: note = Synthetic
  source 1..32: mol_type = other DNA
acatatcgtactctctgatatatcgagtatat

SEQ ID NO: 78 - DNA, 32 bp, synthetic construct
  misc_feature 1..32: note = Synthetic
  source 1..32: mol_type = other DNA
tgtcatgtgtacacacgtgtgcactcacactc

SEQ ID NO: 79 - DNA, 32 bp, synthetic construct
  misc_feature 1..32: note = Synthetic
  source 1..32: mol_type = other DNA
acacgtgtgctctctcgatatacgcgagagag

SEQ ID NO: 80 - DNA, 32 bp, synthetic construct
  misc_feature 1..32: note = Synthetic
  source 1..32: mol_type = other DNA
cgtgtctagcgcgcgcgtgtgagatatatatc

SEQ ID NO: 81 - DNA, 32 bp, synthetic construct
  misc_feature 1..32: note = Synthetic
  source 1..32: mol_type = other DNA
ctcacgtacgtcacacgcgcacgcactacaga

SEQ ID NO: 82 - DNA, 32 bp, synthetic construct
  misc_feature 1..32: note = Synthetic
  source 1..32: mol_type = other DNA
cacacgagatctcatcagacacacacgcacat

SEQ ID NO: 83 - DNA, 32 bp, synthetic construct
  misc_feature 1..32: note = Synthetic
  source 1..32: mol_type = other DNA
gacgagcgtctgagagtgtgtctctgagagta

SEQ ID NO: 84 - DNA, 32 bp, synthetic construct
  misc_feature 1..32: note = Synthetic
  source 1..32: mol_type = other DNA
cacacgcactgagatagatgagtatagacaca

SEQ ID NO: 85 - DNA, 32 bp, synthetic construct
  misc_feature 1..32: note = Synthetic
  source 1..32: mol_type = other DNA
gctgtgtgtgctcgtctctcagatagtctata

SEQ ID NO: 86 - DNA, 32 bp, synthetic construct
  misc_feature 1..32: note = Synthetic
  source 1..32: mol_type = other DNA
acacgcatgacacacttatatacagagtcgag

SEQ ID NO: 87 - DNA, 32 bp, synthetic construct
  misc_feature 1..32: note = Synthetic
  source 1..32: mol_type = other DNA
gcgctctctcacatactatatgctctgtgtga

SEQ ID NO: 88 - DNA, 32 bp, synthetic construct
  misc_feature 1..32: note = Synthetic
  source 1..32: mol_type = other DNA
ctctatatatctcgtcagagagctctctcatc

SEQ ID NO: 89 - DNA, 32 bp, synthetic construct
  misc_feature 1..32: note = Synthetic
  source 1..32: mol_type = other DNA
gcgagagtgagacgcatgctctcgtgtactgt

SEQ ID NO: 90 - DNA, 32 bp, synthetic construct
  misc_feature 1..32: note = Synthetic
  source 1..32: mol_type = other DNA
agcgctgcgacacgcgagacgcgagcgcgtag

SEQ ID NO: 91 - DNA, 32 bp, synthetic construct
  misc_feature 1..32: note = Synthetic
  source 1..32: mol_type = other DNA
gcgtgtgtcgagtgtatgtacgctctctatat

SEQ ID NO: 92 - DNA, 32 bp, synthetic construct
  misc_feature 1..32: note = Synthetic
  source 1..32: mol_type = other DNA
tagagagcgtcgcgtggtgcactcgcgctctc

SEQ ID NO: 93 - DNA, 32 bp, synthetic construct
  misc_feature 1..32: note = Synthetic
  source 1..32: mol_type = other DNA
tatctctcgagtcgcgctcacacatacacgtc

SEQ ID NO: 94 - DNA, 32 bp, synthetic construct
  misc_feature 1..32: note = Synthetic
  source 1..32: mol_type = other DNA
atagtacactctgtgttatctctgtagagtct

SEQ ID NO: 95 - DNA, 32 bp, synthetic construct
  misc_feature 1..32: note = Synthetic
  source 1..32: mol_type = other DNA
gatatatatgtgtgtagtgacacacagagcac

SEQ ID NO: 96 - DNA, 32 bp, synthetic construct
  misc_feature 1..32: note = Synthetic
  source 1..32: mol_type = other DNA
atatgacatacacgcacgtctctcgtctgtgc

SEQ ID NO: 97 - DNA, 32 bp, synthetic construct
  misc_feature 1..32: note = Synthetic
  source 1..32: mol_type = other DNA
acacagtagagcgagcgtcgcgcatagagcgc

SEQ ID NO: 98 - DNA, 32 bp, synthetic construct
  misc_feature 1..32: note = Synthetic
  source 1..32: mol_type = other DNA
ctatctagcactcacacgtgtcactctgcgtg

SEQ ID NO: 99 - DNA, 32 bp, synthetic construct
  misc_feature 1..32: note = Synthetic
  source 1..32: mol_type = other DNA
cgcgcgagtatctcgtagcacacatatagcgc

SEQ ID NO: 100 - DNA, 32 bp, synthetic construct
  misc_feature 1..32: note = Synthetic
  source 1..32: mol_type = other DNA
gtatatatatacgtcttctcacgagagcgcac

SEQ ID NO: 101 - DNA, 32 bp, synthetic construct
  misc_feature 1..32: note = Synthetic
  source 1..32: mol_type = other DNA
tagatgcgagagtagaatagcgacatctctct

SEQ ID NO: 102 - DNA, 32 bp, synthetic construct
  misc_feature 1..32: note = Synthetic
  source 1..32: mol_type = other DNA
gcacgatgtcagcgcgtgtgctctctacacag

SEQ ID NO: 103 - protein, 113 aa, synthetic construct
  REGION 1..113: note = Synthetic
VDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 104 - protein, 92 aa, synthetic construct
  REGION 1..92: note = Synthetic
DSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 105 - protein, 113 aa, synthetic construct
  REGION 1..113: note = Synthetic
VTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHT

SEQ ID NO: 106 - protein, 113 aa, synthetic construct
  REGION 1..113: note = Synthetic
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 107 - protein, 83 aa, synthetic construct
  REGION 1..83: note = Synthetic
DSATHIKFSKRDEDGKELAGATMELRDSSGKTISTWISDGQVKDFYLYPGKYTFVETAAPDGYEVATAITFTVNEQGQVTVNG

SEQ ID NO: 108 - protein, 112 aa, synthetic construct
  REGION 1..112: note = Synthetic
KPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 109 - protein, 98 aa, synthetic construct
  REGION 1..98: note = Synthetic
DYDGQSGDGKELAGATMELRDSSGKTISTWISDGQVKDFYLYPGKYTFVETAAPDGYEVATAITFTVNEQGQVTVNGKATKGGSGGSGGSGEDSATHI

SEQ ID NO: 110 - protein, 98 aa, synthetic construct
  REGION 1..98: note = Synthetic
LSGETGQSGNTTIEEDSTTHVKFSKRDANGKELAGAMIELRNLSGQTIQSWISDGTVKVFYLMPGTYQFVETAAPEGYELAAPITFTIDEKGQIWVDS

SEQ ID NO: 111 - protein, 104 aa, synthetic construct
  REGION 1..104: note = Synthetic
KLGDIEFIKVNKNDKKPLRGAVFSLQKQHPDYPDIYGAIDQNGTYQNVRTGEDGKLTFKNLSDGKYRLFENSEPAGYKPVQNKPIVAFQIVNGEVRDVTSIVPQ

SEQ ID NO: 112 - protein, 104 aa, synthetic construct
  REGION 1..104: note = Synthetic
KLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 113 - protein, 104 aa, synthetic construct
  REGION 1..104: note = Synthetic
KLGEIEFIKVDKTDKKPLRGAVFSLQKQHPDYPDIYGAIDQNGTYQDVRTGEDGKLTFTNLSDGKYRLFENSEPPGYKPVQNKPIVAFQIVDGEVRDVTSIVPQ

SEQ ID NO: 114 - protein, 91 aa, synthetic construct
  REGION 1..91: note = Synthetic
EQDVVFSKVNVAGEEIAGAKIQLKDAQGQVVHSWTSKAGQSETVKLKAGTYTFHEASAPTGYLAVTDITFEVDVQGKVTVKDANGNGVKAD

SEQ ID NO: 115 - protein, 282 aa, synthetic construct
  REGION 1..282: note = Synthetic
ATTVHGETVVNGAKLTVTKNLDLVNSNALIPNTDFTFKIEPDTTVNEDGNKFKGVALNTPMTKVTYTNSDKGGSNTKTAEFDFSEVTFEKPGVYYYKVTEEKIDKVPGVSYDTTSYTVQVHVLWNEEQQKPVATYIVGYKEGSKVPIQFKNSLDSTTLTVKKKVSGTGGDRSKDFNFGLTLKANQYYKASEKVMIEKTTKGGQAPVQTEASIDQLYHFTLKDGESIKVTNLPVGVDYVVTEDDYKSEKYTTNVEVSPQDGAVKNIAGNSTEQETSTDKDMTI

SEQ ID NO: 116 - protein, 113 aa, synthetic construct
  REGION 1..113: note = Synthetic
IDTMSGLSGETGQSGNTTIEEDSTTHVKFSKRDSNGKELAGAMIELRNLSGQTIQSWVSDGTVKDFYLMPGTYQFVETAAPEGYELAAPITFTVNEQGQVTVNGKATKGDAHI

SEQ ID NO: 117 - protein, 112 aa, synthetic construct
  REGION 1..112: note = Synthetic
DTMSGLSGETGQSGNTTIEEDSTTHVKFSKRDSNGKELAGAMIELRNLSGQTIQSWVSDGTVKDFYLMPGTYQFVETAAPEGYELAAPITFTVQDNGEVQIQGKATRGDVPI

SEQ ID NO: 118 - protein, 113 aa, synthetic construct
  REGION 1..113: note = Synthetic
IDTMSGLSGETGQSGNTTIEEDSTTHVKFSKRDSNGKELAGAMIELRNLSGQTIQSWVSDGTVKDFYLMPGTYQFVETAAPEGYELAAPITFTVQDNGEVIIQGRLTRGDVHI

SEQ ID NO: 119 - protein, 14 aa, synthetic construct
  REGION 1..14: note = Synthetic
VPTIVMVDAYKRYK

SEQ ID NO: 120 - protein, 16 aa, synthetic construct
  REGION 1..16: note = Synthetic
RGVPHIVMVDAYKRYK

SEQ ID NO: 121 - protein, 12 aa, synthetic construct
  REGION 1..12: note = Synthetic
KLGSIEFIKVNK

SEQ ID NO: 122 - protein, 23 aa, synthetic construct
  REGION 1..23: note = Synthetic
DIPATYEFTDGKHYITNEPIPPK

SEQ ID NO: 123 - protein, 13 aa, synthetic construct
  REGION 1..13: note = Synthetic
GNKLTVTDQAAPS

SEQ ID NO: 124 - protein, 19 aa, synthetic construct
  REGION 1..19: note = Synthetic
DIPATYEFTNDKHYITNEP

SEQ ID NO: 125 - protein, 16 aa, synthetic construct
  REGION 1..16: note = Synthetic
TDKDMTITFTNKKDAE

SEQ ID NO: 126 - protein, 16 aa, synthetic construct
  REGION 1..16: note = Synthetic
RGVPTIVMVDCYKRYK

SEQ ID NO: 127 - protein, 16 aa, synthetic construct
  REGION 1..16: note = Synthetic
GTPIVIMVDEAKPSLP

SEQ ID NO: 128 - protein, 16 aa, synthetic construct
  REGION 1..16: note = Synthetic
GNPLIVMIDEAEQKEI

SEQ ID NO: 129 - protein, 16 aa, synthetic construct
  REGION 1..16: note = Synthetic
AGGIIVMKDNTTKVSI

SEQ ID NO: 130 - protein, 16 aa, synthetic construct
  REGION 1..16: note = Synthetic
GNPIVTMIDDATLVKI

SEQ ID NO: 131 - protein, 16 aa, synthetic construct
  REGION 1..16: note = Synthetic
GNSTITMVDDTTKVHI

SEQ ID NO: 132 - protein, 16 aa, synthetic construct
  REGION 1..16: note = Synthetic
GTPLIVMVDDTTKVEI

SEQ ID NO: 133 - protein, 10 aa, synthetic construct
  REGION 1..10: note = Synthetic
ATHIKFSKRD

SEQ ID NO: 134 - protein, 13 aa, synthetic construct
  REGION 1..13: note = Synthetic
AHIVMVDAYKPTK

SEQ ID NO: 135 - DNA, 39 bp, synthetic construct
  misc_feature 1..39: note = Synthetic
  source 1..39: mol_type = other DNA
gctcacatcgtgatggtggacgcttacaagcccaccaag

SEQ ID NO: 136 - protein, 13 aa, synthetic construct
  REGION 1..13: note = Synthetic
DPIVMIDNDKPIT

SEQ ID NO: 137 - DNA, 39 bp, synthetic construct
  misc_feature 1..39: note = Synthetic
  source 1..39: mol_type = other DNA
gaccccatcgtgatgatcgacaacgacaagcccatcacc

SEQ ID NO: 138 - protein, 12 aa, synthetic construct
  REGION 1..12: note = Synthetic
KLGDIEFIKVNK

SEQ ID NO: 139 - DNA, 36 bp, synthetic construct
  misc_feature 1..36: note = Synthetic
  source 1..36: mol_type = other DNA
aaactgggtgatattgaatttattaaagttaataaa

SEQ ID NO: 140 - protein, 22 aa, synthetic construct
  REGION 1..22: note = Synthetic
LVTGTAHIVMVDNYKPIVETGD

SEQ ID NO: 141 - DNA, 66 bp, synthetic construct
  misc_feature 1..66: note = Synthetic
  source 1..66: mol_type = other DNA
ctggttaccggcaccgcacatattgttatggttgataactataagccgatcgtggaaaccggtgat

SEQ ID NO: 142 - protein, 15 aa, synthetic construct
  REGION 1..15: note = Synthetic
NTIVMVDKLKEVPPT

SEQ ID NO: 143 - DNA, 60 bp, synthetic construct
  misc_feature 1..60: note = Synthetic
  source 1..60: mol_type = other DNA
accgaagttagcggtaataccattgtgatggtggataaactgaaagaagttccgcctacc

SEQ ID NO: 144 - protein, 20 aa, synthetic construct
  REGION 1..20: note = Synthetic
SENGNPLIVMVDDTTKVKIS

SEQ ID NO: 145 - DNA, 60 bp, synthetic construct
  misc_feature 1..60: note = Synthetic
  source 1..60: mol_type = other DNA
agcgaaaacggcaacccgctgattgtgatggtggatgataccaccaaagtgaaaattagc

SEQ ID NO: 146 - protein, 17 aa, synthetic construct
  REGION 1..17: note = Synthetic
GTPIVIMVDEAKPSLPD

SEQ ID NO: 147 - DNA, 51 bp, synthetic construct
  misc_feature 1..51: note = Synthetic
  source 1..51: mol_type = other DNA
ggcaccccgattgtgattatggtggatgaagcgaaaccgagcctgccggat

SEQ ID NO: 148 - protein, 17 aa, synthetic construct
  REGION 1..17: note = Synthetic
GNPLIVMIDEAEQKEIP

SEQ ID NO: 149 - DNA, 51 bp, synthetic construct
  misc_feature 1..51: note = Synthetic
  source 1..51: mol_type = other DNA
ggcaacccgctgattgtgatgattgatgaagcggaacagaaagaaattccg

SEQ ID NO: 150 - protein, 17 aa, synthetic construct
  REGION 1..17: note = Synthetic
AGGIIVMKDNTTKVSIS

SEQ ID NO: 151 - DNA, 51 bp, synthetic construct
  misc_feature 1..51: note = Synthetic
  source 1..51: mol_type = other DNA
gcgggcggcattattgtgatgaaagataacaccaccaaagtgagcattagc

SEQ ID NO: 152 - protein, 17 aa, synthetic construct
  REGION 1..17: note = Synthetic
GNPIVTMIDDATLVKIS

SEQ ID NO: 153 - DNA, 48 bp, synthetic construct
  misc_feature 1..48: note = Synthetic
  source 1..48: mol_type = other DNA
ggcaacccgattgtgaccatgattgatgatgcgaccctggtgaaaatt

SEQ ID NO: 154 - protein, 17 aa, synthetic construct
  REGION 1..17: note = Synthetic
GNSTITMVDDTTKVHIT

SEQ ID NO: 155 - DNA, 51 bp, synthetic construct
  misc_feature 1..51: note = Synthetic
  source 1..51: mol_type = other DNA
ggcaacagcaccattaccatggtggatgataccaccaaagtgcatattacc

SEQ ID NO: 156 - protein, 17 aa, synthetic construct
  REGION 1..17: note = Synthetic
GTPLIVMVDDTTKVEIS

SEQ ID NO: 157 - DNA, 51 bp, synthetic construct
  misc_feature 1..51: note = Synthetic
  source 1..51: mol_type = other DNA
ggcaccccgctgattgtgatggtggatgataccaccaaagtggaaattagc

SEQ ID NO: 158 - protein, 15 aa, synthetic construct
  REGION 1..15: note = Synthetic
GNPLIVMVNDTTKVK

SEQ ID NO: 159 - DNA, 45 bp, synthetic construct
  misc_feature 1..45: note = Synthetic
  source 1..45: mol_type = other DNA
ggtaatccgctgattgtgatggtgaatgataccaccaaagtgaaa

SEQ ID NO: 160 - protein, 15 aa, synthetic construct
  REGION 1..15: note = Synthetic
GNPLIVMVDDTTKVK

SEQ ID NO: 161 - DNA, 45 bp, synthetic construct
  misc_feature 1..45: note = Synthetic
  source 1..45: mol_type = other DNA
ggcaacccgctgattgtgatggtggatgataccaccaaagtgaaa

SEQ ID NO: 162 - protein, 20 aa, synthetic construct
  REGION 1..20: note = Synthetic
NEKVTGQFEIVKVDANDKTK

SEQ ID NO: 163 - DNA, 45 bp, synthetic construct
  misc_feature 1..45: note = Synthetic
  source 1..45: mol_type = other DNA
ggtcagttcgaaattgttaaagttgatgcaaacgataaaactaaa

SEQ ID NO: 164 - protein, 19 aa, synthetic construct
  REGION 1..19: note = Synthetic
SKSLGQFEIVKVDAQDKTK

SEQ ID NO: 165 - DNA, 57 bp, synthetic construct
  misc_feature 1..57: note = Synthetic
  source 1..57: mol_type = other DNA
agcaaaagcctgggccagtttgaaattgtgaaagtggatgcgcaggataaaaccaaa

SEQ ID NO: 166 - protein, 16 aa, synthetic construct
  REGION 1..16: note = Synthetic
LGQFEIVKVDSQDKTK

SEQ ID NO: 167 - DNA, 48 bp, synthetic construct
  misc_feature 1..48: note = Synthetic
  source 1..48: mol_type = other DNA
ctgggccagtttgaaattgttaaagttgatagccaggataaaaccaaa

SEQ ID NO: 168 - protein, 17 aa, synthetic construct
  REGION 1..17: note = Synthetic
VTGQFEIVKVDAEDKTR

SEQ ID NO: 169 - DNA, 51 bp, synthetic construct
  misc_feature 1..51: note = Synthetic
  source 1..51: mol_type = other DNA
gttaccggtcagtttgaaatcgttaaagttgatgccgaagataagacccgt

SEQ ID NO: 170 - protein, 19 aa, synthetic construct
  REGION 1..19: note = Synthetic
EKVMGQFEIMKVDANDKTK

SEQ ID NO: 171 - DNA, 57 bp, synthetic construct
  misc_feature 1..57: note = Synthetic
  source 1..57: mol_type = other DNA
gaaaaagtgatgggccagttcgaaatcatgaaagttgatgccaacgacaagaccaaa

SEQ ID NO: 172 - protein, 23 aa, synthetic construct
  REGION 1..23: note = Synthetic
DTKQVVKHEDKNDKAQTLVVEKP

SEQ ID NO: 173 - DNA, 69 bp, synthetic construct
  misc_feature 1..69: note = Synthetic
  source 1..69: mol_type = other DNA
gacaccaagcaggtggtgaagcacgaggacaagaacgacaaggcccagaccctggtggtggagaagccc

SEQ ID NO: 174 - protein, 104 aa, synthetic construct
  REGION 1..104: note = Synthetic
GAMVDTLSGLSSEQGQSGDMTIEEDSATHIKESKRDEDGKELAGATMELRDSSGKTISTWISDGQVKDFYLYPGKYTFVETAAPDGYEVATAITFTVNEQGQVT

SEQ ID NO: 175 - DNA, 348 bp, synthetic construct
  misc_feature 1..348: note = Synthetic
  source 1..348: mol_type = other DNA
ggtgcaatggttgataccctgagcggtctgagcagcgaacagggtcagagcggtgatatgaccattgaagaagatagcgcaacccacatcaaattcagcaaacgtgatgaagatggtaaagaactggcaggcgcaacaatggaactgcgtgatagcagcggtaaaaccattagcacctggattagtgatggtcaggtgaaagatttttatctgtaccctggcaaatacacctttgttgaaaccgcagcaccggatggttatgaagttgcaaccgcaattacctttaccgttaatgaacagggccaggttaccgtgaatggtaaagcaaccaaaggtgatgcacatatt

SEQ ID NO: 176 - protein, 104 aa, synthetic construct
  REGION 1..104: note = Synthetic
IDTMSGLSGETGQSGNTTIEEDSTTHVKFSKRDSNGKELAGAMIELRNLSGQTIQSWVSDGTVKDFYLMPGTYQFVETAAPEGYELAAPITFTIDEKGQIWVDS

SEQ ID NO: 177 - DNA, 318 bp, synthetic construct
  misc_feature 1..318: note = Synthetic
  source 1..318: mol_type = other DNA
atgggtattgataccatgagcggtctgagcggtgaaaccggtcagagcggtaataccaccattgaagaggatagcaccacacatgtgaaattcagcaaacgcgatgcaaacggcaaagaactggcaggcgcaatgattgaactgcgtaatctgagtggtcagaccattcagagctgggttagtgatggcaccgttaaagatttttatctgatgcctggcacctatcagtttgttgaaaccgcagcaccggaaggttatgagctggcagcaccgattacctttaccattgatgaaaaaggtcagatttgggttgatagc

SEQ ID NO: 178 - protein, 123 aa, synthetic construct
  REGION 1..123: note = Synthetic
SSGLVPRGSHMKPLRGAVESLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 179 - DNA, 369 bp, synthetic construct
  misc_feature 1..369: note = Synthetic
  source 1..369: mol_type = other DNA
agcagcggcctggtgccgcgcggcagccatatgaagccgctgcgtggtgccgtgtttagcctgcagaaacagcatcccgactatcccgatatctatggcgcgattgatcagaatgggacctatcaaaatgtgcgtaccggcgaagatggtaaactgacctttaagaatctgagcgatggcaaatatcgcctgtttgaaaatagcgaacccgctggctataaaccggtgcagaataagccgattgtggcgtttcagattgtgaatggcgaagtgcgtgatgtgaccagcattgtgccgcaggatattccggctacatatgaatttaccaacggtaaacattatatcaccaatgaaccgataccgccgaaa

SEQ ID NO: 180 - protein, 129 aa, synthetic construct
  REGION 1..129: note = Synthetic
GSLSKYGKVILTKTGTDDLADKTKYNGAQFQVYECTKTASGATLRDSDPSTQTVDPLTIGGEKTFTTAGQGTVEINYLRANDYVNGAKKDQLTDEDYYCLVETKAPEGYNLQADPLPERVLAEKAEKKA

SEQ ID NO: 181 - DNA, 387 bp, synthetic construct
  misc_feature 1..387: note = Synthetic
  source 1..387: mol_type = other DNA
ggtagcctgagcaaatatggtaaagtgattctgaccaaaaccggcaccgatgatctggcagataaaaccaaatataacggtgcacagtttcaggtgtatgaatgtaccaaaacagcaagcggtgcaaccctgcgtgatagcgatccgagcacacagaccgttgatccgctgaccattggtggtgaaaaaacctttaccaccgcaggtcagggcaccgttgaaattaattatctgcgtgccaatgattatgtgaacggtgcaaaaaaagatcagctgaccgatgaagattattactgtctggttgaaaccaaagcaccggaaggttataatctgcaggcagatccgctgccgtttcgtgttctggccgaaaaagcagaaaaaaaagcc

SEQ ID NO: 182 - protein, 102 aa, synthetic construct
  REGION 1..102: note = Synthetic
GSTTKVKLIKVDQDHNRLEGVGFKLVSVARDVSAAAVPLIGEYRYSSSGQVGRTLYTDKNGEIFVTNLPLGNYRFKEVEPLAGYAVTTLDTDVQLVDHQLVT

SEQ ID NO: 183 - DNA, 306 bp, synthetic construct
  misc_feature 1..306: note = Synthetic
  source 1..306: mol_type = other DNA
ggtagcaccaccaaagtgaaactgattaaagttgatcaggatcacaatcgtctggaaggtgttggttttaaactggttagcgttgcacgtgatgttagcgcagcagcagttccgctgattggtgaatatcgttatagcagcagcggtcaggttggtcgtaccctgtataccgataaaaatggcgaaattttcgttaccaatctgccgctgggtaactatcgttttaaagaagttgaaccgctggcaggttatgcagttaccacactggataccgatgttcagctggttgatcatcagctggtgacc

SEQ ID NO: 184 - protein, 94 aa, synthetic construct
  REGION 1..94: note = Synthetic
PRGNVDFMKVDGRTNTSLQGAMEKVMKEESGHYTPVLQNGKEVVVTSGKDGRFRVEGLEYGTYYLWELQAPTGYVQLTSPVSFTIGKDTRKELV

SEQ ID NO: 185 - DNA, 282 bp, synthetic construct
  misc_feature 1..282: note = Synthetic
  source 1..282: mol_type = other DNA
ccgcgtggtaatgttgattttatgaaagttgatggtcgcaccaataccagcctgcagggtgcaatgtttaaagtgatgaaagaagaaagcggtcactatacaccggtgctgcagaatggtaaagaagttgttgttaccagcggtaaagatggtcgttttcgtgttgaaggtctggaatatggcacctattatctgtgggaactgcaggcaccgaccggttatgttcagctgaccagtccggttagttttaccattggcaaagatacccgtaaagaactggtg

SEQ ID NO: 186 - protein, 123 aa, synthetic construct
  REGION 1..123: note = Synthetic
VVTYHGKLKVVKKDGKEAGKVLKGAEFELYQCTSAAVLGKGPLTVDGVKKWTTGDDGTFTIDGLHVTDFEDGKEAAPATKKFCLKETKAPAGYALPDPNVTEIEFTRAKISEKDKFEGDDEVT

SEQ ID NO: 187 - DNA, 369 bp, synthetic construct
  misc_feature 1..369: note = Synthetic
  source 1..369: mol_type = other DNA
gttgttacctatcatggtaaactgaaagtggtgaaaaaagacggtaaagaggcaggcaaagttctgaaaggtgcagaatttgaactgtatcagtgtaccagcgcagcagttttaggtaaaggtccgctgaccgttgatggtgtgaaaaaatggaccaccggtgatgatggcacctttaccattgatggtctgcatgttaccgattttgaagatggtaaagaagccgcaccggcaaccaaaaaattctgtctgaaagaaaccaaagcaccggcaggttatgcactgcctgatccgaatgtgaccgaaattgaatttacccgtgcaaaaatcagcgagaaagataaatttgaaggcgacgatgaagtgacc

SEQ ID NO: 188 - protein, 134 aa, synthetic construct
  REGION 1..134: note = Synthetic
STNDTTTQNVVLTKYGEDKDVTAIDRATDQIWTGDGAKPLQGVDFTIYNVTANYWASPKDYKGSFDSAPVAATGTINDGNVYKGQLTQALPIQSKDASGKTRAAVYLFHETNPRAGYNTSADFWLTLPAKAAAD

SEQ ID NO: 189 - DNA, 402 bp, synthetic construct
  misc_feature 1..402: note = Synthetic
  source 1..402: mol_type = other DNA
agcaccaatgataccaccacacagaatgttgttctgaccaaatatggcttcgataaagatgttaccgcaattgatcgtgcaaccgatcagatttggaccggtgatggtgcaaaaccgctgcagggtgttgattttaccatttataacgtgaccgccaattattgggcaagcccgaaagattataaaggcagctttgatagcgcaccggttgcagccaccggtacaacaaatgataaaggccagctgacccaggcactgccgattcagagcaaagatgcaagcggtaaaacccgtgcagcagtttacctgtttcacgaaaccaatccgcgtgcaggttataataccagcgcagatttttggctgaccctgcctgcaaaagcagcagcagatggtaatgtttat

SEQ ID NO: 190 - protein, 114 aa, synthetic construct
  REGION 1..114: note = Synthetic
TTYERTFVKKDAETKEVLEGAGFKISNSDGKFLKLTDKDGQSVSIGEGFIDVLANNYRLTWVAESDATVFTSDKSGKFGLNGFADNTTTYTAVETNVPDGYDAAANTDEKADNS

SEQ ID NO: 191 - DNA, 342 bp, synthetic construct
  misc_feature 1..342: note = Synthetic
  source 1..342: mol_type = other DNA
accacctatgaacgtacctttgttaaaaaagacgccgaaaccaaagaagttctggaaggcgcaggctttaaaatcagcaatagtgatggcaaattcctgaaactgaccgataaagatggtcagagcgttagcattggtgaaggttttattgatgttctggccaataactatcgtctgacctgggttgcagaaagtgatgcaaccgtttttaccagcgataaaagcggcaaatttggtctgaatggttttgcagataataccaccacctataccgcagttgaaaccaatgttccggatggttatgatgcagcagcaaacaccgatttcaaagccgataatagc

SEQ ID NO: 192 - protein, 107 aa, synthetic construct
  REGION 1..107: note = Synthetic
GQITIKKIDGSTKASLQGAIFVLKNATGQFLNENDTNNVEWGTEANATEYTTGADGIITITGLKEGTYYLVEKKAPLGVNPYNLLDNSQKVILGDGATDTTNSDNLL

SEQ ID NO: 193 - DNA, 321 bp, synthetic construct
  misc_feature 1..321: note = Synthetic
  source 1..321: mol_type = other DNA
ggtcagattaccatcaaaaaaatcgatggtagcaccaaagcaagcctgcagggtgcaatttttgttctgaaaaatgcaaccggtcagttcctgaattttaacgataccaataatgttgaatggggcaccgaagcaaatgccaccgaatataccaccggtgcagatggtattattaccattaccggtctgaaagaaggcacctattacctggttgaaaaaaaagcaccgctgggttataatctgctggataattcacagaaagtgattttaggtgatggtgcaaccgataccaccaatagcgataacctgctggttaatccg

SEQ ID NO: 194 - protein, 91 aa, synthetic construct; identical to SEQ ID NO: 114
  REGION 1..91: note = Synthetic

SEQ ID NO: 195 - DNA, 273 bp, synthetic construct
  misc_feature 1..273: note = Synthetic
  source 1..273: mol_type = other DNA
gaacaggatgttgtgtttagcaaagttaatgttgccggtgaagaaattgcgggtgcaaaaatccagctgaaagatgcacagggtcaagttgttcatagctggaccagcaaagcaggtcagagcgaaaccgttaaactgaaagcaggcacctatacctttcatgaagcaagcgcaccgaccggttatctggcagttaccgatattacctttgaagttgatgttcagggtaaagtgaccgttaaagatgcaaatggtaatggtgtgaaagccgac

SEQ ID NO: 196 - protein, 104 aa, synthetic construct
  REGION 1..104: note = Synthetic
KLGDIEFIKVNKNDKKPLRGAVESLQKQHPDYPDIYGAIDQNGTYQNVRTGEDGKLTFKNLSDGKYRLFENSEPAGYKPVQNKPIVAFQIVNGEVRDVTSIVPQ

SEQ ID NO: 197 - DNA, 312 bp, synthetic construct
  misc_feature 1..312: note = Synthetic
  source 1..312: mol_type = other DNA
aaactgggtgatattgagttcatcaaagtgaacaaaaacgataaaaaaccgctgcgtggtgcagtttttagcctgcagaaacagcatccggattacccggatatttatggtgcaattgatcagaatggcacctatcagaatgttcgtaccggtgaagatggtaaactgacctttaaaaacctgagcgacggtaaatatcgcctgtttgaaaatagcgaaccggcaggttataaaccggttcagaataaaccgattgtggcctttcagattgttaatggtgaagttcgtgatgtgaccagcattgttccgcag

SEQ ID NO: 198 - protein, 130 aa, synthetic construct
  REGION 1..130: note = Synthetic
GSERKGSLTLHKKKGAESEKRATGKEMDDVAGEPLNGVTFKITKLNEDLQNGDWAKFPKTAADAKGHETSTTKEVETSGNGTAVFDNLDLGIYLVEETKAPDGIVTGAPFIVSIPMVNEASDAWNYNVVA

SEQ ID NO: 199 - DNA, 390 bp, synthetic construct
  misc_feature 1..390: note = Synthetic
  source 1..390: mol_type = other DNA
ggtagcgaacgtaaaggtagtctgaccctgcataaaaagaaaggtgcagaaagcgaaaaacgtgcaaccggtaaagaaatggatgatgttgccggtgaaccgctgaatggtgttacctttaaaatcaccaaactgaacttcgatctgcagaatggtgattgggcaaaatttccgaaaaccgcagcagatgcaaaaggtcatgaaaccagcaccaccaaagaagtggaaaccagcggtaatggcaccgcagtttttgataatctggatctgggtatttacctggtggaagaaaccaaagcaccggatggtattgttacaggtgcaccgtttattgttagcattccgatggttaatgaagcaagtgatgcctggaattataacgttgttgca

SEQ ID NO: 200 - protein, 149 aa, synthetic construct
  REGION 1..149: note = Synthetic
NLPEVKDGTLRTTVIADGVNGSSEKEALVSFENSKDGVDVKDTINYEGLVANQNYTLTGTLMHVKADGSLEEIATKTTNVTAGENGNGTWGLDFGNQKLQVGEKYVVFENAESVENLIDTDKDYNLDTKQVVKHEDKNDKAQTLVVEKP

SEQ ID NO: 201 - DNA, 447 bp, synthetic construct
  misc_feature 1..447: note = Synthetic
  source 1..447: mol_type = other DNA
aacctgcccgaggtgaaggacggcaccctgaggaccaccgtgatcgccgacggcgtgaacggcagcagcgagaaggaggccctggtgagcttcgagaacagcaaggacggcgtggacgtgaaggacaccatcaactacgagggcctggtggccaaccagaactacaccctgaccggcaccctgatgcacgtgaaggccgacggcagcctggaggagatcgccaccaagaccaccaacgtgaccgccggcgagaacggcaacggcacctggggcctggacttcggcaaccagaagctgcaggtgggcgagaagtacgtggtgttcgagaacgccgagagcgtggagaacctgatcgacaccgacaaggactacaacctggacaccaagcaggtggtgaagcacgaggacaagaacgacaaggcccagaccctggtggtggagaagccc

SEQ ID NO: 202 - DNA, 3379 bp, unidentified
  misc_feature 1..3379: note = Herpes simplex virus type 1
  source 1..3379: mol_type = genomic DNA
atggcggcggccggcggggagcgccagctagacggacagaaacccggcccgccgcaccttcagcaacccggggaccgaccagccgttccagggagggccgaggcctttttaaattttacgtctatgcacggggtgcagccaatccttaagcgcatccgagagctctcgcaacaacagctcgacggagcgcaagtgccccatctgcagtggttccgggacgtggcggccttagagtcccccgcaggcctgcccctcagggagtttccgttcgcggtgtatcttatcaccggcaacgctggctccggaaagagcacgtgcgtgcagacaatcaacgaggtcttggactgtgtggtgacgggcgccacgcgcattgcggcccaaaacatgtacgccaaactctcgggcgcctttctcagccgacccatcaacaccatctttcatgaatttgggtttcgcgggaatcacgtccaggcccaactgggacagtacccgtacaccctgaccagcaaccccgcctcgctggaggacctgcagcgacgagatctgacgtactactgggaggtgattttggacctcacgaagcgcgccctggccgcctccgggggcgaggagttgcggaacgagtttcgcgccctggccgccctggaacggaccctggggttggccgagggcgccctgacgcggttggccccggccacccacggggcgctgccggcctttacccgcagcaacgtgatcgtcatcgacgaggccgggctccttgggcgtcacctcctcacggccgtggtgtattgctggtggatgattaacgccctgtaccacaccccccagtacgcggcccgcctgcggcccgtgttggtgtgtgtgggctcgccgacgcagacggcgtccctggagtcgaccttcgagcaccagaaactgcggtgttccgtccgccagagcgagaacgtgctcacgtacctcatctgcaaccgcacgctgcgcgagtacgcccgcctctcgtatagctgggccatttttattaacaacaaacggtgcgtcgagcacgagttcggtaacctcatgaaggtgctggagtacggcctgcccatcaccgaggagcacatgcagttcgtggatcgcttcgtcgtcccggaaaactacatcaccaaccccgccaacctccccggctggacgcggctgttctcctcccacaaagaggtgagcgcgtacatggccaagctccacgcctacctgaaggtgacccgtgagggggagttcgtcgtgttcaccctccccgtgcttacgttcgtgtcggtcaaggagtttgacgaataccgacggctgacacaccagcccggcctgacgattgaaaagtggctcacggccaacgccagccgcatcaccaactactcgcagagccaggaccaggacgcggggcacatgcgctgcgaggtgcacagcaaacagcagctggtcgtggcccgcaacgacgtcacttacgtcctcaacagccagatcgcggtgaccgcgcgcctgcgaaaactggtttttgggtttagtgggacgttccgggccttcgaggcagtgttgcgtgacgacagctttgtaaagactcagggggagacttcggtggagtttgcctacaggttcctgtcgcggctcatatttagcgggcttatctccttttacaactttctgcagcgcccgggcctggatgcgacccagaggaccctcgcctacgcccgcatgggagaactaacggcggagattctgtctctgcgccccaaatcttcgggggtgccgacgcaggcgtcggtaatggccgacgcaggcgcccccggcgagcgtgcgtttgattttaagcaactggggccgcgggacgggggcccggacgattttcccgacgacgacctcgacgttattttcgcggggctggacgaacaacagctcgacgtgttttactgccactacacccccggggaaccggagaccaccgccgccgttcacacccagtttgcgctgctgaagcgggccttcctcgggagattccgaatcctccaagagctcttcggggaggcatttgaagtcgccccctttagcacgtacgtggacaacgttatcttccggggctgcgagatgctgaccggctcgccgcgcggggggctgatgtccgtcgccctgcagacggacaattatacgctcatgggatacacgtacgcacgggtgtttgcctttgcggacgagctgcggaggcggcacgcgacggccaacgtggccgagttactggaagaggcccccctgccttacgtggtcttgcgggaccaacacggcttcatgtccgtcgtcaacaccaacatcagcgagtttgtcgagtccattgactctacggagctggccatggccataaacgccgactacggcatcagctccaagcttgccatgaccatcacgcgctcccagggccttagcctggacaaggtcgccatctgctttacgcccggcaacctgcgcctcaacagcgcgtacgtggccatgtcccgcaccacctcctccgaattccttcgcatgaacttaaatccgctccgggagcgccacgagcgcgatgacgtcattagtgagcacatactatcggctctgcgcgatccgaacgtggtcattgtctattaacccgccgtccccttacagttccaccgaacccggcccgggggactcactacccaccgcgagatgtccaatccacagacgaccatcgcgtatagcctatgccacgccagggcctcgctgaccagcgcactgcccgacgccgcgcaggtggtgcatgtttttgagtacggcacccgcgcgatcatggtacggggccgggagcgccaggaccgcctgccgcgcggaggcgttgttatccagcacacccccattgggctgttggtgattatcgactgtcgcgccgaattttgtgcctaccgctttataggccgggacagcaaccagaagctcgaacgcgggtgggacgcccatatgtacgcgtatccgttcgactcctgggtcagctcctcgcgcggcgaaagcgcccggagcgccacggccggcattttgaccgtggtctggaccgcggacaccatttacatcactgcaaccatttacgggtcgcccccagaggagacgccaggcgcggcacacggggtgggcgccgcgcctccacccccgacaaccgcctgccccgggacggccgagtttctccagcccaccgcggacctgctggtagaggtgctgcgggagattcaactgagccccgccctggaatacgcagacaaacttttggggtcctaggatcccggccggatcgcgctcgtcacccgacactgaaatgcccccccccccttgcgggcggtccattaaa

SEQ ID NO: 203 - DNA, 2267 bp, unidentified
  misc_feature 1..2267: note = Herpes simplex virus type 1
  source 1..2267: mol_type = genomic DNA
atggacaccgcagatatcgtgtgggtggaggagagcgtcagcgccattaccctttacgcggtatggctgcccccccgcgctcgcgagtacttccacgccctggtgtattttgtatgtcgcaacgccgcaggggagggtcgcgcgcgctttgcggaggtctccgtcaccgcgacggagctgcgggatttctacggctccgcggacgtctccgtccaggccgtcgtggcggccgcccgcgccgcgacgacgccggccgcctccccgctggagcccctggagaacccgactctgtggcgggcgctgtacgcgtgcgtcctggcggccctggagcgccagaccgggccggtggccctgttcgccccgctgcgtatcggctcggacccacgcacgggactggtggtgaaagttgagagagcgtcgtggggcccgcccgccgcccctcgcgccgctctcctggtcgcggaggccaacattgacatcgaccctatggccctggcggcgcgcgttgccgagcatcccgacgcgcggctggcgtgggcgcgcctggcggccattcgcgacaccccccagtgcgcgtccgccgcttcgctgaccgttaacatcaccaccggaaccgcgctatttgcgcgcgaataccagactcttgcgtttccgccgatcaagaaggagggcgcgttcggggacctggtcgaggtgtgcgaggtgggcctgcggccacgcgggcacccgcaacgagtcacggcacgggtgctgctgccccgcgattacgactactttgtaagcgccggcgagaagttctccgcgccggcgctcgtcgcccttttccggcagtggcataccacggtccacgccgcccccggggccctggcccccgtctttgcctttctggggcccgagtttgaggtccgggggggacccgtcccgtactttgccgtcctggggtttccgggttggcccacgttcaccgtgccggccacggccgagtcggcacgggacctggtgcgcggggccgcggccgcttacgccgcgctcctgggggcctggcccgcggtgggggccagggtcgtcctccccccgcgagcctggcccggcgtggcctcggcggcagccggatgcctcctgcccgcggtgcgggaggcggtggcgcggtggcatcccgccactaaaatcatccaactgttagacccgcccgcggccgtcgggcccgtctggacggcgcggttttgcttccccggacttcgcgcccagctcctggcggccctggccgacctcggggggagcgggctggcggacccccacggccggacgggcctagcaagactggacgcgctggtggtggccgctccctcagagccctgggccggggccgtcttggagcgcctggtcccggacacgtgcaacgcctgccctgcgctgcggcagctcctgggtggggtaatggccgccgtctgcctgcagatcgaggagacggccagctcggtgaagttcgcggtctgcgggggcgatgggggtgcgttctggggtgtctttaacgtggacccccaagacgcggatgcggcttccggggtgatcgaggacgcccggcgggccatcgagacggccgtgggagccgtgcttagggccaacggcctccggctgcggcacccactgtgcctggccctcgagggcgtctacacccacgcagtcgcctggagccaggcgggagtgtggttctggaactcccgcgacaacactgaccatcttgggggatttcctctccgcgggcccgcgtacaccacggcggcaggggtcgtacgcgacacgctgcgacgggtcctgggcctgacaacggcatgcgtgccggaggaggacgcactcacggcccggggccttatggaggacgcctgcgaccgccttatcttggacgcgtttaataaacggttggacgcggagtactggagcgttcgggtgtccccctttgaggccagcgaccccttgccccccactgccttccgcggcggcgccttgctggacgcagagcactactggcggcgcgtcgtgcgtgtctgtcccggaggcggggagtcggtcggcgtccccgtcgatctatacccgcggccccttgtgctcccccccgtggactgcgctcatcacctgcgcgaaatcctgcgcgagattgagttggtgtttaccggggtgctggcgggagtatggggcgagggggggaagtttgtgtatccctttgacgacaagatgtcgtttctgtttgcctgagtttgaccaataaa

SEQ ID NO: 204 - DNA, 4401 bp, unidentified
  misc_feature 1..4401: note = Herpes simplex virus type 1
  source 1..4401: mol_type = genomic DNA
atggggcaggaagacgggaaccgcggggagaggcgggcggccgggactcccgtggaggtgaccgcgctttatgcgaccgacgggtgcgttattacctcttcgatcgccctcctcacaaactctctactgggggccgagccggtttatatattcagctacgacgcatacacgcacgatggccgtgccgacgggcccacggagcaagacaggttcgaagagagtcgggcgctctaccaagcgtcgggcgggctaaatggcgactccttccgagtaaccttttgtttattggggacggaagtgggtgggacccaccaggcccgcgggcgaacccgacccatgttcgtctgtcgcttcgagcgagcggacgacgtcgccgcgctacaggacgccctggcgcacgggaccccgctacaaccggaccacatcgccgccaccctggacgcggaggccacgttcgcgctgcatgcgaacatgatcctggctctcaccgtggccatcaacaacgccagcccccgcaccggacgcgacgccgccgcggcgcagtatgatcagggcgcgtccctacgctcgctcgtggggcgcacgtccctgggacaacgcggccttaccacgctatacgtccaccacgaggtgcgcgtgcttgccgcgtaccgcagggcgtattatggaagcgcgcagagtcccttctggtttcttagcaaattcgggccggacgaaaaaagcctggtgctcaccactcggtactacctgcttcaggcccagcgtctggggggcgcgggggccacgtacgacctgcaggccatcaaggacatctgcgccacctacgcgattccccacgccccccgccccgacaccgtcagcgctgcgtccctgacctcgtttgccgccatcacgcggttctgttgcacgagccagtacgcccgcggggccgcggcggccgggtttccgctttacgtggagcgccgtattgcggccgacgtccgcgagaccagtgcgctggagaagttcataacccacgatcgcagttgcctgcgcgtgtccgaccgtgaattcattacgtacatctacctggcccattttgagtgtttcagccccccgcgcctagccacgcatcttcgggccgtgacgacccacgaccccaaccccgcggccagcacggagcagccctcgcccctgggcagggaggccgtggaacaatttttttgtcacgtgcgcgcccaactgaatatcggggagtacgtcaaacacaacgtgaccccccgggagaccgtcctggatggcgatacggccaaggcctacctgcgcgctcgcacgtacgcgcccggggccctgacgcccgcccccgcgtattgcggggccgtggactccgccaccaaaatgatggggcgtttggcggacgccgaaaagctcctggtcccccgcgggtggcccgcgtttgcgcccgccagtcccggggaggacacggcgggcggcacgccgcccccacagacctgcggaattgtcaagcgcctcctgagactggccgccacggaacagcagggccccacacccccggcgatcgcggcgcttatccgtaatgcggcggtgcagactcccctgcccgtctaccggatatccatggtccccacgggacaggcatttgccgcgctggcctgggacgactgggcccgcataacgcgggacgctcgcctggccgaagcggtcgtgtccgccgaagcggcggcgcaccccgaccacggcgcgctgggcaggcggctcacggatcgcatccgcgcccagggccccgtgatgccccctggcggcctggatgccggggggcagatgtacgtgaatcgcaacgagatattcaacggcgcgctggcaatcacaaacatcatcctggatctcgacatcgccctgaaggagcccgtcccctttcgccggctccacgaggccctgggccactttaggcgcggggctctggctgcggttcagctcctgtttcccgcggcccgcgtggaccccgacgcatatccctgttattttttcaaaagcgcatgtcggcccggcccggcgtccgtgggttccggcagcggactcggcaacgacgacgacggggactggtttccctgctacgacgacgccggtgatgaggagtgggcggaggacccgggcgccatggacacatcccacgatcccccggacgacgaggttgcctactttgacctgtgccacgaagtcggccccacggcggaacctcgcgaaacggattcgcccgtgtgttcctgcaccgacaagatcggactgcgggtgtgcatgcccgtccccgccccgtacgtcgtccacggttctctaacgatgcggggggtggcacgggtcatccagcaggcggtgctgttggaccgagattttgtggaggccatcgggagctacgtaaaaaacttcctgttgatcgatacgggggtgtacgcccacggccacagcctgcgcttgccgtattttgccaaaatcgcccccgacgggcctgcgtgcggaaggctgctgccagtgtttgtgatcccccccgcctgcaaagacgttccggcgtttgtcgccgcgcacgccgacccgcggcgcttccattttcacgccccgcccacctatctcgcttccccccgggagatccgtgtcctgcacagcctgggtggggactatgtgagcttctttgaaaggaaggcgtcccgcaacgcgctggaacactttgggcgacgcgagaccctgacggaggtcctgggtcggtacaacgtacagccggatgcgggggggaccgtcgaggggttcgcatcggaactgctggggcggatagtcgcgtgcatcgaaacccactttcccgaacacgccggcgaatatcaggccgtatccgtccggcgggccgtcagtaaggacgactgggtcctcctacagctagtccccgttcgcggtaccctgcagcaaagcctgtcgtgtctgcgctttaagcacggccgggcgagtcgcgccacggcgcggacattcgtcgcgctgagcgtcggggccaacaaccgcctgtgcgtgtccttgtgtcagcagtgctttgccgccaaatgcgacagcaaccgcctgcacacgctgtttaccattgacgccggcacgccatgctcgccgtccgttccctgcagcacctctcaaccgtcgtcttgataacggcgtacggcctcgtgctcgtgtggtacaccgtcttcggtgccagtccgctgcaccgatgtatttacgcggtacgccccaccggcaccaacaacgacaccgccctcgtgtggatgaaaatgaaccagaccctattgtttctgggggccccgacgcacccccccaacgggggctggcgcaaccacgcccatatctgctacgccaatcttatcgcgggtagggtcgtgcccttccaggtcccacctgacgccatgaatcgtcggatcatgaacgtccacgaggcagttaactgtctggagaccctatggtacacacgggtgcgtctggtggtcgtagggtggttcctgtatctggcgttcgtcgccctccaccaacgccgatgtatgtttggcgtcgtgagtcccgcccacaagatggtggccccggccacctacctcttgaactacgcaggccgcatcgtatcgagcgtgttcctgcagtacccctacacgaaaattacccgcctgctctgcgagctgtcggtccagcggcaaaacctggttcagttgtttgagacggacccggtcaccttcttgtaccaccgccccgccatcggggtcatcgtaggctgcgagttgatgctacgctttgtggccgtgggtctcatcgtcggcaccgctttcatatcccggggggcatgtgcgatcacataccccctgtttctgaccatcaccacctggtgttttgtctccaccatcggcctgacagagctgtattgtattctgcggcggggcccggcccccaagaacgcagacaaggccgccgccccggggcgatccaaggggctgtcgggcgtctgcgggcgctgctgttccatcatcctctcgggcatcgcagtgcgattgtgttatatcgccgtggtggccggggtggtgctcgtggcgcttcactacgagcaggagatccagaggcgcctgtttgatgtatgacgtcacatccaggccggcggaaaccgtaacggcatatgcaaattggaaactgtcctgtcttggggcccacccacccgacgcgtcatatgcaaatgaaaatcggtcccccgaggccacgtgtagcctggatcccaacgaccccgcccatgggtcccaattggccgtcccgttaccaagaccaacccagccagcgtatccacccccgcccgggtccccgcggaagcggaacggggtatgtgatatgctaattaaa

SEQ ID NO: 205 - DNA, 3645 bp, unidentified
  misc_feature 1..3645: note = Herpes simplex virus type 1
  source 1..3645: mol_type = genomic DNA
atggagacaaagcccaagacggcaaccaccatcaaggtcccccccgggcccctgggatacgtgtacgctcgcgcgtgtccgtccgaaggcatcgagcttctggcgttactgtcggcacgcagcggcgattccgacgtcgccgtggcgcccctggtcgtgggcctgaccgtggagagcggctttgaggccaacgtggccgtggtcgtgggttctcgcacgacggggctcgggggtaccgcggtgtccctgaaactgacgccctcgcactacagctcgtccgtgtacgtctttcacggcggccggcacctggaccccagcacccaggccccgaacctgacgcgactttgcgagcgggcacgccgccattttggcttttcggactacaccccccggcccggcgacctcaaacacgagacgacgggggaggcgctgtgtgagcgcctcggcctggacccggaccgcgccctcctgtatctggtcgttaccgagggcttcaaggaggccgtgtgcatcaacaacacctttctgcacctgggaggctcggacaaggtaaccataggcggggcggaggtgcaccgcatacccgtgtacccgttgcagctgttcatgccggattttagccgtgtcatcgcagagccgttcaacgccaaccaccgatcgatcggggagaattttacctacccgcttccgttttttaaccgccccctcaaccgcctcctgttcgaggcggtcgtgggacccgccgccgtggcactgcgatgccgaaacgtggacgccgtggcccgcgcggccgcccacctggcgtttgacgaaaaccacgagggcgccgccctccccgccgacattacgttcacggccttcgaagccagccagggtaagaccccgcggggcgggcgcgacggcggcggcaagggcccggcgggcgggttcgaacagcgcctggcctccgtcatggccggagacgccgccctggccctcgagtctatcgtgtcgatggccgtctttgacgagccgcccaccgacatctccgcgtggccgctgttcgagggccaggacacggccgcggcccgcgccaacgccgtcggggcgtacctggcgcgcgccgcgggactcgtgggggccatggtatttagcaccaactcggccctccatctcaccgaggtggacgacgccggcccggcggacccaaaggaccacagcaaaccctccttttaccgcttcttcctcgtgcccgggacccacgtggcggccaacccacaggtggaccgcgagggacacgtggtgcccgggttcgagggtcggcccaccgcgcccctcgtcggcggaacccaggaatttgccggcgagcacctggccatgctgtgtgggttttccccggcgctgctggccaagatgctgttttacctggagcgctgcgacggcggcgtgatcgtcgggcgccaggagatggacgtgtttcgatacgtcgcggactccaaccagaccgacgtgccctgtaacctatgcaccttcgacacgcgccacgcctgcgtacacacgacgctcatgcgcctccgggcgcgccatccaaagttcgccagcgccgcccgcggagccatcggcgtcttcgggaccatgaacagcatgtacagcgactgcgacgtgctgggaaactacgccgccttctcggccctgaagcgcgcggacggatccgagaccgcccggaccatcatgcaggagacgtaccgcgcggcgaccgagcgcgtcatggccgaactcgagaccctgcagtacgtggaccaggcggtccccacggccatggggcggctggagaccatcatcaccaaccgcgaggccctgcatacggtggtgaacaacgtcaggcaggtcgtggaccgcgaggtggagcagctgatgcgcaacctggtggaggggaggaacttcaagtttcgcgacggtctgggcgaggccaaccacgccatgtccctgacgctggacccgtacgcgtgcgggccgtgccccctgcttcagcttctcgggcggcgatccaacctcgccgtgtaccaggacctggccctgagtcagtgccacggggtgttcgccgggcagtcggtcgaggggcgcaactttcgcaatcaattccaaccggtgctgcggcggcgcgtgatggacatgtttaacaacgggtttctgtcggccaaaacgctgacggtcgcgctctcggagggggcggctatctgcgcccccagcctaacggccggccagacggcccccgccgagagcagcttcgagggcgacgttgcccgcgtgaccctggggtttcccaaggagctgcgcgtcaagagccgcgtgttgttcgcgggcgcgagcgccaacgcgtccgaggccgccaaggcgcgggtcgccagcctccagagcgcctaccagaagcccgacaagcgcgtggacatcctcctcggaccgctgggctttctgctgaagcagttccacgcggccatcttccccaacggcaagcccccggggtccaaccagccgaacccgcagtggttctggacggccctccaacgcaaccagcttcccgcccggctcctgtcgcgcgaggacatcgagaccatcgcgttcattaaaaagttttccctggactacggcgcgataaactttattaacctggcccccaacaacgtgagcgagctggcgatgtactacatggcaaaccagattctgcggtactgcgatcactcgacatacttcatcaacacccttacggccatcatcgcggggtcccgccgtccccccagcgtgcaggctgcggccgcgtggtccgcgcagggcggggcgggcctggaggccggggcccgcgcgctgatggacgccgtggacgcgcatccgggcgcgtggacgtccatgttcgccagctgcaacctgctgcggcccgtcatggcggcgcgccccatggtcgtgttggggttgagcatcagcaagtactacggcatggccggcaacgaccgtgtgtttcaggccgggaactgggccagcctgatgggcggcaaaaacgcgtgcccgctccttatttttgaccgcacccgcaagttcgtcctggcctgtccccgggccgggtttgtgtgcgcggcctcaagcctcggcggcggagcgcacgaaagctcgctgtgcgagcagctccggggcattatctccgagggcggggcggccgtcgccagtagcgtgttcgtggcgaccgtgaaaagcctggggccccgcacccagcagctgcagatcgaggactggctggcgctcctggaggacgagtacctaagcgaggagatgatggagctgaccgcgcgtgccctggagcgcggcaacggcgagtggtcgacggacgcggccctggaggtggcgcacgaggccgaggccctagtcagccaactcggcaacgccggggaggtgtttaactttggggattttggctgcgaggacgacaacgcgacgccgttcggcggcccgggggccccgggaccggcatttgccggccgcaaacgggcgttccacggggatgacccgtttggggaggggccccccgacaaaaagggagacctgacgttggatatgctgtgaggggttggggggtgggggaacctagggcggggcggggaatgtgtgtaaaataaa

SEQ ID NO: 206 - DNA, 5050 bp, unidentified
  misc_feature 1..5050: note = Herpes simplex virus type 1
  source 1..5050: mol_type = genomic DNA
atgcctttcgtggggggcgcggagtcgggagatcctctgggggccgggcgtcccattggggacgacgagtgcgaacagtacacgtcgagcgtatcgctagcgcggatgttgtacgggggggatttggccgaatgggtgccccgggttcacccgaaaacaacgatcgagcggcagcagcacggacccgtcaccttccccaacgcgagcgccccgacggccaggtgcgtgactgtggtccgcgcgccaatggggtcgggaaaaactaccgcgctgatccgctggctgcgggaagcgatccactctccggacacgagtgtgctcgtcgtctcctgtcgtcggagttttacccagaccctagcgacgcggttcgctgagtcaggcctggtcgactttgtcacctacttctcatccaccaattacattatgaacgaccgccccttccaccgacttatcgtccaggtggaaagccttcatcgcgtgggccccaaccttctgaacaactacgacgtcctcgttctggacgaggttatgtcgacgctgggccagctctattcgccaacgatgcagcaactgggccgcgtggatgcgttaatgctacgcctgctgcgcacctgtcctcggatcatcgccatggacgcaaccgccaacgcgcagttggtggacttcctgtgcggtctccggggcgaaaaaaacgtgcatgtggtggtcggcgagtacgccatgcccgggttttcggcgcgccggtgcctgtttctcccgcgtctggggaccgagctcctgcaggctgccctgcgcccgcccgggccgccgagcggcccgtctccggacgcctctccggacgcccggggggccacgttctttggggagctggaagcgcgccttggcgggggcgataacatctgcattttttcgtcgacggtctccttcgcggagatcgtggcccggttctgccgtcagtttacggaccgcgtgctgttgcttcactcgctcacccccctcggggacgtgaccacgtggggccaataccgcgtggttatatacacgacggtcgtaaccgtgggcctcagcttcgatcccctgcactttgatggcatgttcgcctacgtgaaacccatgaactacggaccggacatggtgtccgtgtaccagtccctgggacgggtgcgcaccctccgcaagggggagctactgatttacatggacggctccggggcgcgctcggagcccgtctttacgcccatgctccttaatcacgtggtcagttcctgcggccagtggcccgcgcagttctcccaggtcacaaacctgctgtgtcgccggttcaaggggcgctgtgacgcgtcggcatgcgacacgtcgctggggcgggggtcgcgcatctacaacaaattccgttacaaacactactttgagagatgcacgctggcgtgtctctcggacagccttaacatccttcacatgctgctgaccctaaactgcatacgcgtgcgcttctggggacacgacgataccctgaccccaaaggacttctgtctgtttttgcggggcgtacatttcgacgccctcagggcccagcgcgatctacgggagctgcggtgccgggatcccgaggcgtcgctgccggcccaggccgccgagacggaggaggtgggtcttttcgtcgaaaaatacctccggtccgatgtcgcgccggcggaaattgtcgcgctcatgcgcaacctcaacagcctgatgggacgcacgcggtttatttacctggcgttgctggaggcctgtctccgcgttcccatggccacccgcagcagcgccatatttcggcggatctatgaccactacgccacgggcgtcatccccacgatcaacgtcaccggagagctggagctcgtggccctgccccccaccctgaacgtaacccccgtctgggagctgttgtgcctgtgcagcaccatggccgcgcgcctgcattgggactcggcggccgggggatctgggaggaccttcggccccgatgacgtgctggacctactgaccccccactacgaccgctacatgcagctggtgttcgaactgggccactgtaacgtaaccgacggacttctgctctcggaggaagccgtcaagcgcgtcgccgacgccctaagcggctgtcccccgcgcgggtccgttagcgagacggaccacgcggtggcgctgttcaagataatctggggcgaactgtttggcgtgcagatggccaaaagcacgcagacgtttcccggggcggggcgcgttaaaaacctcaccaaacagacaatcgtggggttgttggacgcccaccacatcgaccacagcgcctgccggacccacaggcagctgtacgccctgcttatggcccacaagcgggagtttgcgggcgcgcgcttcaagctacgcgtgcccgcgtgggggcgctgtttgcgcacgcactcatccagcgccaaccccaacgctgacatcatcctggaggcggcgctgtcggagctccccaccgaggcctggcccatgatgcagggggcggtgaactttagcaccctataagtctcgggaccgcactcgttcggtacgtggtcgtccgcggaccggcggcgctgttgccggaacgcaccgaggggccaagttggcccccggacccgggccgtttcccacccccaccccaaccccaaaaaccgccccccccccgtcaccggtttccgcgacccaccgggcccggccaggcacggcagcatgggacccacagaccgcccgtgatccttaggggccgtgcgatggacaccgcagatatcgtgtgggtggaggagagcgtcagcgccattaccctttacgcggtatggctgcccccccgcgctcgcgagtacttccacgccctggtgtattttgtatgtcgcaacgccgcaggggagggtcgcgcgcgctttgcggaggtctccgtcaccgcgacggagctgcgggatttctacggctccgcggacgtctccgtccaggccgtcgtggcggccgcccgcgccgcgacgacgccggccgcctccccgctggagcccctggagaacccgactctgtggcgggcgctgtacgcgtgcgtcctggcggccctggagcgccagaccgggccggtggccctgttcgccccgctgcgtatcggctcggacccacgcacgggactggtggtgaaagttgagagagcgtcgtggggcccgcccgccgcccctcgcgccgctctcctggtcgcggaggccaacattgacatcgaccctatggccctggcggcgcgcgttgccgagcatcccgacgcgcggctggcgtgggcgcgcctggcggccattcgcgacaccccccagtgcgcgtccgccgcttcgctgaccgttaacatcaccaccggaaccgcgctatttgcgcgcgaataccagactcttgcgtttccgccgatcaagaaggagggcgcgttcggggacctggtcgaggtgtgcgaggtgggcctgcggccacgcgggcacccgcaacgagtcacggcacgggtgctgctgccccgcgattacgactactttgtaagcgccggcgagaagttctccgcgccggcgctcgtcgcccttttccggcagtggcataccacggtccacgccgcccccggggccctggcccccgtctttgcctttctggggcccgagtttgaggtccgggggggacccgtcccgtactttgccgtcctggggtttccgggttggcccacgttcaccgtgccggccacggccgagtcggcacgggacctggtgcgcggggccgcggccgcttacgccgcgctcctgggggcctggcccgcggtgggggccagggtcgtcctccccccgcgagcctggcccggcgtggcctcggcggcagccggatgcctcctgcccgcggtgcgggaggcggtggcgcggtggcatcccgccactaaaatcatccaactgttagacccgcccgcggccgtcgggcccgtctggacggcgcggttttgcttccccggacttcgcgcccagctcctggcggccctggccgacctcggggggagcgggctggcggacccccacggccggacgggcctagcaagactggacgcgctggtggtggccgctccctcagagccctgggccggggccgtcttggagcgcctggtcccggacacgtgcaacgcctgccctgcgctgcggcagctcctgggtggggtaatggccgccgtctgcctgcagatcgaggagacggccagctcggtgaagttcgcggtctgcgggggcgatgggggtgcgttctggggtgtctttaacgtggacccccaagacgcggatgcggcttccggggtgatcgaggacgcccggcgggccatcgagacggccgtgggagccgtgcttagggccaacggcctccggctgcggcacccactgtgcctggccctcgagggcgtctacacccacgcagtcgcctggagccaggcgggagtgtggttctggaactcccgcgacaacactgaccatcttgggggatttcctctccgcgggcccgcgtacaccacggcggcaggggtcgtacgcgacacgctgcgacgggtcctgggcctgacaacggcatgcgtgccggaggaggacgcactcacggcccggggccttatggaggacgcctgcgaccgccttatcttggacgcgtttaataaacggttggacgcggagtactggagcgttcgggtgtccccctttgaggccagcgaccccttgccccccactgccttccgcggcggcgccttgctggacgcagagcactactggcggcgcgtcgtgcgtgtctgtcccggaggcggggagtcggtcggcgtccccgtcgatctatacccgcggccccttgtgctcccccccgtggactgcgctcatcacctgcgcgaaatcctgcgcgagattgagttggtgtttaccggggtgctggcgggagtatggggcgagggggggaagtttgtgtatccctttgacgacaagatgtcgtttctgtttgcctgagtttgaccaataaa

SEQ ID NO: 207 - DNA, 3747 bp, unidentified
  misc_feature 1..3747: note = Herpes simplex virus type 1
  source 1..3747: mol_type = genomic DNA
atgttttccggtggcggcggcccgctgtcccccggaggaaagtcggcggccagggcggcgtccgggttttttgcgcccgccggccctcgcggagccagccggggacccccgccttgtttgaggcaaaacttttacaacccctacctcgccccagtcgggacgcaacagaagccgaccgggccaacccagcgccatacgtactatagcgaatgcgatgaatttcgattcatcgccccgcgggtgctggacgaggatgcccccccggagaagcgcgccggggtgcacgacggtcacctcaagcgcgcccccaaggtgtactgcgggggggacgagcgcgacgtcctccgcgtcgggtcgggcggcttctggccgcggcgctcgcgcctgtggggcggcgtggaccacgccccggcggggttcaaccccaccgtcaccgtctttcacgtgtacgacatcctggagaacgtggagcacgcgtacggcatgcgcgcggcccagttccacgcgcggtttatggacgccatcacaccgacggggaccgtcatcacgctcctgggcctgactccggaaggccaccgggtggccgttcacgtttacggcacgcggcagtacttttacatgaacaaggaggaggtcgacaggcacctacaatgccgcgccccacgagatctctgcgagcgcatggccgcggccctgcgcgagtccccgggcgcgtcgttccgcggcatctccgcggaccacttcgaggcggaggtggtggagcgcaccgacgtgtactactacgagacgcgccccgctctgttttaccgcgtctacgtccgaagcgggcgcgtgctgtcgtacctgtgcgacaacttctgcccggccatcaagaagtacgagggtggggtcgacgccaccacccggttcatcctggacaaccccgggttcgtcaccttcggctggtaccgtctcaaaccgggccggaacaacacgctagcccagccgcgggccccgatggccttcgggacatccagcgacgtcgagtttaactgtacggcggacaacctggccatcgaggggggcatgagcgacctaccggcatacaagctcatgtgcttcgatatcgaatgcaaggcggggggggaggacgagctggcctttccggtggccgggcacccggaggacctggtcatccagatatcctgtctgctctacgacctgtccaccaccgccctggagcacgtcctcctgttttcgctcggttcctgcgacctccccgaatcccacctgaacgagctggcggccaggggcctgcccacgcccgtggttctggaattcgacagcgaattcgagatgctgttggccttcatgacccttgtgaaacagtacggccccgagttcgtgaccgggtacaacatcatcaacttcgactggcccttcttgctggccaagctgacggacatttacaaggtccccctggacgggtacggccgcatgaacggccggggcgtgtttcgcgtgtgggacataggccagagccacttccagaagcgcagcaagataaaggtgaacggcatggtgaacatcgacatgtacgggattataaccgacaagatcaagctctcgagctacaagctcaacgccgtggccgaagccgtcctgaaggacaagaagaaggacctgagctatcgcgacatccccgcctactacgccgccgggcccgcgcaacgcggggtgatcggcgagtactgcatacaggattccctgctggtgggccagctgttttttaagtttttgccccatctggagctctcggccgtcgcgcgcttggcgggtattaacatcacccgcaccatctacgacggccagcagatccgcgtctttacgtgcctgctgcgcctggccgaccagaagggctttattctgccggacacccaggggcgatttaggggcgccgggggggaggcgcccaagcgtccggccgcagcccgggaggacgaggagcggccagaggaggagggggaggacgaggacgaacgcgaggagggcgggggcgagcgggagccggagggcgcgcgggagaccgccggcaggcacgtggggtaccagggggccagggtccttgaccccacttccgggtttcacgtgaaccccgtggtggtgttcgactttgccagcctgtaccccagcatcatccaggcccacaacctgtgcttcagcacgctctccctgagggccgacgcagtggcgcacctggaggcgggcaaggactacctggagatcgaggtgggggggcgacggctgttcttcgtcaaggctcacgtgcgagagagcctcctcagcatcctcctgcgggactggctcgccatgcgaaagcagatccgctcgcggattccccagagcagccccgaggaggccgtgctcctggacaagcagcaggccgccatcaaggtcgtgtgtaactcggtgtacgggttcacgggagtgcagcacggactcctgccgtgcctgcacgttgccgcgacggtgacgaccatcggccgcgagatgctgctcgcgacccgcgagtacgtccacgcgcgctgggcggccttcgaacagctcctggccgatttcccggaggcggccgacatgcgcgcccccgggccctattccatgcgcatcatctacggggacacggactccatctttgtgctgtgccgcggcctcacggccgccgggctgacggccgtgggcgacaagatggcgagccacatctcgcgcgcgctgtttctgccccccatcaaactcgagtgcgaaaagacgttcaccaagctgctgctgatcgccaagaaaaagtacatcggcgtcatctacgggggtaagatgctcatcaagggcgtggatctggtgcgcaaaaacaactgcgcgtttatcaaccgcacctccagggccctggtcgacctgctgttttacgacgataccgtctccggagcggccgccgcgttagccgagcgccccgcggaggagtggctggcgcgacccctgcccgagggactgcaggcgttcggggccgtcctcgtagacgcccatcggcgcatcaccgacccggagagggacatccaggactttgtcctcaccgccgaactgagcagacacccgcgcgcgtacaccaacaagcgcctggcccacctgacggtgtattacaagctcatggcccgccgcgcgcaggtcccgtccatcaaggaccggatcccgtacgtgatcgtggcccagacccgcgaggtagaggagacggtcgcgcggctggccgccctccgcgagctagacgccgccgccccaggggacgagcccgccccccccgcggccctgccctccccggccaagcgcccccgggagacgccgtcgcctgccgaccccccgggaggcgcgtccaagccccgcaagctgctggtgtccgagctggccgaggatcccgcatacgccattgcccacggcgtcgccctgaacacggactattacttctcccacctgttgggggcggcgtgcgtgacattcaaggccctgtttgggaataacgccaagatcaccgagagtctgttaaaaaggtttattcccgaagtgtggcaccccccggacgacgtggccgcgcggctccggaccgcagggttcggggcggtgggtgccggcgctacggcggaggaaactcgtcgaatgttgcatagagcctttgatactctagcatgagccccccgtcgaagctgatgtccctcattttacaataaa

SEQ ID NO: 208 - DNA, 1526 bp, unidentified
  misc_feature 1..1526: note = Herpes simplex virus type 1
  source 1..1526: mol_type = genomic DNA
atgacggattcccctggcggtgtggcccccgcctcccccgtggaggacgcgtcggacgcgtccctcgggcagccggaggagggggcgccctgccaggtggtcctgcagggcgccgaacttaatggaatcctacaggcgtttgccccgctgcgcacgagccttctggactcgcttctggttatgggcgaccggggcatccttatccataacacgatctttggggagcaggtgttcctgcccctggaacactcgcaattcagtcggtatcgctggcgcggacccacggcggcgttcctgtctctcgtggaccagaagcgctccctcctgagcgtgtttcgcgccaaccagtacccggacctacgtcgggtggagttggcgatcacgggccaggccccgtttcgcacgctggttcagcgcatatggacgacgacgtccgacggcgaggccgttgagctagccagcgagacgctgatgaagcgcgaactgacgagctttgtggtgctggttccccagggaacccccgacgttcagttgcgcctgacgaggccgcagctcaccaaggtccttaacgcgaccggggccgatagtgccacgcccaccacgttcgagctcggggttaacggcaaattttccgtgttcaccacgagtacctgcgtcacctttgctgcccgcgaggagggcgtgtcgtccagcaccagcacccaggtccagatcctgtccaacgcgctcaccaaggcgggccaggcggccgccaacgccaagacggtgtacggggaaaatacccatcgcaccttctctgtggtcgtcgacgattgcagcatgcgggcggtgctccggcgactgcaggtcggcgggggcaccctcaagttcttcctcacgacccccgtccccagtctgtgcgtcaccgccaccggtcccaacgcggtatcggcggtatttctcctgaaaccccagaagatttgcctggactggctgggtcatagccaggggtctccttcagccgggagctcggcctcccgggcctctgggagcgagccaacagacagccaggactccgcgtcggacgcggtcagccacggcgatccggaagacctcgatggcgctgcccgggcgggagaggcgggggccttgcatgcctgtccgatgccgtcgtcgaccacgcgggtcactcccacgaccaagcgggggcgctcggggggcgaggatgcgcgcgcggacacggccctaaagaaacctaagacggggtcgcccaccgcacccccgcccgcagatccagtccccctggacacggaggacgactccgatgcggcggacgggacggcggcccgtcccgccgctccagacgcccggagcggaagccgttacgcgtgttactttcgcgacctcccgaccggagaagcaagccccggcgccttctccgccttccgggggggcccccaaaccccgtatggttttggattcccctgacggggcggggccttggcggccgcccaactctcgcaccatcccgggttaatgtaaataaa

SEQ ID NO: 209 - DNA, 1950 bp, unidentified
  misc_feature 1..1950: note = Human papillomavirus type 16
  source 1..1950: mol_type = genomic DNA
atggctgatcctgcaggtaccaatggggaagagggtacgggatgtaatggatggttttatgtagaggctgtagtggaaaaaaaaacaggggatgctatatcagatgacgagaacgaaaatgacagtgatacaggtgaagatttggtagattttatagtaaatgataatgattatttaacacaggcagaaacagagacagcacatgcgttgtttactgcacaggaagcaaaacaacatagagatgcagtacaggttctaaaacgaaagtatttgggtagtccacttagtgatattagtggatgtgtagacaataatattagtcctagattaaaagctatatgtatagaaaaacaaagtagagctgcaaaaaggagattatttgaaagcgaagacagcgggtatggcaatactgaagtggaaactcagcagatgttacaggtagaagggcgccatgagactgaaacaccatgtagtcagtatagtggtggaagtgggggtggttgcagtcagtacagtagtggaagtgggggagagggtgttagtgaaagacacactatatgccaaacaccacttacaaatattttaaatgtactaaaaactagtaatgcaaaggcagcaatgttagcaaaatttaaagagttatacggggtgagtttttcagaattagtaagaccatttaaaagtaataaatcaacgtgttgcgattggtgtattgctgcatttggacttacacccagtatagctgacagtataaaaacactattacaacaatattgtttatatttacacattcaaagtttagcatgttcatggggaatggttgtgttactattagtaagatataaatgtggaaaaaatagagaaacaattgaaaaattgctgtctaaactattatgtgtgtctccaatgtgtatgatgatagagcctccaaaattgcgtagtacagcagcagcattatattggtataaaacaggtatatcaaatattagtgaagtgtatggagacacgccagaatggatacaaagacaaacagtattacaacatagttttaatgattgtacatttgaattatcacagatggtacaatgggcctacgataatgacatagtagacgatagtgaaattgcatataaatatgcacaattggcagacactaatagtaatgcaagtgcctttctaaaaagtaattcacaggcaaaaattgtaaaggattgtgcaacaatgtgtagacattataaacgagcagaaaaaaaacaaatgagtatgagtcaatggataaaatatagatgtgatagggtagatgatggaggtgattggaagcaaattgttatgtttttaaggtatcaaggtgtagagtttatgtcatttttaactgcattaaaaagatttttgcaaggcatacctaaaaaaaattgcatattactatatggtgcagctaacacaggtaaatcattatttggtatgagtttaatgaaatttctgcaagggtctgtaatatgttttgtaaattctaaaagccatttttggttacaaccattagcagatgccaaaataggtatgttagatgatgctacagtgccctgttggaactacatagatgacaatttaagaaatgcattggatggaaatttagtttctatggatgtaaagcatagaccattggtacaactaaaatgccctccattattaattacatctaacattaatgctggtacagattctaggtggccttatttacataatagattggtggtgtttacatttcctaatgagtttccatttgacgaaaacggaaatccagtgtatgagcttaatgataagaactggaaatcctttttctcaaggacgtggtccagattaagtttgcacgaggacgaggacaaggaaaacgatggagactctttgccaacgtttaaatgtgtgtcaggacaaaatactaacacattatga

SEQ ID NO: 210 - DNA, 1098 bp, unidentified
  misc_feature 1..1098: note = Human papillomavirus type 16
  source 1..1098: mol_type = genomic DNA
atggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtggacattacaagacgttagccttgaagtgtatttaactgcaccaacaggatgtataaaaaaacatggatatacagtggaagtgcagtttgatggagacatatgcaatacaatgcattatacaaactggacacatatatatatttgtgaagaagcatcagtaactgtggtagagggtcaagttgactattatggtttatattatgttcatgaaggaatacgaacatattttgtgcagtttaaagatgatgcagaaaaatatagtaaaaataaagtatgggaagttcatgcgggtggtcaggtaatattatgtcctacatctgtgtttagcagcaacgaagtatcctctcctgaaattattaggcagcacttggccaaccaccccgccgcgacccataccaaagccgtcgccttgggcaccgaagaaacacagacgactatccagcgaccaagatcagagccagacaccggaaacccctgccacaccactaagttgttgcacagagactcagtggacagtgctccaatcctcactgcatttaacagctcacacaaaggacggattaactgtaatagtaacactacacccatagtacatttaaaaggtgatgctaatactttaaaatgtttaagatatagatttaaaaagcattgtacattgtatactgcagtgtcgtctacatggcattggacaggacataatgtaaaacataaaagtgcaattgttacacttacatatgatagtgaatggcaacgtgaccaatttttgtctcaagttaaaataccaaaaactattacagtgtctactggatttatgtctatatga

SEQ ID NO: 211 - DNA, 477 bp, unidentified
  misc_feature 1..477: note = Human papillomavirus type 16
  source 1..477: mol_type = genomic DNA
atgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaa

SEQ ID NO: 212 - DNA, 297 bp, unidentified
  misc_feature 1..297: note = Human papillomavirus type 16
  source 1..297: mol_type = genomic DNA
atgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataa

SEQ ID NO: 213 - DNA, 716 bp, synthetic construct
  misc_feature 1..716: note = Synthetic
  source 1..716: mol_type = other DNA
atgacgaagatgagctcagggaatatgaaagacaagcatcgctcctacaaaagaaaagggagtccagaaagaggggagaggaagagacactggcagacaactcatcacaggagcaggagccgcagcccgatccgacacagtggggagagaggctcgggctcatatcatcaggaacacccaatcagccacctattgtcttgcactgcttcgaagacctcagaccaagtgatgaagacgagggagagtacatcggggaaaaaagacaatagaacaaatccatacactgtattcagtcaacacagagcttccaatcctgaagctccagggtggtgtgggttctactggcactctactcgcattgctagagatggtactaattcaatctttaatgaaatgaaacaacagtttcaacagctacaaattgataataaaataggatgggataacactagagaactattgtttaatcaaaagaaaacactagatcaaaaatacagaaatatgttctggcactttagaaataactctgattgtgaaagatgtaattactgggatgatgtgtaccgtagacacttagctaatgtttcctcacagacagaagcagacgagataactgacgaggaaatgctttctgctgctgaaagcatggaagcagatgcctccaattaagagacagcctagagggtgggtgctgcctggatacagatatcttgggc

SEQ ID NO: 214 - DNA, 5299 bp, synthetic construct
  misc_feature 1..5299: note = Synthetic
  source 1..5299: mol_type = other DNA
gccggcagacatattggattccaagatggcgtctgtacaaccacgtcacatataaaataataaatattcacaaggaggagtggttatatgatgtaatccataaccactcccaggaaatgacgtatgatagccaatcagaattaagtattaaacctatataagctgctgcacttcctgattcaatcagactgcatccggtctccggcgagtgaacatctctggaaaaagctccacgcttgtggtgagtctactatggctttcaatcctcctgtgattagagctttttctcaacctgcttttacttatgtcttcaaatttccatatccacaatggaaagaaaaagaatggctgcttcatgcacttttagctcatggaactgaacaatctatgatacaattaagaaactgcgcttctcatccggatgaagacataatccgtgatgacttgcttatttctttagaagatcgccattttggggctgttctctgcaaggctgtttacatggcaacaactactctcatgtcacacaaacaaaggaatatgtttcctcgttgtgacatcatagttcagtctgagctaggagagaaaaacttacactgccatattatagttgggggagaaggactaagcaagaggaatgctaaatcatcctgtgctcagttctatggtttaatactagctgagataattcaacgctgcaaatctcttctggctacacgtccttttgaacctgaggaggctgacatatttcacactctaaaaaaggctgagcgagaggcatggggtggagttactggcggcaacatgcagatccttcaatatagagatcgcagaggagaccttcatgcacaaacagtggatcctcttcgcttcttcaaaaactaccttttacctaaaaatagatgtatttcatcttacagcaaacctgatgtttgtacttctcctgacaactggttcattttagctgaaaaaacttactctcacactcttattaacgggctgccgcttccagaacattacagaaaaaactaccacgcaaccctagataacgaagtcattccagggcctcaagcaatggcctatggaggacgtggtccgtgggaacatcttcctgaggtaggagatcagcgcctagctgcgtcttctgttagcactacttataaacctaacaaaaaagaaaaacttatgctaaacttgctagacaaatgtaaagagctaaatctattagtttatgaagacttagtagctaattgtcctgaactactccttatgcttgaaggtcaaccaggaggggcacgccttatagaacaagtcttgggcatgcaccatattaatgtttgttctaactttacagctctcacatatctttttcatctacatcctgttacttcgcttgactcagacaataaagctttacagcttttgttgattcaaggctataatcctctagccgttggtcacgccctgtgctgtgtcctgaacaaacaattcgggaaacaaaacactgtttgcttttacgggcctgcctcaacaggtaaaacaaatatggccaaggcaatcgtccaagggattagactttatgggtgtgttaatcatttgaacaaaggatttgtatttaatgactgcagacaacgcctagttgtttggtgggaggagtgcttaatgcaccaggattgggtggaacctgcaaagtgtatcttgggcgggacagaatgcagaattgacgtcaagcatagagacagtgtacttttaactcaaacacctgtaattatatccactaaccacgatatctacgcggttgttggtggcaattctgtttctcatgttcacgcggctccattaaaagaaagagtgattcagctaaattttatgaaacaacttcctcaaacatttggagaaatcactgctactgagattgcagctcttctacagtggtgtttcaatgagtacgactgtactctgacaggatttaaacaaaaatggaatttagacaaaattccaaactcatttcctcttggggtcctttgtcctactcattcacaggactttacacttcacgaaaacggatactgcactgattgcggtggttaccttcctcatagtgctgacaattctatgtacactgatcgcgcaagcgaaactagcacaggagacatcacaccaagtaagtaaatacgcatgcgcaagtaattcttttactttcacttcgctatttttaccaatttttacttttaggtgacttgggggattcggacggagaagacaccgagcctgagacatcgcaagtggactattgtccacccaagaaacgtcgtctaactgctccagcaagtcctccaaactcacctgcgagctctgtaagtactattactttctttaacacttggcacgcacagccacgtgacgaagatgagctcagggaatatgaaagacaagcatcgctcctacaaaagaaaagggagtccagaaagaggggagaggaagagacactggcagacaactcatcacaggagcaggagccgcagcccgatccgacacagtggggagagaggctcgggctcatatcatcaggaacacccaatcagccacctatcgtcttgcactgcttcgaagacctcagaccaagtgatgaagacgagggaaagtacatcggggaaaaaagacaatagaacaaatccatacactgtattcagtcaacacagagcttccaatcctgaagctccagggtggtgtgggttctactggcactctactcgcattgctagagatggtactaattcaatctttaatgaaatgaaacaacagtttcaacagctacaaattgataataaaataggatgggataacactagagaactattgtttaatcaaaagaaaacactagatcaaaaatacagaaatatgttctggcactttagaaataactctgattgtgaaagatgtaattactgggatgatgtgtaccgtagacacttagctaatgtttcctcacagacagaagcagacgagataactgacgaggaaatgctttctgctgctgaaagcatggaagcagatgcctccaattaagagacagcctagagggtgggtgctgcctggatacagatatcttgggccatttaatccacttgataacggtgaacctgtaaataacgctgatcgcgctgctcaattacatgatcacgcctactctgaactaataaagagtggtaaaaatccatacctgtatttcaataaagctgatgaaaaattcattgatgatctaaaagacgattggtcaattggtggaattattggatccagtttttttaaaataaagcgcgccgtggctcctgctttgggaaataaagagagagcccaaaaaagacacttttactttgctaactcaaataaaggtgcaaaaaaaacaaaaaaaagtgaacctaaaccaggaacctcaaaaatgtctgacactgacattcaagaccaacaacctgatactgtggacgcaccacaaaacacctcagggggaggaacaggaagtattggaggaggaaaaggatctggtgtggggatttccactggagggtgggtcggaggttctcacttttcagacaaatatgtggttactaaaaacacaagacaatttataaccacaattcaaaatggtcacctctacaaaacagaggccattgaaacaacaaaccaaagtggaaaatcacagcgctgcgtcacaactccatggacatactttaactttaatcaatacagctgtcacttctcaccacaggattggcagcgccttacaaatgaatataagcgcttcagacctaaagcaatgcaagtaaaaatttacaacttgcaaataaaacaaatactttcaaatggtgctgacacaacatacaacaatgacctcacagctggcgttcacatcttttgtgatggagagcatgcttacccaaatgcatctcatccatgggatgaggacgtcatgcctgatcttccatacaagacctggaaactttttcaatatggatatattcctattgaaaatgaactcgcagatcttgatggaaatgcagctggaggcaatgctacagaaaaagcacttctgtatcagatgcctttttttctacttgaaaacagtgaccaccaagtacttagaactggtgagagcactgaatttacttttaactttgactgtgaatgggttaacaatgaaagagcatacattcctcctggactaatgtttaatccaaaagtcccaacaagaagagttcagtacataagacaaaacggaagcacagcagccagcacaggcagaattcagccatactcaaaaccaacaagctggatgacaggacctggcctgctcagtgcacaaagagtaggaccacagtcatcagacactgctccattcatggtttgcactaacccagaaggaacacacataaacacaggtgctgcaggatttggatctggctttgatcctccaagcggatgtctggcaccaactaacctagaatacaaacttcagtggtaccagacaccagaaggaacaggaaataatggaaacataattgcaaacccatcactctcaatgcttagagaccaactcctatacaaaggaaaccagaccacatacaatctagtgggggacatatggatgtttccaaatcaagtctgggacagatttcctatcaccagagaaaatccaatctggtgcaaaaaaccaagggctgacaaacacacaatcatggatccatttgatggatcaattgcaatggatcatcctccaggcactatttttataaaaatggcaaaaattccagttccaactgcctcaaatgcagactcatacctaaacatatactgtactggacaagtcagctgtgaaattgtatgggaggtagaaagatacgcaacaaagaactggcgtccagaaagaagacatactgcactcgggatgtcactgggaggagaaagcaactacacgcctacataccacgtggatccaacaggagcatacatccagcccacgtcatatgatcagtgtatgccagtaaaaacaaacatcaataaagtgttgtaatcttataagcctcttttttgcttctgcttacaagttcctcctcaatggacaagcggaaagtgaagggtgactgtagtcctgagctcatgggttcaagaccacagcccgatggtagtggtgttaccgtctcgaacctagccgacagcccttgtacattgtggggggagctgttttgtttgcttatgcaatcgcgaaactctatatcttttaatgtgttgttgttgtaca